(19) United States
(12) Patent Application Publication
(10) Pub. No.: US 2009/0209536 A1
(43) Pub. Date: Aug. 20, 2009

(54) AMINOQUINAZOLINE CANNABINOID RECEPTOR MODULATORS FOR TREATMENT OF DISEASE

(75) Inventors: Timothy Gahman, San Diego, CA (US); David Thomas, San Diego, CA (US); Hengyuan Lang, San Diego, CA (US); Mark Eben Massari, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 12/140,033

(22) Filed: Jun. 16, 2008

Related U.S. Application Data
(60) Provisional application No. 60/944,510, filed on Jun. 17, 2007.
(60) Provisional application No. 61/037,347, filed on Mar. 18, 2008.

(51) Int. Cl. A61K 31/5377; C07D 239/72; A61K 31/517; A61P 25/02; A61K 31/497; C07D 413/04

(52) U.S. Cl. 514/234.5; 544/283; 544/116; 514/266.2; 514/252.17

(57) ABSTRACT

The present invention relates to compounds and methods useful as modulators of CB2 for the treatment or prevention of disease states including, but not limited to pain, autoimmune disease, malabsorption syndrome, pulmonary disease, osteoporosis, muscle spasm in cancer, neuromuscular disorder, and atherosclerosis progression.

This application claims the benefit of priority of U.S. provisional applications No. 60/944,510, filed Jun. 17, 2007, and No. 61/037,347, filed Mar. 18, 2008, the disclosures of which are hereby incorporated by reference as if written herein in their entirety.

Disclosed herein are new heterocyclic compounds and compositions and their application as pharmaceuticals for the treatment of disease. Methods of modulation of CB2 activity in a human or animal subject are also provided for the treatment diseases mediated by CB2.

Preparations of Cannabis sativa have been used for medicinal and recreational purposes for at least 4,000 years. Recently, cannabinoids have been the subject of renewed interest for their potential therapeutic applications (Mechoulam, R. in “Cannabinoids as Therapeutic Agents” CRC Press, Boca Raton, Fla., 1-19, 1986). The native active constituent, Delta 9-tetrahydrocannabinol (Δ9-THC), is prescribed today, under the generic name Dronabinol, as an anti-emetic and for enhancement of appetite, mainly in AIDS patients. However, separation between the clinically undesirable psychotropic effects and the therapeutically desirable effects, such as vascular hypotension and immunomodulation, has only recently been accomplished. The discovery and molecular cloning of the cannabinoid receptors has helped to elucidate the diverse cannabinoid effects.

Cannabinoids exert their effects by binding to specific receptors located in the cell membrane. Two types of high-affinity cannabinoid receptors have been identified to date by molecular cloning: 1) CB1 receptors (Devane et al., 1988, Mol. Pharmacol., 34:605-613; Matsuda et al., 1990, Nature, 346:561-564; Shire et al., 1995, J. Biol. Chem., 270:3726-3731; Ishac et al., 1996, Br. J. Pharmacol., 118:2023-2028), and 2) CB2 receptors (Munro et al., 1993, Nature, 365:61-65). CB1 and CB2, which share 44% identity at the amino acid level, are members of the G protein-coupled receptor (GPCRs) family. Both CB1 and CB2 couple to the inhibitory G-protein alpha-subunit Gi. Receptor activation thus leads to inhibition of adenylate cyclase as well as to activation of mitogen activated protein kinase (MAPK) (Parolaro, D., Life Sci. 65: 637-44, 1999). CB1 receptors can also modulate ion channels, inhibiting N—, and P/R-type calcium channels, stimulating inwardly rectifying K channels and enhancing the activation of the A-type K channel.

CB1 receptors are primarily, but not exclusively, expressed in the CNS and are believed to mediate the CNS effects of endogenous (e.g., anandamide, 2-arachidonoylglycerol [2-AG]) and exogenously applied cannabinoids. Peripheral areas of expression include, but are not restricted to, the pituitary gland, immune cells, reproductive tissues, gastrointestinal tissues, superior cervical ganglion, heart, lung, urinary bladder, and adrenal gland. CB1 receptors are also located on central and peripheral nerve terminals and, when activated, seem to suppress the neuronal release of a number of excitatory and inhibitory transmitters including acetylcholine, noradrenaline, dopamine, 5-hydroxytryptamine, γ-aminobutyric acid, glutamate and aspartate (Pertwee, 1997, Pharmacol. Ther., 129:74; Ong & Macide, 1999, Neuroscience, 92:1177; Pertwee, 2001, Progr. Neurobiol., 63:569). CB2 receptor expression was originally thought to be restricted to the periphery, mainly in lymphoid organs and cells of the immune system, including spleen, thymus, tonsils, bone marrow, pancreas and mast cells with particularly high levels in B-cells and natural killer cells (Galiégue et al., 1995, Bur. J. Biochem, 54:232). However, recent studies demonstrate that CB2 is expressed in the brain stem, cortex, cerebellum and hippocampus (Onaivi et al., 2006, Ann. N.Y. Acad. Sci., 1074:514-36; Van Sickle et al. 2005, Science, 310 329-32). In addition, there are both electophysiological and in situ hybridization data that demonstrate expression of CB2 receptors in the dorsal root ganglion and primary sensory afferent fibers in the spinal cord (Elmes et al., 2004, Eur. J. Neurosci. 20: 2311-20; Wotherspoon et al., 2005, Neuroscience 135: 235-45; Zhang et al., 2003, Eur. J. Neurosci. 17: 2750-54).

The location of CB2 receptors on the surface of immune cells suggests a role for these receptors in immunomodulation and inflammation. Endogenous cannabinoids have been shown to act as immuno-modulators, generally exerting a negative action on the onset of a variety of parameters of the immune response (Parolaro et al., 2002, Prostaglandins Leukot. Essent. Fatty Acids, 66:319-32). Previous studies have shown that the CB2 receptor plays a very important role in the stimulation of growth of several, if not all, hematopoietic lineages (Valk et al., 1997, Blood, 90:1448-1457; Derocq, 2000, J. Biol. Chem., 275: 15621-15628). The role of the endocannabinoid system in immunosuppression is the focus of many studies (Berdyshev, E. V., Chem. Phys. Lipids 108: 169-90, 2000). Anandamide, Palmitoylethanolamide (PEA) and 2-AG were shown to down-regulate the immune response in a variety of experimental systems and function as anti-inflammatory and immunosuppressive agents.

Analysis of the CB2 knockout mouse has corroborated the evidence for the function of CB2 receptors in modulating the immune system. CB2 does not affect immune cell development and differentiation as determined by FACS analysis of cells from the spleen, lymph node and thymus from CB2 knockout mice, but rather mediates the suppressive effect of Δ9-THC. Therefore, compounds that selectively interact with CB2 receptors offer a unique pharmacotherapy for the treatment of immune and inflammatory disorders.

The psychotropic side-effects caused by Δ9-THC and other nonselective CB agonists are mediated by CB1 receptors. CB1 knockout mice have been shown to be unresponsive to cannabinoids in behavioral assays providing molecular evidence that the psychotropic effects, including sedation, hallucinations and delirium and anti-nociception are manifested through activation of the CB1 receptor, present primarily in the CNS. These CB1 receptor-mediated effects have limited the development and clinical utility of nonselective CB agonists.

Pain is the most common symptom of disease and the most frequent complaint with which patients present to physicians. Pain is commonly segmented by duration (acute vs. chronic), intensity (mild, moderate, and severe), and type (nociceptive vs. neuropathic).

Nociceptive pain is the most well known type of pain, and is caused by tissue injury detected by nociceptors at the site of injury. After the injury, the site becomes a source of ongoing pain and tenderness. This pain and tenderness are considered “acute” nociceptive pain. This pain and tenderness gradually diminish as healing progresses and disappear when healing is complete. Examples of acute nociceptive pain include surgical procedures (post-op pain) and bone fractures. Even though there may be no permanent nerve damage, “chronic” nociceptive pain results from some conditions when pain extends beyond six months. Examples of chronic nociceptive pain include osteoarthritis, rheumatoid arthritis, and musculoskeletal conditions (e.g., back pain), cancer pain, etc.

Neuropathic pain is defined as “pain initiated or caused by a primary lesion or dysfunction in the nervous system” by the International Association for the Study of Pain. Neuropathic pain is not associated with nociceptive stimulation, although the passage of nerve impulses that is ultimately perceived as pain by the brain is the same in both nociceptive and neuropathic pain. The term neuropathic pain encompasses a wide range of pain syndromes of diverse etiologies. The three most commonly diagnosed pain types of neuropathic nature are diabetic neuropathy, cancer neuropathy, and HIV pain. In addition, neuropathic pain is diagnosed in patients with a wide range of other disorders, including trigeminal neuralgia, post-herpetic neuralgia, traumatic neuralgia, phantom limb, as well as a number of other disorders of ill-defined or unknown origin.

Managing the spectrum of pain etiologies remains a major public health problem and both patients and clinicians are seeking improved strategies to effectively manage pain. No currently available therapies or drugs effectively treat all types of nociceptive and neuropathic pain states. The compounds of the present invention are novel CB2 receptor modulators that have utility in treating pain, including nociceptive and neuropathic pain.

Numerous studies have demonstrated that CB2-selective modulators are analgesic in preclinical models of nociceptive and neuropathic pain without causing the adverse side-effects associated with CB1 receptor activation (Malan et al., 2003, Curr. Opin. Pharmacol. 3: 62-7; Ibrahim et al., 2003, Proc. Natl. Acad. Sci. USA 100: 10529-33; Hanus et al., 1999, Proc. Natl. Acad. Sci. USA 96: 14228-33; Elmes et al., 2004, Eur. J. Neurosci. 20: 2311-20; Fox and Bevan, 2005, Expert Opin. Invest. Drugs 14: 695-703). For example, the CB2 receptor-selective compound AM1241 has been shown to be active in several animal models of pain, including spinal nerve ligation, acute thermal pain, carrageenan-induced thermal hyperalgesia and intradermal capsaicin-evoked hyperalgesia (Quartilho et al., 2003, Anesthesiology 99: 955-60; Hohmann et al., 2004, J. Pharmacol. Exp. Ther.: 308, 446-53). The CB2 receptor-selective partial agonist GW405833 has also been shown to be efficacious in inflammatory, neuropathic, and surgical models of pain (Valenzano et al., 2005, Neuropharmacology 48:658-72). A recent study revealed that oral delivery of Lactobacillus acidophilus induced the expression of CB2 receptors in the intestinal epithelium suggesting that CB2 receptor modulators may be useful for the treatment of abdominal pain associated with gastrointestinal diseases such as irritable bowel syndrome (Rousseaux et al., 2007, Nat. Med. 13: 35-37). Therefore, compounds that selectively target CB2 receptors represent an attractive approach for the development of novel analgesics.

Due to the restricted expression of the CB2 receptor in subsets of immune cells and neurons, selective CB2 ligands have therapeutic value (Pertwee, R. G., Curr. Med. Chem. 6: 63 5-64, 1999). Of particular interest are those compounds with high affinity and high specificity for the CB2 receptor. These compounds could afford the benefits of CB2 agonism while avoiding the adverse side effects seen in compounds with affinity for the CB1 receptor. Such compounds could be effective in the treatment of pain as well as autoimmune diseases including but not limited to multiple sclerosis, rheumatoid arthritis, systemic lupus erythematosus, myasthenia gravis, diabetes mellitus type I, inflammatory bowel disease or irritable bowel syndrome, psoriasis and other immune related disorders including but not limited to tissue rejection in organ transplants, malabsorption syndromes such as celiac disease, pulmonary diseases such as asthma and Sjogren's syndrome. The discovery of cannabinoid receptors and the more recent identification of endocannabinoids, endogenous ligands capable of activating the CB receptors, has led to the understanding of the multiplicity of effects exerted by cannabinoids and related compounds. On top of a general neuroprotective effect of certain cannabinoid agonists more specific applications can be found. Thus, for example, evidence for the tonic control of spasticity by the endocannabinoid system suggests that cannabinoid agonists may help in the treatment of muscle spasm and tremor in multiple sclerosis (Baker D. et al., FASEB 3. 15: 300-2, 2001), in addition to the possible moderation of the disease by immuno-modulation through an action on CB2 receptors expressed by immune cells. Cannabinoid agonists may also prove to be of help in the treatment muscle spasm in cancer and REV/AIDS (Hall W. D., Degenhardt L. J. & Currow D., Med. J. Aust. 175: 39-40, 2001) and of neuromuscular disorders.

Recently, studies have demonstrated a potential therapeutic benefit for CB2-selective agonists for the treatment of osteoporosis. CB2 is expressed in osteoblasts, osteocytes and osteoclasts. The CB2-selective agonist HU-308 mitigates ovariectomy-induced bone loss in mice (Ofek et al., 2006, Proc. Natl. Acad. Sci. USA 103 696-701). Consistent with these findings, CB2 knockout mice were shown to have reduced bone mass.

CB2 agonists are also of potential benefit for the treatment of atherosclerosis. Low dose treatment of apoe knockout mice with Δ9-THC has been shown to reduce atherosclerosis progression. Furthermore, these effects are abrogated by treatment with a CB2-selective antagonist (Steffens et al., 2005, Nature 434 782-86).

Liver fibrosis is driven by chronic liver injury and ultimately leads to the development of cirrhosis. Recent studies have shown that CB2 modulators may be of benefit for the treatment of liver diseases such as liver fibrosis, ischemia-reperfusion injury, hepatic encephalopathy and non-alcoholic fatty liver disease (NAFLD). CB2 receptors are expressed in hepatocytes derived from individuals diagnosed with NAFLD but not from normal liver samples (Mendez-Sanchez et al., 2007, Liver Int. 27(2) 215-219). Expression of CB2 has also been shown to be highly upregulated in myofibroblasts isolated from cirrhotic human livers (Julien et al. 2006, Gastroenterology 128 742-755). In a mouse model of liver fibrosis, CB2 knockout animals displayed a significantly enhanced fibrotic phenotype as compared to wild type controls (Lotersztajn et al. 2008, Br. J. Pharmacol. 153(2):286-89). Interestingly, treatment of liver myofibroblasts with a CB2 agonist results in inhibition of cell growth and triggers apoptosis (Julien et al. 2006, Gastroenterology 128 742-755). Thus, activation of CB2 may limit fibrosis by interfering with the growth of liver fibrogenic cells. Taken together, these data suggest that CB2-selective agonists hold promise as therapeutics for a range of liver diseases.

Several synthetic compounds have been shown to bind to the CB2 receptor with a higher affinity than to the CB1 receptor (Pertwee, R. G., Expert Opin. Investig. Drugs 9: 1553-71, 2000). Cannabinoid receptor agonists comprise four main groups of compounds. The classic cannabinoids maintain the dibenzopyran ring system of THC while the non-classical cannabinoids include bicyclic or tricyclic analogs lacking the pyran ring. The aminoalkylindoles and analogs make up the third family and the endocannabinoids including anandamide and other fatty acid derivatives comprise the fourth family. For instance, L5 759656 is a classical cannabinoid analog and HTJ-308 is a bicyclic analog. Both have CB2/CB1 binding affinity ratios of 300-400 and both have been shown to behave as potent and specific CB2 agonists in functional assays (Hand, L. et al., Proc. Natl. Acad. Sci. USA 96: 14228-33, 1999; Ross, R. A. et al., Br. J. Pharmacol. 126: 665-72, 1999).

Compounds disclosed herein are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds are also useful in the treatment of pain associated with acute herpes zoster (shingles), postherpetic neuralgia (PHN), and associated pain syndromes such as ocular pain. The compounds are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, bunionectomy, pain resulting from cancer, muscular pain, mastalgia, pain resulting from dermal injuries, lower back pain, headaches of various etiologies, including migraine, and the like. The compounds are also useful for the treatment of pain-related disorders such as tactile allodynia and hyperalgesia. The compounds are also useful for the treatment of glaucoma. The pain may be somatogenic (either nociceptive or neuropathic), acute and/or chronic.

Novel compounds and pharmaceutical compositions, certain of which have been found to modulate CB2 have been discovered, together with methods of synthesizing and using the compounds including methods for the treatment of CB2-mediated diseases in a patient by administering the compounds.

Disclosed herein is a class of compounds, useful in treating CB2-mediated disorders and conditions, defined by structural Formula I:

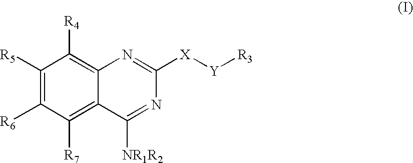

or a salt, ester, or prodrug thereof, wherein:

X is (CR₈R₉)ₙ;

Y is selected from the group consisting of —NR₁₀, —OR₁₀, and a bond;

n is an integer from 0 to 3;

R₁ and R₂ are each independently selected from the group consisting of hydrogen, acyl, lower alkyl, arylalkyl, C-amido, aryl, cycloalkyl, cycloalkylalkyl, lower heteroalkyl, heteroaryl, heteroarylalkyl, heterocycloalkyl, heterocycloalkylalkyl, perhaloalkyl, sulfonyl, and S-sulfonamide, any of which may be optionally substituted; or R¹ and R² together may form heterocycloalkyl or heteroaryl, which may be optionally substituted;

R₃ is selected from the group consisting of hydrogen, null, —C(O)R₁₁, —C(O)NHR₁₁, —S(O)₂R₁₁, —S(O₂)NHR₁₁, aryl, heteroaryl, cycloalkyl, lower alkyl, lower alkoxy, amino, aryloxy, carbamate, carboxy, lower heteroalkyl, haloalkyl, haloalkoxy, heterocycloalkyl, perhaloalkoxy, and perhaloalkyl, any of which may be optionally substituted;

R₄, R₅, R₆, and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₈ and R₉ are each independently selected from the group consisting of hydrogen, lower alkoxy, lower alkyl, lower perhaloalkyl, and halogen, any of which may be optionally substituted, or R₈ and R₉, taken together, are oxy or optionally substituted cycloalkyl;

R₁₀ is selected from the group consisting of hydrogen, —C(O)R₁₁, —C(O)NHR₁₁, —S(O)₂R₁₁, —S(O₂)NHR₁₁, lower alkyl, aryl, cycloalkyl, lower heteroalkyl, heteroaryl, heterocycloalkyl, and perhaloalkyl, any of which may be optionally substituted; or R³ and R¹⁰ together may form heterocycloalkyl which may be optionally substituted; and

R₁₁ is selected from the group consisting of aryl, cycloalkyl, heterocycloalkyl, and heteroaryl, any of which may be optionally substituted.

Compounds disclosed herein possess useful CB2 modulating activity, and may be used in the treatment or prophylaxis of a disease or condition in which CB2 plays an active role. Thus, in broad aspect, also provided herein are pharmaceutical compositions, comprising one or more compounds, disclosed herein together with a pharmaceutically acceptable carrier, as well as methods of making and using the compounds and compositions. In certain embodiments are provided methods for modulating CB2. In other embodiments are provided methods for treating a CB2-mediated disorder in a patient in need of such treatment comprising administering to said patient a therapeutically effective amount of a compound or composition according to the present invention. Also provided is the use of compounds disclosed herein for use in the manufacture of a medicament for the treatment of a disease or condition ameliorated by the modulation of CB2.

In further embodiments provided herein,

n is 1;

Y is a bond;

R₃ is aryl, which may be optionally substituted; and

R₈ and R₉ are each independently selected from the group consisting of hydrogen and lower alkyl.

In further embodiments provided herein, n is 0.

In further embodiments provided herein,

Y is NR₁₀;

R₃ is hydrogen; and

R₁₀ is aryl, which may be optionally substituted.

In other embodiments provided herein,

Y is OR₁₀;

R₃ is null; and

R₁₀ is aryl, which may be optionally substituted.

In certain embodiments provided herein,

n is 1;

Y is NR₁₀;

R₃ is hydrogen;

R₈ and R₉ are each independently hydrogen; and

R₁₀ is selected from the group consisting of CONHR₁₁, SO₂R₁₁, COR₁₁, and aryl.

In other embodiments provided herein, R₁₀ is aryl, which may be optionally substituted.

In further embodiments provided herein,

R₁₀ is —CONHR₁₁; and

R₁₁ is aryl, which may be optionally substituted.

In yet further embodiments provided herein,

R₁₀ is —COR₁₁; and

R₁₁ is aryl, which may be optionally substituted.

In further embodiments provided herein,

R₁₀ is hydrogen; and

R₃ is —SO₂R₁₁.

In certain embodiments, compounds have structural Formula TI:

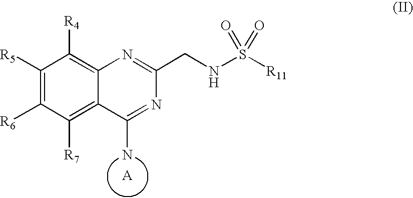

or a salt, ester, or prodrug thereof, wherein:

A is a five- to seven-membered monocyclic heterocycloalkyl which may be optionally substituted;

R₄, R₅, R₆, and R₇ are each independently selected from the group consisting of lower alkyl, lower alkoxy, cyano, cycloalkyl, halogen, hydrogen, hydroxy, lower perhaloalkoxy, lower perhaloalkyl; and

R₁₁ is selected from the group consisting of aryl and heteroaryl, any of which may be optionally substituted.

In further embodiments provided herein, R₁₁ is optionally substituted naphthyl.

In yet further embodiments provided herein,

R₄ is hydrogen; and

R₅, R₆, and R₇ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

In further embodiments provided herein,

R₅, R₆, and R₇ are each independently selected from the group consisting of hydrogen, lower alkoxy, hydroxy, halogen, and lower perhaloalkyl; and

R₁₂ and R₁₃ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

In yet further embodiments provided herein,

R₁₂ is selected from the group consisting of hydrogen, halogen, and lower perhaloalkyl; and

R₁₃ is halogen.

In other embodiments provided herein, R₁₄ is selected from the group consisting of carbamate and acyl.

In certain embodiments provided herein,

n is an integer from 0 to 1;

Y is a bond;

R₃ is selected from the group consisting of aryl, heteroaryl, cycloalkyl, and alkyl, any of which may be optionally substituted; and

R₈ and R₉ are each independently selected from the group consisting of hydrogen and lower alkyl.

In certain embodiments provided herein, n is 0.

In other embodiments provided herein, R₃ is phenyl, which may be optionally substituted by one or more substituents selected from the group consisting of hydrogen, halogen, hydroxy, cyano, nitro, alkyl, haloalkyl, perhaloalkyl, alkoxy, haloalkoxy, perhaloalkoxy, acyloxy, acyl, carboxyl, amino, alkylamino, arylamino, C-amido, N-amido, carbamate, urea, N-sulfonamido, S-sulfonamido, alkylsulfonyl, thiol, and alkylthio.

In further embodiments provided herein, R₁ and R₂ are each independently selected from the group consisting of hydrogen, aryl, arylalkyl, cycloalkyl, cycloalkylalkyl, heteroalkyl, heteroarylalkyl, heterocycloalkylalkyl, and lower alkyl; or R¹ and R² together may form heterocycloalkyl or heteroaryl, which may be optionally substituted.

In further embodiments provided herein, R¹ and R² together form a heterocycloalkyl or heteroaryl, which may be optionally substituted by one or more substituents selected from the group consisting of carbamate, cycloalkyl, C-amido, lower perhaloalkyl, acyl, aryl, alkylsulfonyl, haloalkylalkyl, heteroaryl, and heterocycloalkyl.

In further embodiments provided herein, R¹ and R² together form a 5- or 6-membered monocyclic heterocycloalkyl, which may be optionally substituted by one or more substituents selected from the group consisting of carbamate, cycloalkyl, C-amido, lower perhaloalkyl, acyl, aryl, alkylsulfonyl, haloalkylalkyl, heteroaryl, and heterocycloalkyl.

In certain embodiments, compounds have structural Formula III:

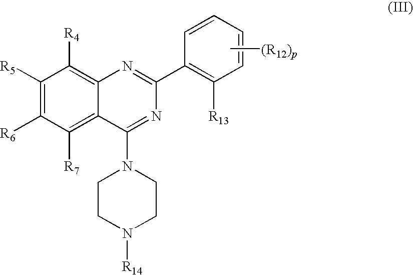

or a salt, ester, or prodrug thereof, wherein:

p is an integer from 0 to 4;

R₄, R₅, R₆, R₁₂, and R₁₃ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₇ is selected from the group consisting of halogen, hydroxy, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and

R₁₄ is selected from the group consisting of carbamate, cycloalkyl, C-amido, perhaloalkyl, acyl, aryl, alkylsulfonyl, haloalkylalkyl heteroaryl, any of which may be optionally substituted.

In certain embodiments provided herein,

R₄ is hydrogen;

R₅ and R₆ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxy, amino, alkylamino, thiol, and alkylthio; and

R₇ is selected from the group consisting of halogen, hydroxy, cyano, nitro, C₂-C₆ alkyl, lower alkenyl, lower alkynyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

In other embodiments provided herein,

R₅ and R₆ are each independently selected from the group consisting of hydrogen, lower alkoxy, hydroxy, halogen, and lower perhaloalkyl;

R₇ is selected from the group consisting of selected from the group consisting of lower alkoxy, lower alkenyl, lower alkynyl, hydroxy, halogen, and lower perhaloalkyl; and

R₁₂ and R₁₃ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

In further embodiments provided herein,

R₁₂ is selected from the group consisting of hydrogen, halogen, and lower perhaloalkyl; and

R₁₃ is halogen.

In yet further embodiments provided herein, R₁₄ is selected from the group consisting of carbamate and acyl.

In certain embodiments, compounds have structural Formula III:

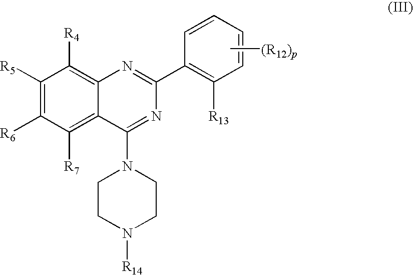

or a salt, ester, or prodrug thereof, wherein:

p is an integer from 0 to 4;

R₄ and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₅ is selected from the group consisting of hydrogen, hydroxy, cyano, nitro, C₂-C₆ alkyl, haloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₆ is selected from the group consisting of hydrogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₁₂ is selected from the group consisting of halogen, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, C₁-C₅ alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₁₃ is selected from the group consisting of hydrogen, halogen, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and

R₁₄ is selected from the group consisting of straight-chain carbamate, lower perhaloalkyl, straight-chain alkylacyl, haloalkylalkyl, heteroaryl, and heterocycloalkyl, any of which may be optionally substituted.

In certain embodiments provided herein,

R₄ is hydrogen;

R₅ is selected from the group consisting of hydrogen, hydroxy, cyano, nitro, C₂-C₆ alkyl, haloalkyl, lower alkoxy, lower haloalkoxy, lower perhaloalkoxy, acyloxy, acyl, amino, alkylamino, thiol, and alkylthio;

R₆ is selected from the group consisting of hydrogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and

R₇ is selected from the group consisting of hydrogen, halogen, hydroxy, cyano, nitro, lower alkyl, lower alkenyl, lower alkynyl, lower haloalkyl, lower perhaloalkyl, lower alkoxy, lower haloalkoxy, lower perhaloalkoxy, acyloxy, acyl, carboxy, amino, alkylamino, thiol, and alkylthio.

In further embodiments provided herein,

R₅ is selected from the group consisting of hydrogen, lower alkoxy, and hydroxy;

R₆ is selected from the group consisting of hydrogen, lower alkoxy, hydroxy, and lower perhaloalkyl;

R₇ is selected from the group consisting of hydrogen, lower alkoxy, lower alkenyl, lower alkynyl, hydroxy, halogen, and lower perhaloalkyl;

R₁₂ is selected from the group consisting of halogen, cyano, nitro, C₂-C₆ alkyl, lower haloalkyl, lower perhaloalkyl, C₁-C₅ alkoxy, lower haloalkoxy, lower perhaloalkoxy, acyloxy, acyl, carboxyl, amino, alkylamino, thiol, and alkylthio; and

R₁₃ is selected from the group consisting of hydrogen, halogen, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

In further embodiments provided herein, R₁₂ is selected from the group consisting of halogen and lower perhaloalkyl; and

R₁₃ is halogen.

In yet further embodiments provided herein, R₁₄ is selected from the group consisting of straight-chain carbamate and acyl.

In certain embodiments, compounds have structural Formula III:

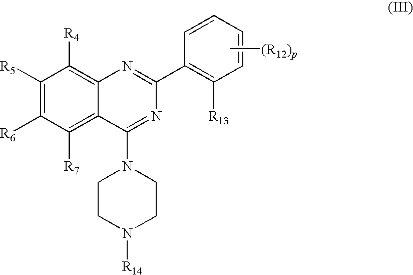

or a salt, ester, or prodrug thereof, wherein:

p is an integer from 0 to 4;

R₄, R₆, and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₅ is selected from the group consisting of hydrogen, halogen, hydroxy, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₁₂ is selected from the group consisting of hydrogen, halogen, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;

R₁₃ is selected from the group consisting of halogen, cyano, nitro, alkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, C₂-C₆ alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and

R₁₄ is selected from the group consisting of straight-chain carbamate, cycloalkyl, C-amido, C₂-C₆ alkyl, lower perhaloalkyl, acyl, aryl, arylsulfonyl, haloalkylalkyl, heteroaryl, and heterocycloalkyl, any of which may be optionally substituted.

In certain embodiments provided herein,

R₄ is hydrogen;

R₅ is selected from the group consisting of hydrogen, halogen, hydroxy, cyano, nitro, C₂-C₆ alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and

R₆ and R₇ are each independently selected from the group consisting of hydrogen, halogen, hydroxy, cyano, nitro, lower alkyl, lower alkenyl, lower alkynyl, lower haloalkyl, lower perhaloalkyl, lower alkoxy, lower haloalkoxy, lower perhaloalkoxy, acyloxy, acyl, carboxy, amino, alkylamino, thiol, and alkylthio.

In further embodiments provided herein,

R₅, R₆, and R₇ are each independently selected from the group consisting of hydrogen, lower alkoxy, lower alkenyl, lower alkynyl, hydroxy, halogen, and lower perhaloalkyl;

R₁₂ is selected from the group consisting of hydrogen, halogen, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and

R₁₃ is selected from the group consisting of halogen, cyano, nitro, lower alkyl, lower perhaloalkyl, C₂-C₆ alkoxy, haloalkoxy, lower perhaloalkoxy, acyloxy, acyl, carboxy, amino, alkylamino, thiol, and alkylthio.

In further embodiments provided herein,

R₁₂ is selected from the group consisting of halogen and lower perhaloalkyl; and

R₁₃ is halogen.

In yet further embodiments provided herein, R₁₄ is selected from the group consisting of straight-chain carbamate and acyl.

In certain embodiments, compounds have structural Formula III:

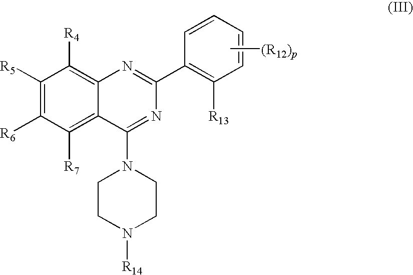

or a salt, ester, or prodrug thereof, wherein:

R₇ and R₁₃ are each independently selected from the group consisting of halogen, hydroxy, lower alkoxy, lower alkenyl, and lower alkynyl; and

R₁₄ is selected from the group consisting of carbamate, cycloalkyl, C-amido, lower perhaloalkyl, acyl, aryl, alkylsulfonyl, haloalkylalkyl, heteroaryl, and heterocycloalkyl, any of which may be optionally substituted.

In further embodiments provided herein, R₁₄ is selected from the group consisting of carbamate and acyl.

In yet further embodiments provided herein, R₇ and R₁₃ are each independently halogen.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“CB2 modulator” is used herein to refer to a compound that exhibits an EC₅₀ with respect to CB2 activity of no more than about 100 μM and more typically not more than about 50 μM, as measured in the CB2 radioligand binding assay and CB2 GTPγ[³⁵S] functional assay-described generally hereinbelow. “EC₅₀” is that concentration of modulator which activates an enzyme (e.g., CB2) to half-maximal level. Certain compounds disclosed herein have been discovered to exhibit modulatory activity against CB2. In certain embodiments, compounds will exhibit an EC₅₀ with respect to CB2 of no more than about 10 μM; in further embodiments, compounds will exhibit an EC₅₀ with respect to CB2 of no more than about 5 μM; in yet further embodiments, compounds will exhibit an EC₅₀ with respect to CB2 of not more than about 1 μM; in yet further embodiments, compounds will exhibit an EC₅₀ with respect to CB2 of not more than about 200 nM, as measured in the CB2 assay described herein. In certain embodiments, said modulators are agonists.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002). The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In certain embodiments, the salts may include hydrochloride or acetate. A salt of a compound can be made by reacting the appropriate compound in the form of the free base with the appropriate acid.

While it may be possible for the compounds disclosed herein to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds disclosed herein with: a) corticosteroids including betamethasone dipropionate (augmented and nonaugemented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocalne, and EMLA Cream [Eutectic Mixture of Local Anesthetics (lidocaine 2.5% and prilocalne 2.5%; an emulsion in which the oil phase is a eutectic mixture of lidocaine and prilocalne in a ratio of 1:1 by weight. This eutectic mixture has a melting point below room temperature and therefore both local anesthetics exist as a liquid oil rather then as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g., duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) other cannabinoids including selective and non-selective CB1/CB2 ligands; and other agents, such as capsaicin.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating CB2-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of CB2-mediated disorders.

Thus, in another aspect, provided herein are methods for treating CB2-mediated disorders in a human or animal subject in need of such treatment comprising administering to said subject an amount of a compound as disclosed herein effective to reduce or prevent said disorder in the subject in combination with at least one additional agent for the treatment of said disorder that is known in the art. In a related aspect, provided herein are therapeutic compositions, comprising at least one compound of the present invention, in combination with one or more additional agents for the treatment of CB2-mediated disorders.

The compounds disclosed herein are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds are also useful in the treatment of pain associated with acute herpes zoster (shingles), postherpetic neuralgia (PHN), and associated pain syndromes such as ocular pain. The compounds are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds are also useful for the treatment of pain-related disorders such as tactile allodynia and hyperalgesia. The pain may be somatogenic (either nociceptive or neuropathic), acute and/or chronic.

Furthermore, the compounds disclosed herein can be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods disclosed herein are useful in the treatment or prevention of drug withdrawal symptoms, for example treatment or prevention of symptoms of withdrawal from opiate, alcohol, or tobacco addiction.

Other disorders or conditions which can be advantageously treated by the compounds disclosed herein include inflammation. The compounds disclosed herein are useful as anti-inflammatory agents with the additional benefit of having significantly less harmful side effects. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as reflux esophagitis, diarrhea, inflammatory bowel disease, Crohn's disease, gastritis, irritable bowel syndrome and ulcerative colitis. The compounds may also be used in the treatment of pulmonary inflammation, such as that associated with viral infections and cystic fibrosis. In addition, compounds disclosed herein are also useful in organ transplant patients either alone or in combination with conventional immunomodulators. Yet further, the compounds disclosed herein are useful in the treatment of pruritis and vitaligo. In addition, the compounds can be used to treat insulin resistance and other metabolic disorders such as atherosclerosis that are typically associated with an exaggerated inflammatory signaling.

The compounds disclosed herein can be used in the treatment ischemia, retinitis of ophthalmic diseases, such as glaucoma, retinal ganglion degeneration, ocular irritation, retinopathies, uveitis, ocular photophobia, and of inflammation and pain associated with acute injury to the eye tissue. Specifically, the compounds can be used to treat glaucomatous retinopathy and/or diabetic retinopathy. The compounds can also be used to treat post-operative inflammation or pain as from ophthalmic surgery such as cataract surgery and refractive surgery.

Still other disorders or conditions advantageously treated by the compounds disclosed herein include the prevention or treatment of hypreproliferative diseases, especially cancers. Hematological and non-hematological malignancies which may be treated or prevented include but are not limited to multiple myeloma, acute and chronic leukemias including Acute Lymphocytic Leukemia (ALL), Chronic Lymphocytic Leukemia (CLL), and Chronic Myelogenous Leukemia (CLL), lymphomas, including Hodgkin's lymphoma and non-Hodgkin's lymphoma (low, intermediate, and high grade), as well as solid tumors and malignancies of the brain, head and neck, breast, lung, reproductive tract, upper digestive tract, pancreas, liver, renal, bladder, prostate and colorectal. The compounds can also be used to treat fibrosis, such as that which occurs with radiation therapy. The present compounds can also be used to treat subjects having adenomatous polyps, including those with familial adenomatous polyposis (FAP). Additionally, the present compounds can be used to prevent polyps from forming in patients at risk of FAP. The compounds may also be used to treat malignancies of the skin including, but not limited to, melanomas.

The compounds disclosed herein may also be used in the treatment of autoimmune diseases including but not limited to multiple sclerosis, rheumatoid arthritis, systemic lupus erythematosus, myasthenia gravis, diabetes mellitus type I, inflammatory bowel disease or irritable bowel syndrome, psoriasis and other immune related disorders including but not limited to tissue rejection in organ transplants, malabsorption syndromes such as celiac disease, pulmonary diseases such as asthma and Sjogren's syndrome.

Besides being useful for human treatment, certain compounds and formulations disclosed herein may also be useful for veterinary treatment of companion animals, exotic animals and farm animals, including mammals, rodents, rabbits, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Synthetic Methods for Preparing Compounds

The following schemes can be used to practice the present invention.

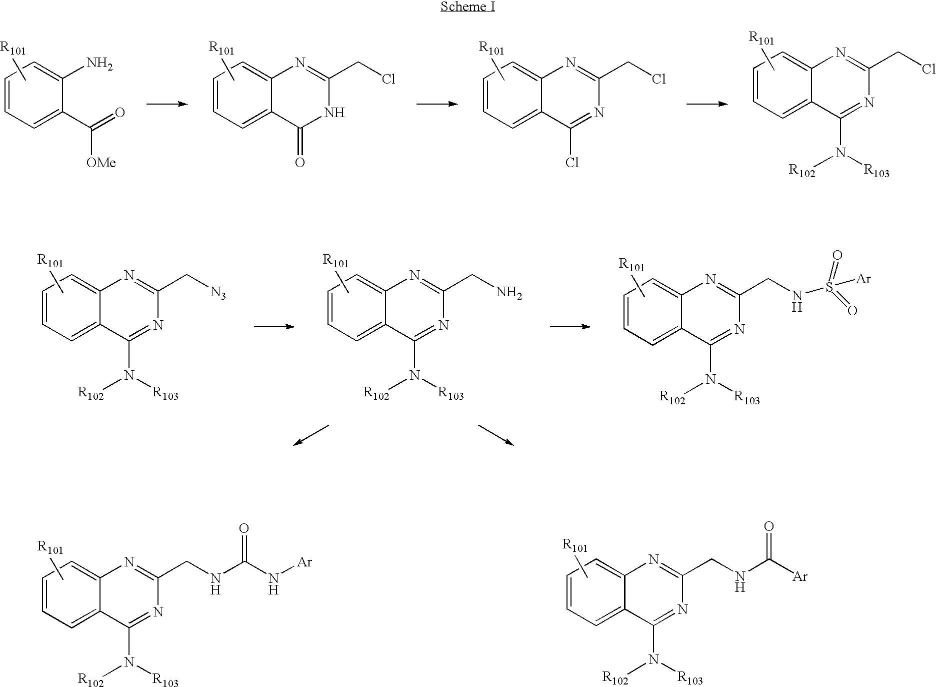

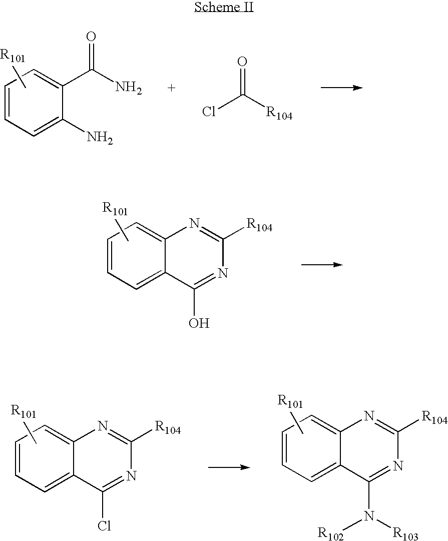

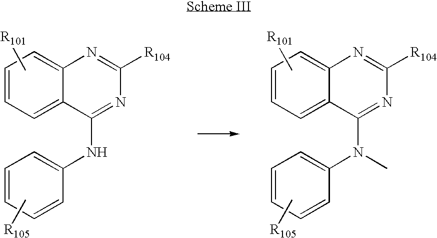

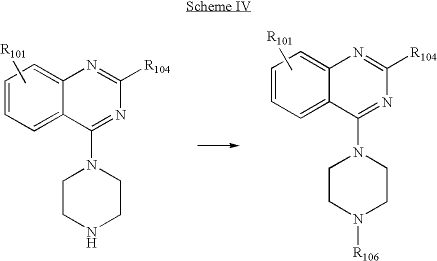

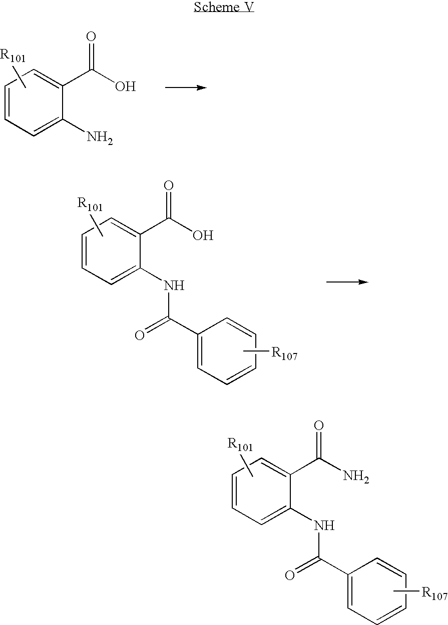

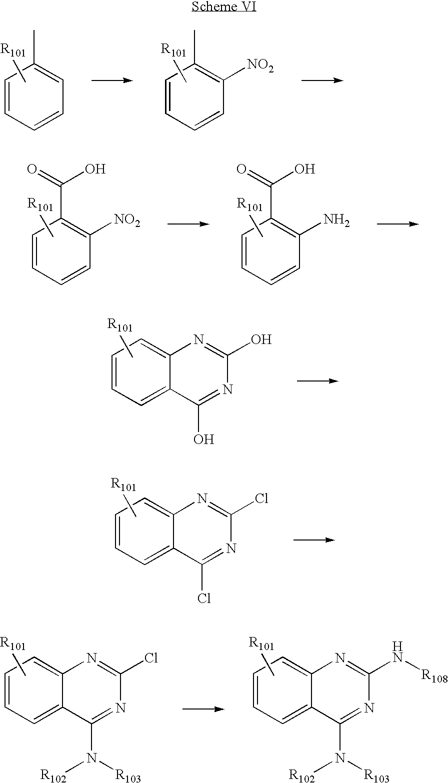

The invention is further illustrated by the following examples.

### EXAMPLE 1

### N-((4-(Pyrrolidin-1-yl)quinazolin-2-yl)methyl)naphthalene-1-sulfonamide

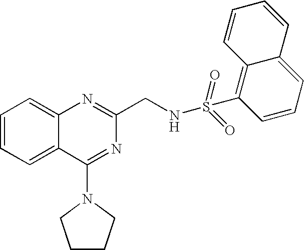

### Step 1

The compound N-((4-(pyrrolidin-1-yl)quinazolin-2-yl)methyl)naphthalene-1-sulfonamide was synthesized according to Scheme I. A mixture of ethyl 2-aminobenzoate (0.9 g, 6 mmol) and chloroacetonitrile in HCl/dioxane (4 M, 6 mL) was stirred at room temperature for 15 hours. Then half of the solvent was evaporated and water (15 mL) was added. The solution was neutralized with sodium carbonate and the resulting precipitate was filtered to give 2-(chloromethyl)quinazolin-4(3H)-one. LCMS: Calc MW=194.62; Found (M/z) M+1=195.02, 197.05.

### Step 2

A mixture of 2-(chloromethyl)quinazolin-4(3H)-one (2.09 g) and POCl₃ (6 ml) in dry dioxane (50 ml) was heated at 90° C. for 5 hours. The solvent was removed and water and ethyl acetate were added. The system was neutralized with NaHCO₃, extracted with ethyl acetate, and dried over Na₂SO₄. Evaporation of the solvent and separation by column chromatography gave 4-chloro-2-(chloromethyl)quinazoline. LCMS: Calc MW=213.06; Found (M/z) M+1=212.85, 214.85.

### Step 3

A solution of 4-chloro-2-(chloromethyl)quinazoline (0.7 g) and pyrrolidine (374 mg) in isopropanol (6 mL) was heated at 75° C. for 4 hours. The solvent was removed and the residue was partitioned between water and ethyl acetate. 2-(Chloromethyl)-4-(pyrrolidin-1-yl)quinazoline was obtained by column chromatography. LCMS: Calc MW=247.72; Found (M/z) M+1=248.13, 250.13.

### Step 4

A mixture of 2-(chloromethyl)-4-(pyrrolidin-1-yl)quinazoline (237 mg) and sodium azide (77 mg) in dry dimethyl formamide (5 mL) was heated at 78° C. for 0.5 hours. The solution was partitioned between water and ethyl acetate. Evaporation of the solvent gave the desired intermediate 2-(azidomethyl)-4-(pyrrolidin-1-yl)quinazoline. LCMS: Calc MW=254.29; Found (M/z) M+1−N2=227.12.

### Step 5

2-(Azidomethyl)-4-(pyrrolidin-1-yl)quinazoline (230 mg) was reduced by hydrogen with Pd/C as catalyst in methanol (30 mL) at room temperature for 1 hour. The catalyst was filtered off and the solvent was removed under reduced pressure to give (4-(pyrrolidin-1-yl)quinazolin-2-yl)methanamine. LCMS: Calc MW=228.29; Found (M/z) M+1=229.18.

### Step 6

A solution of (4-(pyrrolidin-1-yl)quinazolin-2-yl)methanamine (293 mg), 1-naphthalenesulfonyl chloride (293 mg) and pyridine (0.4 mL) in dry tetrahydrofuran (20 mL) was stirred at room temperature for 1 hour and then at 60° C. for 1 hour. The solvent was removed and the residue was partitioned between water and ethyl acetate. The title compound was obtained by column chromatography. ¹H NMR (400 MHz, (DMSO-d₆) δ 8.88 (t, 1H), 8.61 (d, 1H), 8.13 (d, 1H), 8.05 (d, 1H), 7.80 (m, 2H), 7.77 (d, 2H), 7.70 (m, 3H), 7.51 (t, 1H), 7.29 (t, 1H), 4.33 (d, 2H), 3.4-3.1 (m, 4H), 2.0-1.8 (m, 4H). LCMS: Calc MW=418.51; Found (M/z) M+1=419.08.

### EXAMPLE 2

### N-((8-Methyl-4-(pyrrolidin-1-yl)quinazolin-2-yl)methyl)naphthalene-1-sulfonamide

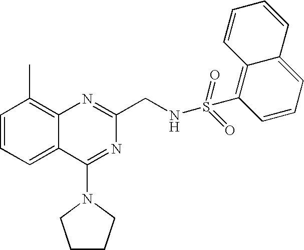

The compound N-((8-methyl-4-(pyrrolidin-1-yl)quinazolin-2-yl)methyl)naphthalene-1-sulfonamide was prepared following the procedures for the preparation of Example 1. LCMS: Calc MW=432.54; Found (M/z) M+1=433.29.

### EXAMPLE 3

### N-((4-(Pyrrolidin-1-yl)quinazolin-2-yl)methyl)benzenesulfonamide

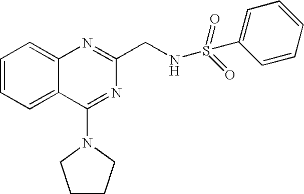

The compound N-((4-(pyrrolidin-1-yl)quinazolin-2-yl)methyl)benzenesulfonamide was prepared following the procedures for Example 1. LCMS: Calc MW=368.45; Found (M/z) M+1=369.08.

### EXAMPLE 4

### 2,4-Difluoro-N-((4-(pyrrolidin-1-yl)quinazolin-2-yl)methyl)benzenesulfonamide

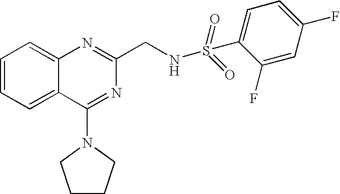

The compound 2,4-difluoro-N-((4-(pyrrolidin-1-yl)quinazolin-2-yl)methyl)benzenesulfonamide was prepared following the procedures described for Example 1 LCMS: Calc MW=404.43; Found (M/z) M+1=405.18.

### EXAMPLE 5

### N-((4-(4-Methylpiperazin-1-yl)quinazolin-2-yl)methyl)naphthalene-1-sulfonamide

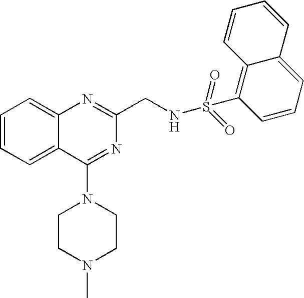

The compound N-((4-(4-methylpiperazin-1-yl)quinazolin-2-yl)methyl)naphthalene-1-sulfonamide was prepared following the procedures described for Example 1 using 1-methylpiperazine in Step 3. ¹H NMR (400 MHz, (CD₃OD) 8.61 (d, 1H), 7.96 (d, 1H), 7.70-7.54 (m, 5H), 7.50-7.38 (m, 3H), 7.05 (t, 1H), 4.29 (s, 2H), 3.40 (t, 4H), 2.46 (t, 4H), 2.32 (s, 3H). LCMS: Calc MW=447.55; Found (M/z) M+1=448.28.

### EXAMPLE 6

### N-((4-(4-methylpiperazin-1-yl)quinazolin-2-yl)methyl)naphthalene-2-sulfonamide

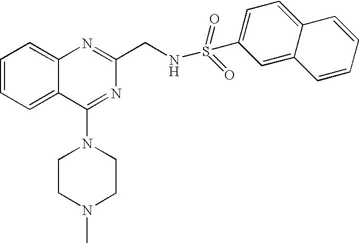

The compound N-((4-(4-methylpiperazin-1-yl)quinazolin-2-yl)methyl)naphthalene-2-sulfonamide was prepared following the procedures described for Example 1 using 1-methylpiperazine in Step 3. ¹H NMR (400 MHz, (CD₃OD) 8.06 (s, 1H), 7.69 (d, 1H), 7.66-7.56 (m, 5H), 7.51 (t, 1H), 7.42 (t, 2H), 7.28 (t, 1H), 4.37 (s, 2H), 3.54 (t, 4H), 2.46 (t, 4H), 2.32 (s, 3H). LCMS: Calc MW=447.55; Found (M/z) M+1=448.27.

### EXAMPLE 7

### Methyl 3-(N-((4-(Pyrrolidin-1-yl)quinazolin-2-yl)methyl)sulfamoyl)thiophene-2-carboxylate

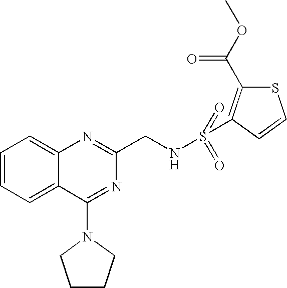

The compound methyl 3-(N-((4-(pyrrolidin-1-yl)quinazolin-2-yl)methyl)sulfamoyl)thiophene-2-carboxylate was prepared following the procedures described for Example 1 using pyrrolidine in Step 3 and methyl 3-(chlorosulfonyl)thiophene-2-carboxylate in Step 6. ¹H NMR (400 MHz, (DMSO-d₆) 8.38 (d, 1H) 8.00 (m, 2H), 7.88 (m, 2H), 7.67 (t, 1H), 7.44 (d, 2H), 4.46 (d, 2H), 4.2-3.7 (m, 7H), 2.06-1.94 (m, 4H). LCMS: Calc MW=432.52; Found (M/z) M+1=432.97.

### EXAMPLE 8

### N-Methyl-3-(N-((4-(pyrrolidin-1-yl)quinazolin-2-yl)methyl) sulfamoyl)thiophene-2-carboxamide

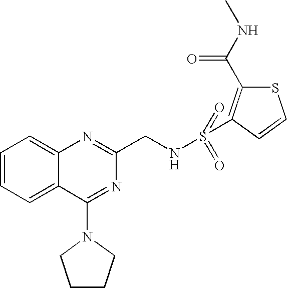

The compound N-methyl-3-(N-((4-(pyrrolidin-1-yl)quinazolin-2-yl)methyl) sulfamoyl)thiophene-2-carboxamide was prepared following the procedures described for Example 1 using methyl 3-(chlorosulfonyl)thiophene-2-carboxamide in Step 6. LCMS: Calc MW=431.53; Found (M/z) M+1=432.00.

### EXAMPLE 9

### N-(2-(Diethylamino)ethyl)-3-(N-((4-(pyrrolidin-1-yl)quinazolin-2-yl)methyl)sulfamoyl)thiophene-2-carboxamide

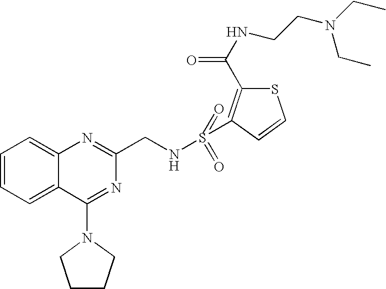

The compound N-(2-(diethylamino)ethyl)-3-(N-((4-(pyrrolidin-1-yl)quinazolin-2-yl)methyl)sulfamoyl)thiophene-2-carboxamide was prepared following the procedure described for Example 1. LCMS: Calc MW=516.68; Found (M/z) M+1=517.33.

### EXAMPLE 10

### 1-(2,4-Dichlorophenyl)-3-((4-(pyrrolidin-1-yl)quinazolin-2-yl)methyl)urea

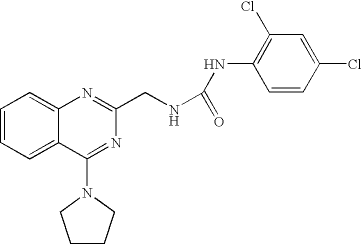

The compound 1-(2,4-dichlorophenyl)-3-((4-(pyrrolidin-1-yl)quinazolin-2-yl)methyl)urea was synthesized according to Scheme I. A solution of 4-(pyrrolidin-1-yl)quinazolin-2-yl)methanamine (31.0 mg) and 2,4-dichlorophenylisocyanate (25.6 mg) in dry tetrahydrofuran (2 mL) was stirred at room temperature for 2 hours. The crude material was purified by reverse-phase HPLC to give the title compound. LCMS: Calc MW=416.30; Found (M/z) M+1=415.98, 417.90.

### EXAMPLE 11

### 1-(4-Chloro-3-(trifluoromethyl)phenyl)-3-((4-(pyrrolidin-1-yl)quinazolin-2-yl)methyl)urea

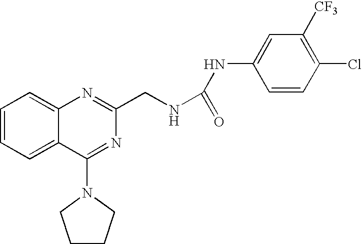

This compound 1-(4-chloro-3-(trifluoromethyl)phenyl)-3-((4-(pyrrolidin-1-yl)quinazolin-2-yl)methyl)urea was prepared following the procedures described for Example 10 using 4-chloro-3-trifluoromethylphenylisocyante. LCMS: Calc MW=449.86; Found (M/z) M+1=450.06.

### EXAMPLE 12

### 4-Chloro-N-((4-(pyrrolidin-1-yl)quinazolin-2-yl)methyl)benzamide

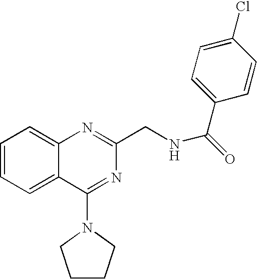

The compound 4-chloro-N-((4-(pyrrolidin-1-yl)quinazolin-2-yl)methyl)benzamide was synthesized according to Scheme I. A solution of (4-(pyrrolidin-1-yl)quinazolin-2-yl)methanamine (46 mg), 4-chlorobenzoyl chloride (38 mg), and pyridine (50 μL) in dry acetonitrile (2 mL) was stirred at room temperature for 2 hours. The crude mixture was purified by reverse-phase HPLC to give the title compound. LCMS: Calc MW=366.84; Found (M/z) M+1=367.03.

### EXAMPLE 13

### 2-(3,4-Difluorophenyl)-4-(pyrrolidin-1-yl)quinazoline

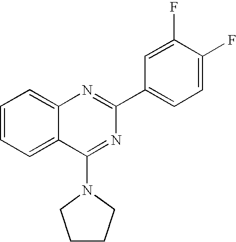

The compound 2-(3,4-Difluorophenyl)-4-(pyrrolidin-1-yl)quinazoline was synthesized according to Scheme II.

### Step 1

Anthranilamide (1.36 g, 9.98 mmol) and triethylamine (4.17 mL, 29.9 mmol) were dissolved in THF (80 mL). Then 3,4-difluorobenzoyl chloride (1.50 mL, 12.0 mmol) was added and the mixture stirred at room temperature. The solvent was then removed in vacuo and to the crude material added 2 N NaOH (50 mL). The mixture was heated to 90° C. for four hours. After cooling to room temperature, 6 M HCl was added until the pH<1. The mixture was then filtered and the solids collected and dried in vacuo to give 2.45 g of 2-(3,4-difluorophenyl)quinazolin-4-ol.

### Step 2

### 2-(3,4-difluorophenyl)quinazolin-4-ol (1.64 g, 6.35 mmol) was dissolved in POCl₃ (80 mL) and heated to 80° C. for 18 hours. The solvent was then removed in vacuo and the residue taken up in CH₂Cl₂ and extracted with NaHCO₃ followed by brine. The organic layer was dried, filtered, concentrated and purified by flash column chromatography (0-30% EtOAc/hexanes) to give 0.31 g of 4-chloro-2-(3,4-difluorophenyl)quinazoline.

### Step 3

### 4-Chloro-2-(3,4-difluorophenyl)quinazoline (0.125 g, 0.484 mmol) was dissolved in dry THF (4 mL) and pyrrolidine (0.12 mL, 1.45 mmol) was added. The mixture was stirred at room temperature for 1 hour. Silica gel was then added and the mixture concentrated to dryness and then purified by flash column chromatography (0-30% EtOAc/hexanes) to give 0.111 g of the title compound. ¹H NMR (400 MHz, CDCl₃) δ 8.40 (m, 1H), 8.34 (m, 1H), 8.17 (d, 1H), 7.88 (d, 1H), 7.69 (t, 1H), 7.37 (m, 1H), 7.25 (m, 1H), 4.03 (m, 4H), 2.09 (m, 4H). MS (M/z, M+1), 312.17.

### EXAMPLE 14

### 2-(3,4-Difluorophenyl)-4-(pyrrolidin-1-yl)quinazoline

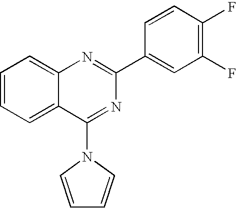

The compound 2-(3,4-Difluorophenyl)-4-(pyrrolidin-1-yl)quinazoline was synthesized according to Scheme II.

### Step 1

Anthranilamide (1.36 g, 9.98 mmol) and triethylamine (4.17 mL, 29.9 mmol) were dissolved in THF (80 mL). Then 3,4-difluorobenzoyl chloride (1.50 mL, 12.0 mmol) was added and the mixture stirred at room temperature. The solvent was then removed in vacuo and to the crude material added 2 N NaOH (50 mL). The mixture was heated to 90° C. for four hours. After cooling to room temperature, 6 M HCl was added until the pH<1. The mixture was then filtered and the solids collected and dried in vacuo to give 2.45 g of 2-(3,4-difluorophenyl)quinazolin-4-ol.

### Step 2

### 2-(3,4-difluorophenyl)quinazolin-4-ol (1.64 g, 6.35 mmol) was dissolved in POCl₃ (80 mL) and heated to 80° C. for 18 hours. The solvent was then removed in vacuo and the residue taken up in CH₂Cl₂ and extracted with NaHCO₃ followed by brine. The organic layer was dried, filtered, concentrated and purified by flash column chromatography (0-30% EtOAc/hexanes) to give 0.31 g of 4-chloro-2-(3,4-difluorophenyl)quinazoline.

### Step 3

### 4-Chloro-2-(3,4-difluorophenyl)quinazoline (0.125 g, 0.484 mmol) was dissolved in dry THF (4 mL) and pyrrolidine (0.12 mL, 1.45 mmol) was added. The mixture was stirred at room temperature for 1 hour. Silica gel was then added and the mixture concentrated to dryness and then purified by flash column chromatography (0-30% EtOAc/hexanes) to give 0.111 g of the title compound. ¹H NMR (400 MHz, CDCl₃) δ 8.40 (m, 1H), 8.34 (m, 1H), 8.17 (d, 1H), 7.88 (d, 1H), 7.69 (t, 1H), 7.37 (m, 1H), 7.25 (m, 1H), 4.03 (m, 4H), 2.09 (m, 4H). MS (M/z, M+1), 312.17.

### EXAMPLE 15

### 2-(3,4-difluorophenyl)-4-(piperidin-1-yl)quinazoline

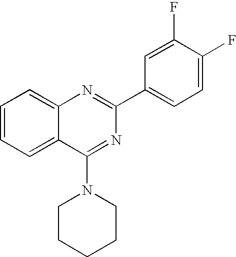

The compound 2-(3,4-difluorophenyl)-4-(piperidin-1-yl)quinazoline was prepared following the procedures described in Step 3 for the preparation of Example 13 using piperidine. ¹H NMR (400 MHz, CDCl₃) δ 8.40 (m, 1H), 8.33 (m, 1H), 7.91 (m, 2H), 7.72 (t, 1H), 7.42 (t, 1H), 7.24 (m, 1H), 3.81 (m, 4H), 1.84 (m, 6H). MS (M/z, M+1), 326.2.

### EXAMPLE 16

### 4-(2-(3,4-difluorophenyl)quinazolin-4-yl)morpholine

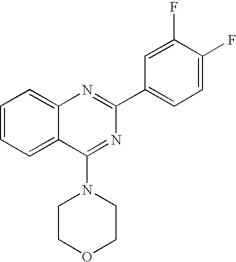

The compound 4-(2-(3,4-difluorophenyl)quinazolin-4-yl)morpholine was prepared following the procedures described in Step 3 for the preparation of Example 13 using morpholine. ¹H NMR (400 MHz, CDCl₃) δ 8.39 (m, 1H), 8.33 (m, 1H), 7.96 (d, 1H), 7.90 (d, 1H), 7.76 (t, 1H), 7.45 (t, 1H), 7.25 (m, 1H), 3.96 (m, 4H), 3.86 (m, 4H). MS (M/z, M+1), 328.2.

### EXAMPLE 17

### N-benzyl-2-(3,4-difluorophenyl)-N-methylquinazolin-4-amine

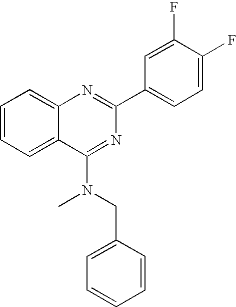

The compound N-benzyl-2-(3,4-difluorophenyl)-N-methylquinazolin-4-amine was prepared following the procedures described in Step 3 for the preparation of Example 13 using N-methylbenzylamine. ¹H NMR (400 MHz, CDCl₃) δ 8.21 (m, 1H), 8.16 (m, 1H), 7.77 (m, 2H), 7.54 (m, 1H), 7.26 (m, 4H), 7.15 (m, 2H), 7.06 (m, 1H), 4.89 (s, 2H), 3.22 (s, 3H). MS (M/z, M+1), 362.2.

### EXAMPLE 18

### 2-(3,4-Difluorophenyl)-4-(4-methylpiperazin-1-yl)quinazoline

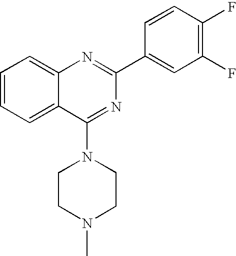

The compound 2-(3,4-Difluorophenyl)-4-(4-methylpiperazin-1-yl)quinazoline was prepared following the procedures described in Step 3 for the preparation of Example 13 using 1-methylpiperazine. ¹H NMR (400 MHz, CDCl₃) δ 8.39 (m, 1H), 8.33 (m, 1H), 7.94 (d, 1H), 7.90 (d, 1H), 7.74 (t, 1H), 7.44 (t, 1H), 7.25 (m, 1H), 3.92 (m, 4H), 2.69 (m, 4H), 2.42 (s, 3H). MS (M/z, M+1), 341.1.

### EXAMPLE 19

### 1-(4-(2-(3,4-Difluorophenyl)quinazolin-4-yl)piperazin-1-yl)ethanone

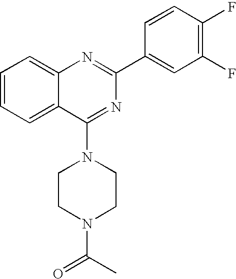

The compound 1-(4-(2-(3,4-Difluorophenyl)quinazolin-4-yl)piperazin-1-yl)ethanone was prepared following the procedures described in Step 3 for the preparation of Example 13 using 1-acetylpiperizine. ¹H NMR (400 MHz, CDCl₃) δ 8.39 (m, 1H), 8.33 (m, 1H), 7.98 (d, 1H), 7.91 (d, 1H), 7.79 (t, 1H), 7.49 (t, 1H), 7.25 (m, 1H), 3.83 (m, 8H), 2.20 (s, 3H). MS (M/z, M+1), 369.5.

### EXAMPLE 20

### N-(4-tert-Butylphenyl)-2-(3,4-difluorophenyl)quinazolin-4-amine

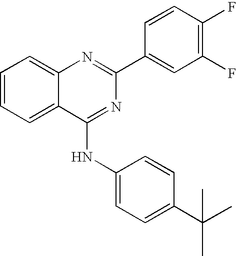

The compound N-(4-tert-Butylphenyl)-2-(3,4-difluorophenyl)quinazolin-4-amine was prepared following the procedures described in Step 3 for the preparation of Example 14 using 4-tert-butylaniline. ¹H NMR (400 MHz, CDCl₃) δ 8.39 (m, 1H), 8.32 (m, 1H), 7.96 (d, 1H), 7.86 (d, 1H), 7.80 (m, 1H), 7.77 (d, 2H), 7.53 (m, 3H), 7.45 (s, 1H), 7.28 (m, 1H), 1.40 (s, 9H). MS (M/z, M+1), 390.2

### EXAMPLE 21

### N-(4-tert-Butylphenyl)-2-(3,4-difluorophenyl)-N-methylquinazolin-4-amine

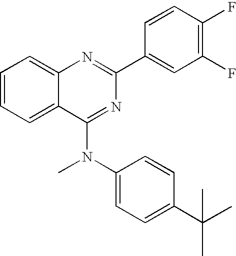

The compound N-(4-tert-butylphenyl)-2-(3,4-difluorophenyl)-N-methylquinazolin-4-amine was synthesized according to Scheme III. N-(4-tert-butylphenyl)-2-(3,4-difluorophenyl)quinazolin-4-amine (115 mg, 0.296 mmol) was dissolved in dry THF (3 mL) and methyl iodide (30 μL, 0.444 mmol) was added followed by sodium hydride (60% mineral oil suspension, 53 mg, 1.33 mmol). The mixture was stirred at room temperature for 1.5 hours. The mixture was then concentrated onto silica gel and purified by column chromatography (0-30% EtOAc/hexanes) to give the title compound. ¹H NMR (400 MHz, CDCl₃) δ 8.47 (m, 1H), 8.40 (m, 1H), 8.87 (d, 1H), 7.58 (m, 1H), 7.41 (d, 2H), 7.29 (m, 1H), 7.15 (d, 2H), 7.02 (m, 2H), 3.74 (s, 3H), 1.36 (s, 9H). MS (M/z, M+1), 404.3.

### EXAMPLE 22

### 2-(2,4-Difluorophenyl)-4-(pyrrolidin-1-yl)quinazoline

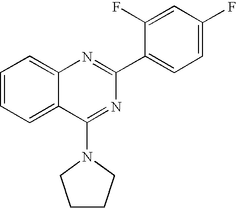

The compound 2-(2,4-difluorophenyl)-4-(pyrrolidin-1-yl)quinazoline was prepared following the procedures described for Example 13 using 2,4-difluorobenzoyl chloride in Step 1 and pyrrolidine in Step 3. ¹H NMR (400 MHz, CDCl₃) δ 8.19 (m, 2H), 7.89 (d, 1H), 7.70 (t, 1H), 7.39 (t, 1H), 6.94 (m, 2H), 4.01 (m, 4H), 2.08 (m, 4H). MS (M/z, M+1), 312.2

### EXAMPLE 23

### 2-(2,4-Difluorophenyl)-4-(piperidin-1-yl)quinazoline

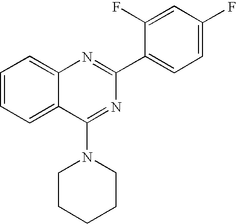

The compound 2-(2,4-difluorophenyl)-4-(piperidin-1-yl)quinazoline was prepared following the procedures described for Example 13 using 2,4-difluorobenzoyl chloride in Step 1 and piperidine in Step 3. ¹H NMR (400 MHz, CDCl₃) δ 8.18 (m, 1H), 7.94 (d, 1H), 7.90 (d, 1H), 7.73 (t, 1H), 7.44 (t, 1H), 6.96 (m, 2H), 3.79 (m, 4H), 1.81 (m, 6H). MS (M/z, M+1), 326.2.

### EXAMPLE 24

### 1-(4-(2-(2,4-Difluorophenyl)quinazolin-4-yl)piperazin-1-yl)ethanone

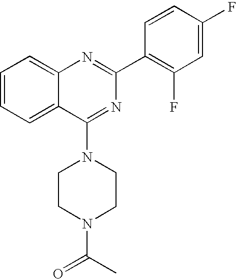

The compound 1-(4-(2-(2,4-difluorophenyl)quinazolin-4-yl)piperazin-1-yl)ethanone was prepared following the procedures described for Example 13 using 2,4-difluorobenzoyl chloride in Step 1 and 1-acetylpiperizine in Step 3. ¹H NMR (400 MHz, CDCl₃) δ 8.20 (m, 1H), 8.00 (d, 1H), 7.92 (d, 1H), 7.89 (t, 1H), 7.51 (t, 1H), 6.96 (m, 2H), 3.87 (m, 6H), 3.74 (m, 2H), 2.18 (s, 3H). MS (M/z, M+1), 369.5.

### EXAMPLE 25

### 4-(2-(2,4-Difluorophenyl)quinazolin-4-yl)morpholine

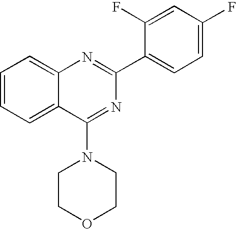

The compound 4-(2-(2,4-difluorophenyl)quinazolin-4-yl)morpholine was prepared following the procedures described for Example 13 using 2,4-difluorobenzoyl chloride in Step 1 and morpholine in Step 3. ¹H NMR (400 MHz, CDCl₃) δ 8.20 (m, 1H), 7.99 (d, 1H), 7.91 (d, 1H), 7.77 (t, 1H), 7.48 (t, 1H), 6.96 (m, 2H), 3.94 (m, 4H), 3.86 (m, 4H). MS (M/z, M+1), 328.2.

### EXAMPLE 26

### Benzyl[2-(2,4-difluorophenyl)quinazolin-4-yl]methylamine

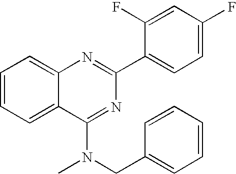

The compound benzyl[2-(2,4-difluorophenyl)quinazolin-4-yl]methylamine was prepared following the procedures described for Example 13 using 2,4-difluorobenzoyl chloride in Step 1 and N-methylbenzylamine in Step 3. ¹H NMR (400 MHz, CDCl₃) δ 8.18 (m, 1H), 7.96 (m, 2H), 7.73 (m, 1H), 7.43 (m, 4H), 7.35 (m, 2H), 6.95 (m, 2H), 5.05 (s, 2H), 3.37 (s, 3H). MS (M/z, M+1), 362.2.

### EXAMPLE 27

### 2-(2,4-Difluorophenyl)-4-(4-methylpiperazin-1-yl)quinazoline

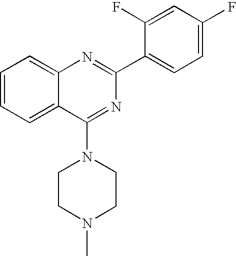

The compound 2-(2,4-difluorophenyl)-4-(4-methylpiperazin-1-yl)quinazoline was prepared following the procedures described for Example 13 using 2,4-difluorobenzoyl chloride in Step 1 and 1-methylpiperazine in Step 3. ¹H NMR (400 MHz, CDCl₃) δ 8.20 (m, 1H), 7.96 (d, 1H), 7.91 (d, 1H), 7.75 (t, 1H), 7.46 (m, 1H), 6.95 (m, 2H), 3.93 (m, 4H), 2.67 (m, 4H), 2.42 (s, 3H). MS (M/z, M+1), 341.1.

### EXAMPLE 28

### 2-(2,4-Dichlorophenyl)-4-pyrrolidin-1-ylquinazoline

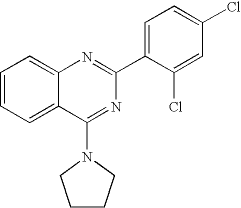

The compound 2-(2,4-dichlorophenyl)-4-pyrrolidin-1-ylquinazoline was prepared following the procedures described for Example 13 using 2,4-dichlorobenzoyl chloride in Step 1 and pyrrolidine in Step 3. ¹H NMR (400 MHz, DMSO-d₆) δ 8.33 (d, 1H), 8.82 (d, 1H), 7.78 (m, 2H), 7.65 (m, 1H) 7.48 (m, 2H), 3.91 (m, 4H), 1.97 (m, 4H). MS (M/z, M+1), 344.1.

### EXAMPLE 29

### 2-(2,4-Dichlorophenyl)-4-piperidin-1-ylquinazoline

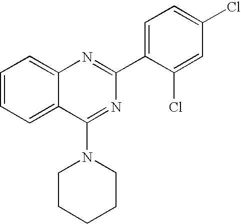

The compound 2-(2,4-dichlorophenyl)-4-piperidin-1-ylquinazoline was prepared following the procedures described for Example 13 using 2,4-dichlorobenzoyl chloride in Step 1 and piperidine in Step 3. ¹H NMR (400 MHz, DMSO-d₆) δ 7.99 (d, 1H), 7.86 (d, 1H), 7.82 (m, 2H), 7.71 (d, 1H), 7.55 (m, 2H), 3.75 (m, 4H), 1.70 (m, 6H). MS (M/z, M+1), 358.1.

### EXAMPLE 30

### Cyclopropylmethyl[2-(2,4-dichlorophenyl)quinazolin-4-yl]amine

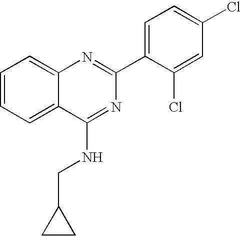

The compound cyclopropylmethyl[2-(2,4-dichlorophenyl)quinazolin-4-yl]amine was prepared following the procedures described for Example 13 using 2,4-dichlorobenzoyl chloride in Step 1 and aminomethylcyclopropane in Step 3. ¹H (400 MHz, DMSO-d₆) δ 8.55 (m, 1H), 8.31 (d, 1H), 7.77 (m, 2H), 7.72 (d, 1H), 7.69 (m, 1H) 7.55 (m, 2H), 3.43 (m, 2H), 1.22 (m, 1H), 0.43 (m, 2H), 0.29 (m, 2H). MS (M/z, M+1), 344.1.

### EXAMPLE 31

### Benzyl[2-(2,4-dichlorophenyl)quinazolin-4-yl]amine

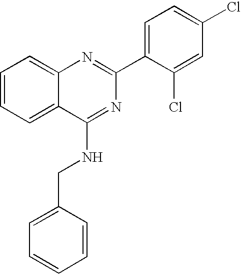

The compound benzyl[2-(2,4-dichlorophenyl)quinazolin-4-yl]amine was prepared following the procedures described for Example 13 using 2,4-dichlorobenzoyl chloride in Step 1 and benzylamine in Step 3. ¹H NMR (400 MHz, DMSO-d₆) δ 9.01 (t, 1H), 8.36 (d, 1H), 7.79 (m, 3H), 7.65 (m, 1H), 7.57 (m, 1H), 7.49 (m, 1H), 7.37 (m, 2H), 7.29 (m, 2H), 7.24 (m, 1H), 4.84 (d, 2H). MS (M/z, M+1), 380.1.

### EXAMPLE 32

### N-Benzyl-2-(2,4-dichlorophenyl)-N-methylquinazolin-4-amine

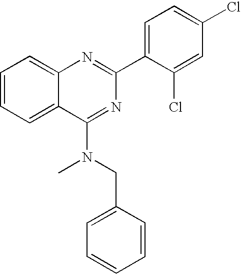

The compound N-benzyl-2-(2,4-dichlorophenyl)-N-methylquinazolin-4-amine was prepared following the procedures described for Example 13 using 2,4-dichlorobenzoyl chloride in Step 1 and N-methylbenzylamine in Step 3. ¹H NMR (400 MHz, DMSO-d₆) δ 8.12 (d, 1H), 7.82 (m, 3H), 7.68 (m, 1H), 7.49 (m, 2H), 7.36 (m, 5H), 5.06 (s, 2H), 3.35 (s, 3H). MS (M/z, M+1), 393.8.

### EXAMPLE 33

### 2-Cyclohexyl-4-(4-methylpiperazin-1-yl)quinazoline

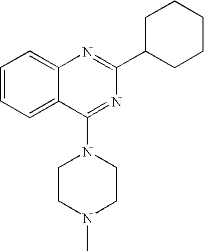

The compound 2-cyclohexyl-4-(4-methylpiperazin-1-yl)quinazoline was prepared following the procedures described for Example 13 using cyclohexanecarbonyl chloride in Step 1 and 1-methylpiperazine in Step 3. ¹H NMR (400 MHz, CDCl₃) δ 7.84 (m, 2H), 7.68 (t, 1H), 7.36 (t, 1H), 3.81 (m, 4H), 2.83 (m, 1H), 2.61 (m, 4H), 2.38 (s, 3H), 2.01 (m, 2H), 1.85 (m, 2H), 1.73 (m, 4H), 1.42 (m, 2H). MS (M/z, M+1), 311.3.

### EXAMPLE 34

### 2-Cyclohexyl-4-(piperidin-1-yl)quinazolines

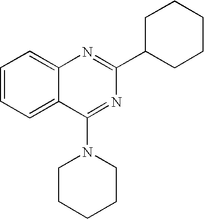

The compound 2-cyclohexyl-4-(piperidin-1-yl)quinazoline was prepared following the procedures described for Example 13 using cyclohexanecarbonyl chloride in Step 1 and piperidine in Step 3. ¹H NMR (400 MHz, CDCl₃) δ 7.83 (m, 2H), 7.66 (t, 1H), 7.35 (m, 1H), 3.70 (m, 4H), 2.81 (m, 1H), 2.01 (m, 2H), 1.77 (m, 1H), 1.39 (m, 3H). MS (M/z, M+1), 296.3.

### EXAMPLE 35

### 4-(2-Cyclohexylquinazolin-4-yl)morpholine

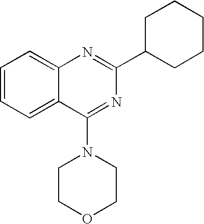

The compound 4-(2-cyclohexylquinazolin-4-yl)morpholine was prepared following the procedures described for Example 13 using cyclohexanecarbonyl chloride in Step 1 and morpholine in Step 3. ¹H NMR (400 MHz, CDCl₃) δ 7.86 (m, 2H), 7.70 (t, 1H), 7.39 (t, 1H), 3.90 (m, 4H), 3.77 (m, 4H), 2.84 (m, 1H), 2.04 (m, 2H), 1.86 (m, 2H), 1.72 (m, 4H), 1.39 (m, 2H). MS (M/z, M+1), 298.3.

### EXAMPLE 36

### 2-Cyclohexyl-4-(pyrrolidin-1-yl)quinazolines

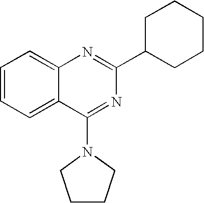

The compound 2-cyclohexyl-4-(pyrrolidin-1-yl)quinazolines was prepared following the procedures described for Example 13 using cyclohexanecarbonyl chloride in Step 1 and pyrrolidine in Step 3. ¹H NMR (400 MHz, DMSO-d₆) δ 8.24 (d, 1H), 7.72 (t, 1H), 7.64 (d, 1H), 7.40 (t, 1H), 3.88 (m, 4H), 2.65 (m, 1H), 1.97 (m, 6H), 1.80 (m, 2H), 1.66 (m, 3H), 1.34 (m, 3H). MS (M/z, M+1), 282.3.

### EXAMPLE 37

### N-(2-Chlorophenyl)-2-cyclohexylquinazolin-4-amine

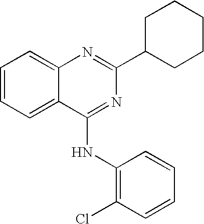

The compound N-(2-chlorophenyl)-2-cyclohexylquinazolin-4-amine was prepared following the procedures described for Example 14 using cyclohexanecarbonyl chloride in Step 1 and 2-chloroaniline in Step 3. ¹H NMR (400 MHz, CDCl₃) δ 9.07 (d, 1H), 8.20 (s, 1H), 7.91 (d, 2H), 7.80 (t, 1H), 7.55 (t, 1H), 7.47 (d, 1H), 7.42 (t, 1H), 7.08 (t, 1H), 2.92 (m, 1H), 2.10 (m, 2H), 1.90 (m, 2H), 1.78 (m, 3H), 1.43 (m, 3H). MS (M/z, M+1), 338.2.

### EXAMPLE 38

### N-Benzyl-2-cyclohexylquinazolin-4-amine

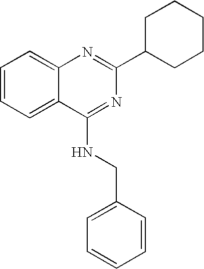

The compound N-benzyl-2-cyclohexylquinazolin-4-amine was prepared following the procedures described for Example 13 using cyclohexanecarbonyl chloride in Step 1 and benzylamine in Step 3. ¹H NMR (400 MHz, CDCl₃) δ 7.82 (d, 1H), 7.68 (m, 2H), 7.43 (m, 2H), 7.35 (m, 4H), 6.03 (m, 1H), 4.91 (d, 2H), 2.82 (m, 1H), 2.02 (m, 2H), 1.86 (m, 4H), 1.42 (m, 4H). MS (M/z, M+1), 318.2.

### EXAMPLE 39

### N-Benzyl-2-cyclohexyl-N-methylquinazolin-4-amine

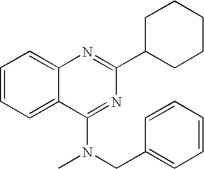

The compound N-benzyl-2-cyclohexyl-N-methylquinazolin-4-amine was prepared following the procedures described for Example 13 using cyclohexanecarbonyl chloride in Step 1 and N-methylbenzylamine in Step 3. ¹H NMR (400 MHz, CDCl₃) δ 7.89 (d, 1H), 7.84 (d, 1H), 7.65 (t, 1H), 7.41 (m, 4H), 7.34 (m, 1H), 7.25 (m, 1H), 4.99 (s, 2H), 3.29 (s, 3H), 2.83 (m, 1H), 2.01 (m, 2H), 1.76 (m, 4H), 1.43 (m, 4H). MS (M/z, M+1), 322.2.

### EXAMPLE 40

### 2-Propyl-4-(pyrrolidin-1-yl)quinazoline

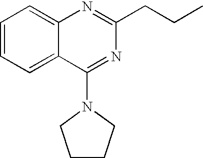

The compound 2-propyl-4-(pyrrolidin-1-yl)quinazoline was prepared following the procedures described for Example 13 using butyryl chloride in Step 1 and pyrrolidine in Step 3. ¹H NMR (400 MHz, CDCl₃) δ 8.12 (d, 1H), 7.81 (d, 1H), 7.65 (t, 1H), 7.31 (t, 1H), 3.95 (m, 4H), 2.82 (t, 2H), 2.04 (m, 4H), 1.90 (m, 2H), 1.03 (t, 3H). MS (M/z, M+1), 242.2.

### EXAMPLE 41

### 4-(4-Methylpiperazin-1-yl)-2-propylquinazoline

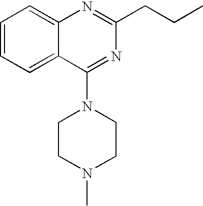

The compound 4-(4-methylpiperazin-1-yl)-2-propylquinazoline was prepared following the procedures described for Example 13 using butyryl chloride in Step 1 and 1-methylpiperazine in Step 3. ¹H NMR (400 MHz, CDCl₃) δ 7.85 (m, 2H), 7.69 (m, 1H), 7.38 (m, 1H), 3.80 (m, 4H), 2.87 (t, 2H), 2.62 (m, 4H), 2.39 (s, 3H), 1.88 (m, 2H), 1.02 (t, 3H). MS (M/z, M+1), 271.2.

### EXAMPLE 42

### 4-(4-Methylpiperazin-1-yl)-2-(4-(trifluoromethyl)phenyl)quinazoline

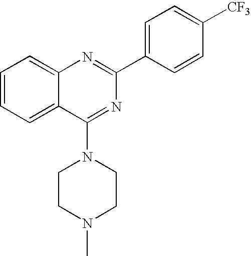

The compound 4-(4-methylpiperazin-1-yl)-2-(4-(trifluoromethyl)phenyl)quinazoline was prepared following the procedures described for Example 13 using 4-trifluoromethylbenzoyl chloride in Step 1 and 1-methylpiperazine in Step 3. ¹H NMR (400 MHz, CDCl₃) δ 8.67 (d, 2H), 7.98 (d, 1H), 7.92 (d, 1H), 7.76 (m, 3H), 7.46 (t, 1H), 3.92 (m, 4H), 2.68 (m, 4H), 2.41 (s, 3H). MS (M/z, M+1), 373.2.

### EXAMPLE 43

### 4-(2-(4-(Trifluoromethyl)phenyl)quinazolin-4-yl)morpholine

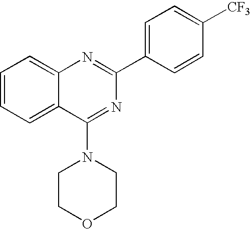

The compound 4-(2-(4-(trifluoromethyl)phenyl)quinazolin-4-yl)morpholine was prepared following the procedures described for Example 13 using 4-trifluoromethylbenzoyl chloride in Step 1 and morpholine in Step 3. ¹H NMR (400 MHz, CDCl₃) δ 8.66 (d, 2H), 8.01 (d, 1H), 7.92 (d, 1H), 7.77 (m, 3H), 7.48 (t, 1H), 3.96 (m, 4H), 3.90 (m, 4H). MS (M/z, M+1), 360.2.

### EXAMPLE 44

### 4-(Piperidin-1-yl)-2-(4-(trifluoromethyl)phenyl)quinazoline

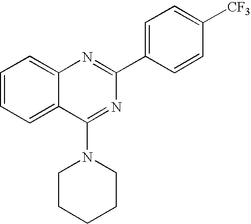

The compound 4-(piperidin-1-yl)-2-(4-(trifluoromethyl)phenyl)quinazoline was prepared following the procedures described for Example 13 using 4-trifluoromethylbenzoyl chloride in Step 1 and piperidine in Step 3. ¹H NMR (400 MHz, CDCl₃) δ 8.67 (d, 2H), 7.96 (d, 1H), 7.91 (d, 1H), 7.74 (m, 3H), 7.44 (t, 1H), 3.83 (m, 4H), 1.82 (m, 6H). MS (M/z, M+1), 358.2.

### EXAMPLE 45

### 4-(Pyrrolidin-1-yl)-2-(4-(trifluoromethyl)phenyl)quinazoline

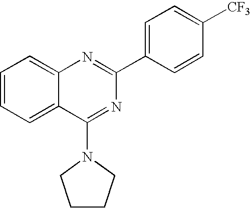

The compound 4-(pyrrolidin-1-yl)-2-(4-(trifluoromethyl)phenyl)quinazoline was prepared following the procedures described for Example 13 using 4-trifluoromethylbenzoyl chloride in Step 1 and pyrrolidine in Step 3. ¹H NMR (400 MHz, CDCl₃) δ 8.66 (d, 2H), 8.19 (d, 1H), 7.93 (d, 1H), 7.71 (m, 3H), 7.39 (t, 1H), 4.06 (m, 4H), 2.10 (m, 4H). MS (M/z, M+1), 344.2.

### EXAMPLE 46

### (4-tert-Butylphenyl)-[2-(4-trifluoromethylphenyl)quinazolin-4-yl]amine

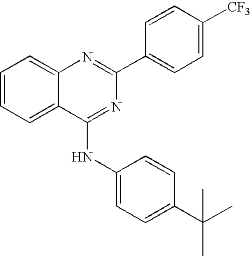

The compound (4-tert-butylphenyl)-[2-(4-trifluoromethylphenyl)quinazolin-4-yl]amine was prepared following the procedures described for Example 14 using 4-trifluoromethylbenzoyl chloride in Step 1 and 4-tert-butylaniline in Step 3. ¹H NMR (400 MHz, DMSO-d₆) δ 9.96 (s, 1H), 8.63 (m, 3H), 7.91 (m, 6H), 7.65 (m, 1H), 7.50 (d, 2H), 1.34 (s, 9H). MS (M/z, M+1), 421.9.

### EXAMPLE 47

### (4-tert-Butylphenyl)methyl-[2-(4-trifluoromethylphenyl)quinazolin-4-yl]amine

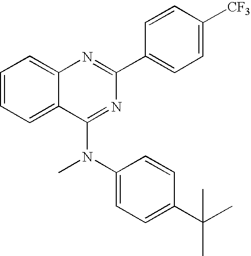

The compound (4-tert-butylphenyl)methyl-[2-(4-trifluoromethylphenyl)quinazolin-4-yl]amine was prepared from (4-tert-butylphenyl)-[2-(4-trifluoromethylphenyl) quinazolin-4-yl]amine following the procedures described for Example 21. ¹H NMR (400 MHz, CDCl₃) δ 8.74 (d, 2H), 7.91 (d, 1H), 7.76 (d, 1H), 7.59 (m, 2H), 7.40 (d, 2H), 7.14 (d, 2H), 7.03 (m, 2H), 3.75 (s, 3H), 1.35 (s, 9H). MS (M/z, M+1), 436.4.

### EXAMPLE 48

### 2-Phenyl-4-(pyrrolidin-1-yl)quinazoline

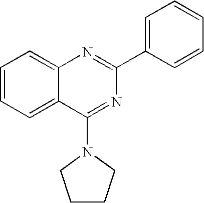

The compound 2-phenyl-4-(pyrrolidin-1-yl)quinazoline was prepared following the procedures described for Example 13 using benzoyl chloride in Step 1 and pyrrolidine in Step 3. ¹H NMR (400 MHz, CDCl₃) δ 8.57 (m, 2H), 8.17 (d, 1H), 7.92 (d, 1H), 7.68 (t, 1H), 7.41 (m, 3H), 7.35 (t, 1H), 4.09 (m, 4H), 2.09 (m, 4H). MS (M/z, M+1), 276.3.

### EXAMPLE 49

### N-(2-Chlorophenyl)-2-phenylquinazolin-4-amine

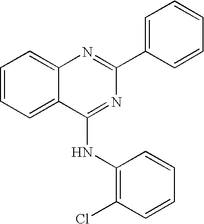

The compound N-(2-chlorophenyl)-2-phenylquinazolin-4-amine was prepared following the procedures described for Example 14 using benzoyl chloride in Step 1 and 2-chloroaniline in Step 3. ¹H NMR (400 MHz, CDCl₃) δ 9.05 (d, 1H), 8.57 (d, 1H), 8.20 (s, 1H), 8.04 (d, 1H), 7.96 (d, 1H), 7.85 (t, 1H), 7.54 (m, 2H), 7.25 (d, 1H), 7.12 (t, 1H), 7.07 (t, 1H), 6.77 (t, 1H), 6.70 (t, 1H). MS (M/z, M+1), 332.1.

### EXAMPLE 50

### N-(Cyclopropylmethyl)-2-phenylquinazolin-4-amine

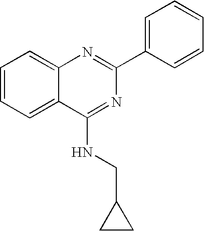

The compound N-(cyclopropylmethyl)-2-phenylquinazolin-4-amine was prepared following the procedures described for Example 13 using benzoyl chloride in Step 1 and methylaminocyclopropane in Step 3. ¹H NMR (400 MHz, CDCl₃) δ 8.36 (m, 2H), 7.86 (d, 1H), 7.76 (d, 1H), 7.48 (t, 1H), 7.28 (m, 3H), 7.19 (t, 1H), 3.45 (m, 2H), 1.06 (m, 1H), 0.41 (m, 2H), 0.21 (m, 2H). MS (M/z, M+1), 276.2.

### EXAMPLE 51

### N-iso-Butyl-2-phenylquinazolin-4-amine

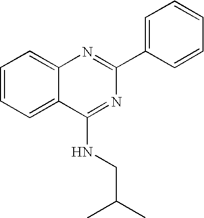

The compound N-iso-butyl-2-phenylquinazolin-4-amine was prepared following the procedures described for Example 13 using benzoyl chloride in Step 1 and isobutylamine in Step 3. ¹H NMR (400 MHz, CDCl₃) δ 8.58 (m, 2H), 7.95 (d, 1H), 7.72 (m, 2H), 7.51 (m, 3H), 7.41 (t, 1H), 3.64 (m, 2H), 2.13 (m, 1H), 1.07 (d, 6H). MS (M/z, M+1), 278.2.

### EXAMPLE 52

### 2-Phenyl-4-(piperidin-1-yl)quinazoline

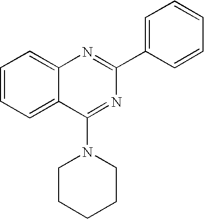

The compound 2-phenyl-4-(piperidin-1-yl)quinazoline was prepared following the procedures described for Example 13 using benzoyl chloride in Step 1 and piperidine in Step 3. ¹H NMR (400 MHz, CDCl₃) δ 8.58 (m, 2H), 7.97 (d, 1H), 7.90 (d, 1H), 7.72 (t, 1H), 7.49 (m, 3H), 7.40 (t, 1H), 3.82 (m, 4H), 1.85 (m, 6H). MS (M/z, M+1), 290.2.

### EXAMPLE 53

### 2-(2-Chlorophenyl)-4-(pyrrolidin-1-yl)quinazoline

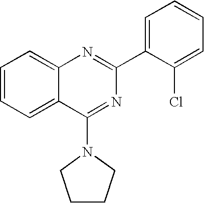

The compound 2-(2-chlorophenyl)-4-(pyrrolidin-1-yl)quinazoline was prepared following the procedures described for Example 13 using 2-chlorobenzoyl chloride in Step 1 and pyrrolidine in Step 3. ¹H NMR (400 MHz, DMSO-d₆) δ 8.33 (d, 1H), 7.75 (m, 3H), 7.49 (m, 4H), 3.91 (m, 4H), 1.97 (m, 4H). MS (M/z, M+1), 310.0.

### EXAMPLE 54

### 2-(2-Chlorophenyl)-4-(piperidin-1-yl)quinazoline

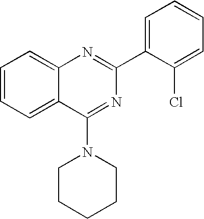

The compound 2-(2-chlorophenyl)-4-(piperidin-1-yl)quinazoline was prepared following the procedures described for Example 13 using 2-chlorobenzoyl chloride in Step 1 and piperidine in Step 3. ¹H NMR (400 MHz, DMSO-d₆) δ 8.00 (d, 1H), 7.81 (m, 3H), 7.56 (m, 2H), 7.47 (m, 2H), 3.74 (m, 4H), 1.71 (m, 6H). MS (M/z, M+1), 324.1.

### EXAMPLE 55

### N-Benzyl-2-(2-chlorophenyl)-N-methylquinazolin-4-amine

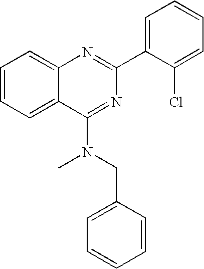

The compound N-benzyl-2-(2-chlorophenyl)-N-methylquinazolin-4-amine was prepared following the procedures described for Example 13 using 2-chlorobenzoyl chloride in Step 1 and N-methylbenzylamine in Step 3. ¹H NMR (400 MHz, DMSO-d₆) δ 8.09 (d, 1H), 7.89 (m, 3H), 7.41 (m, 9H), 5.04 (s, 2H), 3.32 (s, 3H). MS (M/z, M+1), 360.1.

### EXAMPLE 56

### 2-(2-Chlorophenyl)-N-(cyclopropylmethyl)quinazolin-4-amine

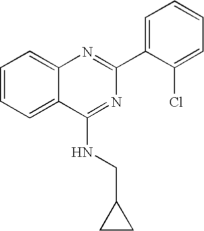

The compound 2-(2-chlorophenyl)-N-(cyclopropylmethyl)quinazolin-4-amine was prepared following the procedures described for Example 13 using 2-chlorobenzoyl chloride in Step 1 and aminomethylcyclopropane in Step 3. ¹H NMR (400 MHz, DMSO-d₆) δ 8.24 (m, 1H), 8.04 (d, 1H), 7.52 (m, 1H), 7.44 (m, 2H), 2.27 (m, 2H), 7.18 (m, 2H), 3.15 (m, 2H), 0.97 (m, 1H), 0.18 (m, 2H), 0.02 (m, 2H). MS (M/z, M+1), 310.1.

### EXAMPLE 57

### 2-(2-Chlorophenyl)-4-(4-methylpiperazin-1-yl)quinazoline

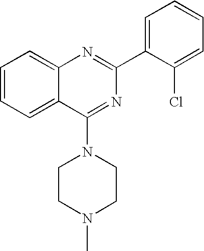

The compound 2-(2-chlorophenyl)-4-(4-methylpiperazin-1-yl)quinazoline was prepared following the procedures described for Example 13 using 2-chlorobenzoyl chloride in Step 1 and 1-methylpiperizine in Step 3. ¹H NMR (400 MHz, CDCl₃) δ 7.98 (d, 1H), 7.92 (d, 1H), 7.86 (m, 1H), 7.74 (t, 1H), 7.46 (m, 2H), 7.36 (m, 2H), 3.89 (m, 4H), 2.63 (m, 4H), 2.38 (s, 3H). MS (M/z, M+1), 339.2.

### EXAMPLE 58

### 2-(2-Chlorophenyl)-4-(4-methylpiperidin-1-yl)quinazoline

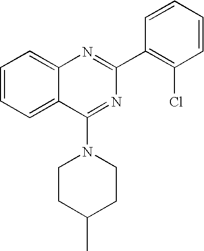

The compound 2-(2-chlorophenyl)-4-(4-methylpiperidin-1-yl)quinazoline was prepared following the procedures described for Example 13 using 2-chlorobenzoyl chloride in Step 1 and 4-methylpiperidine in Step 3. ¹H NMR (400 MHz, CDCl₃) δ 7.95 (d, 1H), 7.92 (d, 1H), 7.85 (m, 1H), 7.73 (t, 1H), 7.45 (m, 2H), 7.35 (m, 2H), 4.43 (m, 2H), 3.14 (t, 2H), 1.82 (m, 2H), 1.71 (m, 1H), 1.51 (m, 2H), 1.03 (d, 3H). MS (M/z, M+1), 338.3.

### EXAMPLE 59

### 2-(2-Chlorophenyl)-4-(2-methylpiperidin-1-yl)quinazoline

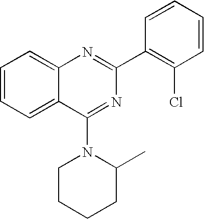

The compound 2-(2-chlorophenyl)-4-(2-methylpiperidin-1-yl)quinazoline was prepared following the procedures described for Example 13 using 2-chlorobenzoyl chloride in Step 1 and 2-methylpiperidine in Step 3. ¹H NMR (400 MHz, CDCl₃) δ 8.15 (m, 1H), 7.89 (m, 2H), 7.76 (t, 1H), 7.47 (m, 2H), 7.38 (m, 2H), 4.94 (m, 1H), 4.35 (m, 1H), 3.45 (m, 1H), 1.94-1.67 (m, 6H), 1.45 (d, 3H). MS (M/z, M+1), 338.3.

### EXAMPLE 60

### 2-(2-Chlorophenyl)-4-(4-(trifluoromethyl)piperidin-1-yl)quinazoline

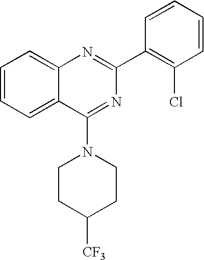

The compound 2-(2-chlorophenyl)-4-(4-(trifluoromethyl)piperidin-1-yl)quinazoline was prepared following the procedures described for Example 13 using 2-chlorobenzoylchloride in Step 1 and 4-trifluoromethylpiperidine in Step 3. ¹H NMR (400 MHz, CDCl₃) δ 7.99 (d, 1H), 7.91 (d, 1H), 7.85 (m, 1H), 7.77 (m, 1H), 7.49 (2H), 7.37 (m, 2H), 4.50 (m, 2H), 3.14 (m, 1H), 2.36 (m, 1H), 2.04 (m, 2H), 1.89 (m, 2H). MS (M/z, M+1), 392.4.

### EXAMPLE 61

### 2-(2-Chlorophenyl)-4-(3,4-dihydroisoquinolin-2(1H)-yl)quinazoline

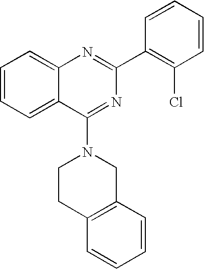

The compound 2-(2-chlorophenyl)-4-(3,4-dihydroisoquinolin-2(1H)-yl)quinazoline was prepared following the procedures described for Example 13 using 2-chlorobenzoyl chloride in Step 1 and 1,2,3,4-tetrahydroisoquinoline in Step 3. ¹H NMR (400 MHz, CDCl₃) δ 8.03 (d, 1H), 7.98 (d, 1H), 7.86 (m, 1H), 7.76 (t, 1H), 7.50 (m, 2H), 7.37 (m, 2H), 7.21 (m, 4H), 5.03 (s, 2H), 4.12 (t, 2H), 3.20 (t, 2H). MS (M/z, M+1), 372.3.

### EXAMPLE 62

### Ethyl 4-(2-(2-Chlorophenyl)quinazolin-4-yl)piperazine-1-carboxylate

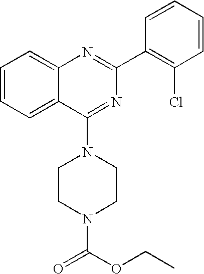

The compound ethyl 4-(2-(2-chlorophenyl)quinazolin-4-yl)piperazine-1-carboxylate was prepared following the procedures described for Example 13 using 2-chlorobenzoyl chloride in Step 1 and ethyl piperazine-1-carboxylate in Step 3. ¹H NMR (400 MHz, CDCl₃) δ 8.00 (d, 1H), 7.93 (d, 1H), 7.86 (m, 1H), 7.78 (t, 1H), 7.50 (m, 2H), 7.37 (m, 2H), 4.18 (q, 2H), 3.82 (m, 4H), 3.72 (m, 4H), 1.29 (t, 3H). MS (M/z, M+1), 397.3.

### EXAMPLE 63

### 2-(2-Chlorophenyl)-N-cyclohexyl-N-methylquinazolin-4-amine

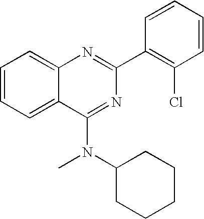

The compound 2-(2-chlorophenyl)-N-cyclohexyl-N-methylquinazolin-4-amine was prepared following the procedures described for Example 13 using 2-chlorobenzoyl chloride in Step 1 and N-methylcyclohexylamine in Step 3. ¹H NMR (400 MHz, CDCl₃) δ 7.96 (d, 1H), 7.92 (d, 1H), 7.85 (m, 1H), 7.70 (t, 1H), 7.47 (m, 1H), 7.41 (t, 1H), 7.34 (m, 2H), 4.91 (m, 1H), 3.25 (s, 3H), 1.99 (m, 2H), 1.86 (m, 2H), 1.66 (m, 4H), 1.40 (m, 2H). MS (M/z, M+1), 352.3.

### EXAMPLE 64

### N-Butyl-2-(2-chlorophenyl)-N-methylquinazolin-4-amine

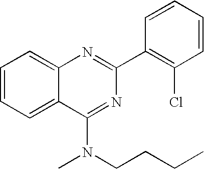

The compound N-butyl-2-(2-chlorophenyl)-N-methylquinazolin-4-amine was prepared following the procedures described for Example 13 using 2-chlorobenzoyl chloride in Step 1 and N-methylbutan-1-amine in Step 3. ¹H NMR (400 MHz, CDCl₃) δ 8.02 (d, 1H), 7.93 (d, 1H), 7.84 (m, 1H), 7.71 (t, 1H), 7.47 (m, 1H), 7.42 (t, 1H), 7.35 (m, 2H), 3.78 (m, 2H), 3.40 (s, 3H), 1.82 (m, 2H), 1.42 (m, 2H), 0.97 (t, 3H). MS (M/z, M+1), 326.3.

### EXAMPLE 65

### 1-(2-(2-Chlorophenyl)quinazolin-4-yl)piperidin-4-one

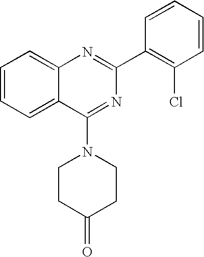

The compound 1-(2-(2-chlorophenyl)quinazolin-4-yl)piperidin-4-one was prepared following the procedures described for Example 13 using 2-chlorobenozyl chloride in Step 1 and 4-piperidone monohydrate hydrochloride in Step 3. ¹H NMR (400 MHz, CDCl₃) δ 8.02 (m, 2H), 7.86 (m, 1H), 7.81 (t, 1H), 7.54 (t, 1H), 7.50 (m, 1H), 7.38 (m, 2H), 4.15 (m, 4H), 2.71 (m, 4H). MS (M/z, M+1), 338.2.

### EXAMPLE 66

### 4-(4-Chlorophenyl)-1-[2-(2-chlorophenyl)quinazolin-4-yl]piperidin-4-ol

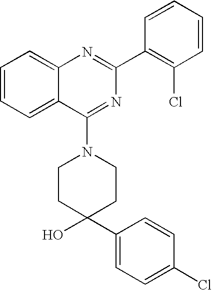

The compound 4-(4-chlorophenyl)-1-[2-(2-chlorophenyl)quinazolin-4-yl]piperidin-4-ol was prepared following the procedures described for Example 13 using 2-chlorobenzoyl chloride in Step 1 and 4-(4-chlorophenyl)piperidin-4-ol in Step 3. ¹H NMR (400 MHz, CDCl₃) δ 7.98 (m, 2H), 7.85 (m, 1H), 7.76 (m, 1H), 7.47 (m, 4H), 7.36 (m, 4H), 4.42 (m, 2H), 3.73 (m, 2H), 2.33 (m, 2H), 1.88 (m, 2H), 1.78 (s, 1H). MS (M/z, M+1), 450.3.

### EXAMPLE 67

### 4-[4-(5-Chloro-2-methylphenyl)piperazin-1-yl]-2-(2-chlorophenyl) quinazoline

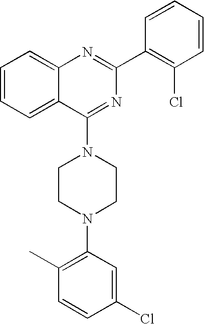

The compound 4-[4-(5-chloro-2-methylphenyl)piperazin-1-yl]-2-(2-chlorophenyl) quinazoline was prepared following the procedures described for Example 13 using 2-chlorobenzoyl chloride in Step 1 and 1-(5-chloro-2-methylphenyl)piperazine in Step 3. ¹H NMR (400 MHz, CDCl₃) δ 8.01 (m, 2H), 7.88 (m, 1H), 7.78 (m, 1H), 7.51 (m, 2H), 7.38 (m, 2H), 7.12 (d, 1H), 7.01 (m, 2H), 3.99 (m, 4H), 3.13 (m, 4H), 2.31 (s, 3H). MS (M/z, M+1), 449.3.

### EXAMPLE 68

### 2-(2-Chlorophenyl)-4-thiomorpholin-4-ylquinazoline

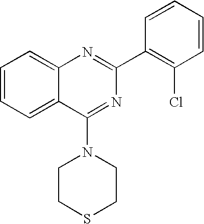

The compound 2-(2-chlorophenyl)-4-thiomorpholin-4-ylquinazoline was prepared following the procedures described for Example 13 using 2-chlorobenozyl chloride in Step 1 and thiomorpholine in Step 3. ¹H NMR (400 MHz, CDCl₃) δ 7.99 (d, 1H), 7.86 (m, 2H), 7.77 (m, 1H), 7.49 (m, 2H), 7.38 (m, 2H), 4.10 (m, 4H), 2.89 (m, 4H). MS (M/z, M+1), 342.2

### EXAMPLE 69

### (4-tert-Butylphenyl)-[2-(2-chlorophenyl)quinazolin-4-yl]amine

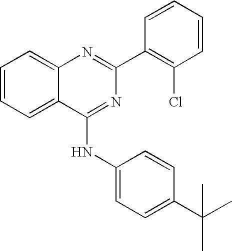

The compound (4-tert-butylphenyl)-[2-(2-chlorophenyl)quinazolin-4-yl]amine was prepared following the procedures described for Example 14 using 2-chlorobenzoyl chloride in Step 1 and 4-tert-butylaniline in Step 3. ¹H NMR (400 MHz, CDCl₃) δ 8.02 (d, 1H), 7.92 (d, 1H), 7.82 (m, 4H), 7.60 (t, 1H), 7.51 (m, 2H), 7.42 (d, 2H), 7.37 (m, 2H), 1.34 (s, 9H). MS (M/z, M+1), 388.3.

### EXAMPLE 70

### 1-(2-(2-Chlorophenyl)quinazolin-4-yl)pyrrolidin-2-one

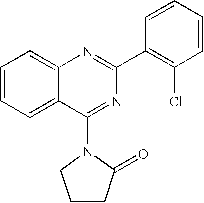

The compound 1-(2-(2-chlorophenyl)quinazolin-4-yl)pyrrolidin-2-one was prepared following the procedures described for Example 14 using 2-chlorobenzoylchloride in Step 1 and pyrrolidinone in Step 3. ¹H NMR (400 MHz, CDCl₃) δ 8.08 (m, 2H), 7.89 (m, 2H), 7.63 (m, 1H), 7.52 (m, 1H), 7.41 (m, 2H), 4.29 (t, 2H), 2.75 (t, 2H), 2.32 (m, 2H). MS (M/z, M+1), 324.4.

### EXAMPLE 71

### 1-(2-(2-Chlorophenyl)quinazolin-4-yl)piperazin-2-one

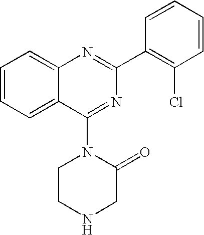

The compound 1-(2-(2-chlorophenyl)quinazolin-4-yl)piperazin-2-one was prepared following the procedures described for Example 14 using 2-chlorobenzoyl chloride in Step 1 and tert-butyl 3-oxopiperazine-1-carboxylate in Step 3. ¹H NMR (400 MHz, CDCl₃) δ 8.14 (d, 1H), 7.89 (m, 3H), 7.65 (m, 1H), 7.52 (m, 1H), 7.42 (m, 2H), 4.11 (m, 2H), 3.83 (m, 2H), 3.48 (m, 1H), 3.37 (m, 2H). MS (M/z, M+1), 339.4.

### EXAMPLE 72

### 4-(2-(2-Chlorophenyl)quinazolin-4-yl)piperazin-2-one

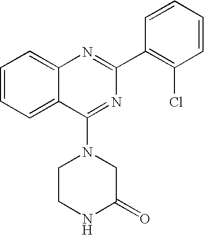

The compound 4-(2-(2-chlorophenyl)quinazolin-4-yl)piperazin-2-one was prepared following the procedures described for Example 13 using 2-chlorobenzoyl chloride in Step 1 and 2-oxopiperazine in Step 3. ¹H NMR (400 MHz, CDCl₃) δ 8.01 (d, 1H), 7.93 (d, 1H), 7.85 (m, 1H), 7.80 (m, 1H), 7.51 (m, 2H), 7.37 (m, 2H), 6.30 (m, 1H), 4.57 (s, 2H), 4.12 (m, 2H), 3.68 (m, 2H). MS (M/z, M+1), 339.4.

### EXAMPLE 73

### 1-(2-(2-Chlorophenyl)quinazolin-4-yl)-4-ethylpiperazin-2-one

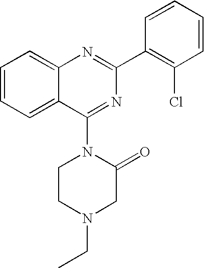

The compound 1-(2-(2-chlorophenyl)quinazolin-4-yl)-4-ethylpiperazin-2-one was prepared from 1-(2-(2-chlorophenyl)quinazolin-4-yl)piperazin-2-one following the procedures described for Example 21 using ethyl iodide. ¹H NMR (400 MHz, CDCl₃) δ 8.13 (d, 1H), 7.89 (m, 3H), 7.64 (m, 1H), 7.52 (m, 1H), 7.41 (m, 2H), 4.13 (m, 2H), 3.45 (s, 2H), 2.97 (m, 2H), 2.61 (q, 2H), 1.20 (t, 3H). MS (M/z, M+1), 367.4.

### EXAMPLE 74

### 1-(2-(2-Chlorophenyl)quinazolin-4-yl)-4-methylpiperazin-2-one

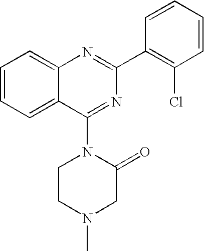

The compound 1-(2-(2-chlorophenyl)quinazolin-4-yl)-4-methylpiperazin-2-one was prepared from 1-(2-(2-chlorophenyl)quinazolin-4-yl)piperazin-2-one following the procedures described for Example 21. ¹H NMR (400 MHz, CDCl₃) δ 8.13 (d, 1H), 7.88 (m, 3H), 7.63 (m, 1H), 7.52 (m, 1H), 7.40 (m, 2H), 4.11 (m, 2H), 3.41 (s, 2H), 2.93 (m, 2H), 2.47 (s, 3H). MS (M/z, M+1), 353.4.

### EXAMPLE 75

### tert-Butyl 4-(2-(2-Chlorophenyl)quinazolin-4-yl)-3-oxopiperazine-1-carboxylate

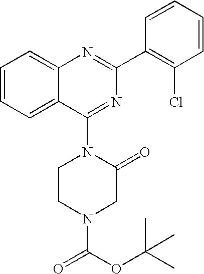

The compound tert-butyl 4-(2-(2-chlorophenyl)quinazolin-4-yl)-3-oxopiperazine-1-carboxylate was prepared following the procedures described for Example 14 using tert-butyl 3-oxopiperazine-1-carboxylate. ¹H NMR (400 MHz, CDCl₃) δ 8.15 (d, 1H), 7.94 (m, 1H), 7.88 (m, 2H), 7.67 (m, 1H), 7.53 (m, 1H), 7.42 (m, 2H), 4.40 (s, 2H), 4.14 (m, 2H), 3.93 (m, 2H), 1.53 (s, 9H). MS (M/z, M+1), 439.6.

### EXAMPLE 76

### 4-(2-(2-Chlorophenyl)quinazolin-4-yl)-1-methylpiperazin-2-one

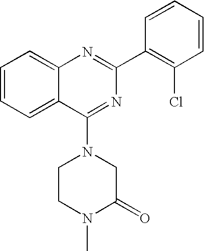

The compound 4-(2-(2-chlorophenyl)quinazolin-4-yl)-1-methylpiperazin-2-one was prepared from 4-(2-(2-chlorophenyl)quinazolin-4-yl)piperazin-2-one following the procedures described for Example 21. ¹H NMR (400 MHz, CDCl₃) δ 7.99 (d, 1H), 7.92 (d, 1H), 7.83 (m, 1H), 7.78 (m, 1H), 7.49 (m, 2H), 7.37 (m, 2H), 4.56 (s, 2H), 4.14 (m, 2H), 3.62 (m, 2H), 3.01 (s, 3H). MS (M/z, M+1), 353.4.

### EXAMPLE 77

### 2-(2-Chlorophenyl)-4-((3S,5R)-3,5-dimethylpiperazin-1-yl)quinazoline

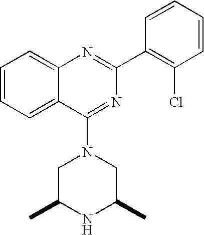

The compound 2-(2-chlorophenyl)-4-((3S,5R)-3,5-dimethylpiperazin-1-yl)quinazoline was prepared following the procedures for Example 13 using 2-chlorobenzoyl chloride in Step 1 and cis-2,6-dimethylpiperazine in Step 3. ¹H NMR (400 MHz, CDCl₃) δ 7.97 (d, 1H), 7.93 (d, 1H), 7.85 (m, 1H), 7.74 (m, 1H), 7.47 (m, 2H), 7.36 (m, 2H), 4.37 (m, 2H), 3.14 (m, 2H), 2.82 (m, 2H), 1.13 (d, 6H). MS (M/z, M+1), 353.5.

### EXAMPLE 78

### 2-(2-Chlorophenyl)-4-((3S,5R)-3,4,5-trimethylpiperazin-1-yl)quinazoline

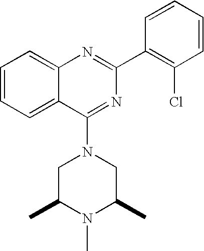

The compound 2-(2-chlorophenyl)-4-((3S,5R)-3,4,5-trimethylpiperazin-1-yl)quinazoline was prepared from 2-(2-chlorophenyl)-4-((3S,5R)-3,5-dimethylpiperazin-1-yl)quinazoline following the procedures described for Example 21. ¹H NMR (400 MHz, CDCl₃) δ 7.96 (d, 1H), 7.91 (d, 1H), 7.86 (m, 1H), 7.74 (m, 1H), 7.47 (m, 2H), 7.35 (m, 2H), 4.29 (m, 2H), 3.09 (m, 2H), 2.46 (m, 2H), 2.33 (s, 3H), 1.16 (d, 6H). MS (M/z, M+1), 367.5.

### EXAMPLE 79

### 2-(2-Chlorophenyl)-4-((3S,5R)-3,5-dimethyl-4-propylpiperazin-1-yl)quinazoline

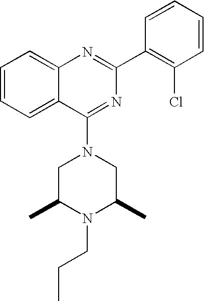

The compound 2-(2-chlorophenyl)-4-((3S,5R)-3,5-dimethyl-4-propylpiperazin-1-yl)quinazoline was prepared from 2-(2-chlorophenyl)-4-((3S,5R)-3,5-dimethylpiperazin-1-yl)quinazoline following the procedures described for Example 21 using iodopropane. ¹H NMR (400 MHz, CDCl₃) δ 7.96 (d, 1H), 7.93 (d, 1H), 7.87 (m, 1H), 7.75 (m, 1H), 7.47 (m, 2H), 7.37 (m, 2H), 4.31 (m, 2H), 3.08 (m, 2H), 2.88 (m, 2H), 2.77 (m, 2H), 1.45 (m, 2H), 1.16 (d, 6H), 0.86 (t, 3H). MS (M/z, M+1), 395.5.

### EXAMPLE 80

### 2-(2-Chlorophenyl)-4-((3S,5R)-4-ethyl-3,5-dimethylpiperazin-1-yl)quinazoline

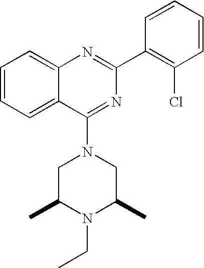

The compound 2-(2-chlorophenyl)-4-((3S,5R)-4-ethyl-3,5-dimethylpiperazin-1-yl)quinazoline was prepared from 2-(2-chlorophenyl)-4-((3S,5R)-3,5-dimethylpiperazin-1-yl)quinazoline following the procedures described for Example 21 using ethyl iodide. ¹H NMR (400 MHz, CDCl₃) δ 7.98 (d, 1H), 7.93 (d, 1H), 7.87 (m, 1H), 7.75 (m, 1H), 7.48 (m, 2H), 7.38 (m, 2H), 4.33 (m, 2H), 3.08 (m, 2H), 2.98 (q, 2H), 2.90 (m, 2H), 1.17 (d, 6H), 0.94 (t, 3H). MS (M/z, M+1), 381.5.

### EXAMPLE 81

### (4-tert-Butylphenyl)-[2-(2-chlorophenyl)quinazolin-4-yl]methylamine

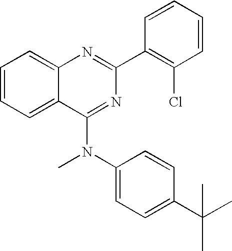

The compound (4-tert-butylphenyl)-[2-(2-chlorophenyl)quinazolin-4-yl]methylamine was prepared from (4-tert-butylphenyl)-[2-(2-chlorophenyl)quinazolin-4-yl]amine following the procedures described for Example 21. ¹H NMR (400 MHz, CDCl₃) δ 7.97 (d, 1H), 7.91 (d, 1H), 7.59 (m, 1H), 7.52 (d, 1H), 7.39 (m, 4H), 7.17 (d, 2H), 7.07 (m, 2H), 3.70 (s, 3H), 1.36 (s, 9H). MS (M/z, M+1), 402.2.

### EXAMPLE 82

### 2-(2-Chlorophenyl)-4-piperazin-1-ylquinazoline

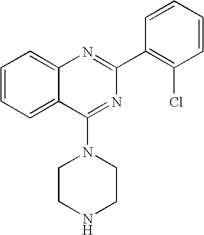

The compound 2-(2-chlorophenyl)-4-piperazin-1-ylquinazoline was prepared following the procedures described for Example 13 using 2-chlorobenzoyl chloride in Step 1 and piperizine in Step 3. ¹H NMR (400 MHz, CDCl₃) δ 7.98 (d, 1H), 7.93 (d, 1H), 7.86 (m, 1H), 7.75 (m, 1H), 7.48 (m, 2H), 7.36 (m, 2H), 3.83 (m, 4H), 3.11 (m, 4H). MS (M/z, M+1), 325.3.

### EXAMPLE 83

### 2-(2-Chlorophenyl)-4-[4-(propane-2-sulfonyl)piperazin-1-yl]quinazoline

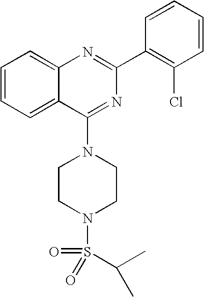

The compound 2-(2-chlorophenyl)-4-[4-(propane-2-sulfonyl)piperazin-1-yl]quinazoline was synthesized according to Scheme IV. 2-(2-Chlorophenyl)-4-piperazin-1-ylquinazoline (71 mg, 0.22 mmol) and triethylamine (90 uL, 0.66 mmol) were dissolved in THF (2 mL). Isopropylsulfonyl chloride (22 uL, 0.20 mmol) was added and the reaction stirred at room temperature. The mixture was then concentrated onto silica gel and purified by column chromatography (0-4% MeOH/CH₂Cl₂) to give 65 mg of the title compound. ¹H NMR (400 MHz, CDCl₃) δ 8.02 (d, 1H), 7.91 (d, 1H), 7.85 (m, 1H), 7.80 (m, 1H), 7.51 (m, 2H), 7.38 (m, 2H), 3.91 (m, 4H), 3.59 (m, 4H), 3.24 (m, 1H), 1.37 (d, 6H). MS (M/z, M+1), 431.3.

### EXAMPLE 84

### 2-(2-Chlorophenyl)-4-(4-(propylsulfonyl)piperazin-1-yl)quinazoline

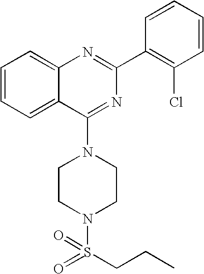

The compound 2-(2-chlorophenyl)-4-(4-(propylsulfonyl)piperazin-1-yl)quinazoline was prepared following the procedures described for Example 83 using 1-propanesulfonyl chloride. ¹H NMR (400 MHz, CDCl₃) δ 8.02 (d, 1H), 7.91 (d, 1H), 7.86 (m, 1H), 7.80 (m, 1H), 7.51 (m, 2H), 7.39 (m, 2H), 3.94 (m, 4H), 3.50 (m, 4H), 2.92 (m, 2H), 1.89 (m, 2H), 1.07 (t, 3H). MS (M/z, M+1), 431.3.

### EXAMPLE 85

### 2-(2-Chlorophenyl)-4-(4-(ethylsulfonyl)piperazin-1-yl)quinazoline

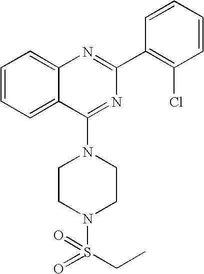

The compound 2-(2-chlorophenyl)-4-(4-(ethylsulfonyl)piperazin-1-yl)quinazoline was prepared following the procedures described for Example 83 using ethanesulfonyl chloride. ¹H NMR (400 MHz, CDCl₃) δ 8.02 (d, 1H), 7.91 (d, 1H), 7.85 (m, 1H), 7.80 (m, 1H), 7.51 (m, 2H), 7.38 (m, 2H), 3.94 (m, 4H), 3.52 (m, 4H), 3.00 (q, 2H), 1.40 (t, 3H). MS (M/z, M+1), 417.3.

### EXAMPLE 86

### 2-(2-Chlorophenyl)-4-(4-(methylsulfonyl)piperazin-1-yl)quinazoline

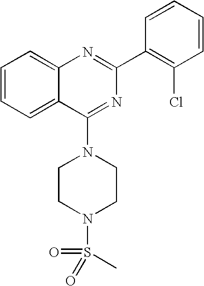

The compound 2-(2-chlorophenyl)-4-(4-(methylsulfonyl)piperazin-1-yl)quinazoline was prepared following the procedures described for Example 83 using methanesulfonyl chloride. ¹H NMR (400 MHz, CDCl₃) δ 8.02 (d, 1H), 7.92 (d, 1H), 7.85 (m, 1H), 7.80 (m, 1H), 7.51 (m, 2H), 7.39 (m, 2H), 3.97 (m, 4H), 3.45 (m, 4H), 2.82 (s, 3H). MS (M/z, M+1), 403.3

### EXAMPLE 87

### 4-(2-(2-chlorophenyl)quinazolin-4-yl)-N-ethylpiperazine-1-carboxamide

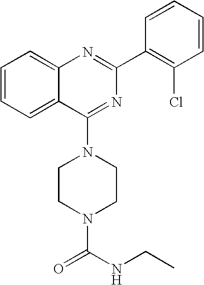

The compound 4-(2-(2-chlorophenyl)quinazolin-4-yl)-N-ethylpiperazine-1-carboxamide synthesized according to Scheme IV. 2-(2-Chlorophenyl)-4-piperazin-1-ylquinazoline (88 mg, 0.27 mmol) was dissolved in THF (2 mL) and ethylisocyanate (21 μL, 0.27 mmol) was added and the mixture stirred at room temperature. The mixture was then concentrated onto silica gel and purified by flash column chromatography (0-4% MeOH/CH₂Cl₂) to give the title compound. ¹H NMR (400 MHz, CDCl₃) δ 8.00 (d, 1H), 7.95 (d, 1H), 7.85 (m, 1H), 7.78 (m, 1H), 7.49 (m, 2H), 7.37 (m, 2H), 4.46 (m, 1H), 3.89 (m, 4H), 3.62 (m, 4H), 3.32 (m, 2H), 1.17 (t, 3H). MS (M/z, M+1), 396.3

### EXAMPLE 88

### 4-(2-(2-Chlorophenyl)quinazolin-4-yl)-N-methylpiperazine-1-carboxamide

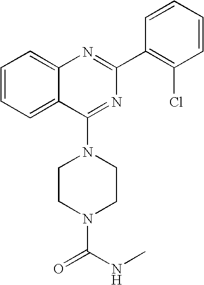

The compound 4-(2-(2-chlorophenyl)quinazolin-4-yl)-N-methylpiperazine-1-carboxamide was prepared following the procedures described for Example 87 using methylisocyanate. ¹H NMR (400 MHz, CDCl₃) δ 8.01 (d, 1H), 7.94 (d, 1H), 7.85 (m, 1H), 7.78 (m, 1H), 7.49 (m, 2H), 7.38 (m, 2H), 4.54 (m, 1H), 3.88 (m, 4H), 3.62 (m, 4H), 2.85 (d, 3H). MS (M/z, M+1), 382.3.

### EXAMPLE 89

### N-tert-Butyl-4-(2-(2-chlorophenyl)quinazolin-4-yl)piperazine-1-carboxamide

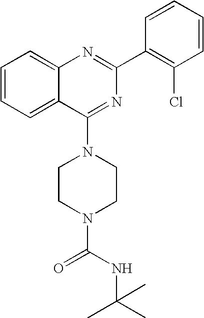

The compound N-tert-butyl-4-(2-(2-chlorophenyl)quinazolin-4-yl)piperazine-1-carboxamide was prepared following the procedures described for Example 87 using tert-butylisocyanate. ¹H NMR (400 MHz, CDCl₃) δ 8.01 (d, 1H), 7.95 (d, 1H), 7.86 (m, 1H), 7.78 (m, 1H), 7.50 (m, 2H), 7.37 (m, 2H), 4.36 (s, 1H), 3.89 (m, 4H), 3.58 (m, 4H), 1.38 (m, 9H). MS (M/z, M+1), 424.4.

### EXAMPLE 90

### 4-(2-(2-Chlorophenyl)quinazolin-4-yl)-N-isopropylpiperazine-1-carboxamide

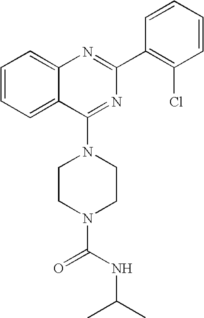

The compound 4-(2-(2-chlorophenyl)quinazolin-4-yl)-N-isopropylpiperazine-1-carboxamide was prepared following the procedures described for Example 87 using isopropylisocyanate. ¹H NMR (400 MHz, CDCl₃) δ 8.00 (d, 1H), 7.94 (d, 1H), 7.86 (m, 1H), 7.78 (m, 1H), 7.49 (m, 2H), 7.37 (m, 2H), 4.28 (d, 1H), 4.02 (m, 1H), 3.89 (m, 4H), 3.61 (m, 4H), 1.13 (d, 6H). MS (M/z, M+1), 410.4.

### EXAMPLE 91

### 4-(2-(2-Chlorophenyl)quinazolin-4-yl)-N-propylpiperazine-1-carboxamide

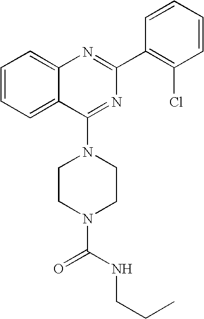

The compound 4-(2-(2-chlorophenyl)quinazolin-4-yl)-N-propylpiperazine-1-carboxamide was prepared following the procedures described for Example 87 using propylisocyanate. ¹H NMR (400 MHz, CDCl₃) δ 8.21 (d, 1H), 8.04 (d, 1H), 7.95 (t, 1H), 7.83 (d, 1H), 7.69 (t, 1H) 7.51 (m, 2H), 7.44 (m, 1H), 4.35 (m, 4H), 3.73 (m, 4H), 3.23 (m, 2H), 1.55 (m, 2H), 0.93 (t, 3H). MS (M/z, M+1), 410.4.

### EXAMPLE 92

### Methyl 4-(2-(2-Chlorophenyl)quinazolin-4-yl)piperazine-1-carboxylate

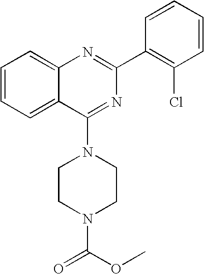

The compound methyl 4-(2-(2-chlorophenyl)quinazolin-4-yl)piperazine-1-carboxylate was prepared following the procedures described for Example 83 using methyl chloroformate. ¹H NMR (400 MHz, CDCl₃) δ 8.00 (d, 1H), 7.93 (d, 1H), 7.86 (m, 1H), 7.78 (m, 1H), 7.50 (m, 2H), 7.37 (m, 2H), 3.81 (m, 4H), 3.74 (s, 3H), 3.70 (m, 4H). MS (M/z, M+1), 383.4.

### EXAMPLE 93

### Isopropyl 4-(2-(2-chlorophenyl)quinazolin-4-yl)piperazine-1-carboxylate

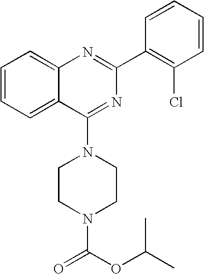

The compound isopropyl 4-(2-(2-chlorophenyl)quinazolin-4-yl)piperazine-1-carboxylate was prepared following the procedures described for Example 83 using isopropylchloroformate. ¹H NMR (400 MHz, CDCl₃) δ 7.99 (d, 1H), 7.93 (d, 1H), 7.85 (m, 1H), 7.77 (m, 1H), 7.49 (m, 2H), 7.36 (m, 2H), 4.95 (m, 1H), 3.81 (m, 4H), 3.71 (m, 4H), 1.26 (d, 6H). MS (M/z, M+1), 411.5.

### EXAMPLE 94

### Propyl 4-(2-(2-chlorophenyl)quinazolin-4-yl)piperazine-1-carboxylate

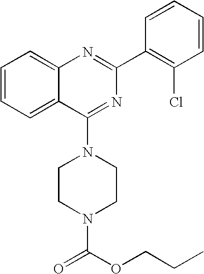

The compound propyl 4-(2-(2-chlorophenyl)quinazolin-4-yl)piperazine-1-carboxylate was prepared following the procedures described for Example 83 using propylchloroformate. ¹H NMR (400 MHz, CDCl₃) δ 8.00 (d, 1H), 7.93 (d, 1H), 7.85 (m, 1H), 7.78 (m, 1H), 7.49 (m, 2H), 7.37 (m, 2H), 4.07 (t, 2H), 3.82 (m, 4H), 3.72 (m, 4H), 1.68 (m, 2H), 0.97 (t, 3H). MS (M/z, M+1), 411.5.

### EXAMPLE 95

### 1-(4-(2-(2-Chlorophenyl)quinazolin-4-yl)piperazin-1-yl)butan-1-one

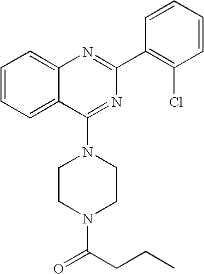

The compound 1-(4-(2-(2-chlorophenyl)quinazolin-4-yl)piperazin-1-yl)butan-1-one was prepared following the procedures described for Example 83 using butyryl chloride. ¹H NMR (400 MHz, CDCl₃) δ 8.01 (d, 1H), 7.94 (d, 1H), 7.86 (m, 1H), 7.79 (m, 1H), 7.51 (m, 2H), 7.38 (m, 2H), 3.84 (m, 6H), 3.73 (m, 2H), 2.37 (m, 2H), 1.70 (m, 2H), 1.00 (t, 3H). MS (M/z, M+1), 395.3.

### EXAMPLE 96

### 1-(4-(2-(2-Chlorophenyl)quinazolin-4-yl)piperazin-1-yl)-2-methylpropan-1-one

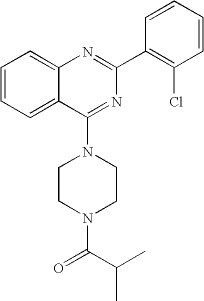

The compound 1-(4-(2-(2-chlorophenyl)quinazolin-4-yl)piperazin-1-yl)-2-methylpropan-1-one was prepared following the procedures described for Example 83 using isobutyryl chloride. ¹H NMR (400 MHz, CDCl₃) δ 8.02 (d, 1H), 7.95 (d, 1H), 7.87 (m, 1H), 7.80 (m, 1H), 7.54 (m, 1H), 7.50 (m, 1H), 7.38 (m, 2H), 3.79 (m, 8H), 2.86 (m, 1H), 1.18 (d, 6H). MS (M/z, M+1), 395.5.

### EXAMPLE 97

### (4-(2-(2-Chlorophenyl)quinazolin-4-yl)piperazin-1-yl)(cyclopropyl)methanone

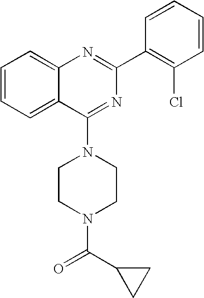

The compound (4-(2-(2-chlorophenyl)quinazolin-4-yl)piperazin-1-yl)(cyclopropyl)methanone was prepared following the procedures described for Example 83 using cyclopropanecarbonyl chloride. ¹H NMR (400 MHz, CDCl₃) δ 8.01 (d, 1H), 7.95 (d, 1H), 7.86 (m, 1H), 7.79 (m, 1H), 7.50 (m, 2H), 7.37 (m, 2H), 3.89 (m, 8H), 1.78 (m, 1H), 1.03 (m, 2H), 0.82 (m, 2H). MS (M/z, M+1), 393.5.

### EXAMPLE 98

### 1-(4-(2-(2-Chlorophenyl)quinazolin-4-yl)piperazin-1-yl)propan-1-one

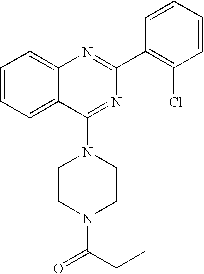

The compound 1-(4-(2-(2-chlorophenyl)quinazolin-4-yl)piperazin-1-yl)propan-1-one was prepared following the procedures described for Example 13 using propionyl chloride. ¹H NMR (400 MHz, CDCl₃) δ 8.01 (d, 1H), 7.94 (d, 1H), 7.86 (m, 1H), 7.79 (m, 1H), 7.50 (m, 2H), 7.37 (m, 2H), 3.85 (m, 6H), 3.72 (m, 2H), 2.41 (q, 2H), 1.20 (t, 3H). MS (M/z, M+1), 381.3.

### EXAMPLE 99

### 1-(4-(2-(2-Chlorophenyl)quinazolin-4-yl)piperazin-1-yl)ethanone

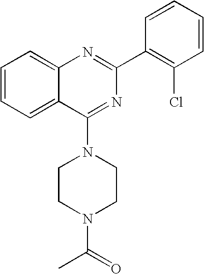

The compound 1-(4-(2-(2-chlorophenyl)quinazolin-4-yl)piperazin-1-yl)ethanone was synthesized following the procedures described for Example 13 using 1-acetylpiperizine. ¹H NMR (400 MHz, CDCl₃) δ 8.02 (d, 1H), 7.95 (d, 1H), 7.86 (m, 1H), 7.81 (m, 1H), 7.51 (m, 2H), 7.39 (m, 2H), 3.88 (m, 2H), 3.84 (m, 4H), 3.74 (m, 2H), 2.17 (s, 3H). MS (M/z, M+1), 367.4.

### EXAMPLE 100

### (4-(2-(2-Chlorophenyl)quinazolin-4-yl)piperazin-1-yl)(2,2,3,3-tetramethylcyclopropyl)methanone

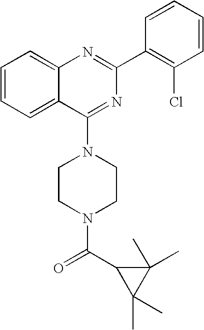

The compound (4-(2-(2-chlorophenyl)quinazolin-4-yl)piperazin-1-yl)(2,2,3,3-tetramethylcyclopropyl)methanone was synthesized according to Scheme IV. 2-(2-Chlorophenyl)-4-piperazin-1-ylquinazoline (110 mg, 0.339 mmol) 2,2,3,3-tetramethylcyclopropane carboxylic acid (58 mg, 0.407 mmol) and N-ethyl-N″-(3-dimethylaminopropyl)carbodiimide hydrochloride (78 mg, 0.407 mmol) were dissolved in THF (3 mL) and stirred at room temperature for 18 hours. The mixture was then concentrated onto silica gel and purified by flash column chromatography (0-8% MeOH/CH₂Cl₂) to give the title compound. ¹H NMR (400 MHz, CDCl₃) δ 8.01 (d, 1H), 7.96 (d, 1H), 7.86 (m, 1H), 7.81 (m, 1H), 7.52 (m, 2H), 7.39 (m, 2H), 3.83 (m, 8H), 1.21 (s, 12H), 1.10 (s, 1H). MS (M/z, M+1), 449.6.

### EXAMPLE 101

### 1-(4-(2-(2-Chlorophenyl)quinazolin-4-yl)piperazin-1-yl)-2-(dimethylamino)ethanone

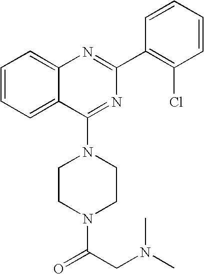

The compound 1-(4-(2-(2-chlorophenyl)quinazolin-4-yl)piperazin-1-yl)-2-(dimethylamino)ethanone was prepared following the procedures described in Example 100 using N,N-dimethylglycine. ¹H NMR (400 MHz, CDCl₃) δ 8.01 (d, 1H), 7.95 (d, 1H), 7.86 (m, 1H), 7.80 (m, 1H), 7.51 (m, 2H), 7.39 (m, 2H), 3.86 (m, 8H), 3.18 (s, 2H), 2.30 (s, 6H). MS (M/z, M+1), 410.5.

### EXAMPLE 102

### 1-(2-(2-Chlorophenyl)quinazolin-4-yl)-N,N-dimethylpiperidine-4-carboxamide

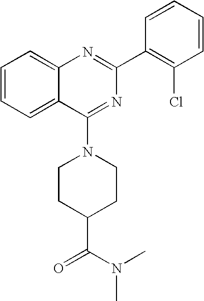

The compound 1-(2-(2-chlorophenyl)quinazolin-4-yl)-N,N-dimethylpiperidine-4-carboxamide was prepared following the procedures described in Example 100 using dimethylamine and 1-(2-(2-chlorophenyl)quinazolin-4-yl)piperidine-4-carboxylic acid. ¹H NMR (400 MHz, CDCl₃) δ 7.97 (m, 2H), 7.86 (m, 1H), 7.76 (m, 1H), 7.49 (m, 2H), 7.36 (m, 2H), 4.47 (m, 2H), 3.21 (m, 2H), 3.13 (s, 3H), 3.00 (s, 3H), 2.87 (m, 1H), 2.12 (m, 2H), 1.90 (m, 2). MS (M/z, M+1), 395.5.

### EXAMPLE 103

### 1-(4-(2-(2-Chlorophenyl)quinazolin-4-yl)piperazin-1-yl)-2,2,2-trifluoroethanone

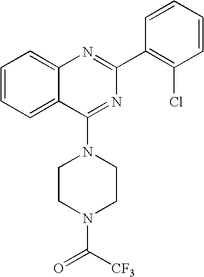

The compound 1-(4-(2-(2-chlorophenyl)quinazolin-4-yl)piperazin-1-yl)-2,2,2-trifluoroethanone was synthesized according to Scheme IV. 2-(2-Chlorophenyl)-4-piperazin-1-ylquinazoline (123 mg, 0.379 mmol) was dissolved in 1,4-dioxane (3 mL) and trifluoroacetic anhydride (0.26 mL, 1.90 mmol) was added. The mixture was heated to 80° C. for 18 hours. The mixture was then concentrated onto silica gel and purified by flash column chromatography (0-50% EtOAc/hexanes) to give the title compound. ¹H NMR (400 MHz, CDCl₃) δ 8.28 (d, 1H), 8.00 (m, 2H), 7.87 (d, 1H), 7.71 (t, 1H), 7.55 (m, 2H), 7.47 (m, 1H), 4.35 (m, 4H), 3.94 (m, 4H). MS (M/z, M+1), 421.4.

### EXAMPLE 104

### 2-(2-Chlorophenyl)-4-piperazin-1-ylquinazoline

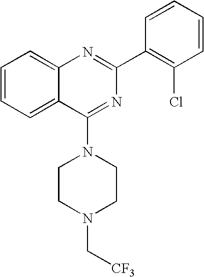

2-(2-Chlorophenyl)-4-piperazin-1-ylquinazoline (119 mg, 0.367 mmol) and 2,2,2-trifluoroethyl trifluoromethanesulfonate (103 mg, 0.440 mmol) were dissolved in THF (3 mL). Triethylamine (0.26 mL, 1.84 mmol) was then added and the mixture heated to 50° C. for 18 hours. The mixture was then concentrated onto silica gel and purified by flash column chromatography (0-8% MeOH/CH₂Cl₂) to give 2-(2-chlorophenyl)-4-(4-(2,2,2-trifluoroethyl)piperazin-1-yl)quinazoline. ¹H NMR (400 MHz, CDCl₃) δ 7.98 (d, 1H), 7.92 (d, 1H), 7.85 (m, 1H), 7.76 (m, 1H), 7.49 (m, 2H), 7.36 (m, 2H), 3.88 (m, 4H), 3.06 (q, 2H), 2.90 (m, 4H). MS (M/z, M+1), 407.5.

### EXAMPLE 105

### 2-(2-Chlorophenyl)-4-piperazin-1-ylquinazoline

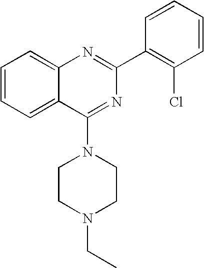

2-(2-Chlorophenyl)-4-piperazin-1-ylquinazoline (169 mg, 0.521 mmol) and ethyl iodide (0.08 mL, 1.04 mmol) were dissolved in acetonitrile (5 mL). Potassium carbonate (215 mg, 1.56 mmol) was added and the mixture heated to 50° C. for 18 hours. The mixture was concentrated onto silica gel and purified by flash column chromatography (0-8% MeOH/CH₂Cl₂) to give 2-(2-chlorophenyl)-4-(4-ethylpiperazin-1-yl)quinazoline. ¹H NMR (400 MHz, CDCl₃) δ 7.98 (d, 1H), 7.95 (d, 1H), 7.87 (m, 1H), 7.76 (m, 1H), 7.49 (m, 2H), 7.37 (m, 2H), 3.92 (m, 4H), 2.68 (m, 4H), 2.53 (q, 2H), 1.15 (t, 3H). MS (M/z, M+1), 353.1.

### EXAMPLE 106

### 2-(2-Chlorophenyl)-4-(4-(trifluoromethyl)piperazin-1-yl)quinazoline

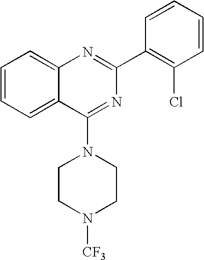

The compound 2-(2-chlorophenyl)-4-(4-(trifluoromethyl)piperazin-1-yl)quinazoline was synthesized according to Scheme IV.

### Step 1

### 2-(2-Chlorophenyl)-4-piperazin-1-ylquinazoline (196 mg, 0.604 mmol) and carbon disulfide (0.04 mL, 0.725 mmol) were dissolved in THF (6 mL) and the mixture cooled to 0° C. Then lithium hexamethyldisilylamide (1.0M/TMBE, 0.73 mL, 0.725 mmol) was added and the mixture stirred for 30 minutes. Methyl iodide (0.05 mL, 0.725 mmol) was then added and the mixture stirred at room temperature for 30 minutes. The mixture was poured into H₂O and extracted with EtOAc. The organic phase was washed with brine and then dried over Na₂SO₄, filtered, and concentrated. The crude material was then purified by flash column chromatography (0-50% EtOAc/hexanes) to give methyl 4-(2-(2-chlorophenyl)quinazolin-4-yl)piperazine-1-carbodithioate. MS (M/z, M+1), 415.0.

### Step 2

Methyl 4-(2-(2-chlorophenyl)quinazolin-4-yl)piperazine-1-carbodithioate (99 mg, 0.239 mmol) was dissolved in dry CH₂Cl₂ (3 mL) and the mixture cooled to −78° C. Then polyvinyl(hydrogen fluoride-pyridinium) (300 mg, 2.39 mmol) was added followed by N-bromosuccinimide (170 mg, 0.956 mmol). The mixture was stirred at −78° C. for one hour then stirred at room temperature for one hour. The mixture was poured into Na₂S₂O₃ (20 mL) and NaHCO₃ (20 mL) and stirred for 15 minutes. The mixture was extracted with CH₂Cl₂ and the organic phase dried over Na₂SO₄, filtered, and concentrated. The crude material was then purified by flash column chromatography (0-8% MeOH/CH₂Cl₂) to give the title compound. ¹H NMR (400 MHz, CDCl₃) δ 8.02 (d, 1H), 7.92 (d, 1H), 7.87 (m, 1H), 7.79 (m, 1H), 7.50 (m, 2H), 7.38 (m, 2H), 3.89 (m, 4H), 3.16 (m, 4H). MS (M/z, M+1), 393.1.

### EXAMPLE 107

### 2-(2-Chlorophenyl)-4-piperazin-1-ylquinazoline

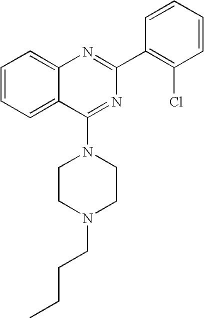

2-(2-Chlorophenyl)-4-piperazin-1-ylquinazoline (147 mg, 0.453 mmol) was dissolved MeOH (4 mL) and butyraldehyde (0.05 mL, 0.544 mmol) was added followed by NaBH(OAc)₃ (134 mg, 0.634 mmol). The reaction was stirred at room temperature for 3 hours. The mixture was then concentrated onto silica gel and purified by flash column chromatography (0-8% MeOH/CH₂Cl₂) to give the title compound. ¹H NMR (400 MHz, CDCl₃) δ 7.94 (m, 2H), 7.84 (m, 1H), 7.74 (m, 1H), 7.46 (m, 2H), 7.35 (m, 2H), 3.89 (m, 4H), 2.66 (m, 4H), 2.41 (m, 2H), 1.51 (m, 2H), 1.35 (m, 2H), 0.92 (t, 3H). MS (M/z, M+1), 381.5.

### EXAMPLE 108

### 2-(2-Chlorophenyl)-4-(4-propylpiperazin-1-yl)quinazoline

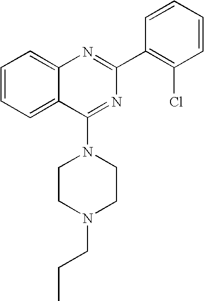

The compound 2-(2-chlorophenyl)-4-(4-propylpiperazin-1-yl)quinazoline was prepared following the procedures described for Example 107 using propionaldehyde. ¹H NMR (400 MHz, CDCl₃) δ 7.95 (m, 2H), 7.85 (m, 1H), 7.73 (m, 1H), 7.46 (m, 2H), 7.36 (m, 2H), 3.89 (m, 4H), 2.66 (m, 4H), 2.39 (m, 2H), 1.56 (m, 2H), 0.94 (t, 3H). MS (M/z, M+1), 367.5.

### EXAMPLE 109

### 2-(4-tert-Butylphenyl)-4-(4-methylpiperazin-1-yl)quinazoline

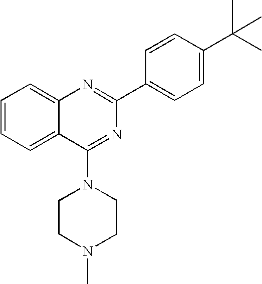

The compound 2-(4-tert-butylphenyl)-4-(4-methylpiperazin-1-yl)quinazoline was prepared following the procedures described for Example 13 using 4-tert-butylbenzoyl chloride in Step 1 and 1-methylpiperazine in Step 3. ¹H NMR (400 MHz, CDCl₃) δ 8.48 (d, 2H), 7.97 (d, 1H), 7.89 (d, 1H), 7.72 (t, 1H), 7.52 (d, 2H), 7.40 (t, 1H), 3.99 (m, 4H), 2.67 (m, 4H), 2.40 (s, 3H), 1.38 (s, 9H). MS (M/z, M+1), 361.2.

### EXAMPLE 110

### 2-(4-tert-Butylphenyl)-4-(pyrrolidin-1-yl)quinazoline

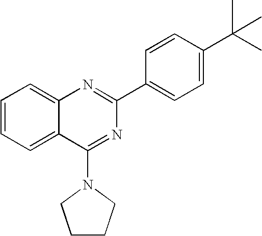

The compound 2-(4-tert-butylphenyl)-4-(pyrrolidin-1-yl)quinazoline was prepared following the procedure described for Example 13 using 4-tert-butylbenzoyl chloride in Step 1 and pyrrolidine in Step 3. ¹H NMR (400 MHz, CDCl₃) δ 8.46 (d, 2H), 8.17 (d, 1H), 7.90 (d, 1H), 7.67 (t, 1H), 7.50 (d, 2H), 7.33 (t, 1H), 4.05 (m, 4H), 2.08 (m, 4H), 1.37 (s, 9H). MS (M/z, M+1), 332.3.

### EXAMPLE 111

### 2-(4-tert-Butylphenyl)-4-(piperidin-1-yl)quinazoline

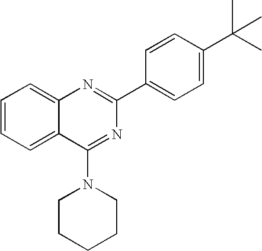

The compound 2-(4-tert-butylphenyl)-4-(piperidin-1-yl)quinazoline was prepared following the procedure described for Example 13 using 4-tert-butylbenzoyl chloride in Step 1 and piperidine in Step 3. ¹H NMR (400 MHz, CDCl₃) δ 8.48 (d, 2H), 7.95 (d, 1H), 7.88 (d, 1H), 7.70 (t, 1H), 7.53 (d, 2H), 7.39 (t, 1H), 3.80 (m, 4H), 1.82 (m, 6H), 1.39 (s, 9H). MS (M/z, M+1), 346.3.

### EXAMPLE 112

### 4-(2-(4-tert-Butylphenyl)quinazolin-4-yl)morpholine

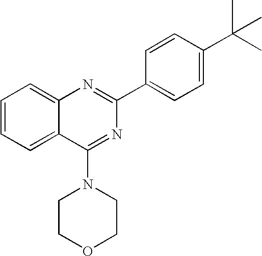

The compound 4-(2-(4-tert-butylphenyl)quinazolin-4-yl)morpholine was prepared following the procedure described for Example 13 using 4-tert-butylbenzoyl chloride in Step 1 and morpholine in Step 3. ¹H NMR (400 MHz, CDCl₃) δ 8.47 (d, 2H), 7.99 (d, 1H), 7.89 (d, 1H), 7.74 (t, 1H), 7.53 (d, 2H), 7.42 (t, 1H), 3.95 (m, 4H), 3.85 (m, 4H), 1.38 (s, 9H). MS (M/z, M+1), 348.3.

### EXAMPLE 113

### N-Benzyl-2-(4-tert-butylphenyl)-N-methylquinazolin-4-amine

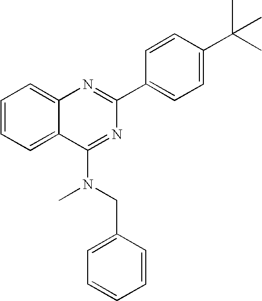

The compound N-benzyl-2-(4-tert-butylphenyl)-N-methylquinazolin-4-amine was prepared following the procedures described for Example 13 using 4-tert-butylbenzoyl chloride in Step 1 and N-methylbenzylamine in Step 3. ¹H NMR (400 MHz, CDCl₃) δ 8.48 (d, 2H), 7.96 (m, 2H), 7.69 (t, 1H), 7.51 (d, 2H), 7.44 (m, 4H), 7.31 (m, 2H), 5.07 (s, 2H), 3.38 (s, 3H), 1.39 (s, 9H). MS (M/z, M+1), 382.3.

### EXAMPLE 114

### 2-(4-tert-Butylphenyl)-4-thiomorpholin-4-ylquinazoline

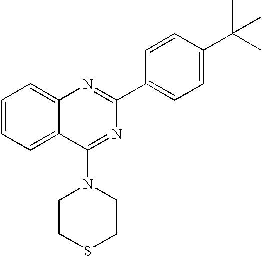

The compound 2-(4-tert-butylphenyl)-4-thiomorpholin-4-ylquinazoline was prepared following the procedures described for Example 13 using 4-tert-butylbenzoyl chloride in Step 1 and thiomorpholine in Step 3. ¹H NMR (400 MHz, CDCl₃) δ 8.45 (d, 2H), 7.97 (d, 1H), 7.83 (d, 1H), 7.72 (t, 1H), 7.51 (d, 2H), 7.41 (t, 1H), 4.10 (m, 4H), 2.91 (m, 4H), 1.37 (s, 9H). MS (M/z, M+1), 364.3.

### EXAMPLE 115

### 2-(4-tert-Butylphenyl)-N-(2,4-difluorophenyl)quinazolin-4-amine

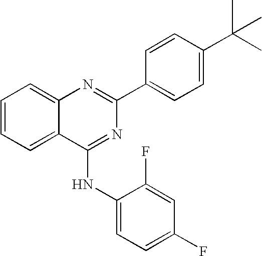

The compound 2-(4-tert-butylphenyl)-N-(2,4-difluorophenyl)quinazolin-4-amine was prepared following the procedures described for Example 14 using 4-tert-butylbenzoyl chloride in Step 1 and 2,4-difluoroaniline in Step 3. ¹H NMR (400 MHz, CDCl₃) δ 8.79 (m, 1H), 8.43 (d, 2H), 8.01 (d, 1H), 7.89 (d, 1H), 7.83 (t, 1H), 7.56 (m, 4H), 7.07 (m, 1H), 7.00 (m, 1H), 1.39 (s, 9H). MS (M/z, M+1), 390.3.

### EXAMPLE 116

### 2-(4-tert-Butylphenyl)-N-(2,4-difluorophenyl)-N-methylquinazolin-4-amine

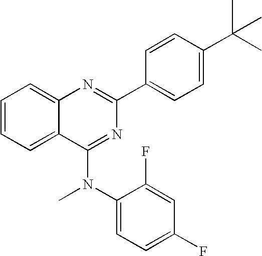

The compound 2-(4-tert-butylphenyl)-N-(2,4-difluorophenyl)-N-methylquinazolin-4-amine was prepared following the procedure described for Example 21 using 2-(4-tert-butylphenyl)-N-(2,4-difluorophenyl)quinazolin-4-amine. ¹H NMR (400 MHz, CDCl₃) δ 8.54 (d, 2H), 7.95 (d, 1H), 7.62 (m, 1H), 7.55 (d, 2H), 7.17 (m, 1H), 7.08 (d, 2H), 6.99 (m, 1H), 6.89 (m, 1H), 3.69 (s, 3H), 1.40 (s, 9H). MS (M/z, M+1), 404.3.

### EXAMPLE 117

### 2-(4-tert-Butylphenyl)-N-(3,4-difluorophenyl)quinazolin-4-amine

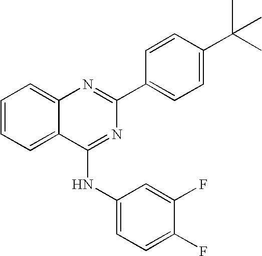

The compound 2-(4-tert-butylphenyl)-N-(3,4-difluorophenyl)quinazolin-4-amine was prepared following the procedures described for Example 14 using 4-tert-butylbenzoyl chloride in Step 1 and 3,4-difluoroaniline in Step 3. ¹H NMR (400 MHz, CDCl₃) δ 8.43 (d, 2H), 8.08 (m, 1H), 7.98 (d, 1H), 7.79 (m, 2H), 7.54 (d, 2H), 7.48 (t, 1H), 7.43 (s, 1H), 7.36 (m, 1H), 7.20 (m, 1H), 1.39 (s, 9H). MS (M/z, M+1), 390.2.

### EXAMPLE 118

### N,2-bis(4-tert-butylphenyl)quinazolin-4-amine

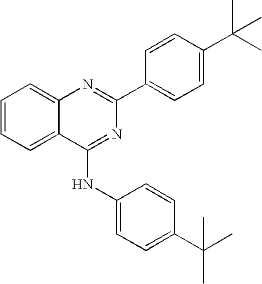

The compound N,2-bis(4-tert-butylphenyl)quinazolin-4-amine was prepared following the procedures described for Example 14 using 4-tert-butylbenzoyl chloride in Step 1 and 4-tert-butylaniline in Step 3. ¹H NMR (400 MHz, DMSO-d₆) δ 8.68 (d, 1H), 8.27 (d, 2H), 7.98 (m, 2H), 7.87 (d, 2H), 7.76 (m, 1H), 7.64 (d, 2H), 7.54 (d, 2H), 1.35 (s, 9H), 1.34 (s, 9H). MS (M/z, M+1), 410.4.

### EXAMPLE 119

### N,2-Bis(4-tert-butylphenyl)-N-methylquinazolin-4-amine

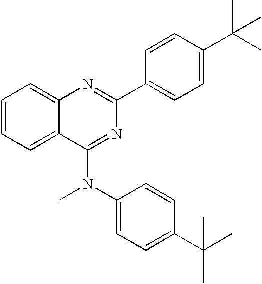

The compound N,2-bis(4-tert-butylphenyl)-N-methylquinazolin-4-amine was prepared from N,2-bis(4-tert-butylphenyl)quinaolin-4-amine following the procedures described for Example 21. ¹H NMR (400 MHz, DMSO-d₆) δ 8.53 (d, 2H), 7.89 (d, 1H), 7.53 (m, 3H), 7.39 (d, 2H), 7.12 (d, 2H), 7.03 (m, 1H), 6.97 (m, 1H), 3.74 (s, 3H), 1.38 (s, 9H), 1.35 (s, 9H). MS (M/z, M+1), 424.5.

### EXAMPLE 120

### N-Benzyl-2-(4-chlorophenyl)-N-methylquinazolin-4-amine

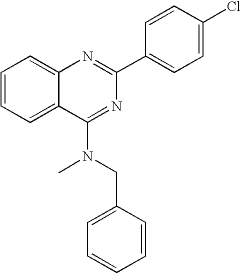

The compound N-benzyl-2-(4-chlorophenyl)-N-methylquinazolin-4-amine was prepared following the procedures described for Example 13 using 4-chlorobenzoyl chloride in Step 1 and N-methylbenzylamine in Step 3. ¹H NMR (400 MHz, CDCl₃) δ 8.50 (d, 2H), 7.93 (d, 2H), 7.70 (t, 1H), 7.43 (m, 6H), 7.31 (m, 2H), 5.06 (s, 2H), 3.37 (s, 3H). MS (M/z, M+1), 360.2.

### EXAMPLE 121

### N-Benzyl-2-(4-chlorophenyl)quinazolin-4-amine

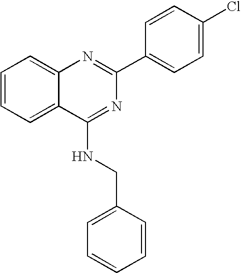

The compound N-benzyl-2-(4-chlorophenyl)quinazolin-4-amine was prepared following the procedures described for Example 13 using 4-chlorobenzoyl chloride in Step 1 and benzylamine in Step 3. ¹H NMR (400 MHz, CDCl₃) δ 8.51 (d, 2H), 7.91 (d, 1H), 7.74 (t, 1H), 7.68 (d, 1H), 7.37 (m, 8H), 5.97 (m, 1H), 4.99 (d, 2H). MS (M/z, M+1), 346.2.

### EXAMPLE 122

### 2-(4-Chlorophenyl)-4-(4-methylpiperazin-1-yl)quinazoline

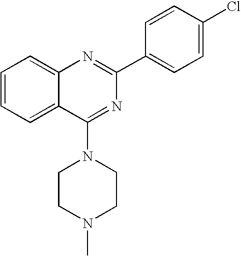

The compound 2-(4-chlorophenyl)-4-(4-methylpiperazin-1-yl)quinazoline was prepared following the procedures described for Example 13 using 4-chlorobenzoyl chloride in Step 1 and 1-methylpiperizine in Step 3. ¹H NMR (400 MHz, CDCl₃) δ 8.50 (d, 2H), 7.94 (d, 1H), 7.89 (d, 1H), 7.73 (t, 1H), 7.43 (m, 3H), 3.90 (m, 4H), 2.66 (m, 4H), 2.40 (s, 3H). MS (M/z, M+1), 339.3.

### EXAMPLE 123

### 2-(4-Chlorophenyl)-4-(3,4-dihydro-1H-isoquinolin-2-yl)quinazoline

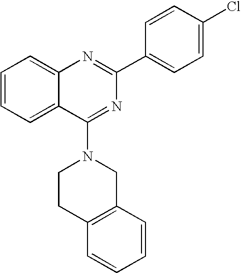

The compound 2-(4-chlorophenyl)-4-(3,4-dihydro-1H-isoquinolin-2-yl)quinazoline was prepared following the procedures described for Example 13 using 4-chlorobenzoyl chloride in Step 1 and 1,2,3,4-tetrahydroisoquinoline in Step 3. ¹H NMR (400 MHz, CDCl₃) δ 8.53 (d, 2H), 7.99 (d, 1H), 7.95 (d, 1H), 7.74 (t, 1H), 7.46 (m, 3H), 7.24 (m, 4H), 5.50 (s, 2H), 4.13 (t, 2H), 3.22 (m, 2H). MS (M/z, M+1), 372.3

### EXAMPLE 124

### 2-(4-Chlorophenyl)-4-(2-methylpiperidin-1-yl)quinazoline

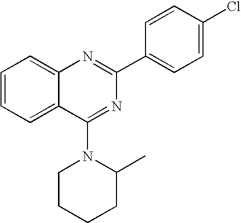

The compound 2-(4-chlorophenyl)-4-(2-methylpiperidin-1-yl)quinazoline was prepared following the procedures described for Example 13 using 4-chlorobenzoyl chloride in Step 1 and 2-methylpiperidine in Step 3. ¹H NMR (400 MHz, CDCl₃) δ 8.50 (d, 2H), 7.91 (d, 1H), 7.84 (d, 1H), 7.69 (t, 1H), 7.44 (d, 2H), 7.38 (t, 1H), 4.81 (m, 1H), 4.22 (m, 1H), 3.44 (m, 1H), 1.76 (m, 6H), 1.43 (d, 3H). MS (M/z, M+1), 338.3.

### EXAMPLE 125

### 2-(4-Chlorophenyl)-4-(4-methylpiperidin-1-yl)quinazoline

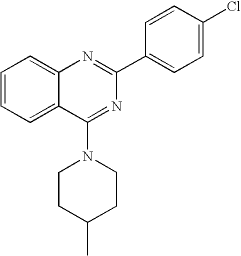

The compound 2-(4-chlorophenyl)-4-(4-methylpiperidin-1-yl)quinazoline was prepared following the procedures described for Example 13 using 4-chlorobenzoyl chloride in Step 1 and 4-methylpiperidine in Step 3. ¹H NMR (400 MHz, CDCl₃) δ 8.50 (d, 2H), 7.92 (d, 1H), 7.88 (d, 1H), 7.70 (t, 1H), 7.44 (d, 2H), 7.39 (t, 1H), 4.45 (m, 2H), 3.18 (m, 2H), 1.85 (m, 2H), 1.76 (m, 1H), 1.49 (m, 2H), 1.05 (d, 3H). MS (M/z, M+1), 338.3.

### EXAMPLE 126

### 4-(2-(4-Chlorophenyl)quinazolin-4-yl)morpholine

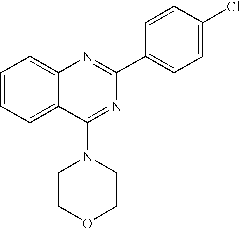

The compound 4-(2-(4-chlorophenyl)quinazolin-4-yl)morpholine was prepared following the procedures described for Example 13 using 4-chlorobenzoyl chloride in Step 1 and morpholine in Step 3. ¹H NMR (400 MHz, CDCl₃) δ 8.50 (d, 2H), 7.96 (d, 1H), 7.89 (d, 1H), 7.74 (t, 1H), 7.44 (m, 3H), 3.39 (m, 4H), 3.86 (m, 4H). MS (M/z, M+1), 326.2.

### EXAMPLE 127

### 2-(4-Chlorophenyl)-4-(piperidin-1-yl)quinazoline

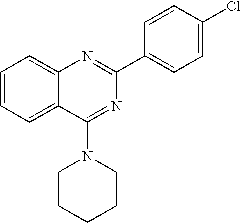

The compound 2-(4-chlorophenyl)-4-(piperidin-1-yl)quinazoline was prepared following the procedures described for Example 13 using 4-chlorobenzoyl chloride in Step 1 and piperidine in Step 3. ¹H NMR (400 MHz, CDCl₃) δ 8.51 (d, 2H), 7.92 (d, 1H), 7.88 (d, 1H), 7.70 (t, 1H), 7.42 (m, 3H), 3.80 (m, 4H), 1.82 (m, 6H). MS (M/z, M+1), 324.2.

### EXAMPLE 128

### 2-(4-Chlorophenyl)-4-(pyrrolidin-1-yl)quinazoline

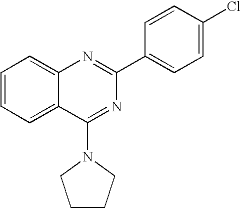

The compound 2-(4-chlorophenyl)-4-(pyrrolidin-1-yl)quinazoline was prepared following the procedures described for Example 13 using 4-chlorobenzoyl chloride in Step 1 and pyrrolidine in Step 3. ¹H NMR (400 MHz, CDCl₃) δ 8.50 (d, 2H), 8.16 (d, 1H), 7.90 (d, 1H), 7.68 (t, 1H), 7.42 (d, 2H), 7.35 (t, 1H), 4.03 (m, 4H), 2.08 (m, 4H). MS (M/z, M+1), 310.2.

### EXAMPLE 129

### 2-(4-tert-Butylphenyl)quinazolin-4-yl cyclohexylamine

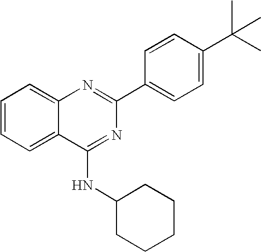

The compound 2-(4-tert-butylphenyl)quinazolin-4-yl cyclohexylamine was prepared following the procedures described for Example 13 using 4-tert-butylbenzoyl chloride in Step 1 and cyclohexylamine in Step 3. ¹H NMR (400 MHz, CDCl₃) δ 8.46 (d, 2H), 7.90 (d, 1H), 7.71 (t, 1H), 7.67 (d, 1H), 7.52 (d, 2H), 7.40 (t, 1H), 5.51 (d, 1H), 4.42 (m, 1H), 2.25 (m, 2H), 1.84 (m, 2H), 1.73 (m, 1H), 1.57 (m, 2H), 1.38 (m, 12H). MS (M/z, M+1), 361.2.

### EXAMPLE 130

### 2-(4-tert-Butylphenyl)quinazolin-4-yl cyclopropylmethylamine

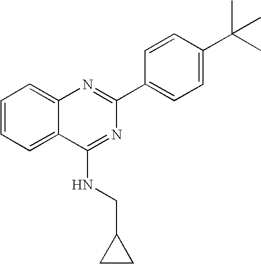

The compound 2-(4-tert-butylphenyl)quinazolin-4-yl cyclopropylmethylamine was prepared following the procedures described for Example 13 using 4-tert-butylbenzoyl chloride in Step 1 and aminomethylcyclopropane in Step 3. ¹H NMR (400 MHz, CDCl₃) δ 8.47 (d, 2H), 7.92 (d, 1H), 7.72 (m, 2H), 7.52 (d, 2H), 7.42 (t, 1H), 5.80 (m, 1H), 3.66 (m, 2H), 1.26 (m, 1H), 0.62 (m, 2H), 0.41 (m, 2H). MS (M/z, M+1), 332.3.

### EXAMPLE 131

### 2-(4-tert-Butylphenyl)quinazolin-4-yl cyclohexylmethylamine

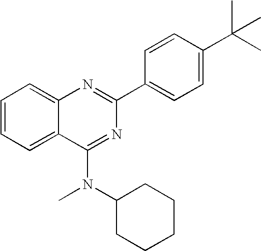

The compound 2-(4-tert-butylphenyl)quinazolin-4-yl cyclohexylmethylamine was prepared following the procedures described for Example 13 using 4-tert-butylbenzoyl chloride in Step 1 and N-methylcyclohexylamine in Step 3. ¹H NMR (400 MHz, CDCl₃) δ 8.46 (d, 2H), 7.92 (d, 2H), 7.67 (t, 1H), 7.52 (d, 2H), 7.34 (t, 1H), 4.51 (m, 1H), 3.27 (s, 3H), 2.03 (m, 2H), 1.92 (m, 2H), 1.71 (m, 4H), 1.46 (m, 11H). MS (M/z, M+1), 374.3.

### EXAMPLE 132

### 2-(4-tert-Butylphenyl)quinazolin-4-yl cyclopropylmethyl-N-methylamine

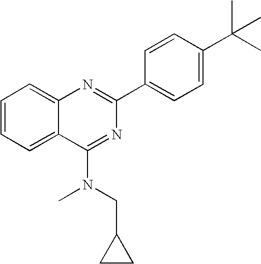

The compound 2-(4-tert-butylphenyl)quinazolin-4-yl cyclopropylmethyl-N-methylamine was prepared from 2-(4-tert-butylphenyl)quinazolin-4-yl cyclopropylmethylamine following the procedures described for Example 21. ¹H NMR (400 MHz, CDCl₃) δ 8.47 (d, 2H), 8.04 (d, 1H), 7.94 (d, 1H), 7.69 (, t, 1H), 7.52 (d, 2H), 7.37 (t, 1H), 3.72 (d, 2H), 3.50 (s, 3H), 1.37 (s, 9H), 1.30 (m, 1H), 0.64 (m, 2H), 0.35 (m, 2H). MS (M/z, M+1), 346.3.

### EXAMPLE 133

### 2-(4-tert-Butylphenyl)quinazolin-4-yl 2-chlorophenylamine

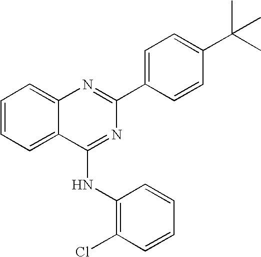

The compound 2-(4-tert-butylphenyl)quinazolin-4-yl 2-chlorophenylamine was prepared following the procedures described for Example 14 using 4-tert-butylbenzoyl chloride in Step 1 and 2-chloroaniline in Step 3. ¹H NMR (400 MHz, CDCl₃) δ 9.07 (d, 1H), 8.48 (d, 2H), 8.04 (s, 1H), 8.01 (d, 1H), 7.94 (d, 1H), 7.83 (t, 1H), 7.57 (m, 3H), 7.49 (m, 2H), 7.11 (m, 1H), 1.40 (s, 9H). MS (M/z, M+1), 388.3.

### EXAMPLE 134

### 2-(4-tert-Butylphenyl)quinazolin-4-yl 2-chlorophenyl-N-methylamine

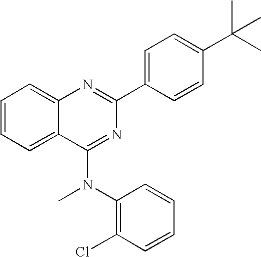

The compound 2-(4-tert-butylphenyl)quinazolin-4-yl 2-chlorophenyl-N-methylamine was prepared from 2-(4-tert-butylphenyl)quinazolin-4-yl 2-chlorophenylamine following the procedures described for Example 21. ¹H NMR (400 MHz, CDCl₃) δ 8.54 (d, 2H), 7.90 (d, 1H), 7.55 (m, 4H), 7.28 (m, 2H), 7.16 (d, 1H), 6.99 (m, 1H), 6.88 (d, 1H), 3.67 (s, 3H), 1.39 (s, 9H). MS (M/z, M+1), 402.3.

### EXAMPLE 135

### 2-Cyclopentyl-4-pyrrolidin-1-ylquinazoline

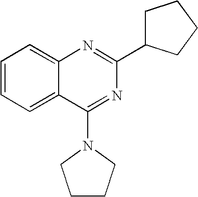

The compound 2-cyclopentyl-4-pyrrolidin-1-ylquinazoline was prepared following the procedures described for Example 13 using cyclopentanecarbonyl chloride in Step 1 and pyrrolidine in Step 3. ¹H NMR (400 MHz, CDCl₃) δ 8.12 (d, 1H), 7.77 (d, 1H), 7.63 (t, 1H), 7.30 (t, 1H), 3.94 (m, 4H), 3.25 (qt, 1H), 2.03 (m, 8H), 1.86 (m, 2H), 1.69 (m, 2H). MS (M/z, M+1), 269.3.

### EXAMPLE 136

### 2-Cyclopentyl-4-piperidin-1-ylquinazoline

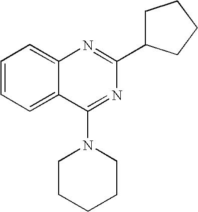

The compound 2-cyclopentyl-4-piperidin-1-ylquinazoline was prepared following the procedures described for Example 13 using cyclopentanecarbonyl chloride in Step 1 and piperidine in Step 3. ¹H NMR (400 MHz, CDCl₃) δ 7.83 (m, 2H), 7.66 (t, 1H), 7.35 (t, 1H), 3.71 (m, 4H), 3.31 (qt, 1H), 2.10-1.70 (m, 14H). MS (M/z, M+1), 282.3.

### EXAMPLE 137

### 2-Cyclopentyl-4-morpholin-4-ylquinazoline

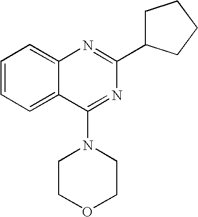

The compound 2-cyclopentyl-4-morpholin-4-ylquinazoline was prepared following the procedures described for Example 13 using cyclopentanecarbonyl chloride in Step 1 and morpholine in Step 3. ¹H NMR (400 MHz, CDCl₃) δ 7.86 (m, 2H), 7.72 (t, 1H), 7.39 (t, 1H), 3.98 (m, 4H), 3.78 (m, 4H), 3.39 (qt, 1H), 2.09 (m, 2H), 1.98 (m, 2H), 1.85 (m, 2H), 1.70 (m, 2H). MS (M/z, M+1), 284.3.

### EXAMPLE 138

### 2-Cyclopentyl-4-(4-methylpiperazin-1-yl)quinazoline

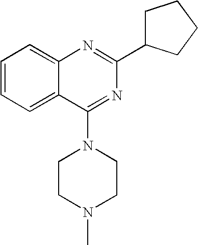

The compound 2-cyclopentyl-4-(4-methylpiperazin-1-yl)quinazoline was prepared following the procedures described for Example 13 using cyclopentanecarbonyl chloride in Step 1 and morpholine in Step 3. ¹H NMR (400 MHz, CDCl₃) δ 7.85 (m, 2H), 7.68 (t, 1H), 7.37 (t, 1H), 3.81 (m, 4H), 3.32 (qt, 1H), 2.59 (m, 4H), 2.38 (s, 3H), 2.08 (m, 2H), 1.99 (m, 2H), 1.87 (m, 2H), 1.72 (m, 2H). MS (M/z, M+1), 297.3.

### EXAMPLE 139

### 4-Pyrrolidin-1-yl-2-(4-trifluoromethoxyphenyl)quinazoline

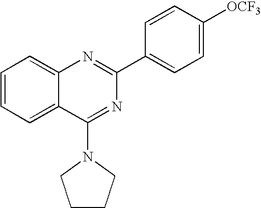

The compound 4-pyrrolidin-1-yl-2-(4-trifluoromethoxyphenyl)quinazoline was prepared following the procedures described for Example 13 using 4-trifluoromethoxybenzoyl chloride and Step 1 and pyrrolidine in Step 3. ¹H NMR (400 MHz, CDCl₃) δ 8.58 (d, 2H), 8.15 (d, 1H), 7.88 (d, 1H), 7.68 (t, 1H), 7.34 (t, 1H), 7.29 (d, 2H), 4.02 (m, 4H), 2.07 (m, 4H). MS (M/z, M+1), 360.2.

### EXAMPLE 140

### 4-Piperidin-1-yl-2-(4-trifluoromethoxyphenyl)quinazoline

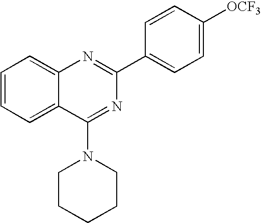

The compound 4-piperidin-1-yl-2-(4-trifluoromethoxyphenyl)quinazoline was prepared following the procedures described for Example 13 using 4-trifluoromethoxybenzoyl chloride and Step 1 and piperidine in Step 3. ¹H NMR (400 MHz, CDCl₃) δ 8.59 (d, 2H), 7.93 (d, 1H), 7.88 (d, 1H), 7.71 (t, 1H), 7.41 (t, 1H), 7.30 (d, 2H), 3.80 (m, 4H), 1.82 (m, 6H). MS (M/z, M+1), 374.2.

### EXAMPLE 141

### 4-Morpholin-4-yl-2-(4-trifluoromethoxyphenyl)quinazoline

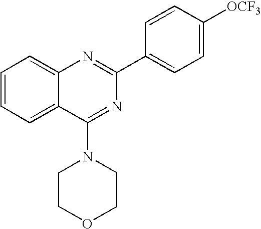

The compound 4-morpholin-4-yl-2-(4-trifluoromethoxyphenyl)quinazoline was prepared following the procedures described for Example 13 using 4-trifluoromethoxybenzoyl chloride and Step 1 and morpholine in Step 3. ¹H NMR (400 MHz, CDCl₃) δ 8.58 (d, 2H), 7.97 (d, 1H), 7.89 (d, 1H), 7.75 (t, 1H), 7.44 (t, 1H), 7.33 (d, 2H), 3.95 (m, 4H), 3.86 (m, 4H). MS (M/z, M+1), 376.2.

### EXAMPLE 142

### 4-(4-Methylpiperazin-1-yl)-2-(4-trifluoromethoxyphenyl)quinazoline

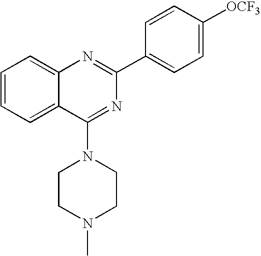

The compound 4-(4-methylpiperazin-1-yl)-2-(4-trifluoromethoxyphenyl)quinazoline was prepared following the procedures described for Example 13 using 4-trifluoromethoxybenzoyl chloride and Step 1 and 1-methylpiperizine in Step 3. ¹H NMR (400 MHz, CDCl₃) δ 8.58 (d, 2H), 7.95 (d, 1H), 7.89 (d, 1H), 7.73 (t, 1H), 7.42 (t, 1H), 7.31 (d, 2H), 3.90 (m, 4H), 2.66 (m, 4H), 2.40 (s, 3H). MS (M/z, M+1), 389.2.

### EXAMPLE 143

### (4-tert-Butylphenyl)-[2-(4-trifluoromethoxyphenyl)-quinazolin-4-yl]amine

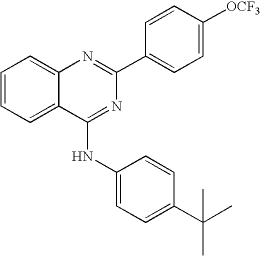

The compound (4-tert-butylphenyl)-[2-(4-trifluoromethoxyphenyl)-quinazolin-4-yl]amine was prepared following the procedures described for Example 14 using 4-trifluoromethoxybenzoyl chloride in Step 1 and 4-tert-butylaniline in Step 3. ¹H NMR (400 MHz, DMSO-d₆) δ 8.69 (d, 1H), 8.48 (d, 2H), 7.98 (m, 2H), 7.88 (d, 2H), 7.73 (m, 1H), 7.60 (d, 2H), 7.52 (d, 2H), 1.34 (s, 9H). MS (M/z, M+1), 438.9.

### EXAMPLE 144

### (4-tert-Butylphenyl)methyl-[2-(4-trifluoromethoxyphenyl)quinazolin-4-yl]amine

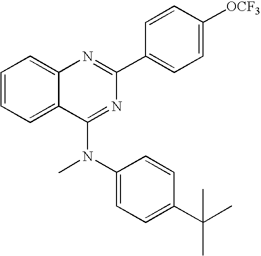

The compound (4-tert-butylphenyl)methyl-[2-(4-trifluoromethoxyphenyl)quinazolin-4-yl]amine was prepared from (4-tert-butylphenyl)-[2-(4-trifluoromethoxyphenyl) quinazolin-4-yl]amine following the procedures described for Example 21. ¹H NMR (400 MHz, CDCl₃) δ 8.66 (d, 2H), 7.88 (d, 1H), 7.57 (t, 1H), 7.40 (d, 2H), 7.35 (d, 2H), 7.14 (d, 2H), 7.01 (m, 2H), 3.74 (s, 3H), 1.35 (s, 9H). MS (M/z, M+1), 452.4.

### EXAMPLE 145

### 2-Isopropyl-4-pyrrolidin-1-ylquinazoline

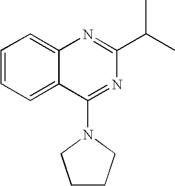

The compound 2-isopropyl-4-pyrrolidin-1-ylquinazoline was prepared following the procedures described for Example 13 using isobutyryl chloride in Step 1 and pyrrolidine in Step 3. ¹H NMR (400 MHz, CDCl₃) δ 8.12 (d, 1H), 7.77 (d, 1H), 7.62 (t, 1H), 7.29 (t, 1H), 3.94 (m, 4H), 3.07 (m, 1H), 2.04 (m, 4H), 1.35 (d, 6H). MS (M/z, M+1), 242.2.

### EXAMPLE 146

### 2-Isopropyl-4-piperidin-1-ylquinazoline

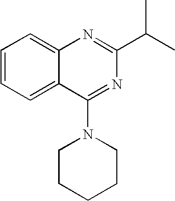

The compound 2-isopropyl-4-piperidin-1-ylquinazoline was prepared following the procedures described for Example 13 using isobutyryl chloride in Step 1 and piperidine in Step 3. ¹H NMR (400 MHz, CDCl₃) δ 7.83 (m, 2H), 7.65 (t, 1H), 7.35 (t, 1H), 3.71 (m, 4H), 3.14 (m, 1H), 1.77 (m, 6H), 1.36 (d, 6H). MS (M/z, M+1), 256.3.

### EXAMPLE 147

### 2-Isopropyl-4-morpholin-4-ylquinazoline

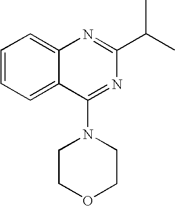

The compound 2-isopropyl-4-morpholin-4-ylquinazoline was prepared following the procedures described for Example 13 using isobutyryl chloride in Step 1 and morpholine in Step 3. ¹H NMR (400 MHz, CDCl₃) δ 7.86 (m, 2H), 7.69 (t, 1H), 7.39 (t, 1H), 3.88 (m, 4H), 3.78 (m, 4H), 3.16 (m, 1H), 1.36 (d, 6H). MS (M/z, M+1), 258.3.

### EXAMPLE 148

### 2-Isopropyl-4-(4-methylpiperazin-1-yl)quinazoline

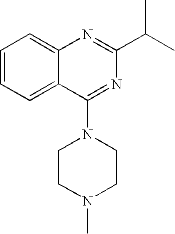

The compound 2-isopropyl-4-(4-methylpiperazin-1-yl)quinazoline was prepared following the procedures described for Example 13 using isobutyryl chloride in Step 1 and 1-methylpiperizine in Step 3. ¹H NMR (400 MHz, CDCl₃) δ 7.84 (m, 2H), 7.68 (t, 1H), 7.37 (t, 1H), 3.81 (m, 4H) 3.15 (m, 1H), 2.61 (m, 4H), 2.38 (s, 3H), 1.36 (d, 6H). MS (M/z, M+1), 271.3.

### EXAMPLE 149

### 2-(2-Fluorophenyl)-4-morpholin-4-ylquinazoline

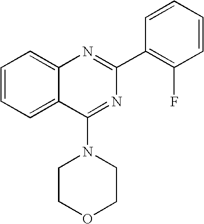

The compound 2-(2-fluorophenyl)-4-morpholin-4-ylquinazoline was prepared following the procedures described for Example 13 using 2-fluorobenzoyl chloride in Step 1 and morpholine in Step 3. ¹H NMR (400 MHz, CDCl₃) δ 8.14 (m, 1H), 8.00 (d, 1H), 7.90 (d, 1H), 7.56 (m, 1H), 7.47 (m, 1H), 7.41 (m, 1H), 7.26 (m, 1H), 7.19 (m, 1H), 3.93 (m, 4H), 3.86 (m, 4H). MS (M/z, M+1), 310.3.

### EXAMPLE 150

### 2-(2-Fluorophenyl)-4-piperidin-1-ylquinazoline

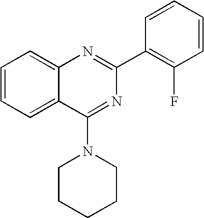

The compound 2-(2-fluorophenyl)-4-piperidin-1-ylquinazoline was prepared following the procedures described for Example 13 using 2-fluorobenzoyl chloride in Step 1 and piperidine in Step 3. ¹H NMR (400 MHz, CDCl₃) δ 8.13 (m, 1H), 7.98 (d, 1H), 7.90 (d, 1H), 7.72 (m, 1H), 7.39 (m, 2H), 7.24 (m, 1H), 7.17 (m, 1H), 3.78 (m, 4H), 1.80 (m, 6H). MS (m/z, M+1), 308.3.

### EXAMPLE 151

### 2-(2-Fluorophenyl)-4-pyrrolidin-1-ylquinazoline

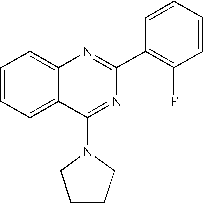

The compound 2-(2-fluorophenyl)-4-pyrrolidin-1-ylquinazoline was prepared following the procedures described for Example 13 using 2-fluorobenzoyl chloride in Step 1 and pyrrolidine in Step 3. ¹H NMR (400 MHz, CDCl₃) δ 8.18 (d, 1H), 8.12 (m, 1H), 7.91 (d, 1H), 7.69 (m, 1H), 7.38 (m, 2H), 7.23 (m, 1H), 7.17 (m, 1H), 4.01 (m, 4H), 2.06 (m, 4H). MS (M/z, M+1), 294.3.

### EXAMPLE 152

### 2-(2-Fluorophenyl)-4-(4-methylpiperazin-1-yl)quinazoline

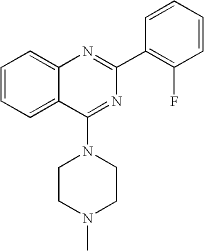

The compound 2-(2-fluorophenyl)-4-(4-methylpiperazin-1-yl)quinazoline was prepared following the procedures described for Example 13 using 2-fluorobenzoyl chloride in Step 1 and 1-methylpiperizine in Step 3. ¹H NMR (400 MHz, CDCl₃) δ 8.14 (m, 1H), 7.98 (d, 1H), 7.91 (d, 1H), 7.73 (m, 1H), 7.40 (m, 2H), 7.25 (m, 1H), 7.18 (m, 1H), 3.89 (m, 4H), 2.64 (m, 4H), 2.83 (s, 3H). MS (M/z, M+1), 323.3.

### EXAMPLE 153

### 1-(4-(2-(2-Fluorophenyl)quinazolin-4-yl)piperazin-1-yl)ethanone

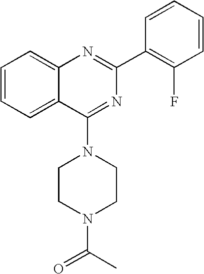

The compound 1-(4-(2-(2-fluorophenyl)quinazolin-4-yl)piperazin-1-yl)ethanone was prepared following the procedures described for Example 13 using 2-fluorobenzoyl chloride in Step 1 and 1-acetylpiperizine in Step 3. ¹H NMR (400 MHz, CDCl₃) δ 8.15 (m, 1H), 8.03 (d, 1H), 7.92 (d, 1H), 7.79 (t, 1H), 7.51 (t, 1H), 7.43 (m, 1H), 7.27 (m, 1H), 7.18 (m, 1H), 3.89 (m, 2H), 3.85 (m, 4H), 3.74 (m, 2H), 2.18 (s, 3H). MS (M/z, M+1), 351.5.

### EXAMPLE 154

### 1-(4-(2-(3-Fluorophenyl)quinazolin-4-yl)piperazin-1-yl)ethanone

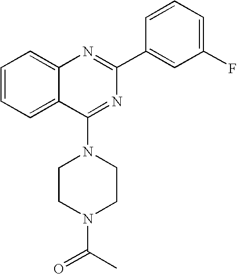

The compound 1-(4-(2-(3-fluorophenyl)quinazolin-4-yl)piperazin-1-yl)ethanone was prepared following the procedures described for Example 13 using 3-fluorobenzoyl chloride in Step 1 and 1-acetylpiperizine in Step 3. ¹H NMR (400 MHz, CDCl₃) δ 8.35 (d, 1H), 8.24 (m, 1H), 8.02 (m, 1H), 7.91 (d, 1H), 7.79 (t, 1H), 7.47 (m, 2H), 7.17 (m, 1H), 3.87 (m, 6H), 3.78 (m, 2H), 2.20 (s, 3H). MS (M/z, M+1), 351.5.

### EXAMPLE 155

### 4-Pyrrolidin-1-yl-2-o-tolylquinazoline

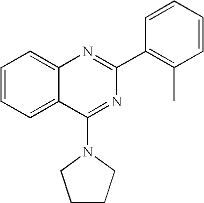

The compound 4-pyrrolidin-1-yl-2-o-tolylquinazoline was prepared following the procedures described for Example 13 using o-tolyl chloride in Step 1 and pyrrolidine in Step 3. ¹H NMR (400 MHz, CDCl₃) δ 8.19 (d, 1H), 7.88 (m, 2H), 7.68 (t, 1H), 7.37 (t, 1H), 7.28 (m, 3H), 4.02 (m, 4H), 2.61 (s, 3H), 2.05 (m, 4H).

### EXAMPLE 156

### 4-Morpholin-4-yl-2-o-tolylquinazoline

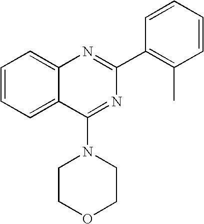

The compound 4-morpholin-4-yl-2-o-tolylquinazoline was prepared following the procedures described for Example 13 using o-tolyl chloride in Step 1 and morpholine in Step 3. ¹H NMR (400 MHz, CDCl₃) δ 7.98 (d, 1H), 7.91 (m, 2H), 7.76 (t, 1H), 7.47 (t, 1H), 7.31 (m, 3H), 3.93 (m, 4H), 3.81 (m, 4H), 2.60 (s, 3H).

### EXAMPLE 157

### 4-(4-Methylpiperazin-1-yl)-2-o-tolylquinazoline

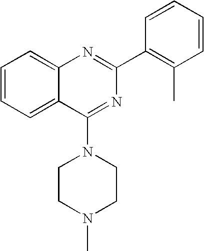

The compound 4-(4-methylpiperazin-1-yl)-2-o-tolylquinazoline was prepared following the procedures described for Example 13 using o-tolyl chloride in Step 1 and 1-methylpiperizine in Step 3. ¹H NMR (400 MHz, CDCl₃) δ 7.93 (m, 3H), 7.73 (t, 1H), 7.44 (t, 1H), 7.30 (m, 3H), 3.85 (m, 4H), 2.65 (m, 4H), 2.39 (s, 3H).

### EXAMPLE 158

### 2-Cyclohexyl-7-methoxy-4-(4-methylpiperazin-1-yl)quinazoline

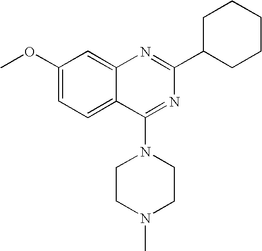

The compound 2-cyclohexyl-7-methoxy-4-(4-methylpiperazin-1-yl)quinazoline was prepared following the procedures described for Example 13 using 2-amino-4-methoxybenzamide and cyclohexanecarbonyl chloride in Step 1 and 1-methylpiperazine in Step 3. ¹H NMR (400 MHz, CDCl₃) δ 7.72 (d, 1H), 7.32 (s, 1H), 6.99 (m, 1H), 3.93 (s, 3H), 3.85 (m, 4H), 2.86 (m, 1H), 2.64 (m, 4H), 2.40 (s, 3H), 2.01 (m, 2H), 1.76 (m, 5H), 1.39 (m, 3H). MS (M/z, M+1), 341.3.

### EXAMPLE 159

### 2-Cyclohexyl-7-methoxy-4-(pyrrolidin-1-yl)quinazoline

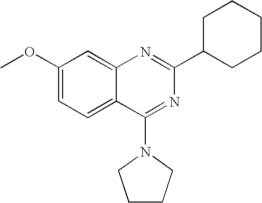

The compound 2-cyclohexyl-7-methoxy-4-(pyrrolidin-1-yl)quinazoline was prepared following the procedures described for Example 13 using 2-amino-4-methoxybenzamide and cyclohexanecarbonyl chloride in Step 1 and pyrrolidine in Step 3. ¹H NMR (400 MHz, CDCl₃) δ 8.08 (m, 2H), 7.06 (m, 1H), 4.10 (m, 4H), 3.99 (s, 3H), 3.27 (m, 1H), 2.22 (m, 2H), 2.07 (m, 4H), 1.74 (m, 5H), 1.48 (m, 3H). MS (M/z, M+1), 312.3.

### EXAMPLE 160

### 7-Chloro-2-cyclohexyl-4-(pyrrolidin-1-yl)quinazoline

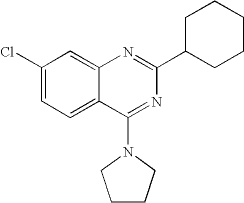

The compound 7-chloro-2-cyclohexyl-4-(pyrrolidin-1-yl)quinazoline was prepared following the procedures described for Example 13 using 2-amino-4-chlorobenzamide and cyclohexanecarbonyl chloride in Step 1 and pyrrolidine in Step 3. ¹H NMR (400 MHz, CDCl₃) δ 8.00 (m, 1H), 7.75 (d, 1H), 7.21 (m, 1H), 3.90 (m, 4H), 2.71 (m, 1H), 2.03 (m, 6H), 1.75 (m, 5H), 1.39 (m, 3H). MS (M/z, M+1), 318.2.

### EXAMPLE 161

### 6-Chloro-2-cyclohexyl-4-(4-methylpiperazin-1-yl)quinazoline

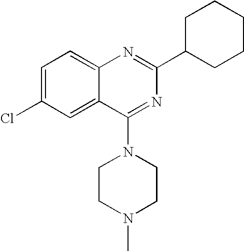

The compound 6-chloro-2-cyclohexyl-4-(4-methylpiperazin-1-yl)quinazoline was prepared following the procedures described for Example 13 using 2-amino-5-chlorobenzamide and cyclohexanecarbonyl chloride in Step 1 and 1-methylpiperazine in Step 3. ¹H NMR (400 MHz, CDCl₃) δ 7.78 (m, 2H), 7.61 (m, 1H), 3.79 (m, 4H), 2.81 (m, 1H), 2.61 (m, 4H), 2.39 (s, 3H), 2.01 (m, 2H), 1.86 (m, 2H), 1.70 (m, 3H), 1.37 (m, 3H). MS (M/z, M+1), 345.3.

### EXAMPLE 162

### 2-Cyclohexyl-6-methoxy-4-(pyrrolidin-1-yl)quinazoline

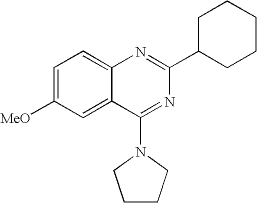

The compound 2-cyclohexyl-6-methoxy-4-(pyrrolidin-1-yl)quinazoline was prepared following the procedures described for Example 13 using 2-amino-5-methoxybenzamide and cyclohexanecarbonyl chloride in Step 1 and pyrrolidine in Step 3. ¹H NMR (400 MHz, CDCl₃) δ 7.73 (d, 1H), 7.49 (d, 1H), 7.32 (dd, 1H), 3.95 (m, 4H), 3.88 (s, 3H), 2.73 (m, 1H), 2.03 (m, 6H), 1.76 (m, 5H), 1.36 (m, 3H). MS (M/z, M+1), 312.3.

### EXAMPLE 163

### 2-Cyclohexyl-6-methoxy-4-(4-methylpiperazin-1-yl)quinazoline

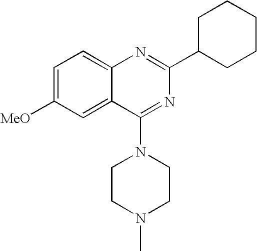

The compound 2-cyclohexyl-6-methoxy-4-(4-methylpiperazin-1-yl)quinazoline was prepared following the procedures described for Example 13 using 2-amino-5-methoxybenzamide and cyclohexanecarbonyl chloride in Step 1 and 1-methylpiperazine in Step 3. ¹H NMR (400 MHz, CDCl₃) δ 7.79 (d, 1H), 7.36 (dd, 1H), 7.13 (d, 1H), 3.90 (s, 3H), 3.74 (m, 4H), 2.81 (m, 1H), 2.62 (m, 4H), 2.39 (s, 3H), 2.01 (m, 2H), 1.79 (m, 5H), 1.40 (m, 3H). MS (M/z, M+1), 341.3.

### EXAMPLE 164

### 7-Chloro-2-cyclohexyl-4-(piperidin-1-yl)quinazoline

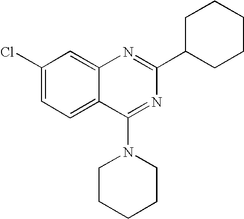

The compound 7-chloro-2-cyclohexyl-4-(piperidin-1-yl)quinazoline was prepared following the procedures described for Example 13 using 2-amino-4-chlorobenzamide and cyclohexanecarbonyl chloride in Step 1 and piperidine in Step 3. ¹H NMR (400 MHz, CDCl₃) δ 7.71 (d, 1H), 7.63 (d, 1H), 7.17 (dd, 1H), 3.60 (m, 4H), 2.69 (m, 1H), 1.91 (m, 2H), 1.67 (m, 11H), 1.26 (m, 3H). MS (M/z, M+1), 330.2.

### EXAMPLE 165

### 4-(7-Chloro-2-cyclohexylquinazolin-4-yl)morpholine

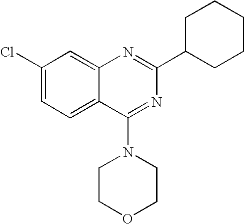

The compound 4-(7-chloro-2-cyclohexylquinazolin-4-yl)morpholine was prepared following the procedures described for Example 13 using 2-amino-4-chlorobenzamide and cyclohexanecarbonyl chloride in Step 1 and morpholine in Step 3. ¹H NMR (400 MHz, CDCl₃) δ 7.82 (d, 1H), 7.71 (d, 1H), 7.28 (dd, 1H), 3.85 (m, 4H), 3.75 (m, 4H), 2.78 (m, 1H), 1.98 (m, 2H), 1.81 (m, 2H), 1.66 (m, 3H), 1.37 (m, 3H). MS (M/z, M+1), 332.2.

### EXAMPLE 166

### 7-Chloro-2-cyclohexyl-4-(4-methylpiperazin-1-yl)quinazoline

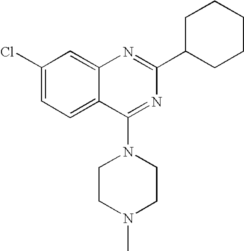

The compound 7-chloro-2-cyclohexyl-4-(4-methylpiperazin-1-yl)quinazoline was prepared following the procedures described for Example 13 using 2-amino-4-chlorobenzamide and cyclohexanecarbonyl chloride in Step 1 and 1-methylpiperizine in Step 3. ¹H NMR (400 MHz, CDCl₃) δ 7.76 (d, 1H), 7.69 (d, 1H), 7.22 (dd, 1H), 3.74 (m, 4H), 2.73 (m, 1H), 2.53 (m, 4H), 2.31 (s, 3H), 1.95 (m, 2H), 1.79 (m, 2H), 1.65 (m, 3H), 1.34 (m, 3H). MS (M/z, M+1), 345.2.

### EXAMPLE 167

### 6-Chloro-2-cyclohexyl-4-(pyrrolidin-1-yl)quinazoline

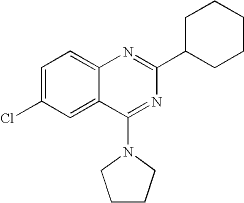

The compound 6-chloro-2-cyclohexyl-4-(pyrrolidin-1-yl)quinazoline was prepared following the procedures described for Example 13 using 2-amino-5-chlorobenzamide and cyclohexanecarbonyl chloride in Step 1 and pyrrolidine in Step 3. ¹H NMR (400 MHz, CDCl₃) δ 8.08 (d, 1H), 7.71 (d, 1H), 7.56 (dd, 1H), 3.93 (m, 4H), 2.73 (m, 1H), 2.06 (m, 4H), 1.98 (m, 2H), 1.76 (m, 5H), 1.37 (m, 3H). MS (M/z, M+1), 316.2.

### EXAMPLE 168

### 6-tert-Butyl-2-(2-chlorophenyl)-4-(4-methylpiperazin-1-yl)quinazoline

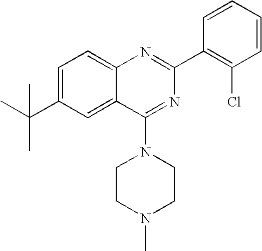

The compound 6-tert-butyl-2-(2-chlorophenyl)-4-(4-methylpiperazin-1-yl)quinazoline was prepared following the procedures described for Example 13 using 2-amino-5-tert-butylbenzamide and 2-chlorobenzoyl chloride in Step 1 and 1-methylpiperizine in Step 3. ¹H NMR (400 MHz, CDCl₃) δ 7.92 (d, 1H), 7.85 (3H), 7.47 (m, 1H), 7.35 (m, 2H), 3.85 (m, 4H), 2.66 (m, 4H), 2.39 (s, 3H), 1.43 (s, 9H). MS (M/z, M+1), 395.4.

### EXAMPLE 169

### 4-(6-tert-Butyl-2-(2-chlorophenyl)quinazolin-4-yl)thiomorpholine

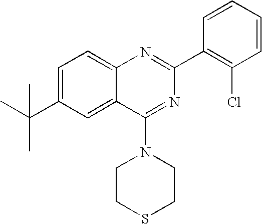

The compound 4-(6-tert-butyl-2-(2-chlorophenyl)quinazolin-4-yl)thiomorpholine was prepared following the procedures described for Example 13 using 2-amino-5-tert-butylbenzamide and 2-chlorobenzoyl chloride in Step 1 and thiomorpholine in Step 3. ¹H NMR (400 MHz, CDCl₃) δ 7.94 (d, 1H), 7.88 (d, 1H), 7.85 (m, 1H), 7.83 (m, 1H), 7.48 (m, 1H), 7.36 (m, 2H), 4.07 (m, 4H), 2.91 (m, 4H), 1.43 (s, 9H). MS (M/z, M+1), 398.3.

### EXAMPLE 170

### 6-tert-Butyl-2-(2-chlorophenyl)-4-(piperidin-1-yl)quinazoline

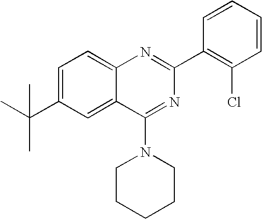

The compound 6-tert-butyl-2-(2-chlorophenyl)-4-(piperidin-1-yl)quinazoline was prepared following the procedures described for Example 13 using 2-amino-5-tert-butylbenzamide and 2-chlorobenzoyl chloride in Step 1 and piperidine in Step 3. ¹H NMR (400 MHz, CDCl₃) δ 7.89 (m, 2H), 7.83 (m, 2H), 7.46 (m, 1H), 7.33 (m, 2H), 3.75 (m, 4H), 1.80 (m, 6H), 1.44 (s, 9H). MS (M/z, M+1), 380.3.

### EXAMPLE 171

### 6-tert-Butyl-2-(2-chlorophenyl)-4-(pyrrolidin-1-yl)quinazoline

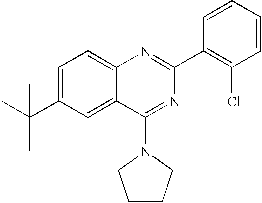

The compound 6-tert-butyl-2-(2-chlorophenyl)-4-(pyrrolidin-1-yl)quinazoline was prepared following the procedures described for Example 13 using 2-amino-5-tert-butylbenzamide and 2-chlorobenzoyl chloride in Step 1 and pyrrolidine in Step 3. ¹H NMR (400 MHz, CDCl₃) δ 8.17 (d, 1H), 7.86 (d, 1H), 7.81 (d, 1H), 7.79 (m, 1H), 7.45 (m, 1H), 7.32 (m, 2H), 4.02 (m, 4H), 2.05 (m, 4H), 1.41 (s, 9H). MS (M/z, M+1), 366.3.

### EXAMPLE 172

### 4-(2-(2-Chlorophenyl)-6-methoxyquinazolin-4-yl)thiomorpholine

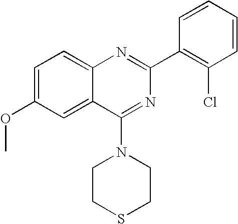

The compound 4-(2-(2-chlorophenyl)-6-methoxyquinazolin-4-yl)thiomorpholine was prepared following the procedures described for Example 13 using 2-amino-5-methoxybenzamide and 2-chlorobenzoyl chloride in Step 1 and thiomorpholine in Step 3. ¹H NMR (400 MHz, CDCl₃) δ 7.93 (d, 1H), 7.84 (m, 1H), 7.46 (m, 2H), 7.36 (m, 2H), 7.15 (d, 1H), 4.02 (m, 4H), 3.94 (s, 3H), 2.89 (m, 4H). MS (M/z, M+1), 372.2.

### EXAMPLE 173

### 2-(2-Chlorophenyl)-6-ethyl-4-(4-methylpiperazin-1-yl)quinazoline

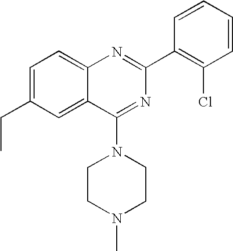

The compound 2-(2-chlorophenyl)-6-ethyl-4-(4-methylpiperazin-1-yl)quinazoline was prepared following the procedures described for Example 13 using 2-amino-5-ethylbenzamide and 2-chlorobenzoyl chloride in Step 1 and 1-methylpiperizine in Step 3. ¹H NMR (400 MHz, CDCl₃) δ 7.90 (d, 1H), 7.84 (m, 1H), 7.70 (m, 1H), 7.62 (m, 1H), 7.47 (m, 1H), 7.34 (m, 2H), 3.86 (m, 4H), 2.83 (q, 2H), 2.64 (m, 4H), 2.39 (s, 3H), 1.34 (t, 3H). MS (M/z, M+1), 367.1.

### EXAMPLE 174

### 4-(2-(2-Chlorophenyl)-6-ethylquinazolin-4-yl)thiomorpholine

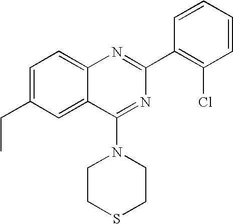

The compound 4-(2-(2-chlorophenyl)-6-ethylquinazolin-4-yl)thiomorpholine was prepared following the procedures described for Example 13 using 2-amino-5-ethylbenzamide and 2-chlorobenzoyl chloride in Step 1 and thiomorpholine in Step 3. ¹H NMR (400 MHz, CDCl₃) δ 7.92 (d, 1H), 7.85 (m, 1H), 7.64 (m, 2H), 7.48 (m, 1H), 7.36 (m, 2H), 4.07 (m, 4H), 2.89 (m, 4H), 2.85 (q, 2H), 1.34 (t, 3H). MS (M/z, M+1), 370.3.

### EXAMPLE 175

### 2-(2-Chlorophenyl)-6-ethyl-4-(piperidin-1-yl)quinazoline

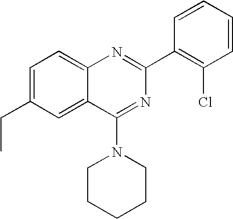

The compound 2-(2-chlorophenyl)-6-ethyl-4-(piperidin-1-yl)quinazoline was prepared following the procedures described for Example 13 using 2-amino-5-ethylbenzamide and 2-chlorobenzoyl chloride in Step 1 and piperidine in Step 3. ¹H NMR (400 MHz, CDCl₃) δ 7.88 (d, 1H), 7.84 (m, 1H), 7.70 (m, 1H), 7.60 (dd, 1H), 7.47 (m, 1H), 7.33 (m, 2H), 3.75 (m, 4H), 2.83 (q, 2H), 1.79 (m, 6H), 1.34 (t, 3H). MS (M/z, M+1), 352.3

### EXAMPLE 176

### 2-(2-Chlorophenyl)-6-ethyl-4-(pyrrolidin-1-yl)quinazoline

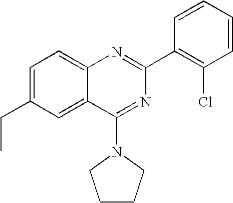

The compound 2-(2-chlorophenyl)-6-ethyl-4-(pyrrolidin-1-yl)quinazoline was prepared following the procedures described for Example 13 using 2-amino-5-ethylbenzamide and 2-chlorobenzoyl chloride in Step 1 and pyrrolidine in Step 3. ¹H NMR (400 MHz, CDCl₃) δ 7.99 (m, 1H), 7.84 (d, 1H), 7.80 (m, 1H), 7.57 (dd, 1H), 7.45 (m, 1H), 7.32 (m, 2H), 4.01 (m, 4H), 2.80 (q, 2H), 2.04 (m, 4H), 1.32 (t, 3H). MS (M/z, M+1), 338.3.

### EXAMPLE 177

### 2-(2-Chlorophenyl)-6-methoxy-4-(4-methylpiperazin-1-yl)quinazoline

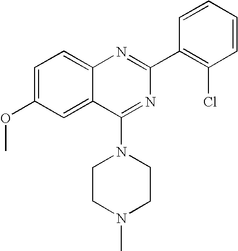

The compound 2-(2-chlorophenyl)-6-methoxy-4-(4-methylpiperazin-1-yl)quinazoline was prepared following the procedures described for Example 13 using 2-amino-5-methoxybenzamide and 2-chlorobenzoyl chloride in Step 1 and 1-methylpiperizine in Step 3. ¹H NMR (400 MHz, CDCl₃) δ 7.92 (d, 1H), 7.85 (m, 1H), 7.48 (m, 1H), 7.42 (dd, 1H), 7.35 (m, 2H), 7.21 (d, 1H), 3.94 (s, 3H), 3.81 (m, 4H), 2.65 (m, 4H), 2.39 (s, 3H). MS (M/z, M+1), 369.3.

### EXAMPLE 178

### 2-(2-Chlorophenyl)-6-methoxy-4-(piperidin-1-yl)quinazoline

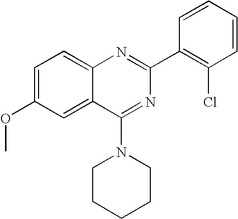

The compound 2-(2-chlorophenyl)-6-methoxy-4-(piperidin-1-yl)quinazoline was prepared following the procedures described for Example 13 using 2-amino-5-methoxybenzamide and 2-chlorobenzoyl chloride in Step 1 and piperidine in Step 3. ¹H NMR (400 MHz, CDCl₃) δ 7.90 (d, 1H), 7.84 (dd, 1H), 7.46 (dd, 1H), 7.41 (dd, 1H), 7.34 (m, 2H), 7.21 (d, 1H), 3.93 (s, 3H), 3.69 (m, 4H), 1.77 (m, 6H). MS (M/z, M+1), 354.3.

### EXAMPLE 179

### 2-(2-Chlorophenyl)-6-methoxy-4-(pyrrolidin-1-yl)quinazoline

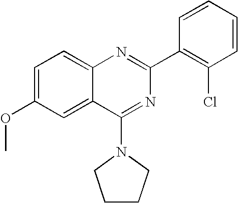

The compound 2-(2-chlorophenyl)-6-methoxy-4-(pyrrolidin-1-yl)quinazoline was prepared following the procedures described for Example 13 using 2-amino-5-methoxybenzamide and 2-chlorobenzoyl chloride in Step 1 and pyrrolidine in Step 3. ¹H NMR (400 MHz, CDCl₃) δ 7.86 (d, 1H), 7.81 (m, 1H), 7.57 (d, 1H), 7.45 (d, 1H), 7.38 (dd, 1H), 7.32 (m, 2H), 4.00 (m, 4H), 3.91 (s, 3H), 2.04 (m, 4H). MS (M/z, M+1), 340.2.

### EXAMPLE 180

### 2-(2-Chlorophenyl)-4-(4-methylpiperazin-1-yl)-6-(trifluoromethoxy) quinazoline

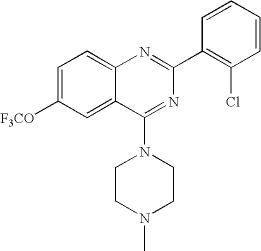

The compound 2-(2-chlorophenyl)-4-(4-methylpiperazin-1-yl)-6-(trifluoromethoxy) quinazoline was prepared following the procedures described for Example 13 using 2-amino-5-trifluoromethoxybenzamide and 2-chlorobenzoyl chloride in Step 1 and 1-methylpiperizine in Step 3. ¹H NMR (400 MHz, CDCl₃) δ 8.01 (d, 1H), 7.85 (m, 1H), 7.74 (s, 1H), 7.62 (m, 1H), 7.49 (m, 1H), 7.37 (m, 2H), 3.87 (m, 4H), 2.64 (m, 4H), 2.39 (s, 3H). MS (M/z, M+1), 423.3.

### EXAMPLE 181

### 2-(2-Chlorophenyl)-4-(piperidin-1-yl)-6-(trifluoromethoxy) quinazoline

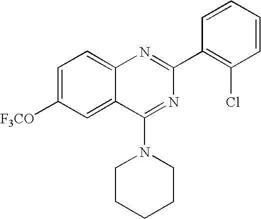

The compound 2-(2-chlorophenyl)-4-(piperidin-1-yl)-6-(trifluoromethoxy) quinazoline was prepared following the procedures described for Example 13 using 2-amino-5-trifluoromethoxybenzamide and 2-chlorobenzoyl chloride in Step 1 and piperidine in Step 3. ¹H NMR (400 MHz, CDCl₃) δ 7.99 (d, 1H), 7.84 (m, 1H), 7.74 (s, 1H), 7.59 (m, 1H), 7.48 (m, 1H), 7.36 (m, 2H), 3.77 (m, 4H), 1.81 (m, 6H). MS (M/z, M+1), 408.3.

### EXAMPLE 182

### 2-(2-Chlorophenyl)-4-(pyrrolidin-1-yl)-6-(trifluoromethoxy) quinazoline

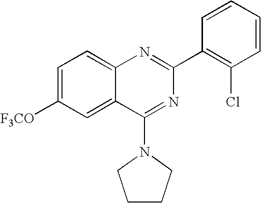

The compound 2-(2-chlorophenyl)-4-(pyrrolidin-1-yl)-6-(trifluoromethoxy) quinazoline was prepared following the procedures described for Example 13 using 2-amino-5-trifluoromethoxybenzamide and 2-chlorobenzoyl chloride in Step 1 and pyrrolidine in Step 3. ¹H NMR (400 MHz, CDCl₃) δ 8.06 (m, 1H), 7.94 (d, 1H), 7.80 (m, 1H), 7.58 (m, 1H), 7.48 (m 1H), 7.34 (m, 2H), 4.00 (m, 4H), 2.08 (m, 4H). MS (M/z, M+1), 394.2.

### EXAMPLE 183

### 6-Bromo-2-(2-chlorophenyl)-4-(4-methylpiperazin-1-yl)quinazoline

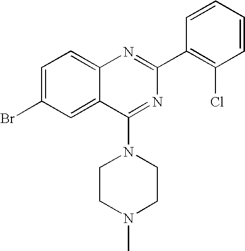

The compound 6-bromo-2-(2-chlorophenyl)-4-(4-methylpiperazin-1-yl)quinazoline was prepared following the procedures described for Example 13 using 2-amino-5-bromobenzamide and 2-chlorobenzoyl chloride in Step 1 and 1-methylpiperizine in Step 3. ¹H NMR (400 MHz, CDCl₃) δ 8.05 (d, 1H), 7.83 (m, 3H), 7.48 (m, 1H), 7.36 (m, 2H), 3.89 (m, 4H), 2.63 (m, 4H), 2.38 (s, 3H). MS (M/z, M+1), 419.4.

### EXAMPLE 184

### 6-Bromo-2-(2-chlorophenyl)-4-(pyrrolidin-1-yl)quinazoline

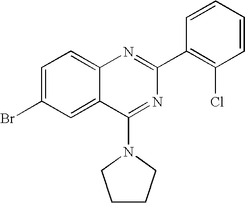

The compound 6-bromo-2-(2-chlorophenyl)-4-(pyrrolidin-1-yl)quinazoline was prepared following the procedures described for Example 13 using 2-amino-5-bromobenzamide and 2-chlorobenzoyl chloride in Step 1 and pyrrolidine in Step 3. ¹H NMR (400 MHz, CDCl₃) δ 8.33 (m, 1H), 7.81 (m, 1H), 7.77 (m, 2H), 7.46 (m, 1H), 7.34 (m, 2H), 3.99 (m, 4H), 2.06 (m, 4H). MS (M/z, M+1), 390.3.

### EXAMPLE 185

### 1-(4-(6-Chloro-2-cyclohexylquinazolin-4-yl)piperazin-1-yl)ethanone

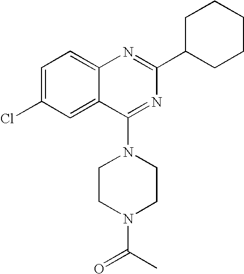

The compound 1-(4-(6-chloro-2-cyclohexylquinazolin-4-yl)piperazin-1-yl)ethanone was prepared following the procedures described for Example 13 using 2-amino-5-chlorobenzamide and 2-chlorobenzoyl chloride in Step 1 and 1-acetylpiperizine in Step 3. ¹H NMR (400 MHz, CDCl₃) δ 7.81 (d, 1H), 7.80 (s, 1H), 7.65 (m, 1H), 3.76 (m, 8H), 2.81 (m, 1H), 2.17 (s, 3H), 1.98 (m, 2H), 1.85 (m, 2H), 1.71 (m, 3H), 1.40 (m, 3H). MS (M/z, M+1), 373.5.

### EXAMPLE 186

### 1-(4-(2-Cyclohexyl-6-methoxyquinazolin-4-yl)piperazin-1-yl)ethanone

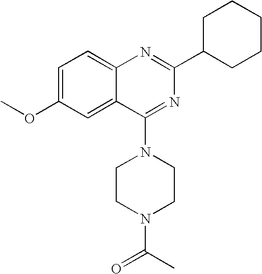

The compound 1-(4-(2-cyclohexyl-6-methoxyquinazolin-4-yl)piperazin-1-yl)ethanone was prepared following the procedures described for Example 13 using 2-amino-5-methoxybenzamide and 2-chlorobenzoyl chloride in Step 1 and 1-acetylpiperizine in Step 3. ¹H NMR (400 MHz, CDCl₃) δ 7.82 (d, 1H), 7.40 (dd, 1H), 7.12 (d, 1H), 3.91 (s, 3H), 3.81 (m, 2H), 3.71 (m, 6H), 2.83 (m, 1H), 2.18 (s, 3H), 2.00 (m, 2H), 1.85 (m, 2H), 1.70 (m, 3H), 1.41 (m, 3H). MS (M/z, M+1), 369.5.

### EXAMPLE 187

### 4-Piperidin-1-yl-2-o-tolylquinazoline

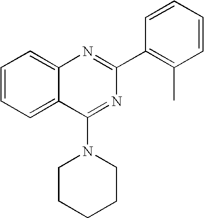

The compound 4-piperidin-1-yl-2-o-tolylquinazoline was prepared following the procedures described for Example 13 using o-tolyl chloride in Step 1 and piperidine in Step 3. ¹H NMR (400 MHz, CDCl₃) δ 7.90 (m, 3H), 7.72 (m, 1H), 7.43 (m, 1H), 7.29 (m, 3H), 3.74 (m, 4H), 2.60 (s, 3H), 178 (m, 6H).

### EXAMPLE 188

### 2-(2-Chlorophenyl)-N-((tertrahydrofuran-2-yl)methyl)quinzaoline-4-amine

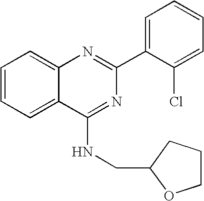

The compound 2-(2-chlorophenyl)-N-((tertrahydrofuran-2-yl)methyl)quinzaoline-4-amine was synthesized according to Scheme II. 4-Chloro-2-(2-chlorophenyl)quinazoline (100 mg, 0.36 mmol) was dissolved in dry DMSO (1.5 mL). (Tetrahydrofuran-2-yl)methananime (36.9 mg, 0.36 mmol) and N,N-diispropylethylamine (54.6 mg, 0.54 mmol) were added and the reaction was heated to 130° C. for 18 hours. The mixture was then concentrated onto silica gel and purified by flash column chromatography (0-100% EtOAc/Hex) to give the title compound. MS (M/z, M+1), 340.56.

### EXAMPLE 189

### 2-(2-Chlorophenyl)-N-(3,3-dimethylbutyl)quinazolin-4-amine

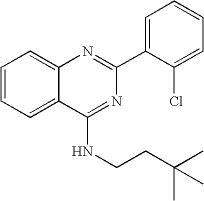

The compound 2-(2-chlorophenyl)-N-(3,3-dimethylbutyl)quinazolin-4-amine was prepared following the procedure described for Example 188 using 2,2 dimethylbutylamine. ¹H NMR (400 MHz, DMSO-d₆) 8.33 (t, 1H), 8.23 (dd, 1H), 7.78-7.68 (m, 3H), 7.54-7.49 (m, 2H), 7.45-7.39 (m, 2H), 3.57 (m, 2H), 1.60 (m, 2H), 0.92 (t, 9H). MS (M/z, M+1), 340.60.

### EXAMPLE 190

### 2-(2-Chlorophenyl)-N-(1-adamanthylmethyl)quinazolin-4-amine

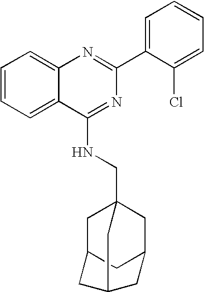

The compound 2-(2-chlorophenyl)-N-(1-adamanthylmethyl)quinazolin-4-amine was prepared following the procedure described for Example 188 using 1-adamantane methylamine. ¹H NMR (400 MHz, DMSO-d₆) 8.38 (dd, 1H), 8.12 (t, 1H), 7.79-7.75 (m, 1H), 7.72-7.65 (m, 2H), 7.54-7.50 (m, 2H), 7.45-7.41 (m, 2H), 3.34 (d, 2H), 1.90 (s, 3H), 1.65-1.55 (m, 12H). MS (M/z, M+1), 404.61.

### EXAMPLE 191

### 2-(2-Chlorophenyl)-N-(2-(pyridine-2-yl)ethyl)quinazolin-4-amine

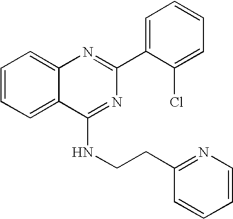

The compound 2-(2-chlorophenyl)-N-(2-(pyridine-2-yl)ethyl)quinazolin-4-amine was prepared following the procedure described for Example 188 using 2-(2-aminoethyl)pyridine. ¹H NMR (400 MHz, DMSO-d₆) 8.52-8.48 (m, 2H), 8.22 (d, 1H), 7.80-7.76 (m, 1H), 7.73-7.64 (m, 2H), 7.68-7.64 (m, 1H), 7.54-7.50 (m, 2H), 7.46-7.41 (m, 2H), 7.24 (d, 1H), 7.21-7.18 (m, 1H), 3.91-3.86 (m, 2H), 3.17-3.13 (m, 2H). MS (M/z, M+1), 361.53.

### EXAMPLE 192

### 2-(2-Chlorophenyl)-N-(3-morpholinopropyl)quinazolin-4-amine

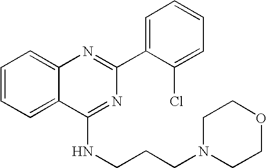

The compound 2-(2-chlorophenyl)-N-(3-morpholinopropyl)quinazolin-4-amine was prepared following the procedure described for Example 188 using 4-(3-aminopropyl)morpholine. ¹H NMR (400 MHz, DMSO-d₆) δ 8.42 (t, 1H), 8.29 (dd, 1H), 7.79-7.75 (m, 1H), 7.71-7.67 (m, 2H), 7.54-7.50 (m, 2H), 7.46-7.39 (m, 2H), 3.57 (q, 2H), 3.48 (t, 4H), 2.53 (d, 1H) 2.37-2.33 (m, 5H), 1.85-1.78 (m, 2H). MS (M/z, M+1), 383.57.

### EXAMPLE 193

### 2-(2-Chlorophenyl)-N-(cyclohexylmethyl)quinazolin-4-amine

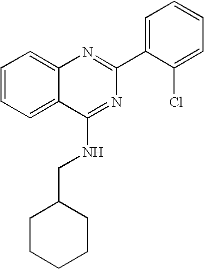

The compound 2-(2-chlorophenyl)-N-(cyclohexylmethyl)quinazolin-4-amine was prepared following the procedure described for Example 188 using 4-cyclohexylmethylamine. ¹H NMR (400 MHz, DMSO-d₆) 8.35 (t, 1H), 8.30 (dd, 1H), 7.79-7.74 (m, 1H), 7.68 (m, 2H), 7.53-7.49 (m, 2H), 7.46-7.40 (m, 2H), 7.41-7.36 (dd, 2H), 1.81-1.73 (m, 3H), 1.65-1.60 (m, 3H), 1.24-1.11 (m, 3H), 0.99-0.93 (m, 2H). MS (M/z, M+1), 352.59.

### EXAMPLE 194

### 2-(2-Chlorophenyl)-N-(cyclohexylmethyl)-N-methylquinazolin-4-amine

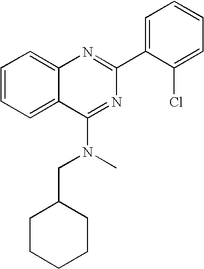

The compound 2-(2-chlorophenyl)-N-(cyclohexylmethyl)-N-methylquinazolin-4-amine was synthesized according to Scheme III. 2-(2-chlorophenyl)-N-(cyclohexylmethyl)quinazolin-4-amine (30 mg, 0.09 mmol) was dissolved in dry DMSO (1.5 mL) and iodomethane (10.6 μL, 0.17 mmol) was added followed by sodium hydride (60% mineral oil suspension, 4.1 mg, 0.17 mmol). The mixture was stirred at room temperature for 1.5 hours. The mixture was purified by reverse phase column chromatography (0-90% acetonitrile/water) to give the title compound. MS (M/z, M+1), 366.51.

### EXAMPLE 195

### 4-(Piperidin-1-yl)-2-(4-(trifluoromethyl)benzyl)quinazoline

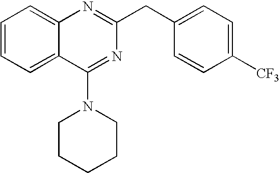

The compound 4-(piperidin-1-yl)-2-(4-(trifluoromethyl)benzyl)quinazoline was synthesized according to Scheme II.

### Step 1

Anthranilamide (1.33 g, 9.80 mmol), 1-ethyl-3-(3-dimethylaminopropyl) carbodiimide hydrochloride (2.25 g, 11.78 mmol), and hydroxybenzotriazole (1.99 g, 14.7 mmol) were dissolved in DMF (15 mL). Tetrafluoro-p-tolyl acetic acid (2.00 g, 9.80 mmol) was then added and stirred at room temperature. The mixture was purified by flash column chromatography (0-100% EtOAc/Hex) to give 2-(4-(trifloromethyl)benzyl)quinazolin-4(3H)-one.

### Step 2

2-(4-(trifloromethyl)benzyl)quinazolin-4(3H)-one (0.4 g, 1.31 mmol) was dissolved in toluene (10 mL). Then POCl₃ (127.5 μL, 1.31 mmol) and PCl₅ (171 μL, 1.31 mmol) were added. The mixture was stirred and heated to 60° C. The mixture was extracted into ethyl acetate, washed with brine, and dried over Na₂SO₄. The solvent was removed in vacuo to afford 4-chloro-2-(4-(trifluoromethyl)benzyl)quinazoline. The material was used without further purification.

### Step 3

### 4-Chloro-2-(4-(trifluoromethyl)benzyl)quinazoline (0.211 g, 0.65 mmol) from Step 2 was dissolved in THF (5 mL). N,N-diispropylethylamine (182.6 μL, 1.31 mmol) and piperidine (129.4 μL, 1.31 mmol) were added and stirred at room temperature overnight. The mixture was purified by reverse phase column chromatography (0-90% acetonitrile/water) to give the title compound. MS (M/z, M+1), 372.57.

### EXAMPLE 196

### Ethyl 4-(2-(3-Methoxybenzyl)quinazolin-4-yl)piperazine-1-carboxylate

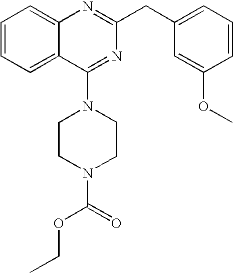

The compound ethyl 4-(2-(3-methoxybenzyl)quinazolin-4-yl)piperazine-1-carboxylate was prepared following the procedures described for Example 195 using 3-methoxyphenyl acetic acid in Step 1 and ethyl N-piperazine carboxylate in Step 3. MS (M/z, M+1), 407.62.

### EXAMPLE 197

### 2-(2-Chlorophenyl)-4-(4-(2-chloropyrimidin-4-yl)piperazin-1-yl)quinazoline

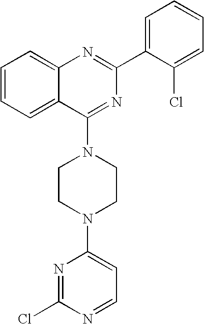

The compound 2-(2-chlorophenyl)-4-(4-(2-chloropyrimidin-4-yl)piperazin-1-yl)quinazoline was synthesized according to Scheme II. 2-(2-Chlorophenyl)-4-piperazin-1-ylquinazoline (0.1 g, 0.31 mmol) was dissolved in dry THF (5 mL). 2,4-Dichloropyrimidine (0.046 g, 0.31 mmol) and N,N-diispropylethylamine (64 μL, 0.46 mmol) were added to the solution and the mixture was stirred at room temperature for 5 hours. The mixture was purified by flash column chromatography (0-10% MeOH/CH₂Cl₂) to give the title compound. MS (M/z, M+1), 437.54.

### EXAMPLE 198

### 2-(2-Chlorophenyl)-4-(4-chloropyrimidin-2-yl)piperazin-1-yl)quinazoline

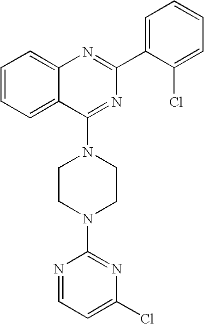

The compound 2-(2-Chlorophenyl)-4-(4-chloropyrimidin-2-yl)piperazin-1-yl)quinazoline was synthesized during the preparation of Example 197 and isolated during purification. MS (M/z, M+1), 437.54.

### EXAMPLE 199

### 1-(4-(2-(4-Chlorophenyl)quinazolin-4-yl)piperazin-1-yl)ethanone

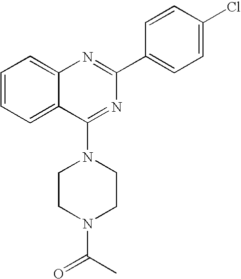

The compound 1-(4-(2-(4-chlorophenyl)quinazoline-4-yl)-piperazin-1-yl)ethanone was prepared following the procedures described for Example 13 using 4-chlorobenzoyl chloride in Step 1 and 1-acetylpiperazine in Step 3. ¹H NMR (400 MHz, CDCl₃) δ 8.50 (d, 2H), 7.98 (d, 1H), 7.90 (d, 1H), 7.78 (m, 1H), 7.47 (m, 3H), 3.88 (m, 4H), 3.84 (m, 2H), 3.77 (m, 2H), 2.20 (s, 3H). MS (M/z, M+1), 367.4.

### EXAMPLE 200

### Ethyl 4-(2-(4-chlorophenyl)quinazolin-4-yl)piperazine-1-carboxylate

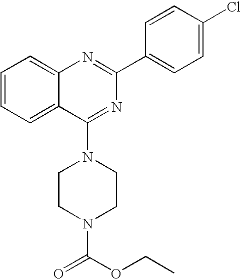

The compound ethyl 4-(2-(4-chlorophenyl)quinazoline-4-yl)piperazine-1-carboxylate was prepared following the procedures described for Example 13 using 4-chlorobenzoyl chloride in Step 1 and ethyl 1-piperazine carboxylate in Step 3. ¹H NMR (400 MHz, CDCl₃) δ 8.50 (d, 2H), 7.98 (d, 1H), 7.91 (d, 1H), 7.77 (m, 1H), 7.47 (m, 3H), 4.21 (q, 2H), 3.82 (m, 4H), 3.77 (m, 4H), 1.31 (t, 3H). MS (M/z, M+1) 397.5.

### EXAMPLE 201

### 1-(4-(2-(4-tert-Butylphenyl)quinazolin-4-yl)piperazin-1-yl)ethanone

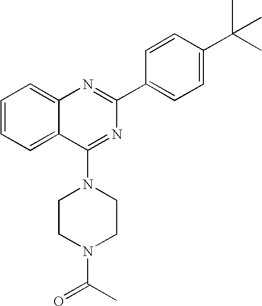

The compound 1-(4-(2-(4-tert-butylphenyl)quinazoline-4-yl)piperazine-1-yl)ethanone was prepared following the procedures described for Example 13 using 4-tert-butylbenzoyl chloride in Step 1 and 1-acetylpiperazine in Step 3. ¹H NMR (400 MHz, CDCl₃) δ 8.46 (d, 2H), 8.00 (d, 1H), 7.89 (d, 1H), 7.76 (m, 1H), 7.52 (d, 2H), 7.45 (m, 1H), 3.87 (m, 4H), 3.82 (m, 2H), 3.81 (m, 2H), 2.19 (s, 3H), 1.37 (s, 9H). MS (M/z, M+1), 389.5.

### EXAMPLE 202

### 1-(4-(2-(2,4-Dichlorophenyl)quinazolin-4-yl)piperazin-1-yl)ethanone

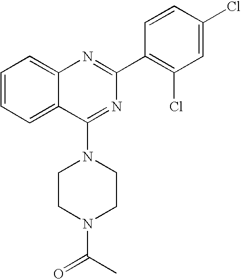

The compound 1-(4-(2-(2,4-dichlorophenyl)quinazolin-4-yl)piperazin-1-yl)ethanone was prepared following the procedures described for Example 13 using 2,4-dichlorobenzoyl chloride in Step 1 and 1-acetylpiperazine in Step 3. ¹H NMR (400 MHz, CDCl₃) δ 8.01 (d, 1H), 7.94 (d, 1H), 7.85 (d, 1H), 7.81 (m, 1H), 7.54 (m, 1H), 7.52 (d, 1H), 7.38 (m, 1H), 3.88 (m, 2H), 3.84 (m, 4H), 3.73 (m, 2H), 2.18 (s, 3H). MS (M/z, M+1), 401.4.

### EXAMPLE 203

### Ethyl 4-(2-(2,4-dichlorophenyl)quinazolin-4-yl)piperazine-1-carboxylate

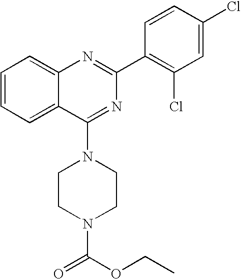

The compound ethyl 4-(2-(2,4-dichlorophenyl)quinazoline-4-yl)piperazine-1-carboxylate was prepared following the procedures described for Example 13 using 2,4-dichlorobenzoyl chloride in Step 1 and ethyl 1-piperazine carboxylate in Step 3. ¹H NMR (400 MHz, CDCl₃) δ 7.99 (d, 1H), 7.94 (d, 1H), 7.85 (d, 1H), 7.98 (m, 1H), 7.52 (m, 2H), 7.36 (m, 1H), 4.20 (q, 2H), 3.82 (m, 4H), 3.73 (m, 4H), 1.30 (t, 3H). MS (M/z, M+1), 431.5.

### EXAMPLE 204

### 4-(2-(2-Chlorophenyl)-6-(trifluoromethoxy)quinazolin-4-yl)thiomorpholine

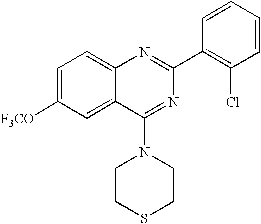

The compound 4-(2-(2-chlorophenyl)-6-(trifluoromethoxy)quinazolin-4-yl)thiomorpholine was prepared following the procedures described for Example 13 using 2-amino-5-trifluoromethoxybenzamide and 2-chlorobenzoyl chloride in Step 1 and thiomorpholine in Step 3. ¹H NMR (400 MHz, CDCl₃) δ 8.03 (d, 1H), 7.85 (m, 1H), 7.70 (m, 1H), 7.63 (m, 1H), 7.49 (m, 1H), 7.38 (m, 2H), 4.09 (m, 4H), 2.89 (m, 4H). MS (M/z, M+1), 426.2.

### EXAMPLE 205

### 6-Chloro-2-(2-chlorophenyl)-4-(pyrrolidin-1-yl)quinazoline

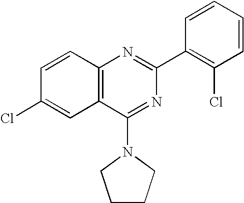

The compound 6-chloro-2-(2-chlorophenyl)-4-(pyrrolidin-1-yl)quinazoline was prepared following the procedures described for Example 13 using 2-amino-5-chlorobenzamide and 2-chlorobenzoyl chloride in Step 1 and pyrrolidine in Step 3. ¹H NMR (400 MHz, CDCl₃) δ 8.13 (m, 1H), 7.85 (m, 2H), 7.35 (m, 4H), 3.98 (m, 4H), 2.06 (m, 4H). MS (M/z, M+1), 344.4.

### EXAMPLE 206

### Ethyl 4-(6-chloro-2-(2-chlorophenyl)quinazolin-4-yl)piperazine-1-carboxylate

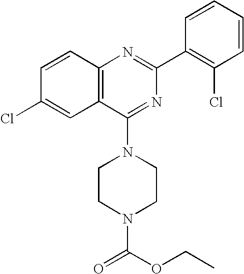

The compound ethyl 4-(6-chloro-2-(2-chlorophenyl)quinazolin-4-yl)piperazine-1-carboxylate was prepared following the procedures described for Example 13 using 2-amino-5-chlorobenzamide and 2-chlorobenozyl chloride in Step 1 and ethyl 1-piperazine carboxylate in Step 3. ¹H NMR (400 MHz, CDCl₃) δ 7.99 (d, 1H), 7.86 (m, 2H), 7.50 (m, 1H), 7.45 (m, 1H), 7.38 (m, 2H), 4.17 (q, 2H), 3.83 (m, 4H), 3.72 (m, 4H), 1.29 (t, 3H). MS (M/z, M+1), 431.5.

### EXAMPLE 207

### 4-(6-Chloro-2-(2-chlorophenyl)quinazolin-4-yl)thiomorpholine

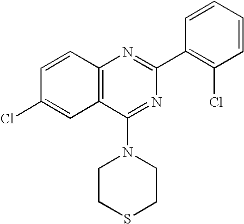

The compound 4-(6-chloro-2-(2-chlorophenyl)quinazolin-4-yl)thiomorpholine was prepared following the procedures described for Example 13 using 2-amino-5-chlorobenzamide and 2-chlorobenozyl chloride in Step 1 and thiomorpholine in Step 3. ¹H NMR (400 MHz, CDCl₃) δ 7.98 (d, 1H), 7.86 (m, 1H), 7.81 (d, 1H), 7.50 (m, 1H), 7.43 (m, 1H), 7.38 (m, 2H), 4.10 (m, 4H), 2.89 (m, 4H). MS (M/z, M+1), 376.3.

### EXAMPLE 208

### 6-Chloro-2-(2-chlorophenyl)-4-(4-methylpiperazin-1-yl)quinazoline

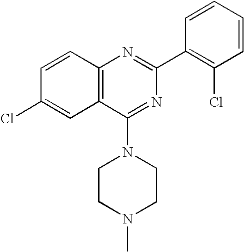

The compound 6-chloro-2-(2-chlorophenyl)-4-(4-methylpiperazin-1-yl)quinazoline was prepared following the procedures described for Example 13 using 2-amino-5-chlorobenzamide and 2-chlorobenzoyl chloride in Step 1 and 1-methylpiperazine in Step 3. ¹H NMR (400 MHz, CDCl₃) δ 7.96 (d, 1H), 7.86 (m, 2H), 7.48 (m, 1H), 7.38 (m, 3H), 3.89 (m, 4H), 2.62 (m, 4H), 2.38 (s, 3H). MS (M/z, M+1), 373.1.

### EXAMPLE 209

### 6-Chloro-2-(4-chlorophenyl)-4-(pyrrolidin-1-yl)quinazoline

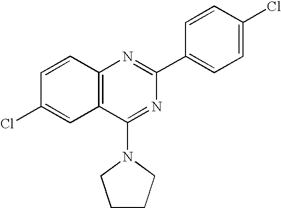

The compound 6-chloro-2-(4-chlorophenyl)-4-(pyrrolidin-1-yl)quinazoline was prepared following the procedures described for Example 13 using 2-amino-5-chlorobenzamide and 4-chlorobenozyl chloride in Step 1 and pyrrolidine in Step 3. ¹H NMR (400 MHz, CDCl₃) δ 8.45 (d, 2H), 8.06 (d, 1H), 7.86 (d, 1H), 7.42 (d, 2H), 7.28 (m, 1H), 3.99 (m, 4H), 2.08 (m, 4H). MS (M/z, M+1), 344.4.

### EXAMPLE 210

### Ethyl 4-(6-chloro-2-(4-chlorophenyl)quinazolin-4-yl)piperazine-1-carboxylate

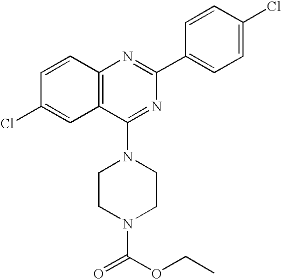

The compound ethyl 4-(6-chloro-2-(4-chlorophenyl)quinazolin-4-yl)piperazine-1-carboxylate was prepared following the procedures described for Example 13 using 2-amino-5-chlorobenzamide and 4-chlorobenzoyl chloride in Step 1 and ethyl 1-piperazine carboxylate in Step 3. ¹H NMR (400 MHz, CDCl₃) δ 8.46 (m, 2H), 7.95 (d, 1H), 7.80 (d, 1H), 7.44 (d, 2H), 7.38 (m, 1H), 4.20 (q, 2H), 3.81 (m, 4H), 3.75 (m, 4H), 1.31 (t, 3H). MS (M/z, M+1), 344.4.

### EXAMPLE 211

### 4-(6-Chloro-2-(4-chlorophenyl)quinazolin-4-yl)thiomorpholine

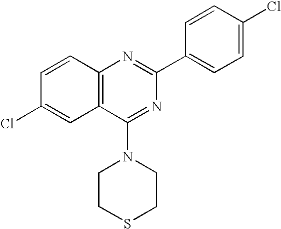

The compound 4-(6-chloro-2-(4-chlorophenyl)quinazolin-4-yl)thiomorpholine was prepared following the procedures described for Example 13 using 2-amino-5-chlorobenzamide and 4-chlorobenzoyl chloride in Step 1 and thiomorpholine in Step 3. ¹H NMR (400 MHz, CDCl₃) δ 8.46 (d, 2H), 7.94 (d, 1H), 7.75 (d, 1H), 7.45 (d, 2H), 7.36 (m, 1H), 4.11 (m, 4H), 2.90 (m, 4H). MS (M/z, M+1), 376.3.

### EXAMPLE 212

### 6-Chloro-2-(4-chlorophenyl)-4-(4-methylpiperazin-1-yl)quinazoline

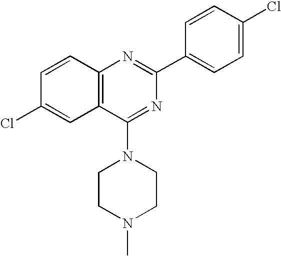

The compound 6-chloro-2-(4-chlorophenyl)-4-(4-methylpiperazin-1-yl)quinazoline was prepared following the procedures described for Example 13 using 2-amino-5-chlorobenzamide and 4-chlorobenozyl chloride in Step 1 and 1-methylpiperazine in Step 3. ¹H NMR (400 MHz, CDCl₃) δ 8.47 (d, 2H), 7.92 (d, 1H), 7.80 (d, 1H), 7.44 (d, 2H), 7.34 (m, 1H), 3.89 (m, 4H), 2.64 (m, 4H), 2.39 (s, 3H). MS (M/z, M+1), 373.4.

### EXAMPLE 213

### Ethyl 4-(2-(4-tert-butylphenyl)quinazolin-4-yl)piperazine-1-carboxylate

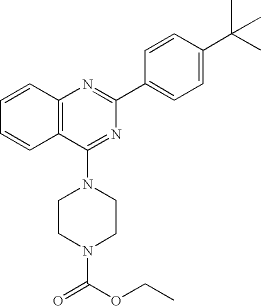

The compound ethyl 4-(2-(4-tert-butylphenyl)quinazolin-4-yl)piperazine-1-carboxylate was prepared following the procedures described for Example 13 using 4-tert-butylbenzoyl chloride in Step 1 and ethyl 1-piperazine carboxylate in Step 3. ¹H NMR (400 MHz, CDCl₃) δ 8.46 (d, 2H), 7.99 (d, 1H), 7.89 (d, 1H), 7.75 (m, 1H), 7.52 (d, 2H), 7.44 (m, 1H), 4.21 (q, 2H), 3.81 (m, 4H), 3.75 (m, 4H), 1.38 (s, 9H), 1.32 (t, 3H). MS (M/z, M+1), 419.2.

### EXAMPLE 214

### Ethyl 4-(2-(4-(trifluoromethoxy)phenyl)quinazolin-4-yl)piperazine-1-carboxylate

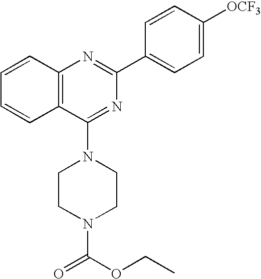

The compound ethyl 4-(2-(4-(trifluoromethoxy)phenyl)quinazolin-4-yl)piperazine-1-carboxylate was prepared following the procedures described for Example 13 using 4-(trifluoromethoxy)benzoyl chloride in Step 1 and ethyl 1-piperazine carboxylate in Step 3. ¹H NMR (400 MHz, CDCl₃) δ 8.59 (d, 2H), 7.98 (d, 1H), 7.89 (d, 1H), 7.77 (m, 1H), 7.47 (m, 1H), 7.32 (d, 2H), 4.21 (q, 2H), 3.83 (m, 4H), 3.75 (m, 4H), 1.31 (t, 3H). MS (M/z, M+1), 447.1.

### EXAMPLE 215

### Ethyl 4-(2-(3-fluorophenyl)quinazolin-4-yl)piperazine-1-carboxylate

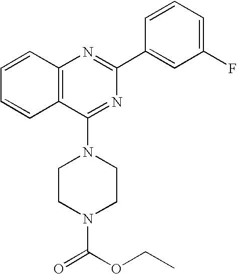

The compound ethyl 4-(2-(3-fluorophenyl)quinazolin-4-yl)piperazine-1-carboxylate was prepared following the procedures described for Example 13 using 3-fluorobenzoyl chloride in Step 1 and ethyl 1-piperazine carboxylate in Step 3. ¹H NMR (400 MHz, CDCl₃) δ 8.34 (m, 1H), 8.24 (m, 1H), 7.99 (d, 1H), 7.89 (d, 1H), 7.76 (m, 1H), 7.46 (m, 2H), 7.16 (m, 1H), 4.20 (q, 2H), 3.83 (m, 4H), 3.75 (m, 4H), 1.31 (t, 3H). MS (M/z, M+1), 381.2.

### EXAMPLE 216

### Ethyl 4-(2-(2,4-difluorophenyl)quinazolin-4-yl)piperazine-1-carboxylate

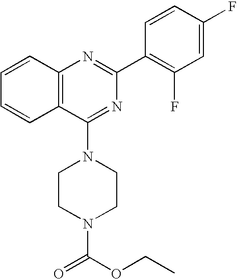

The compound ethyl 4-(2-(2,4-difluorophenyl)quinazolin-4-yl)piperazine-1-carboxylate was prepared following the procedures described for Example 13 using 2,4-difluorobenozyl chloride in Step 1 and ethyl 1-piperazine carboxylate in Step 3. ¹H NMR (400 MHz, CDCl₃) δ 8.19 (m, 1H), 7.98 (d, 1H), 7.89 (d, 1H), 7.77 (m, 1H), 7.49 (m, 1H), 6.96 (m, 2H), 4.19 (q, 2H), 3.81 (m, 4H), 3.73 (m, 4H), 1.30 (t, 3H). MS (M/z, M+1), 399.1.

### EXAMPLE 217

### Ethyl 4-(2-(2-chlorophenyl)-6-methoxyquinazolin-4-yl)piperazine-1-carboxylate

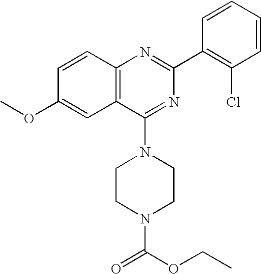

The compound ethyl 4-(2-(2-chlorophenyl)-6-methoxyquinazolin-4-yl)piperazine-1-carboxylate was prepared following the procedures described for Example 13 using 2-amino-5-methoxybenzamide and 2-chlorobenozyl chloride in Step 1 and ethyl 1-piperazine carboxylate in Step 3. ¹H NMR (400 MHz, CDCl₃) δ 7.96 (d, 1H), 7.85 (m, 1H), 7.47 (m, 2H), 7.37 (m, 2H), 7.20 (d, 1H), 4.19 (q, 2H), 3.95 (s, 3H), 3.74 (m, 8H), 1.30 (t, 3H). MS (M/z, M+1), 427.5.

### EXAMPLE 218

### Ethyl 4-(2-cyclohexyl-6-methoxyquinazolin-4-yl)piperazine-1-carboxylate

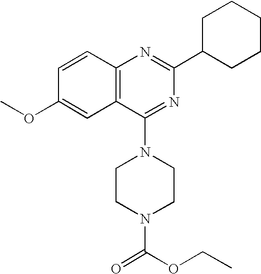

The compound ethyl 4-(2-cyclohexyl-6-methoxyquinazolin-4-yl)piperazine-1-carboxylate was prepared following the procedures described for Example 13 using 2-amino-5-methoxybenzamide and cyclohexanecarbonyl chloride in Step 1 and ethyl 1-piperazine carboxylate in Step 3. ¹H NMR (400 MHz, CDCl₃) δ 7.73 (d, 1H), 7.20 (d, 1H), 7.01 (dd, 1H), 4.19 (q, 2H), 3.93 (s, 3H), 3.69 (m, 8H), 2.80 (m, 1H), 2.02 (m, 2H), 1.85 (m, 2H), 1.69 (m, 4H), 1.42 (m, 2H), 1.30 (t, 3H). MS (M/z, M+1), 399.5.

### EXAMPLE 219

### Ethyl 4-(6-tert-butyl-2-(2-chlorophenyl)quinazolin-4-yl)piperazine-1-carboxylate

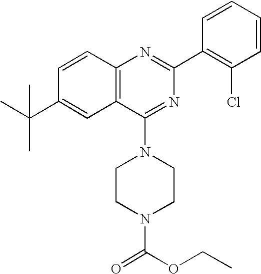

The compound ethyl 4-(6-tert-butyl-2-(2-chlorophenyl)quinazolin-4-yl)piperazine-1-carboxylate was prepared following the procedures described for Example 13 using 2-amino-5-tert-butylbenzamide and 2-chlorobenozyl chloride in Step 1 and ethyl 1-piperazine carboxylate in Step 3. ¹H NMR (400 MHz, CDCl₃) δ 7.96 (d, 1H), 7.89 (d, 1H), 7.86 (m, 2H), 7.49 (m, 1H), 7.36 (m, 2H), 4.20 (q, 2H), 3.80 (m, 4H), 3.75 (m, 4H), 1.44 (s, 9H), 1.31 (t, 3H). MS (M/z, M+1), 453.6.

### EXAMPLE 220

### Ethyl 4-(2-(3,4-difluorophenyl)quinazolin-4-yl)piperazine-1-carboxylate

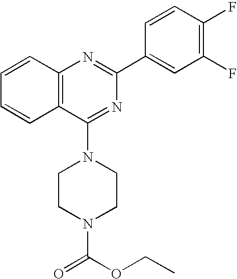

The compound ethyl 4-(2-(3,4-difluorophenyl)quinazolin-4-yl)piperazine-1-carboxylate was prepared following the procedures described for Example 13 using 3,4-difluorobenozyl chloride in Step 1 and ethyl 1-piperazine carboxylate in Step 3. ¹H NMR (400 MHz, CDCl₃) δ 8.39 (m, 1H), 8.33 (m, 1H), 7.97 (d, 1H), 7.90 (d, 1H), 7.77 (m, 1H), 7.48 (m, 1H), 7.25 (m, 1H), 4.21 (q, 2H), 3.83 (m, 4H), 3.76 (m, 4H), 1.32 (t, 3H). MS (M/z, M+1), 399.5.

### EXAMPLE 221

### Ethyl 4-(6-chloro-2-cyclohexylquinazolin-4-yl)piperazine-1-carboxylate

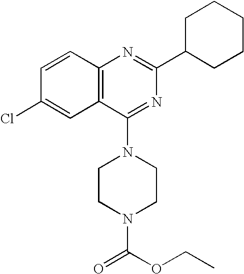

The compound ethyl 4-(6-chloro-2-cyclohexylquinazolin-4-yl)piperazine-1-carboxylate was prepared following the procedures described for Example 13 using 2-amino-5-chlorobenzamide and cyclohexanecarbonyl chloride in Step 1 and ethyl 1-piperazine carboxylate in Step 3. ¹H NMR (400 MHz, CDCl₃) δ 7.79 (m, 2H), 7.63 (m, 1H), 4.19 (q, 2H), 3.70 (m, 8H), 2.80 (m, 1H), 2.00 (m, 2H), 1.86 (m, 2H), 1.76 (m, 2H), 1.65 (m, 2H), 1.42 (m, 2H), 1.30 (t, 3H). MS (M/z, M+1), 403.5.

### EXAMPLE 222

### 2-(4-Chlorophenyl)-4-(pyrrolidin-1-yl)-6-(trifluoromethoxy)quinazoline

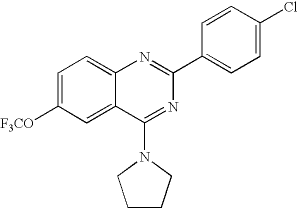

The compound 2-(4-chlorophenyl)-4-(pyrrolidin-1-yl)-6-(trifluoromethoxy) quinazoline was prepared following the procedures described for Example 13 using 2-amino-5-(trifluoromethoxy)benzamide and 4-chlorobenzoyl chloride in Step 1 and pyrrolidine in Step 3. ¹H NMR (400 MHz, CDCl₃) δ 8.48 (d, 2H), 8.00 (m, 1H), 7.90 (d, 1H), 7.55 (m, 1H), 7.44 (d, 2H), 4.02 (m, 4H), 2.11 (m, 4H). MS (M/z, M+1), 394.5.

### EXAMPLE 223

### Ethyl 4-(2-(4-chlorophenyl)-6-(trifluoromethoxy)quinazolin-4-yl)piperazine-1-carboxylate

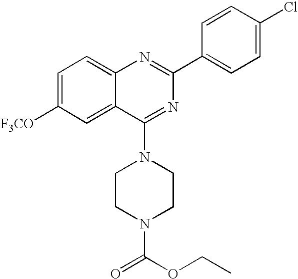

The compound ethyl 4-(2-(4-chlorophenyl)-6-(trifluoromethoxy)quinazolin-4-yl)piperazine-1-carboxylate was prepared following the procedure described for Example 13 using 2-amino-5-(trifluoromethoxy)benzamide and 4-chlorobenzoyl chloride in Step 1 and ethyl 1-piperazine carboxylate in Step 3. ¹H NMR (400 MHz, CDCl₃) δ 8.48 (d, 2H), 8.01 (d, 1H), 7.69 (m, 1H), 7.62 (m, 1H), 7.45 (d, 2H), 4.20 (q, 2H), 3.81 (m, 4H), 3.76 (m, 4H), 1.31 (t, 3H). MS (M/z, M+1), 481.6.

### EXAMPLE 224

### 4-(2-(4-Chlorophenyl)-6-(trifluoromethoxy)quinazolin-4-yl)thiomorpholine

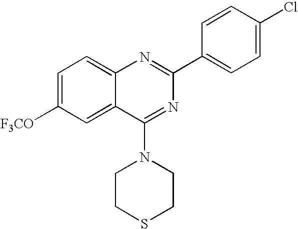

The compound 4-(2-(4-chlorophenyl)-6-(trifluoromethoxy)quinazolin-4-yl) thiomorpholine was prepared following the procedures described for Example 13 using 2-amino-4-(trifluoromethoxy)benzamide and 4-chlorobenozyl chloride in Step 1 and thiomorpholine in Step 3. ¹H NMR (400 MHz, CDCl₃) δ 8.48 (d, 2H), 7.99 (d, 1H), 7.65 (m, 1H), 7.60 (m, 1H), 7.46 (d, 2H), 4.10 (m, 4H), 2.92 (m, 4H). MS (M/z, M+1), 426.5.

### EXAMPLE 225

### 2-(4-Chlorophenyl)-4-(4-methylpiperazin-1-yl)-6-(trifluoromethoxy)quinazoline

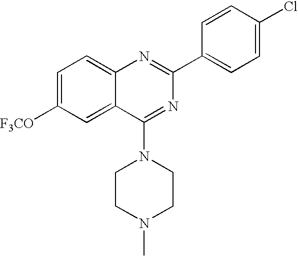

The compound 2-(4-chlorophenyl)-4-(4-methylpiperazin-1-yl)-6-(trifluoromethoxy)quinazoline was prepared following the procedures described for Example 13 using 2-amino-5-(trifluoromethoxy)benzamide and 4-chlorobenozyl chloride in Step 1 and 1-methylpiperazine in Step 3. ¹H NMR (400 MHz, CDCl₃) δ 8.48 (d, 2H), 7.97 (d, 1H), 7.70 (m, 1H), 7.58 (m, 1H), 7.46 (d, 2H), 3.87 (m, 4H), 2.67 (m, 4H), 2.40 (s, 3H). MS (M/z, M+1), 423.6.

### EXAMPLE 226

### Methyl 4-(2-(2-chlorophenyl)quinazolin-4-yl)piperazine-1-carboxylate

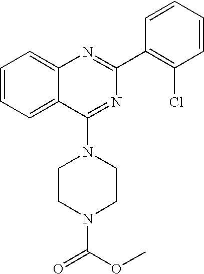

The compound methyl 4-(2-(2-chlorophenyl)quinazolin-4-yl)piperazine-1-carboxylate was prepared following the procedures described for Example 83 using methyl chloroformate. ¹H NMR (400 MHz, CDCl₃) δ 8.00 (d, 1H), 7.92 (d, 1H), 7.85 (m, 1H), 7.78 (m, 1H), 7.50 (m, 2H), 7.37 (m, 2H), 3.82 (m, 4H), 3.75 (s, 3H), 3.72 (m, 4H). MS (M/z, M+1), 383.4.

### EXAMPLE 227

### 8-Chloro-2-(2-chlorophenyl)-4-(pyrrolidin-1-yl)quinazoline

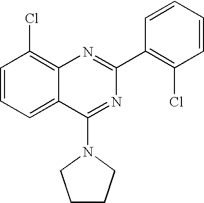

The compound 8-chloro-2-(2-chlorophenyl)-4-(pyrrolidin-1-yl)quinazoline was prepared following the procedures described for Example 13 using 2-amino-3-chlorobenzamide and 2-chlorobenzoyl chloride in Step 1 and pyrrolidine in Step 3. ¹H NMR (400 MHz, CDCl₃) δ 8.11 (m, 1H), 8.00 (m, 1H), 7.82 (m, 1H), 7.47 (m, 1H), 7.33 (m, 3H), 4.01 (m, 4H), 2.06 (m, 4H). MS (M/z, M+1), 344.4.

### EXAMPLE 228

### 8-Chloro-2-(2-chlorophenyl)-4-(4-methylpiperazin-1-yl)quinazoline

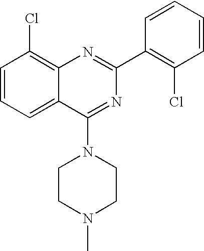

The compound 8-chloro-2-(2-chlorophenyl)-4-(4-methylpiperazin-1-yl)quinazoline was prepared following the procedures described for Example 13 using 2-amino-3-chlorobenzoyl chloride and 2-chlorobenozyl chloride in Step 1 and 1-methylpiperazine in Step 3. ¹H NMR (400 MHz, CDCl₃) δ 8.02 (m, 1H), 7.84 (m, 2H), 7.47 (m, 1H), 7.36 (m, 3H), 3.89 (m, 4H), 2.62 (m, 4H), 2.37 (s, 3H). MS (M/z, M+1), 373.4.

### EXAMPLE 229

### 2-(2-Chlorophenyl)-N-(2,2-dimethoxyethyl)-N-methylquinazolin-4-amine

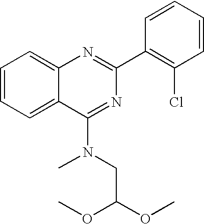

The compound 2-(2-chlorophenyl)-N-(2,2-dimethoxyethyl)-N-methylquinazolin-4-amine was following the procedures described for Example 21 using methyl iodide. ¹H NMR (400 MHz, CDCl₃) δ 8.19 (d, 1H), 7.94 (d, 1H), 7.79 (m, 1H), 7.74 (m, 1H), 7.45 (m, 2H), 7.35 (m, 2H), 4.90 (t, 1H), 3.93 (d, 2H), 3.60 (s, 3H), 3.43 (s, 6H). MS (M/z, M+1), 358.4.

### EXAMPLE 230

### 2-(2-Chlorophenyl)-N-(2,2-dimethoxyethyl)quinazolin-4-amine

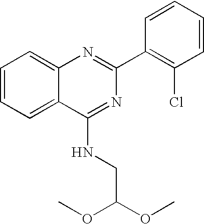

The compound 2-(2-chlorophenyl)-N-(2,2-dimethoxyethyl)quinazolin-4-amine was prepared following the procedures described for Example 13 using 2-chlorobenzoyl chloride in Step 1 and aminoacetaldehyde dimethylacetal in Step 3. ¹H NMR (400 MHz, CDCl₃) δ 7.94 (m, 1H), 7.82 (m, 1H), 7.77 (m, 2H), 7.50 (m, 2H), 7.36 (m, 2H), 6.02 (m, 1H), 4.66 (t, 1H), 3.90 (m, 2H), 3.47 (s, 6H). MS (M/z, M+1), 312.4.

### EXAMPLE 231

### Propyl 4-(5-chloro-2-(2-chlorophenyl)quinazolin-4-yl)piperazine-1-carboxylate

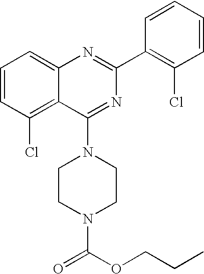

The compound propyl 4-(5-chloro-2-(2-chlorophenyl)quinazolin-4-yl)piperazine-1-carboxylate was prepared following the procedures described for Example 83 using propyl chloroformate. ¹H NMR (400 MHz, CDCl₃) δ 7.89 (m, 2H), 7.65 (m, 1H), 7.51 (m, 2H), 7.38 (m, 2H), 4.08 (t, 2H), 3.81 (m, 2H), 3.68 (m, 4H), 3.54 (m, 2H), 1.67 (m, 2H), 0.96 (t, 3H). MS (M/z, M+1), 445.3.

### EXAMPLE 232

### 1-(4-(5-Chloro-2-(2-chlorophenyl)quinazolin-4-yl)piperazin-1-yl)butan-1-one

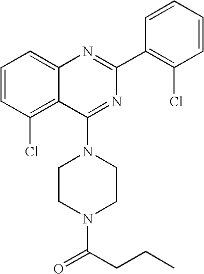

The compound 1-(4-(5-Chloro-2-(2-chlorophenyl)quinazolin-4-yl)piperazin-1-yl)butan-1-one was prepared following the procedures described for Example 83 using butyryl chloride. ¹H NMR (400 MHz, CDCl₃) δ 7.90 (m, 2H), 7.66 (m, 1H), 7.51 (m, 2H), 7.39 (m, 2H), 3.68 (m, 8H), 2.35 (t, 2H), 1.69 (m, 2H), 0.99 (t, 3H). MS (M/z, M+1), 429.3.

### EXAMPLE 233

### 1-(4-(7-Chloro-2-(2-chlorophenyl)quinazolin-4-yl)piperazin-1-yl)ethanone

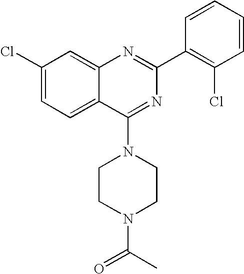

The compound 1-(4-(7-chloro-2-(2-chlorophenyl)quinazolin-4-yl)piperazin-1-yl)ethanone was prepared following the procedures described for Example 13 using 2-amino-4-chlorobenzamide and 2-chlorobenzoyl chloride in Step 1 and 1-acetylpiperazine in Step 3. ¹H NMR (400 MHz, CDCl₃) δ 8.00 (d, 1H), 7.86 (m, 2H), 7.50 (m, 1H), 7.45 (m, 1H), 7.39 (m, 2H), 3.89 (m, 2H), 3.84 (m, 4H), 3.72 (m, 2H), 2.17 (s, 3H). MS (M/z, M+1), 401.0.

### EXAMPLE 234

### Ethyl 4-(7-chloro-2-(2-chlorophenyl)quinazolin-4-yl)piperazine-1-carboxylate

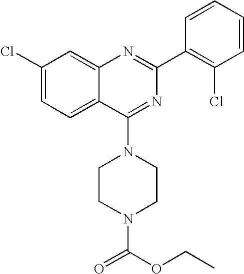

The compound ethyl 4-(7-chloro-2-(2-chlorophenyl)quinazolin-4-yl)piperazine-1-carboxylate was prepared following the procedures described for Example 13 using 2-amino-4-chlorobenzamide and 2-chlorobenzoyl chloride in Step 1 and ethyl 1-piperazine carboxylate in Step 3. ¹H NMR (400 MHz, CDCl₃) δ 7.99 (d, 1H), 7.85 (m, 2H), 7.50 (m, 1H), 7.45 (m, 1H), 7.38 (m, 2H), 4.19 (q, 2H), 3.82 (m, 4H), 3.72 (m, 4H), 1.30 (t, 3H). MS (M/z, M+1), 431.0.

### EXAMPLE 235

### 7-Chloro-2-(2-chlorophenyl)-4-(pyrrolidin-1-yl)quinazoline

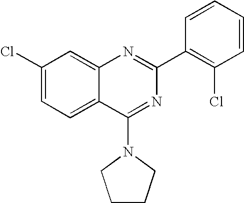

The compound 7-chloro-2-(2-chlorophenyl)-4-(pyrrolidin-1-yl)quinazoline was prepared following the procedures described for Example 13 using 2-amino-4-chlorobenzamide and 2-chlorobenozyl chloride in Step 1 and pyrrolidine in Step 1. ¹H NMR (400 MHz, CDCl₃) δ 8.12 (d, 1H), 7.90 (d, 1H), 7.81 (m, 1H), 7.47 (m, 1H), 7.35 (m, 3H), 3.99 (m, 4H), 2.06 (m, 4H). MS (M/z, M+1), 344.0.

### EXAMPLE 236

### 5-Chloro-2-(2-chlorophenyl)-4-(piperazin-1-yl)quinazoline

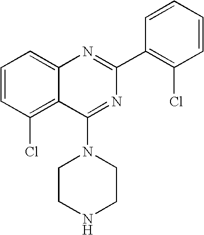

The compound 5-chloro-2-(2-chlorophenyl)-4-(piperazin-1-yl)quinazoline was prepared following the procedures described for Example 13 using 2-amino-6-chlorobenzamide and 2-chlorobenozyl chloride in Step 1 and piperazine in Step 3. ¹H NMR (400 MHz, CDCl₃) δ 7.89 (m, 2H), 7.64 (m, 1H), 7.51 (m, 2H), 7.40 (m, 2H), 3.84 (m, 2H), 3.60 (m, 2H), 3.04 (m, 4H). MS (M/z, M+1), 358.9.

### EXAMPLE 237

### 1-(4-(5-Chloro-2-(2-fluorophenyl)quinazolin-4-yl)piperazin-1-yl)ethanone

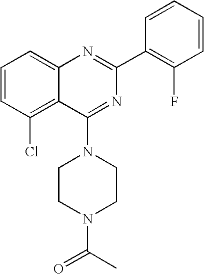

The compound 1-(4-(5-Chloro-2-(2-fluorophenyl)quinazolin-4-yl)piperazin-1-yl)ethanone was prepared following the procedures described for Example 13 using 2-amino-6-chlorobenzamide and 2-fluorobenozyl chloride in Step 1 and 1-acetylpiperazine in Step 3. ¹H NMR (400 MHz, CDCl₃) δ 8.17 (m, 1H), 7.90 (m, 1H), 7.65 (m, 1H), 7.50 (m, 1H), 7.44 (m, 1H), 7.27 (m, 1H), 7.19 (m, 1H), 3.81 (m, 4H), 3.69 (m, 3H), 3.47 (m, 1H), 2.16 (s, 3H). MS (M/z, M+1), 385.5.

### EXAMPLE 238

### Ethyl 4-(5-chloro-2-(2-fluorophenyl)quinazolin-4-yl)piperazine-1-carboxylate

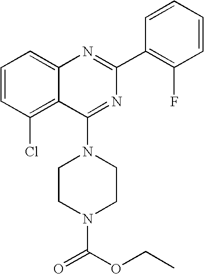

The compound ethyl 4-(5-chloro-2-(2-fluorophenyl)quinazolin-4-yl)piperazine-1-carboxylate was prepared following the procedures described for Example 13 using 2-amino-6-chlorobenzamide and 2-fluorobenozyl chloride in Step 1 and ethyl 1-piperazine carboxylate in Step 3. ¹H NMR (400 MHz, CDCl₃) δ 8.16 (m, 1H), 7.89 (m, 1H), 7.64 (m, 1H), 7.49 (m, 1H), 7.45 (m, 1H), 7.26 (m, 1H), 7.19 (m, 1H), 4.16 (q, 2H), 3.68 (m, 8H), 1.29 (t, 3H). MS (M/z, M+1), 415.5.

### EXAMPLE 239

### 5-Chloro-2-(2-fluorophenyl)-4-(pyrrolidin-1-yl)quinazoline

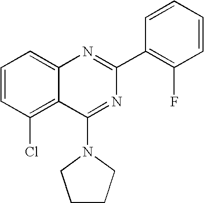

The compound 5-chloro-2-(2-fluorophenyl)-4-(pyrrolidin-1-yl)quinazoline was prepared following the procedures described for Example 13 using 2-amino-6-chlorobenzamide and 2-fluorobenozyl chloride in Step 1 and pyrrolidine in Step 3. ¹H NMR (400 MHz, CDCl₃) δ 8.12 (m, 1H), 7.77 (m, 1H), 7.56 (m, 1H), 7.40 (m, 2H), 7.26 (m, 1H), 7.17 (m, 1H), 3.87 (m, 4H), 1.93 (m, 4H). MS (M/z, M+1), 328.4.

### EXAMPLE 240

### 1-(4-(2-(2-Chlorophenyl)-5-(trifluoromethyl)quinazolin-4-yl)piperazin-1-yl)ethanone

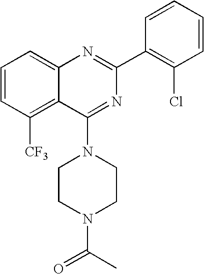

The compound 1-(4-(2-(2-chlorophenyl)-5-(trifluoromethyl)quinazolin-4-yl)piperazin-1-yl)ethanone was prepared following the procedures described for Example 13 using 2-amino-6-(trifluoromethyl)benzamide and 2-chlorobenozyl chloride in Step 1 and 1-acetylpiperazine in Step 3. ¹H NMR (400 MHz, CDCl₃) δ 8.16 (d, 1H), 7.93 (m, 2H), 7.51 (m, 1H), 7.41 (m, 2H), 3.66 (m, 7H), 3.37 (m, 1H), 2.13 (s, 3H). MS (M/z, M+1), 435.5.

### EXAMPLE 241

### Ethyl 4-(2-(2-chlorophenyl)-5-(trifluoromethyl)quinazolin-4-yl)piperazine-1-carboxylate

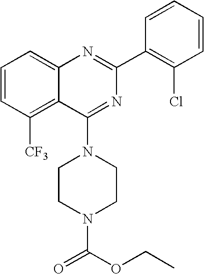

The compound ethyl 4-(2-(2-chlorophenyl)-5-(trifluoromethyl)quinazolin-4-yl)piperazine-1-carboxylate was prepared following the procedures described for Example 13 using 2-amino-6-(trifluoromethyl)benzamide and 2-chlorobenzoyl chloride in Step 1 and ethyl 1-piperazine carboxylate in Step 3. ¹H NMR (400 MHz, CDCl₃) δ 8.14 (d, 1H), 7.92 (m, 1H) 7.89 (m, 1H), 7.82 (m, 1H), 7.51 (m, 1H), 7.39 (m, 2H), 4.16 (q, 2H), 3.60 (m, 8H), 1.27 (t, 3H). MS (M/z, M+1), 465.6.

### EXAMPLE 242

### 2-(2-Chlorophenyl)-4-(pyrrolidin-1-yl)-5-(trifluoromethyl)quinazoline

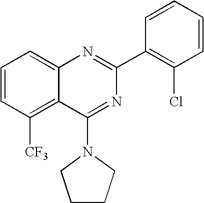

The compound 2-(2-chlorophenyl)-4-(pyrrolidin-1-yl)-5-(trifluoromethyl)quinazoline was prepared following the procedures described for Example 13 using 2-amino-6-(trifluoromethyl)benzamide and 2-chlorobenozyl chloride in Step 1 and pyrrolidine in Step 3. ¹H NMR (400 MHz, CDCl₃) δ 7.99 (m, 1H), 7.81 (m, 1H), 7.72 (m, 2H), 7.48 (m, 1H), 7.34 (m, 2H), 3.70 (m, 4H), 1.89 (m, 4H). MS (M/z, M+1), 378.5.

### EXAMPLE 243

### 1-(4-(2-(2-Chlorophenyl)-5-methoxyquinazolin-4-yl)piperazin-1-yl)ethanone

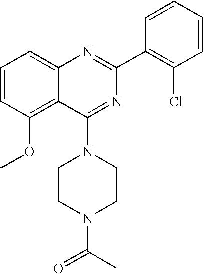

The compound 1-(4-(2-(2-chlorophenyl)-5-methoxyquinazolin-4-yl)piperazin-1-yl)ethanone was prepared following the procedures described for Example 13 using 2-amino-6-methoxybenzamide and 2-chlorobenzoyl chloride in Step 1 and 1-acetylpiperazine in Step 3. ¹H NMR (400 MHz, CDCl₃) δ 7.87 (m, 1H), 7.69 (m, 1H), 7.57 (m, 1H), 7.47 (m, 1H), 7.36 (m, 2H), 6.90 (m, 1H), 4.01 (s, 3H), 3.76 (m, 4H), 3.65 (m, 2H), 3.59 (m, 2H), 2.16 (s, 3H). MS (M/z, M+1), 397.5.

### EXAMPLE 244

### Ethyl 4-(2-(2-chlorophenyl)-5-methoxyquinazolin-4-yl)piperazine-1-carboxylate

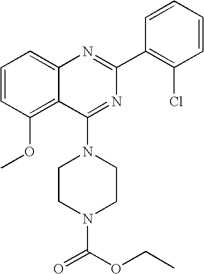

The compound ethyl 4-(2-(2-chlorophenyl)-5-methoxyquinazolin-4-yl)piperazine-1-carboxylate was prepared following the procedures described for Example 13 using 2-amino-6-methoxybenzamide and 2-chlorobenzoyl chloride in Step 1 and ethyl 1-piperazine carboxylate in Step 3. ¹H NMR (400 MHz, CDCl₃) δ 7.86 (m, 1H), 7.68 (m, 1H), 7.56 (m, 1H), 7.47 (m, 1H), 7.36 (m, 2H), 6.88 (m, 1H), 4.16 (q, 2H), 4.00 (s, 3H), 3.66 (m, 8H), 1.28 (t, 3H). MS (M/z, M+1), 427.6.

### EXAMPLE 245

### 2-(2-Chlorophenyl)-5-methoxy-4-(pyrrolidin-1-yl)quinazoline

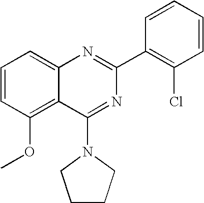

The compound 2-(2-chlorophenyl)-5-methoxy-4-(pyrrolidin-1-yl)quinazoline was prepared following the procedures described for Example 13 using 2-amino-6-methoxybenzamide and 2-chlorobenozyl chloride in Step 1 and pyrrolidine in Step 3. ¹H NMR (400 MHz, CDCl₃) δ 7.80 (m, 1H), 7.61 (m, 1H), 7.45 (m, 2H), 7.32 (m, 2H), 6.80 (m, 1H), 3.98 (s, 3H), 3.75 (m, 4H), 1.91 (m, 4H). MS (M/z, M+1), 340.5.

### EXAMPLE 246

### 1-(4-(2-(2-Chlorophenyl)-5-fluoroquinazolin-4-yl)piperazin-1-yl)ethanone

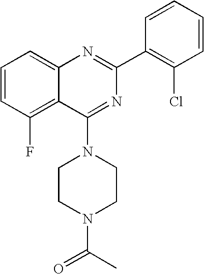

The compound 1-(4-(2-(2-chlorophenyl)-5-fluoroquinazolin-4-yl)piperazin-1-yl)ethanone was prepared following the procedures described for Example 13 using 2-amino-6-fluorobenzamide and 2-chlorobenozyl chloride in Step 1 and 1-acetylpiperazine in Step 3. ¹H NMR (400 MHz, CDCl₃) δ 7.86 (m, 1H), 7.79 (m, 1H), 7.73 (m, 1H), 7.49 (m, 1H), 7.38 (m, 2H), 7.17 (m, 1H), 3.82 (m, 2H), 3.75 (m, 2H), 3.67 (m, 4H), 2.15 (s, 3H). MS (M/z, M+1), 385.5.

### EXAMPLE 247

### Ethyl 4-(2-(2-chlorophenyl)-5-fluoroquinazolin-4-yl)piperazine-1-carboxylate

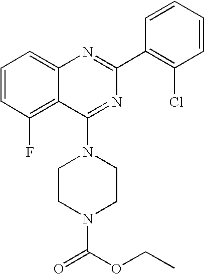

The compound ethyl 4-(2-(2-chlorophenyl)-5-fluoroquinazolin-4-yl)piperazine-1-carboxylate was prepared following the procedures described for Example 13 using 2-amino-6-fluorobenzamide and 2-chlorobenzoyl chloride in Step 1 and ethyl 1-piperazine carboxylate in Step 3. ¹H NMR (400 MHz, CDCl₃) δ 7.85 (m, 1H), 7.77 (m, 1H), 7.71 (m, 1H), 7.48 (m, 1H), 7.37 (m, 2H), 7.15 (m, 1H), 4.17 (q, 2H), 3.69 (m, 8H), 1.28 (t, 3H). MS (M/z, M+1), 415.5.

### EXAMPLE 248

### 2-(2-Chlorophenyl)-5-fluoro-4-(pyrrolidin-1-yl)quinazoline

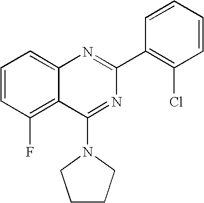

The compound 2-(2-chlorophenyl)-5-fluoro-4-(pyrrolidin-1-yl)quinazoline was prepared following the procedures described for Example 13 using 2-amino-6-fluorobenzoyl chloride and 2-chlorobenozyl chloride in Step 1 and pyrrolidine in Step 3. ¹H NMR (400 MHz, CDCl₃) δ 7.80 (m, 1H), 7.66 (m, 2H), 7.46 (m, 1H), 7.34 (m, 2H), 7.06 (m, 1H), 3.81 (m, 4H), 1.96 (m, 4H). MS (M/z, M+1), 328.4.

### EXAMPLE 249

### 1-(4-(2-(4-tert-Butylphenyl)-6-methoxyquinazolin-4-yl)piperazin-1-yl)ethanone

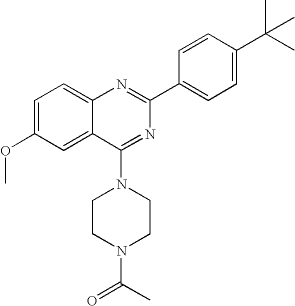

The compound 1-(4-(2-(4-tert-butylphenyl)-6-methoxyquinazolin-4-yl)piperazin-1-yl)ethanone was prepared following the procedures described for Example 13 using 2-amino-5-methoxybenzamide and 4-tert-butylbenozyl chloride in Step 1 and 1-acetylpiperazine in Step 3. ¹H NMR (400 MHz, CDCl₃) δ 8.42 (d, 2H), 7.93 (d, 1H), 7.51 (d, 2H), 7.42 (m, 1H), 7.15 (d, 1H), 3.92 (s, 3H), 3.85 (m, 2H), 3.79 (m, 2H), 3.74 (m, 4H), 2.19 (s, 3H), 1.37 (s, 9H). MS (M/z, M+1), 419.6.

### EXAMPLE 250

### Ethyl 4-(2-(4-tert-butylphenyl)-6-methoxyquinazolin-4-yl)piperazine-1-carboxylate

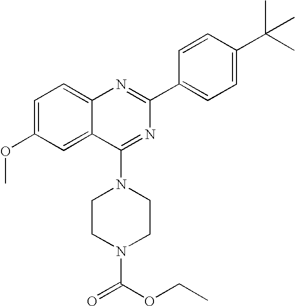

The compound ethyl 4-(2-(4-tert-butylphenyl)-6-methoxyquinazolin-4-yl)piperazine-1-carboxylate was prepared following the procedures described for Example 13 using 2-amino-5-methoxybenzamide and 4-tert-butylbenzoyl chloride in Step 1 and ethyl 1-piperazine carboxylate in Step 3. ¹H NMR (400 MHz, CDCl₃) δ 8.42 (d, 2H), 7.92 (d, 1H), 7.50 (d, 2H), 7.42 (m, 1H), 7.16 (d, 1H), 4.20 (q, 2H), 3.93 (s, 3H), 3.75 (m, 8H), 1.37 (s, 9H), 1.31 (t, 3H). MS (M/z, M+1), 449.6.

### EXAMPLE 251

### 2-(4-tert-Butylphenyl)-6-methoxy-4-(pyrrolidin-1-yl)quinazoline

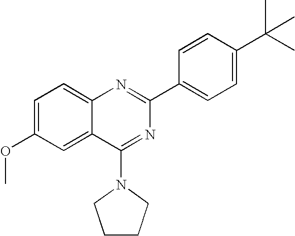

The compound 2-(4-tert-butylphenyl)-6-methoxy-4-(pyrrolidin-1-yl)quinazoline was prepared following the procedures described for Example 13 using 2-amino-5-methoxybenzamide and 4-tert-butylbenozyl chloride in Step 1 and pyrrolidine in Step 3. ¹H NMR (400 MHz, CDCl₃) δ 8.42 (d, 2H), 7.85 (d, 1H), 7.53 (d, 1H), 7.48 (d, 2H), 7.35 (m, 1H), 4.03 (m, 4H), 3.90 (s, 3H), 2.06 (m, 4H), 1.36 (s, 9H). MS (M/z, M+1), 362.5.

### EXAMPLE 252

### Ethyl 4-(2-(4-tert-butylphenyl)-6-chloroquinazolin-4-yl)piperazine-1-carboxylate

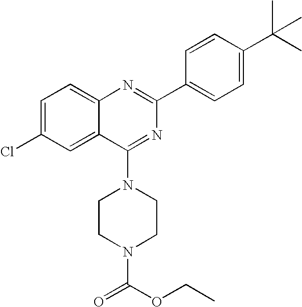

The compound ethyl 4-(2-(4-tert-butylphenyl)-6-chloroquinazolin-4-yl)piperazine-1-carboxylate was prepared following the procedures described for Example 13 using 2-amino-5-chlorobenzamide and 4-tert-butylbenzoyl chloride in Step 1 and ethyl 1-piperazine carboxylate in Step 3. ¹H NMR (400 MHz, CDCl₃) δ 8.43 (d, 2H), 7.97 (d, 1H), 7.79 (d, 1H), 7.51 (d, 2H), 7.36 (m, 1H), 4.20 (q, 2H), 3.80 (m, 4H), 3.73 (m, 4H), 1.38 (s, 9H), 1.31 (t, 3H). MS (M/z, M+1), 453.6.

### EXAMPLE 253

### 1-(4-(6-Chloro-2-(4-chlorophenyl)quinazolin-4-yl)piperazin-1-yl)ethanone

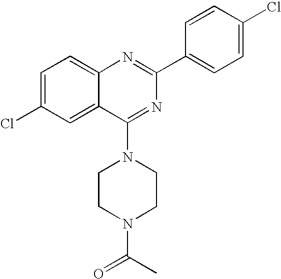

The compound 1-(4-(6-chloro-2-(4-chlorophenyl)quinazolin-4-yl)piperazin-1-yl)ethanone was prepared following the procedures described for Example 13 using 2-amino-5-chlorobenzamide and 4-chlorobenzoyl chloride in Step 1 and 1-acetylpiperazine in Step 3. ¹H NMR (400 MHz, CDCl₃) δ 8.46 (d, 2H), 7.96 (d, 1H), 7.81 (d, 1H), 7.45 (d, 2H), 7.39 (m, 1H), 3.81 (m, 8H), 2.19 (s, 3H). MS (M/z, M+1), 401.4.

### EXAMPLE 254

### 1-(4-(2-(2-Chlorophenyl)-5-methylquinazolin-4-yl)piperazin-1-yl)ethanone

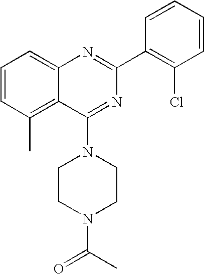

The compound 1-(4-(2-(2-Chlorophenyl)-5-methylquinazolin-4-yl)piperazin-1-yl)ethanone was prepared following the procedures described for Example 13 using 2-amino-6-methylbenzamide and 2-chlorobenzoyl chloride in Step 1 and 1-acetylpiperazine in Step 3. ¹H NMR (400 MHz, CDCl₃) δ 7.89 (m, 1H), 7.84 (m, 1H), 7.67 (m, 1H), 7.49 (m, 1H), 7.36 (m, 3H), 3.67 (m, 7H), 3.36 (m, 1H), 2.82 (s, 3H), 2.14 (s, 3H). MS (M/z, M+1), 381.4.

### EXAMPLE 255

### Ethyl 4-(2-(2-chlorophenyl)-5-methylquinazolin-4-yl)piperazine-1-carboxylate

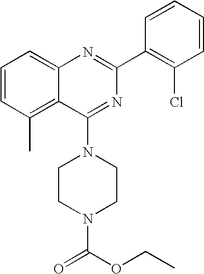

The compound ethyl 4-(2-(2-chlorophenyl)-5-methylquinazolin-4-yl)piperazine-1-carboxylate was prepared as described for Example 13 using 2-amino-6-methylbenzamide and 2-chlorobenzoyl chloride in Step 1 and ethyl 1-piperazine carboxylate in Step 3. ¹H NMR (400 MHz, CDCl₃) δ 7.90 (m, 1H), 7.83 (m, 1H), 7.66 (m, 1H), 7.49 (m, 1H), 7.36 (m, 3H), 4.17 (q, 2H), 3.59 (m, 8H), 2.82 (s, 3H), 1.28 (t, 3H). MS (M/z, M+1), 411.5.

### EXAMPLE 256

### 2-(2-Chlorophenyl)-5-methyl-4-(pyrrolidin-1-yl)quinazoline

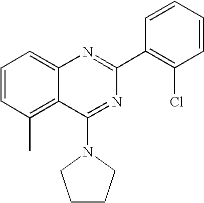

The compound 2-(2-Chlorophenyl)-5-methyl-4-(pyrrolidin-1-yl)quinazoline was prepared following the procedures described for Example 13 using 2-amino-6-methylbenzamide and 2-chlorobenzoyl chloride in Step 1 and pyrrolidine in Step 3. ¹H NMR (400 MHz, CDCl₃) δ 7.82 (m, 1H), 7.69 (m, 1H), 7.58 (m, 1H), 7.46 (m, 1H), 7.33 (m, 2H), 7.24 (m, 1H), 3.72 (m, 4H), 2.69 (s, 3H), 1.88 (m, 4H). MS (M/z, M+1), 324.4.

### EXAMPLE 257

### 2-(2-Chlorophenyl)-4-(4-(1-methyl-1H-imidazol-2-yl)piperazin-1-yl)quinazoline

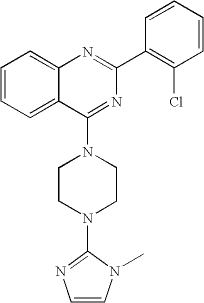

The compound 2-(2-chlorophenyl)-4-(4-(1-methyl-1H-imidazol-2-yl)piperazin-1-yl)quinazoline was prepared from 2-(2-chlorophenyl)-4-(piperazin-1-yl)quinazoline. 2-(2-chlorophenyl)-4-(piperazine-1-yl)quinazoline (91 mg, 0.281 mmol) was dissolved in NMP (3 mL) in a microwave reactor vial. Then 2-bromo-1-methylimidazole (0.05 mL, 0.561 mmol) was added and the mixture heated at 200° C. for 10 minutes in a microwave reactor. The mixture was poured into H₂O, extracted with CH₂Cl₂, dried over Na₂SO₄, filtered, and concentrated in vacuo. The crude material was then purified by reverse phase HPLC (10%-90% MeCN/H₂O) to give the title compound. ¹H NMR (400 MHz, CDCl₃) δ 8.23 (m, 1H), 8.13 (m, 1H), 8.02 (m, 1H), 7.92 (m, 2H), 7.67 (m, 1H), 7.46 (m, 2H), 7.16 (d, 1H), 6.83 (d, 1H), 4.32 (m, 4H), 3.69 (m, 5H), 3.39 (m, 2H). MS (M/z, M+1), 405.5.

### EXAMPLE 258

### N-(3-(1H-Imidazol-1-yl)propyl)-2-(2-chlorophenyl)quinazolin-4-amine

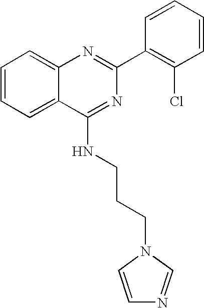

The compound N-(3-(1H-imidazol-1-yl)propyl)-2-(2-chlorophenyl)quinazolin-4-amine was prepared as described for Example 13 using 2-chlorobenzoyl chloride in Step 1 and 1-(3-aminopropyl)imidazole in Step 3. ¹H NMR (400 MHz, CDCl₃) δ 7.91 (m, 2H), 7.77 (m, 2H), 7.48 (m, 3H), 7.36 (m, 2H), 7.07 (m, 1H), 6.97 (m, 2H), 4.08 (t, 2H), 3.69 (q, 2H), 2.72 (m, 2H). MS (M/z, M+1), 364.4.

### EXAMPLE 259

### N-(3-(1H-Imidazol-1-yl)propyl)-2-(2-chlorophenyl)-N-methylquinazolin-4-amine

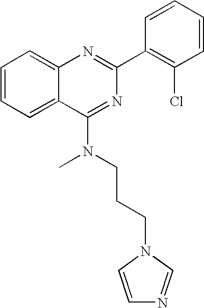

The compound N-(3-(1H-imidazol-1-yl)propyl)-2-(2-chlorophenyl)-N-methylquinazolin-4-amine was prepared following the procedures described for Example 21 using methyl iodide. ¹H NMR (400 MHz, CDCl₃) δ 8.02 (d, 1H), 7.96 (d, 1H), 7.79 (m, 1H), 7.75 (m, 1H), 7.43 (m, 5H), 7.15 (m, 1H), 6.94 (m, 1H), 4.06 (t, 2H), 3.80 (m, 2H), 3.44 (s, 3H), 2.33 (m, 2H). MS (M/z, M+1), 377.9.

### EXAMPLE 260

### 1-(4-(5-Chloro-2-(2-chlorophenyl)quinazolin-4-yl)piperazin-1-yl)propan-1-one

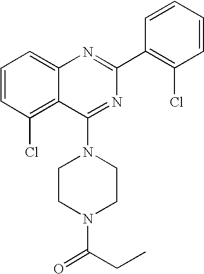

The compound 1-(4-(5-chloro-2-(2-chlorophenyl)quinazolin-4-yl)piperazin-1-yl)propan-1-one was prepared as described for Example 83 using propionyl chloride. ¹H NMR (400 MHz, CDCl₃) δ 7.88 (m, 2H), 7.65 (m, 1H), 7.49 (m, 2H), 7.37 (m, 2H), 3.74 (m, 8H), 2.38 (q, 2H), 1.17 (t, 3H). MS (M/z, M+1), 415.4.

### EXAMPLE 261

### Methyl 4-(5-chloro-2-(2-chlorophenyl)quinazolin-4-yl)piperazine-1-carboxylate

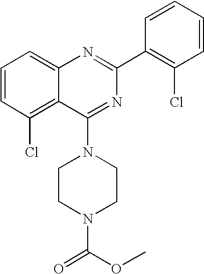

The compound methyl 4-(5-chloro-2-(2-chlorophenyl)quinazolin-4-yl)piperazine-1-carboxylate was prepared as described for Example 83 using methyl chloroformate. ¹H NMR (400 MHz, CDCl₃) δ 8.01 (m, 1H), 7.90 (m, 1H), 7.67 (m, 1H), 7.50 (m, 2H), 7.40 (m, 2H), 3.71 (m, 11H). MS (M/z, M+1), 417.4.

### EXAMPLE 262

### (4-(2-(2-Chlorophenyl)quinazolin-4-yl)piperazin-1-yl)(1H-imidazol-2-yl)methanone

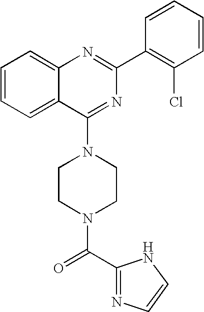

The compound (4-(2-(2-chlorophenyl)quinazolin-4-yl)piperazin-1-yl)(1H-imidazol-2-yl)methanone was prepared as described for Example 100 using 1H-imidizole-2-carboxylic acid. ¹H NMR (400 MHz, CDCl₃) δ 8.00 (d, 1H), 7.96 (d, 1H), 7.85 (m, 1H), 7.78 (m, 1H), 7.50 (m, 2H), 7.37 (m, 2H), 7.28 (m, 1H), 7.13 (m, 1H), 4.90 (m, 1H), 3.99 (m, 8H). MS (M/z, M+1), 419.4.

### EXAMPLE 263

### (4-(2-(2-chlorophenyl)quinazolin-4-yl)piperazin-1-yl)(1-methyl-1H-imidazol-2-yl)methanone

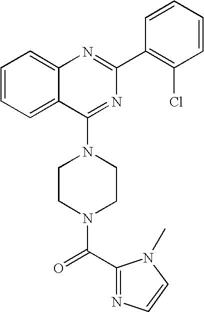

The compound (4-(2-(2-chlorophenyl)quinazolin-4-yl)piperazin-1-yl)(1-methyl-1H-imidazol-2-yl)methanone was prepared as described for Example 21 using methyl iodide. ¹H NMR (400 MHz, CDCl₃) δ 7.98 (m, 2H), 7.86 (m, 1H), 7.89 (m, 1H), 7.50 (m, 2H), 7.35 (m, 2H), 7.06 (d, 1H), 6.96 (d, 1H), 4.37 (m, 2H), 3.98 (m, 6H), 3.91 (s, 3H). MS (M/z, M+1), 433.5.

### EXAMPLE 264

### 2-(4-(2-(2-Chlorophenyl)quinazolin-4-yl)piperazin-1-yl)thiazole

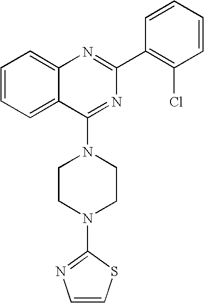

The compound 2-(4-(2-(2-chlorophenyl)quinazolin-4-yl)piperazin-1-yl)thiazole was prepared from 2-(2-chlorophenyl)-4-(piperazin-1-yl)quinazoline. 2-(2-Chlorophenyl)-4-(piperazin-1-yl)quinazoline (99 mg, 0.305 mmol), Pd₂(dba)₃ (28 mg, 0.031 mmol), X-Phos (30 mg, 0.062 mmol), sodium tert-butoxide (44 mg, 0.458 mmol), and 2-bromothiazole (0.03 mL, 0.366 mmol) were dissolved in toluene (3 mL) and the mixture was heated to 110° C. for 3 h. The mixture was filtered through Celite and the filtrate was concentrated. The crude material was then purified by flash chromatography (0-8% MeOH/CH₂Cl₂) to give the title compound. ¹H NMR (400 MHz, CDCl₃) δ 8.00 (m, 2H), 7.86 (m, 1H), 7.80 (m, 1H), 7.52 (m, 2H), 7.37 (m, 2H), 7.24 (d, 1H), 6.63 (d, 1H), 3.97 (m, 4H), 3.74 (m, 4H). MS (M/z, M+1), 408.4.

### EXAMPLE 265

### 1-(4-(5-Chloro-2-(2-chlorophenyl)quinazolin-4-yl)piperazin-1-yl)ethanone

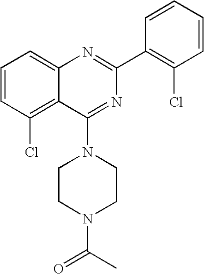

The compound 1-(4-(5-chloro-2-(2-chlorophenyl)quinazolin-4-yl)piperazin-1-yl)ethanone was prepared as described for Example 13 using 2-amino-6-chlorobenzamide and 2-chlorobenzoyl chloride in Step 1 and 1-acetylpiperazine in Step 3. ¹H NMR (400 MHz, CDCl₃) δ 7.90 (m, 2H), 7.66 (m, 1H), 7.51 (m, 2H), 7.39 (m, 2H), 3.68 (m, 8H), 2.15 (s, 3H). MS (M/z, M+1), 401.3.

### EXAMPLE 266

### Ethyl 4-(5-chloro-2-(2-chlorophenyl)quinazolin-4-yl)piperazine-1-carboxylate

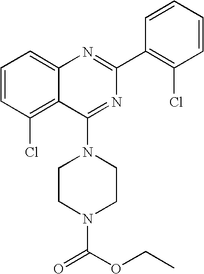

The compound ethyl 4-(5-chloro-2-(2-chlorophenyl)quinazolin-4-yl)piperazine-1-carboxylate was prepared following the procedures described for Example 13 using 2-amino-6-chlorobenzamide and 2-chlorobenozyl chloride in Step 1 and ethyl 1-piperazine carboxylate in Step 3. ¹H NMR (400 MHz, CDCl₃) δ 7.89 (m, 2H), 7.65 (m, 1H), 7.51 (m, 2H), 7.38 (m, 2H), 4.17 (q, 2H), 3.81 (m, 2H), 3.68 (m, 4H), 3.54 (m, 2H), 1.28 (t, 3H). MS (M/z, M+1), 431.3.

### EXAMPLE 267

### 5-Chloro-2-(2-chlorophenyl)-4-(pyrrolidin-1-yl)quinazoline

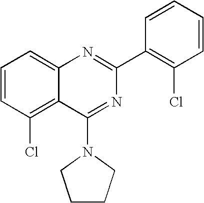

The compound 5-chloro-2-(2-chlorophenyl)-4-(pyrrolidin-1-yl)quinazoline was prepared following the procedures described for Example 13 using 2-amino-6-chlorobenzamide and 2-chlorobenzoyl chloride in Step 1 and pyrrolidine in Step 3. ¹H NMR (400 MHz, CDCl₃) δ 7.82 (m, 1H), 7.75 (m, 1H), 7.57 (m, 1H), 7.48 (m, 1H), 7.41 (m, 1H) 7.35 (m, 2H), 3.86 (m, 4H), 1.93 (m, 4H). MS (M/z, M+1), 344.3.

### EXAMPLE 268

### 1-(4-(2-(2-Chlorophenyl)-6-fluoroquinazolin-4-yl)piperazin-1-yl)ethanone

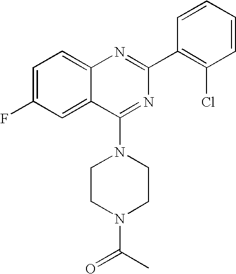

The compound 1-(4-(2-(2-chlorophenyl)-6-fluoroquinazolin-4-yl)piperazin-1-yl)ethanone was prepared following the procedures described for Example 13 using 1-amino-5-fluorobenzamide and 2-chlorobenzoyl chloride in Step 1 and 1-acetylpiperazine in Step 3. ¹H NMR (400 MHz, CDCl₃) δ 8.04 (m, 1H), 7.86 (m, 1H), 7.57 (m, 2H), 7.51 (m, 1H), 7.39 (m, 2H), 3.79 (m, 8H), 2.17 (s, 3H). MS (M/z, M+1), 385.4.

### EXAMPLE 269

### Ethyl 4-(2-(2-chlorophenyl)-6-fluoroquinazolin-4-yl)piperazine-1-carboxylate

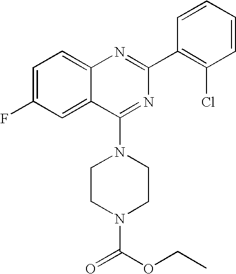

The compound ethyl 4-(2-(2-chlorophenyl)-6-fluoroquinazolin-4-yl)piperazine-1-carboxylate was prepared following the procedures described for Example 13 using 2-amino-5-fluorobenzamide and 2-chlorobenzoyl chloride in Step 1 and ethyl 1-piperazinecarboxylate in Step 3. ¹H NMR (400 MHz, CDCl₃) δ 8.02 (m, 1H), 7.85 (m, 1H), 7.56 (m, 2H), 7.50 (m, 1H), 7.39 (m, 2H), 4.19 (q, 2H), 3.78 (m, 4H), 7.73 (m, 4H), 1.30 (t, 3H). MS (M/z, M+1), 415.5.

### EXAMPLE 270

### 2-(2-Chlorophenyl)-6-fluoro-4-(pyrrolidin-1-yl)quinazoline

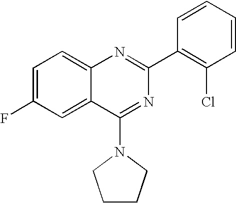

The compound 2-(2-chlorophenyl)-6-fluoro-4-(pyrrolidin-1-yl)quinazoline was prepared following the procedures described for Example 13 using 2-amino-5-fluorobenzamide and 2-chlorobenzoyl chloride in Step 1 and pyrrolidine in Step 3. ¹H NMR (400 MHz, CDCl₃) δ 7.88 (m, 2H), 7.81 (m, 1H), 7.48 (m, 2H), 7.35 (m, 2H), 4.00 (m, 4H), 2.07 (m, 4H). MS (M/z, M+1), 328.4.

### EXAMPLE 271

### 1-(4-(6-Chloro-2-(2-fluorophenyl)quinazolin-4-yl)piperazin-1-yl)ethanone

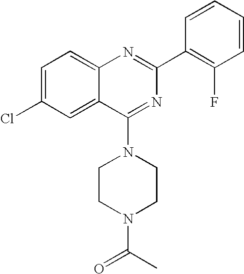

The compound 1-(4-(6-Chloro-2-(2-fluorophenyl)quinazolin-4-yl)piperazin-1-yl)ethanone was prepared following the procedures described for Example 13 using 2-amino-5-chlorobenzamide and 2-fluorobenozyl chloride in Step 1 and 1-acetylpiperazine in Step 3. ¹H NMR (400 MHz, CDCl₃) δ 8.13 (m, 1H), 7.95 (d, 1H), 7.87 (d, 1H), 7.71 (m, 1H), 7.44 (m, 1H), 7.26 (m, 1H), 7.19 (m, 1H), 3.83 (m, 6H), 3.73 (m, 2H), 2.17 (s, 3H). MS (M/z, M+1), 385.4.

### EXAMPLE 272

### Ethyl 4-(6-chloro-2-(2-fluorophenyl)quinazolin-4-yl)piperazine-1-carboxylate

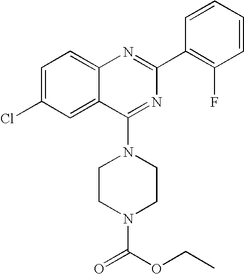

The compound ethyl 4-(6-chloro-2-(2-fluorophenyl)quinazolin-4-yl)piperazine-1-carboxylate was prepared following the procedures described for Example 13 using 2-amino-5-chlorobenzamide and 2-fluorobenozyl chloride in Step 1 and ethyl 1-piperazine carboxylate in Step 3. ¹H NMR (400 MHz, CDCl₃) δ 8.13 (m, 1H), 7.94 (d, 1H), 7.86 (d, 1H), 7.70 (m, 1H), 7.43 (m, 1H), 7.26 (m, 1H), 7.16 (m, 1H), 4.19 (q, 2H), 3.80 (m, 4H), 3.73 (m, 4H), 1.30 (t, 3H). MS (M/z, M+1), 415.5.

### EXAMPLE 273

### 6-Chloro-2-(2-fluorophenyl)-4-(pyrrolidin-1-yl)quinazoline

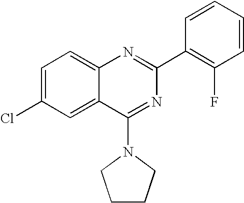

The compound 6-chloro-2-(2-fluorophenyl)-4-(pyrrolidin-1-yl)quinazoline was prepared following the procedures described for Example 13 using 2-amino-5-chlorobenzamide and 2-fluorobenzoyl chloride in Step 1 and pyrrolidine in Step 3. ¹H NMR (400 MHz, CDCl₃) δ 8.12 (m, 2H), 7.85 (d, 1H), 7.63 (m, 1H), 7.39 (m, 1H), 7.23 (m, 1H), 7.16 (m, 1H), 3.99 (m, 4H), 2.07 (m, 4H). MS (M/z, M+1), 328.4.

### EXAMPLE 274

### 1-(4-(2-(2-Fluorophenyl)-6-methoxyquinazolin-4-yl)piperazin-1-yl)ethanone

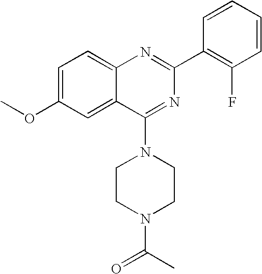

The compound 1-(4-(2-(2-fluorophenyl)-6-methoxyquinazolin-4-yl)piperazin-1-yl)ethanone was prepared following the procedures described for Example 13 using 2-amino-5-methoxybenzamide and 2-fluorobenozyl chloride in Step 1 and 1-acetylpiperazine in Step 3. ¹H NMR (400 MHz, CDCl₃) δ 8.12 (m, 1H), 7.96 (d, 1H), 7.45 (m, 1H), 7.41 (m, 1H), 7.25 (m, 1H), 7.18 (m, 2H), 3.94 (s, 3H), 3.81 (m, 8H), 2.17 (s, 3H). MS (M/z, M+1), 381.5.

### EXAMPLE 275

### Ethyl 4-(2-(2-fluorophenyl)-6-methoxyquinazolin-4-yl)piperazine-1-carboxylate

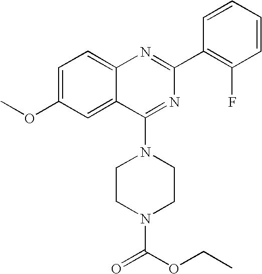

The compound ethyl 4-(2-(2-fluorophenyl)-6-methoxyquinazolin-4-yl)piperazine-1-carboxylate was prepared following the procedures described for Example 13 using 2-amino-5-methoxybenzamide and 2-fluorobenozyl chloride in Step 1 and ethyl 1-piperazine carboxylate in Step 3. ¹H NMR (400 MHz, CDCl₃) δ 8.12 (m, 1H), 7.95 (d, 1H), 7.44 (m, 1H), 7.41 (m, 1H), 7.24 (m, 1H), 7.18 (m, 2H), 4.19 (q, 2H), 3.94 (s, 3H), 3.74 (m, 8H), 1.30 (t, 3H). MS (M/z, M+1), 411.5.

### EXAMPLE 276

### 2-(2-Fluorophenyl)-6-methoxy-4-(pyrrolidin-1-yl)quinazoline

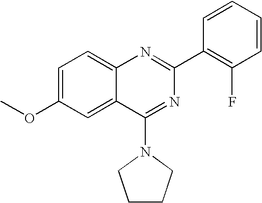

The compound 2-(2-fluorophenyl)-6-methoxy-4-(pyrrolidin-1-yl)quinazoline was prepared following the procedures described for Example 13 using 2-amino-5-methoxybenzamide and 2-fluorobenzoyl chloride in Step 1 and pyrrolidine in Step 3. ¹H NMR (400 MHz, CDCl₃) δ 8.11 (m, 1H), 7.86 (d, 1H), 7.55 (d, 1H), 7.37 (m, 2H), 7.21 (m, 1H), 7.15 (m, 1H), 4.01 (m, 4H), 3.99 (s, 3H), 2.06 (m, 4H). MS (M/z, M+1), 324.4.

### EXAMPLE 277

### 1-(4-(2-(4-tert-Butylphenyl)-6-chloroquinazolin-4-yl)piperazin-1-yl)ethanone

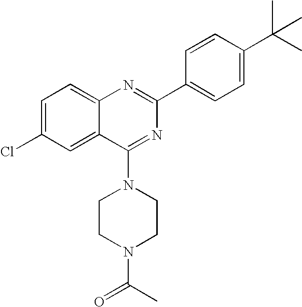

The compound 1-(4-(2-(4-tert-butylphenyl)-6-chloroquinazolin-4-yl)piperazin-1-yl)ethanone was prepared following the procedures described for Example 13 using 2-amino-5-chlorobenzamide and 4-tert-butylbenozyl chloride in Step 1 and 1-acetylpiperazine in Step 3. ¹H NMR (400 MHz, CDCl₃) δ 8.45 (d, 2H), 7.99 (d, 1H), 7.81 (d, 1H), 7.52 (d, 2H), 7.38 (m, 1H), 3.83 (m, 8H), 2.19 (s, 3H), 1.38 (s, 9H). MS (M/z, M+1), 423.5.

### EXAMPLE 278

### 2-(4-tert-Butylphenyl)-6-chloro-4-(pyrrolidin-1-yl)quinazoline

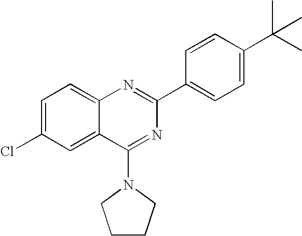

The compound 2-(4-tert-butylphenyl)-6-chloro-4-(pyrrolidin-1-yl)quinazoline was prepared following the procedures described for Example 13 using 2-amino-5-chlorobenzamide and 4-tert-butylbenzoyl chloride in Step 1 and pyrrolidine in Step 3. ¹H NMR (400 MHz, CDCl₃) δ 8.44 (d, 2H), 8.06 (d, 1H), 7.89 (d, 1H), 7.50 (s, 2H), 7.25 (m, 1H), 4.01 (m, 4H), 2.08 (m, 4H), 1.38 (s, 9H). MS (M/z, M+1), 366.5.

### EXAMPLE 279

### Ethyl 4-(2-(2-fluorophenyl)quinazolin-4-yl)piperazine-1-carboxylate

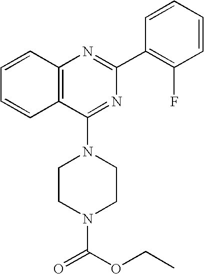

The compound ethyl 4-(2-(2-fluorophenyl)quinazolin-4-yl)piperazine-1-carboxylate was prepared following the procedures described for Example 13 using 2-fluorobenzoyl chloride in Step 1 and ethyl 1-piperazine carboxylate in Step 3. ¹H NMR (400 MHz, CDCl₃) δ 8.14 (m, 1H), 8.01 (d, 1H), 7.91 (d, 1H), 7.77 (m, 1H), 7.49 (m, 1H), 7.42 (m, 1H), 7.26 (m, 1H), 7.18 (m, 1H), 4.19 (q, 2H), 3.82 (m, 4H), 3.73 (m, 4H), 1.30 (t, 3H). MS (M/z, M+1), 381.1.

### EXAMPLE 280

### 1-(4-(5-Fluoro-2-(2-fluorophenyl)quinazolin-4-yl)piperazin-1-yl)ethanone

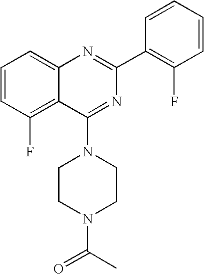

The compound 1-(4-(5-fluoro-2-(2-fluorophenyl)quinazolin-4-yl)piperazin-1-yl)ethanone was prepared following the procedures described for Example 13 using 2-amino-6-fluorobenzamide and 2-fluorobenzoyl chloride in Step 1 and 1-acetylpiperazine in Step 3. ¹H NMR (400 MHz, CDCl₃) δ 8.15 (m, 1H), 7.80 (m, 1H), 7.72 (m, 1H), 7.45 (m, 1H), 7.27 (m, 1H), 7.17 (m, 2H), 3.83 (m, 2H), 3.75 (m, 2H), 3.70 (m, 4H), 2.17 (s, 3H). MS (M/z, M+1), 369.3.

### EXAMPLE 281

### Ethyl 4-(5-fluoro-2-(2-fluorophenyl)quinazolin-4-yl)piperazine-1-carboxylate

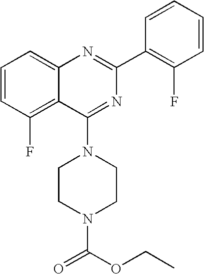

The compound ethyl 4-(5-fluoro-2-(2-fluorophenyl)quinazolin-4-yl)piperazine-1-carboxylate was prepared following the procedures described for Example 13 using 2-amino-6-fluorobenzamide and 2-fluorobenozyl chloride in Step 1 and ethyl 1-piperazine carboxylate in Step 3. ¹H NMR (400 MHz, CDCl₃) δ 8.15 (m, 1H), 7.79 (m, 1H), 7.71 (m, 1H), 7.44 (m, 1H), 7.26 (m, 1H), 7.16 (m, 2H), 4.19 (q, 2H), 3.70 (m, 8H), 1.28 (t, 3H). MS (m/z, M+1), 399.3.

### EXAMPLE 282

### 5-Fluoro-2-(2-fluorophenyl)-4-(pyrrolidin-1-yl)quinazoline

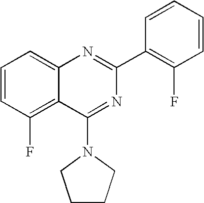

The compound 5-fluoro-2-(2-fluorophenyl)-4-(pyrrolidin-1-yl)quinazoline was prepared following the procedures described for Example 13 using 2-amino-6-fluorobenzamide and 2-fluorobenzoyl chloride in Step 1 and pyrrolidine in Step 3. ¹H NMR (400 MHz, CDCl₃) δ 8.12 (m, 1H), 7.64 (m, 2H), 7.41 (m, 1H), 7.24 (m, 1H), 7.17 (m, 1H), 7.05 (m, 1H), 3.82 (m, 4H), 1.97 (m, 4H). MS (M/z, M+1), 312.2.

### EXAMPLE 283

### 1-(4-(5-Chloro-2-(2-chlorophenyl)quinazolin-4-yl)piperazin-1-yl)-3-methylbutan-1-one

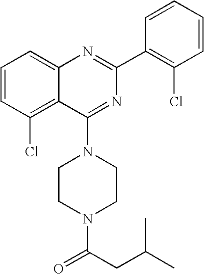

The compound 1-(4-(5-chloro-2-(2-chlorophenyl)quinazolin-4-yl)piperazin-1-yl)-3-methylbutan-1-one was prepared following the procedures described for Example 83 using isovaleryl chloride. ¹H NMR (400 MHz, CDCl₃) δ 7.90 (m, 2H), 7.66 (m, 1H), 7.52 (m, 2H), 7.39 (m, 2H), 3.69 (m, 8H), 2.26 (d, 2H), 2.14 (m, 1H), 0.99 (d, 6H). MS (M/z, M+1), 443.3.

### EXAMPLE 284

### 4-(5-Chloro-2-(2-chlorophenyl)quinazolin-4-yl)-N-propylpiperazine-1-carboxamide

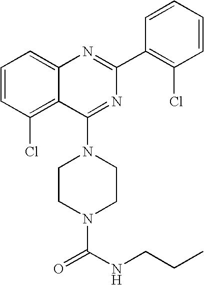

The compound 4-(5-chloro-2-(2-chlorophenyl)quinazolin-4-yl)-N-propylpiperazine-1-carboxamide was prepared following the procedures described for Example 87 using propylisocyanate. ¹H NMR (400 MHz, CDCl₃) δ 7.88 (m, 2H), 7.64 (m, 1H), 7.50 (m, 2H), 7.38 (m, 2H), 4.54 (m, 1H), 3.84 (m, 2H), 3.56 (m, 6H), 3.23 (m, 2H), 1.55 (m, 2H), 0.94 (t, 3H). MS (M/z, M+1), 444.3.

### EXAMPLE 285

### 5-Chloro-2-(2-chlorophenyl)-4-(4-(propylsulfonyl)piperazin-1-yl)quinazoline

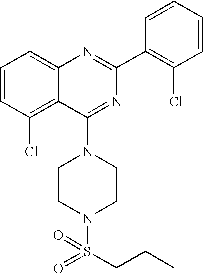

The compound 5-chloro-2-(2-chlorophenyl)-4-(4-(propylsulfonyl)piperazin-1-yl)quinazoline was prepared following the procedures described for Example 83 using propylsulfonyl chloride. ¹H NMR (400 MHz, CDCl₃) δ 7.89 (m, 2H), 7.67 (m, 1H), 7.53 (m, 2H), 7.40 (m, 2H), 3.88 (m, 2H), 3.70 (m, 2H), 3.48 (m, 4H), 2.91 (m, 2H), 1.87 (m, 2H), 1.07 (t, 3H). MS (M/z, M+1), 465.3.

### EXAMPLE 286

### Ethyl 4-(7-tert-butyl-2-(phenylamino)quinazolin-4-yl)piperazine-1-carboxylate

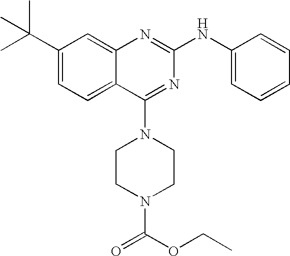

The compound ethyl 4-(7-tert-butyl-2-(phenylamino)quinazolin-4-yl)piperazine-1-carboxylate was synthesized according to Scheme VI.

### Step 1

1-tert-Butyl-4-methylbenzene (50 g, 337.8 mmol) was dissolved in H₂SO₄ (85%, 125 g). Then 98% HNO₃ and 80% H₂SO₄ (1:1.5, 58 g) at 10° C. The mixture was warmed to 60° C. and followed to completion by TLC. The mixture was extracted with EtOAc and the organic phase then washed with NaHCO₃, dried with Na₂SO₄ and concentrated in vacuo to give 4-tert-butyl-1-methyl-2-nitrobenzene.

### Step 2

The crude compound obtained from Step 1 was dissolved in Pyridine:H₂O (1:1 v/v, 500 mL). Then KMnO₄ (570 g) in portions. The mixture was heated to 110° C. and the reaction was followed to completion by TLC. The mixture was filtered and NaOH (1 N) was added to the filtrate to bring the pH to 13. The mixture was extracted with EtOAc. To the aqueous layer was added HCl (1 N) until pH=3 and then extracted with EtOAc. The organic layers were combined, dried with Na₂SO₄, and concentrated in vacuo to give 4-tert-butyl-2-nitrobenzoic acid.

### Step 3

4-tert-Butyl-2-nitrobenzoic acid (60 g, 269.1 mmol) was dissolved in MeOH (200 mL) and Pd/C (6 g) was added. The flask was fitted with a balloon of H₂ gas. The mixture was stirred overnight at room temperature then filtered through Celite and the filtrate concentrated in vacuo to give 2-amino-4-tert-butylbenzoic acid.

### Step 4

2-Amino-4-tert-butylbenzoic acid (6 g, 31.1 mmol) was dissolved in acetic acid (10 mL). Then H₂O (200 mL) was added followed by a solution of KOCN (5 g) in H₂O (50 mL) added dropwise. NaOH (1 N) was added to bring the pH to 13 and the mixture was heated to 90° C. and followed by TLC. After cooling to room temperature HCl (1 N) was added until pH=6. The mixture was filtered and the filter cake washed with Et₂O. The solid was oven dried under reduced pressure to give 7-tert-butylquinazoline-2,4-diol.

### Step 5

POCl₃ (215 g) was added to 7-tert-butylquinazoline-2,4-diol (9.5 g, 43.4 mmol) and the mixture was heated to 110° C. and the reaction followed to completion by TLC. The solvent was removed in vacuo and then CH₂Cl₂ was added. The mixture was washed with NaHCO₃, dried over Na₂SO₄, filtered, and concentrated in vacuo. The crude material was purified on silica gel (0-30% EtOAc/hexanes) to give 7-tert-butyl-2,4-dichloroquinazoline. MS (M/z, M+1), 255.0.

### Step 6

The compound ethyl 4-(7-tert-butyl-2-chloroquinazolin-4-yl)piperazine-1-carboxylate was prepared following the procedures described in Step 3 for the preparation of Example 13 using 7-tert-butyl-2,4-dichloroquinazoline and ethyl piperazine-1-carboxylate. ¹H NMR (400 MHz, DMSO-d₆) δ 7.98 (d, 1H), 7.63-7.60 (m, 2H), 4.07 (q, 2H), 3.85-3.80 (m, 4H), 3.60-3.55 (m, 4H), 1.34 (s, 9H), 1.19 (t, 3H). MS (M/z, M+1), 377.6.

### Step 7

The compounds ethyl 4-(7-tert-butyl-2-chloroquinazolin-4-yl)piperazine-1-carboxylate (50 mg) and aniline (15 μL) were dissolved in NMP (1.5 mL) and heated to 200° C. in a microwave reactor for 10 minutes. The crude solution was then poured into water and extracted with CH₂Cl₂. The organic layers were combined, dried over Na₂SO₄, filtered, dry loaded on silica gel and purified by column chromatography (SiO₂) to give the title compound. ¹H NMR (400 MHz, CDCl₃) δ 7.73 (d, 2H), 7.68 (d, 1H), 7.61 (d, 1H), 7.35 (t, 2H), 7.28-7.26 (m, 1H), 7.02 (t, 1H), 6.97 (s, 1H), 4.20 (q, 2H), 3.72-3.67 (m, 8H), 1.39 (s, 9H), 1.30 (t, 3H). MS (M/z, M+1), 434.7.

### EXAMPLE 287

### 7-tert-Butyl-N-phenyl-4-(pyrrolidin-1-yl)quinazolin-2-amine

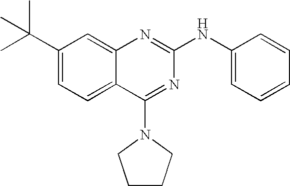

The compound 7-tert-butyl-N-phenyl-4-(pyrrolidin-1-yl)quinazolin-2-amine was prepared following the procedures described for the preparation of Example 286 using 7-tert-butyl-2-chloro-4-(pyrrolidin-1-yl)quinazoline. ¹H NMR (400 MHz, CDCl₃) δ 10.74 (s, 1H), 7.97 (d, 1H), 7.71-7.68 (m, 2H), 7.50 (d, 1H), 7.35-7.31 (m, 3H), 7.11 (t, 1H), 4.18-3.90 (m, 4H), 2.18-2.08 (m, 4H), 1.35 (s, 9H). MS (M/z, M+1), 347.7.

### EXAMPLE 288

### 7-tert-Butyl-N-(2,4-difluorophenyl)-4-(pyrrolidin-1-yl)quinazolin-2-amine

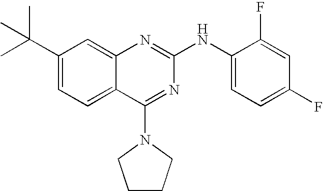

The compound 7-tert-butyl-N-(2,4-difluorophenyl)-4-(pyrrolidin-1-yl)quinazolin-2-amine was prepared following the procedures described for the preparation of Example 286 using 7-tert-butyl-2-chloro-4-(pyrrolidin-1-yl)quinazoline and 2,4-difluoroaniline. ¹H NMR (400 MHz, CDCl₃) δ 8.71-8.65 (m, 1H), 7.99 (d, 1H), 7.54 (d, 1H), 7.21 (dd, 1H), 6.92-6.82 (m, 3H), 3.94-3.90 (m, 4H), 2.06-2.03 (m, 4H), 1.38 (s, 9H). MS (M/z, M+1), 383.3.

### EXAMPLE 289

### 7-tert-Butyl-N-(3,4-difluorophenyl)-4-(pyrrolidin-1-yl)quinazolin-2-amine

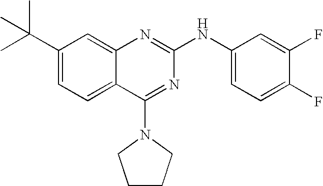

The compound 7-tert-butyl-N-(3,4-difluorophenyl)-4-(pyrrolidine-1-yl)quinazolin-2-amine was prepared following the procedures described for the preparation of Example 286 using 7-tert-butyl-2-chloro-4-(pyrrolidin-1-yl)quinazoline and 3,4-difluoroaniline. ¹H NMR (400 MHz, CDCl₃) δ 8.05-8.01 (m, 1H), 7.99 (d, 1H), 7.53 (d, 1H), 7.21 (dd, 1H), 7.10-7.02 (m, 2H), 6.85 (s, 1H), 3.95-3.92 (m, 4H), 2.07-2.04 (m, 4H), 1.38 (s, 9H). MS (M/z, M+1), 383.3.

### EXAMPLE 290

### 7-tert-Butyl-N-(3-chlorophenyl)-4-(pyrrolidin-1-yl)quinazolin-2-amine

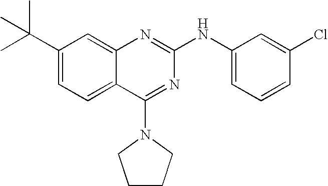

The compound 7-tert-Butyl-N-(3-chlorophenyl)-4-(pyrrolidin-1-yl)quinazolin-2-amine was prepared following the procedures described for the preparation of Example 286 using 7-tert-butyl-2-chloro-4-(pyrrolidin-1-yl)quinazoline and 3-chloroaniline. ¹H NMR (400 MHz, CDCl₃) δ 10.99 (s, 1H), 8.00-7.98 (m, 2H), 7.50 (d, 1H), 7.48-7.45 (m, 1H), 7.37 (dd, 1H), 7.25 (t, 1H), 7.08-7.06 (m, 1H), 4.05 (br s, 4H), 2.16 (br s, 4H), 1.36 (s, 9H). MS (M/z, M+1), 381.2.

### EXAMPLE 291

### 2-Fluoro-N-((4-morpholinoquinazolin-2-yl)methyl)benzenesulfonamide

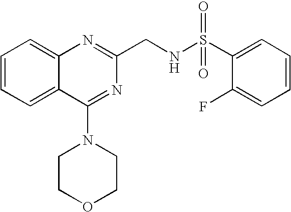

The compound 2-fluoro-N-((4-morpholinoquinazolin-2-yl)methyl)benzenesulfonamide was synthesized according to Scheme I. (4-Morpholinoquinazolin-2-yl)methanamine (60.0 mg, 0.25 mmol) and TEA (85.58 μL, 0.61 mmol) were dissolved in DCM (3.0 mL). The solution was purged with nitrogen. 2-Fluorobenzene-1-sulfonyl chloride (52.58 mg, 0.27 mmol) was added. The solution was stirred overnight. The solution was neutralized with 2 M sodium carbonate. The compound was extracted with ethyl acetate and brine. The organic layer was then concentrated onto silica gel and purified by flash column chromatography (0-100% EtOAc/hexane) to give 0.006 g of the title compound. MS (M/z, M+1), 464.48.

### EXAMPLE 292

### 2-Bromo-N-((4-morpholinoquinazolin-2-yl)methyl)benzenesulfonamide

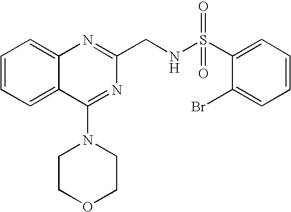

The compound 2-bromo-N-((4-morpholinoquinazolin-2-yl)methyl)benzenesulfonamide was prepared following the procedure described for Example 291 using 2-bromobenzene-1-sulfonyl chloride. ¹H NMR (400 MHz, DMSO-d₆) δ 8.11 (t, 1H), 7.87 (m, 2H), 7.74 (m, 1H), 7.63-7.61 (m, 2H), 7.45 (m, 1H), 7.28 (m, 2H), 4.25 (d, 2H), 3.73 (t, 4H), 3.63 (t, 4H). MS (M/z, M+1), 464.36.

### EXAMPLE 293

### 4-Chloro-N-((4-morpholinoquinazolin-2-yl)methyl)benzenesulfonamide

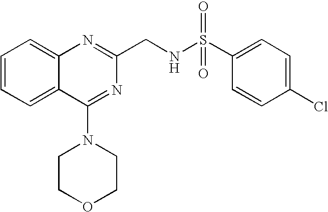

The compound 4-chloro-N-((4-morpholinoquinazolin-2-yl)methyl)benzenesulfonamide was prepared following the procedure described for Example 291 using 4-chlorobenzene-1-sulfonyl chloride. ¹H NMR (400 MHz, DMSO-d₆) δ 8.22 (s, 1H), 7.84 (d, 1H), 7.71 (m, 1H), 7.57 (m, 3H), 7.41 (m, 1H), 7.27 (m, 2H), 4.13 (s, 2H), 3.67 (t, 4H), 3.58 (t, 4H). MS (M/z, M+1), 420.23.

### EXAMPLE 294

### 2,4-Dichloro-N-((4-morpholinoquinazolin-2-yl)methyl)benzenesulfonamide

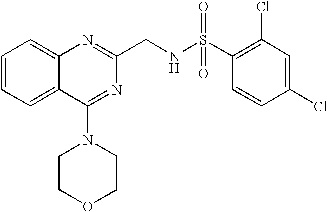

The compound 2,4-dichloro-N-((4-morpholinoquinazolin-2-yl)methyl)benzenesulfonamide was prepared following the procedure described for Example 291 using 2,4-dichlorobenzene-1-sulfonyl chloride. ¹H NMR (400 MHz, DMSO-d₆) δ 8.42 (t, 1H), 7.87 (d, 1H), 7.74 (m, 1H), 7.70 (d, 1H), 7.55 (d, 1H), 7.53 (d, 1H), 7.44 (m, 1H), 7.18 (dd, 1H), 4.25 (d, 2H), 3.74 (t, 4H), 3.64 (t, 4H). MS (M/z, M+1), 454.77.

### EXAMPLE 295

### 3-Chloro-2-methyl-N-((4-morpholinoquinazolin-2-yl)methyl)benzenesulfonamide

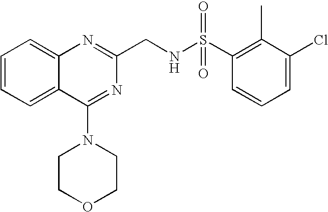

The compound 3-chloro-2-methyl-N-((4-morpholinoquinazolin-2-yl)methyl)benzenesulfonamide was prepared following the procedure described for Example 291 using 3-chloro-2-methylbenzene-1-sulfonyl chloride. ¹H NMR (400 MHz, DMSO-d₆) δ 8.53 (t, 1H), 7.94 (d, 1H), 7.78 (m, 1H), 7.69 (m, 1H), 7.48 (m, 1H), 7.19 (d, 2H), 6.99 (d, 2H). 4.24 (d, 2H), 3.75 (t, 2H), 3.68 (t, 2H), 2.34 (s, 3H). MS (M/z, M+1), 433.84.

### EXAMPLE 296

### 2-(4-tert-Butylphenoxy)-6,7-dimethoxy-4-(pyrrolidin-1-yl)quinazoline

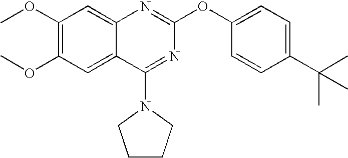

The compound 2-(4-tert-butylphenoxy)-6,7-dimethoxy-4-(pyrrolidin-1-yl)quinazoline was synthesized according to the scheme outlined in Scheme VI.

### Step 1

### 2,4-Dichloro-6,7-dimethoxyquinazoline (750.0 mg, 2.90 mmol) was dissolved in DMSO (5.0 mL). Pyrrolidine (0.32 mL, 2.90 mmol) and DIEA (0.67 mL, 2.90 mmol) were added to the solution. The mixture was heated to 80° C. for an hour.

The compound was extracted with ethyl acetate and brine. The compound was then collected by filtration and dried in vacuo to give 0.664 g of 2-chloro-6,7-dimethoxy-4-(pyrrolidin-1-yl)quinazoline.

### Step 2

2-Chloro-6,7-dimethoxy-4-(pyrrolidin-1-yl)quinazoline (75.0 mg, 0.25 mmol) was dissolved in DMSO (3.0 mL). 4-tert-butylphenol (45.87 mg, 0.31 mmol) and sodium hydride (60% mineral oil suspension, 20.4 mg, 0.51 mmol) were added to the solution. The mixture was heated to 100° C. until completion as determined by LC/MS. The compound was extracted with ethyl acetate and brine. The organic layer was then concentrated onto silica gel and purified by flash column chromatography (0-100% EtOAc/hexane) to give 0.012 g of the title compound. ¹H NMR (400 MHz, DMSO-d₆) δ 7.52 (s, 1H), 7.38 (d, 2H), 7.06 (d, 2H), 6.80 (s, 1H), 3.85 (m, 10H), 1.94 (s, 4H), 1.30 (s, 9H). MS (M/z, M+1), 408.39.

### EXAMPLE 297

### 6,7-Dimethoxy-2-(4-methoxyphenoxy)-4-(pyrrolidin-1-yl)quinazoline

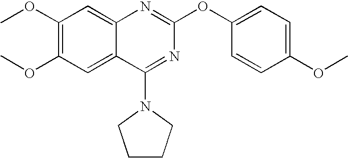

The compound 6,7-dimethoxy-2-(4-methoxyphenoxy)-4-(pyrrolidin-1-yl)quinazoline was prepared by following the procedure described for Example 296 using 4-methoxyphenol. ¹H NMR (400 MHz, DMSO-d₆) δ 7.51 (s, 1H), 7.06 (d, 2H), 6.92 (d, 2H), 6.79 (s, 1H), 3.82 (m, 13H), 1.93 (t, 4H). MS (M/z, M+1), 382.20.

### EXAMPLE 298

### N-(4-tert-butylphenyl)-6,7-dimethoxy-4-(pyrrolidin-1-yl)quinazolin-2-amine

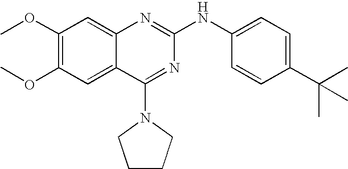

The compound N-(4-tert-butylphenyl)-6,7-dimethoxy-4-(pyrrolidin-1-yl)quinazolin-2-amine was synthesized according to Scheme VI.

### Step 1

2,4-Dichloro-6,7-dimethoxyquinazoline (750.0 mg, 2.90 mmol) was dissolved in DMSO (5.0 mL). Pyrrolidine (0.32 mL, 2.90 mmol) and DIEA (0.67 mL, 2.90 mmol) were added to the solution. The mixture was heated to 80° C. for an hour.

The compound was extracted with ethyl acetate and brine. The compound was then collected by filtration and dried in vacuo to give 0.664 g of 2-chloro-6,7-dimethoxy-4-(pyrrolidin-1-yl)quinazoline.

### Step 2

2-Chloro-6,7-dimethoxy-4-(pyrrolidin-1-yl)quinazoline (75.0 mg, 0.25 mmol) was dissolved in ethanol (1.0 mL). 4-tert-Butylaniline (48.0 μL, 0.31 mmol) was added to the solution. The mixture was heated to 120° C. until completion as determined by LC/MS. The compound was extracted with ethyl acetate and brine. The organic layer was then concentrated onto silica gel and purified by flash column chromatography (0-100% EtOAc/hexane) to give 0.034 g of the title compound. ¹H NMR (400 MHz, DMSO-d₆) δ 8.67 (s, 1H), 7.77 (d, 2H), 7.43 (s, 1H), 7.23 (s, 2H), 6.82 (s, 1H), 3.83 (m, 10H), 1.95 (s, 4H), 1.25 (s, 9H). MS (M/z, M+1), 407.19.

### EXAMPLE 299

### N-((4-(Pyrrolidin-1-yl)quinazolin-2-yl)methyl)naphthalene-2-sulfonamide

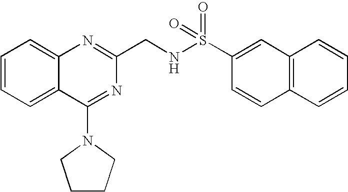

A solution of (4-(pyrrolidin-1-yl)quinazolin-2-yl)methanamine (70 mg) 2-naphthalenesulfonyl chloride (70 mg) and pyridine (0.1 mL) in dry THF (4 mL) was stirred at room temperature for 1 h and at 60° C. for 0.5 h. The solvent was removed in vacuo and the residue was partitioned between water and ethyl acetate. The crude material was purified by column chromatography to give the title compound. ¹H NMR (400 MHz, (DMSO-d₆) δ 8.22 (d, 1H,), 8.02 (s, 1H), 7.90 (d, 1H), 7.80-7.86 (m, 3H), 7.72 (m, 1H), 7.55-7.62 (m, 2H), 7.40-7.52 (m, 2H), 7.27 (m, 1H), 4.10 (s, 2H), 3.53 (m, 4H), 1.8 (m, 4H). MS (M/z, M+1), 419.22.

### EXAMPLE 300

### N-((4-Morpholinoquinazolin-2-yl)methyl)naphthalene-1-sulfonamide

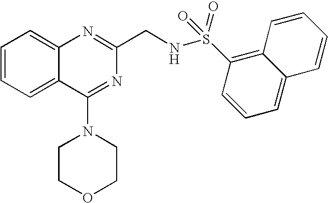

This compound was prepared following the procedures described for Example 299. ¹H NMR (400 MHz, (DMSO-d₆) δ 8.20 (m, 2H), 7.87 (m, 2H), 7.81 (d, 1H), 7.62-7.70 (m, 3H), 7.56-7.62 (m, 2H), 7.51-7.53 (m, 1H), 7.30-7.36 (m, 1H), 4.20 (d, 2H), 3.59 (m, 4H), 3.46 (m, 4H). MS (M/z, M+1), 435.22).

### EXAMPLE 301

### N-((4-Morpholinoquinazolin-2-yl)methyl)naphthalene-2-sulfonamide

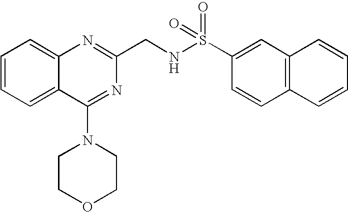

The compound N-((4-morpholinoquinazolin-2-yl)methyl)naphthalene-2-sulfonamide was prepared following the procedures described for Example 299 using 2-napthalenesulfonyl chloride. ¹H NMR (400 MHz, (DMSO-d₆) δ 8.64 (d, 1H), 8.42 (t, 1H), 7.95 (m, 1H), 7.47 (m, 2H), 7.68 (d, 1H), 7.53-7.67 (m, 3H), 7.40 (m, 2H), 7.26 (m, 1H), 4.20 (d, 2H), 3.60 (m, 4H), 3.34 (m, 4H). MS (M/z, M+1), 435.25.

### EXAMPLE 302

### N-((4-(Phenylamino)quinazolin-2-yl)methyl)naphthalene-1-sulfonamide

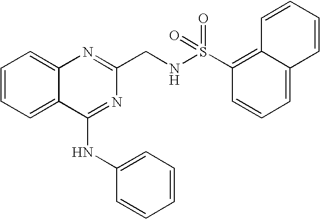

### Step 1

A solution of 4-chloro-2-(chloromethyl)quinazoline (443 mg), aniline (196 mg) and diisopropylethylamine (0.36 mL) in DMSO (4 mL) was stirred at room temperature overnight. The solution was partitioned between water and ethyl acetate. The crude material was purified by column chromatography to give 2-(chloromethyl)-N-phenylquinazoline-4-amine. MS (M/z, M+1), 270.12.

### Step 2

A mixture of 2-(chloromethyl)-N-phenylquinazolin-4-amine (250 mg) and sodium azide (90 mg) in dry DMSO (3 mL) was heated at 60° C. for 1 h. Water was added and the precipitate was collected by filtration to give (2-(azidomethyl)-N-phenylquinazolin-4-amine. MS (M/z, M+1-N2), 249.16. This crude material was hydrogenated with palladium on carbon as catalyst in methanol at room temperature for 20 h. 2-(aminomethyl)-N-phenylquinazolin-4-amine was obtained by column chromatography. MS (M/z, M+1), 251.17.

### Step 3

A solution of 2-(aminomethyl)-N-phenylquinazolin-4-amine (78 mg), 1-naphthalenesulfonyl chloride (71 mg), and pyridine (0.1 mL) in dry THF (6 mL) was stirred at room temperature for 1 h and then at 55° C. for 1 h. The solvent was removed in vacuo and the residue was partitioned between water and ethyl acetate. The crude material was purified by column chromatography to give the title compound. ¹H NMR (400 MHz, DMSO-d₆) δ 9.60 (s, 1H), 8.68 (d, 1H), 8.40 (m, 2H), 8.08 (d, 1H), 7.98 (d, 1H), 7.92 (d, 1H), 7.90 (d, 1H), 8.84 (d, 2H), 7.48-7.74 (m, 4H), 7.31-7.40 (m, 3H), 7.09 (t, 1H), 4.17 (d, 2H). MS (M/z, M+1), 441.24.

### EXAMPLE 303

### N-Phenyl-2-((phenylamino)methyl)quinazolin-4-amine

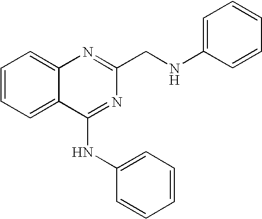

A solution of 4-chloro-2-(chloromethyl)quinazoline (0.440 g), aniline (190 mg), and diisopropylethylamine (0.36 mL) in DMSO (4 ml) was stirred at room temperature overnight. The solution was partitioned between water and ethyl acetate. The crude material was purified by column chromatography to give the title compound. MS (M/z, M+1), 327.21.

### EXAMPLE 304

### N-((4-(Isopropylamino)quinazolin-2-yl)methyl)naphthalene-1-sulfonamide

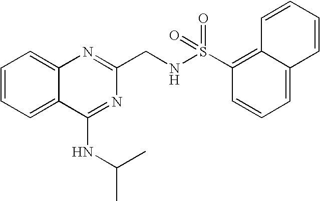

### Step 1

A solution of 4-chloro-2-(chloromethyl)quinazoline (0.41 g), isopropylamine (116 mg), and diisopropylethylamine in DMSO (4 mL) was stirred at room temperature for 1 h. The solution was partitioned between water and ethyl acetate. The crude material was purified by column chromatography to give 2-(chloromethyl)-N-isopropylquinazoline-4-amine. MS (M/z, M+1), 236.12.

### Step 2

A mixture of 2-(chloromethyl)-N-isopropylquinazolin-4-amine (256 mg) and sodium azide (100 mg) in dry DMSO (3 mL) was heated at 60° C. for 1 h. Water was added and the precipitate was collected by filtration to give (2-(azidomethyl)-N-isopropyl-quinazolin-4-amine. The crude compound was hydrogenated with palladium on carbon in methanol at room temperature for 20 h. 2-(Aminomethyl)-N-isopropylquinazoline was obtained by column chromatography. MS (M/z, M+1), 217.16.

### Step 3

A solution of 2-(aminomethyl)-N-isopropylquinazolin-4-amine (172 mg), 1-naphthalenesulfonyl chloride (180 mg), and pyridine (0.18 mL) in dry THF (6 mL) was stirred at room temperature for 30 min and 55° C. for 2 h. The solvent was removed in vacuo and the residue was partitioned between water and ethyl acetate. The crude material was purified by column chromatography to give the title compound. ¹H NMR (400 MHz, DMSO-d₆) δ 8.70 (d, 1H), 8.22 (t, 1H), 8.11 (m, 2H), 8.02 (d, 1H), 7.94 (d, 1H), 7.71 (d, 1H), 7.63-7.68 (m, 1H), 7.55-7.62 (m, 2H), 7.42 (t, 1H), 7.35-7.38 (m, 1H), 7.25 (m, 1H), 4.20 (m, 1H), 4.08 (d, 2H). 1.10 (d, 6H). MS (M/z, M+1), 407.24.

### EXAMPLE 305

### N-((4-(Cyclopentylamino)quinazolin-2-yl)methyl)naphthalene-1-sulfonamide

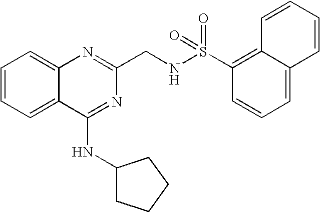

The compound N-((4-(cyclopentylamino)quinazolin-2-yl)methyl)naphthalene-1-sulfonamide was prepared following the procedures outline for Example 299. MS (M/z, M+1), 433.28.

### EXAMPLE 306

### 2-((1H-Benzo[d]imidazol-2-ylamino)methyl)-N-cyclopentylquinazolin-4-amine

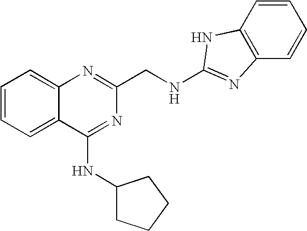

A solution of 2-(aminomethyl)-N-cyclopentylquinazolin-4-amine hydrochloride (33 mg), 2-chlorobenzimidazole (26 mg), and diisopropylethylamine (0.2 mL) in ethoxyethanol (1 mL) was stirred in a sealed tube at 200° C. for 1 h. The solvent was removed and the residue was partitioned between water and ethyl acetate. The crude material was purified by reverse phase HPLC to give the title compound. MS (M/z, M+1), 359.24.

### EXAMPLE 307

### N-((6,7-Dimethoxy-4-(pyrrolidin-1-yl)quinazolin-2-yl)methyl)naphthalene-1-sulfonamide

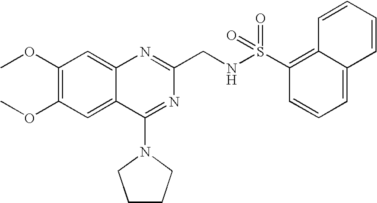

The compound N-((6,7-Dimethoxy-4-(pyrrolidin-1-yl)quinazolin-2-yl)methyl)naphthalene-1-sulfonamide was prepared following the procedures described for Example 299. MS (M/z, M+1), 479.29.

### EXAMPLE 308

### N-((4-(isoindolin-2-yl)quinazolin-2-yl)methyl)naphthalene-1-sulfonamide

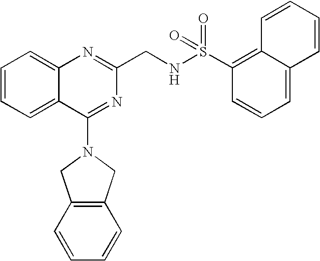

The compound N-((4-(isoindolin-2-yl)quinazolin-2-yl)methyl)naphthalene-1-sulfonamide was prepared following the procedures described for Example 299 using isoindoline in Step 1 and 1-naphthalenesulfonyl chloride in Step 3. MS (M/z, M+1), 467.27.

### EXAMPLE 309

### N-((7-Chloro-4-(pyrrolidin-1-yl)quinazolin-2-yl)methyl)naphthalene-1-sulfonamide

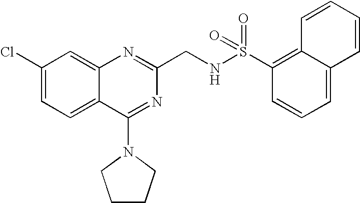

The compound N-((7-chloro-4-(pyrrolidin-1-yl)quinazolin-2-yl)methyl)naphthalene-1-sulfonamide was prepared following the procedures described for Example 299. MS (M/z, M+1), 453.24.

### EXAMPLE 310

### Methyl 2-(2-chlorophenyl)-4-(4-methylpiperazin-1-yl)quinazoline-6-carboxylate

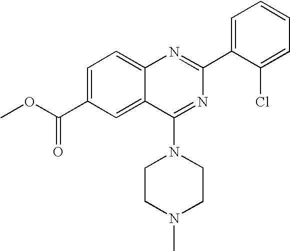

The compound methyl 2-(2-chlorophenyl)-4-(4-methylpiperazin-1-yl)quinazoline-6-carboxylate was prepared following the procedure described for Example 13 using 1-methylpiperazine in Step 3. ¹H NMR (400 MHz, DMSO-d₆) δ 8.66 (d, 1H), 8.28 (m, 1H), 7.93 (d, 1H), 7.86 (m, 1H), 7.45 (m, 1H), 7.31-7.36 (m, 2H), 3.97 (m, 7H), 2.61 (t, 4H), 2.35 (s, 3H). MS (M/z, M+1), 397.53.

### EXAMPLE 311

### 2-(2-Chlorophenyl)-N,N-diethyl-4-(4-methylpiperazin-1-yl)quinazoline-6-carboxamide

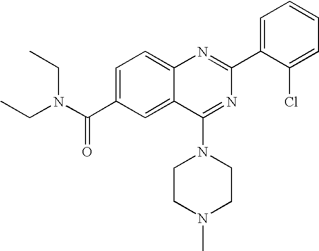

### Step 1

A solution of methyl 2-(2-chlorophenyl)-4-(4-methylpiperazin-1-yl)quinazoline-6-carboxylate (349 mg) and LiOH (1 N, 1.6 mL) in methanol (3 mL) was stirred at room temperature for 2 h. The solvent was removed in vacuo and the residue was neutralized with hydrochloric acid and partitioned between water and ethyl acetate. The crude material was purified by column chromatography to give 2-(2-chlorophenyl)-4-(4-methylpiperazin-1-yl)quinazoline-6-carboxylic acid. MS (M/z, M+1), 383.51.

### Step 2

A mixture of 2-(2-chlorophenyl)-4-(4-methylpiperazin-1-yl)quinazoline-6-carboxylic acid (80 mg), diethylamine (40 μL), N-(3-dimethylamino)-N′-ethylcarbodiimide hydrochloride (60 mg), and 1-hydroxybenzotriazole (28 mg) in dimethylformamide (1 mL) was stirred at room temperature for 20 h. The solution was partitioned between water and ethyl acetate. The crude material was purified by column chromatography to give the title compound. ¹H NMR (400 MHz, DMSO-d₆) δ 8.03 (d, 1H), 7.95 (d, 1H), 7.86-7.91 (m, 2H), 7.60 (m, 1H), 7.40-7.55 (m, 2H), 7.31-7.36 (m, 2H), 4.50 (m, 2H), 3.20-3.70 (m, 10H), 2.86 (s, 3H), 1.10 (m, 6H). MS (M/z, M+1), 438.62.

### EXAMPLE 312

### 2-(2-Chlorophenyl)-N-cyclohexyl-4-(4-methylpiperidin-1-yl)quinazoline-6-carboxamide

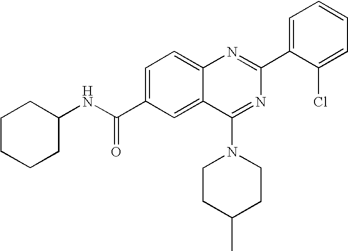

The compound 2-(2-chlorophenyl)-N-cyclohexyl-4-(4-methylpiperidin-1-yl)quinazoline-6-carboxamide was prepared following the procedures described for Example 311 using 4-methylpiperidine and cyclohexylamine. ¹H NMR (400 MHz, DMSO-d₆) δ 8.43 (m, 2H), 8.20 (m, 1H), 7.79-7.84 (m, 2H), 7.52-7.55 (m, 1H), 7.40-7.48 (m, 2H), 4.40 (d, 2H), 3.80 (m, 1H), 3.22 (t, 2H), 1.1-1.9 (m, 15H), 0.95 (d, 3H). MS (M/z, M+1), 463.64.

### EXAMPLE 313

### 2-(2-Chlorophenyl)-N-(4-methoxybenzyl)-4-(4-methylpiperidin-1-yl)quinazoline-6-carboxamide

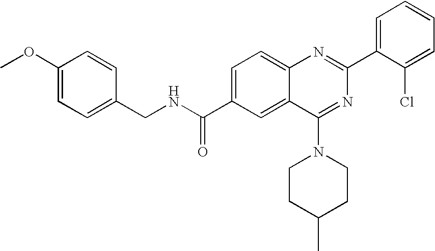

This compound was prepared according to the procedure for Example 311. ¹H NMR (400 MHz, DMSO-d₆) δ 9.22 (m, 1H), 8.49 (s, 1H), 8.22 (d, 1H), 7.80 (m, 2H), 7.40-7.55 (m, 3H), 7.27 (d, 2H), 6.88 (d, 2H), 4.45 (m, 4H), 3.71 (s, 3H), 3.21 (t, 2H), 1.76 (m, 3H), 1.18 (m, 2H), 0.95 (d, 3H). MS (M/z, M+1), 501.68.

### EXAMPLE 314

### Ethyl 4-(2-(2-chlorophenyl)-6-(diethylcarbamoyl)quinazolin-4-yl)piperazine-1-carboxylate

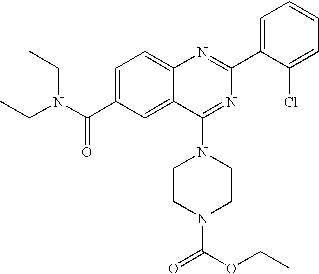

This compound was prepared according to the procedure for Example 311. ¹H NMR (400 MHz, CD₃OD) δ 8.27 (d, 1H), 8.05 (m, 1H), 7.92 (d, 1H), 7.85 (m, 1H), 7.66-7.70 (m, 2H), 7.50-7.61 (m, 1H), 4.38 (m, 4H), 4.17 (q, 2H), 3.40-3.58 (m, 8H), 1.19-1.31 (m, 9H). MS (M/z, M+1), 496.68.

### EXAMPLE 315

### 2-(2-Chlorophenyl)-N,N-diethyl-4-morpholinoquinazoline-6-carboxamide

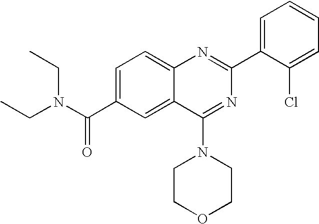

This compound was prepared according to the procedure for Example 311. ¹H NMR (400 MHz, CD₃OD) δ 8.24 (d, 1H), 8.04 (m, 1H), 7.90 (d, 1H), 7.83 (m, 1H), 7.66-7.69 (m, 2H), 7.50-7.61 (m, 1H), 4.38 (t, 4H), 3.89 (t, 4H), 3.34-3.62 (m, 4H), 1.18-1.35 (m, 6H). MS (M/z, M+1), 425.57.

### EXAMPLE 316

### 4-(4-Acetylpiperazin-1-yl)-2-(2-chlorophenyl)-N,N-diethylquinazoline-6-carboxamide

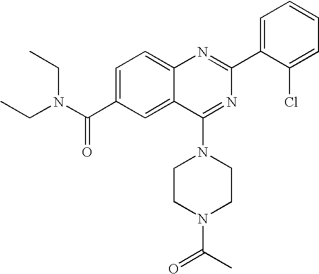

This compound was prepared according to the procedure for Example 311. ¹H NMR (400 MHz, CD₃OD) δ 8.28 (d, 1H), 8.06 (m, 1H), 7.92 (d, 1H), 7.85 (m, 1H), 7.66-7.69 (m, 2H), 7.50-7.61 (m, 1H), 4.43 (m, 4H), 3.88 (m, 4H), 3.30-3.62 (m, 4H), 2.17 (s, 3H), 1.19-1.35 (m, 6H). MS (M/z, M+1), 466.65.

### EXAMPLE 317

### Methyl 2-(2-chlorophenyl)-4-(4-(ethoxycarbonyl)piperazin-1-yl)quinazoline-7-carboxylate

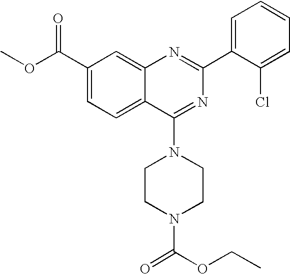

This compound was prepared according to the procedure for Example 311. ¹H NMR (400 MHz, DMSO-d₆) δ 8.30 (d, 1H), 8.18 (d, 1H) 7.98 (m, 1H), 7.85 (m, 1H), 7.55 (m, 1H), 7.40-7.50 (m, 2H), 4.06 (q, 2H), 3.92 (s, 3H), 3.85 (m, 4H), 3.60 (m, 4H), 1.19 (t, 3H). MS (M/z, M+1), 455.59.

### EXAMPLE 318

### 2-(2-Chlorophenyl)-4-(4-(ethoxycarbonyl)piperazin-1-yl)quinazoline-7-carboxylic acid

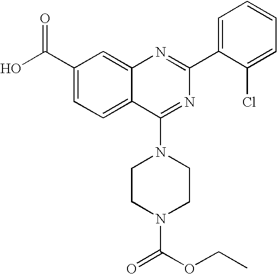

This compound was prepared according to the procedure for Step 1 of Example 311. MS (M/z, M+1), 441.57.

### EXAMPLE 319

### Ethyl 4-(2-(2-chlorophenyl)-7-(diethylcarbamoyl)quinazolin-4-yl)piperazine-1-carboxylate

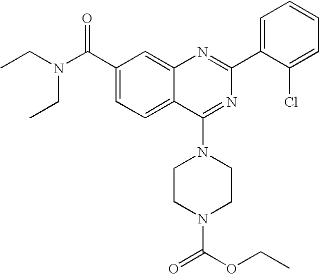

This compound was prepared according to the procedure for Examples 311. ¹H NMR (400 MHz, CDCl₃) δ 8.09 (s, 1H), 8.04 (d, 1H), 7.83 (d, 1H) 7.65 (d, 1H), 7.50-7.58 (m, 2H), 7.41-7.47 (m, 1H), 4.28 (m, 4H), 4.20 (q, 2H), 3.77 (m, 4H), 3.57 (m, 2H), 3.29 (m, 2H), 1.25-1.35 (m, 6H), 1.15 (t, 3H). MS (M/z, M+1), 455.59.

### EXAMPLE 320

### Ethyl 4-(2-(2-chlorophenyl)-7-(isopropylcarbamoyl)quinazolin-4-yl)piperazine-1-carboxylate

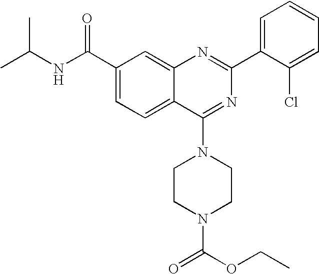

This compound was prepared according to the procedure for Examples 311. MS (M/z, M+1), 482.59. ¹H NMR (400 MHz, CDCl₃) δ 8.94 (s, 1H), 8.24 (d, 1H), 8.06 (d, 1H) 7.83 (d, 1H), 7.54-7.65 (m, 3H), 7.45-7.50 (m, 1H), 4.33 (m, 4H), 4.22-4.30 (m, 3H), 3.79 (m, 4H), 1.28-1.33 (m, 9H).

### EXAMPLE 321

### Ethyl 4-(2-(2-chlorophenyl)-7-(methylcarbamoyl)quinazolin-4-yl)piperazine-1-carboxylate

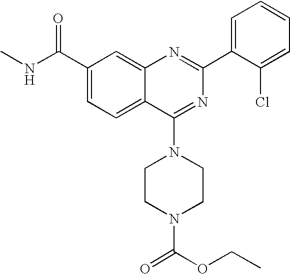

This compound was prepared according to the procedure for Examples 311. MS (M/z, M+1), 454.54.

### EXAMPLE 322

### Methyl 4-(4-acetylpiperazin-1-yl)-2-(2-chlorophenyl)quinazoline-7-carboxylate

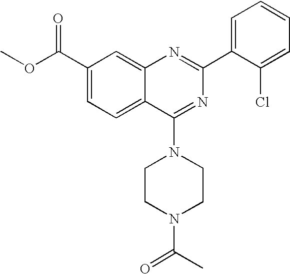

This compound was prepared according to the procedure for Example 13. MS (M/z, M+1), 425.50.

### EXAMPLE 323

### 4-(4-Acetylpiperazin-1-yl)-2-(2-chlorophenyl)-N,N-diethylquinazoline-7-carboxamide

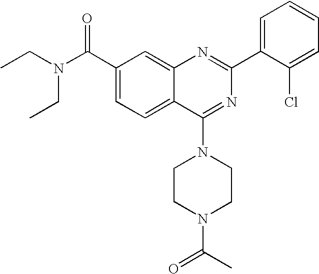

This compound was prepared according to the procedure for Examples 311. ¹H NMR (400 MHz, CDCl₃) δ 8.06 (d, 1H), 8.04 (s, 1H), 7.81 (d, 1H), 7.60 (d, 1H), 7.48-7.56 (m, 2H), 7.40-7.46 (m, 1H), 4.30 (m, 4H), 3.82 (m, 4H), 3.55 (q, 2H), 3.26 (q, 2H), 2.18 (s, 3H), 1.25 (t, 3H), 1.14 (t, 3H). MS (M/z, M+1), 466.64.

### EXAMPLE 324

### 1-(4-(2-(2-Chlorophenyl)-7-(piperidine-1-carbonyl)quinazolin-4-yl)piperazin-1-yl)ethanone

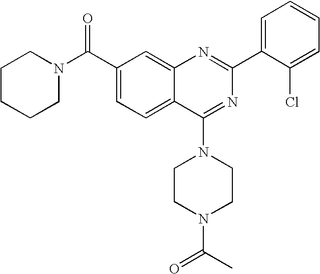

This compound was prepared according to the procedure for Example 311. ¹H NMR (400 MHz, CDCl₃) δ 8.08 (s, 1H), 8.04 (d, 1H), 7.83 (d, 1H), 7.63 (d, 1H), 7.48-7.56 (m, 2H), 7.41-7.47 (m, 1H), 4.25 (m, 4H), 3.79 (m, 6H), 3.35 (s, 2H), 2.18 (s, 3H), 1.70 (m, 4H), 1.55 (m, 2H). MS (M/z, M+1), 478.67.

### EXAMPLE 325

### 4-(4-Acetylpiperazin-1-yl)-2-(2-chlorophenyl)-N-(4-methoxybenzyl)quinazoline-7-carboxamide

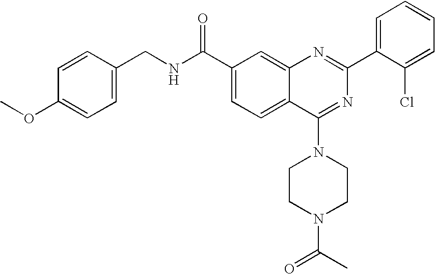

This compound was prepared according to the procedure for Example 311. ¹H NMR (400 MHz, CDCl₃) δ 9.04 (s, 1H), 8.21 (d, 1H), 8.05 (m, 2H), 7.83 (m, 1H), 7.44-7.58 (m, 3H), 7.33 (d, 2H), 6.87 (d, 2H), 4.58 (d, 2H) 4.23-4.31 (m, 4H), 3.86-3.92 (m, 2H), 3.70-3.82 (m, 5H), 2.18 (s, 3H). MS (M/z, M+1), 530.69.

### EXAMPLE 326

### Methyl 2-(2-chlorophenyl)-4-(3-(trifluoromethoxy)phenylamino)quinazoline-6-carboxylate

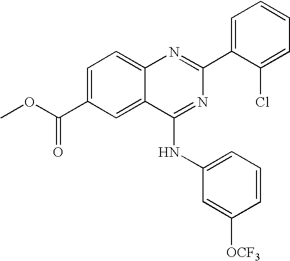

A solution of methyl 4-chloro-2-(2-chlorophenyl)quinazoline-6-carboxylate (60 mg), 3-trifluoromethoxyaniline (33 mg) and triethylamine (50 μL) in DMSO (1.5 mL) was stirred at 115° C. for 15 min. The solution was partitioned between water and ethyl acetate. The crude material was purified by column chromatography to give the title compound. ¹H NMR (400 MHz, DMSO-d₆) δ 10.69 (s, 1H), 9.36 (d, 1H), 8.38 (m, 1H), 8.16 (s, 1H), 7.92 (m, 2H), 7.76 (m, 1H), 7.59 (m, 1H), 7.43-7.53 (m, 3H), 7.12 (m, 1H), 3.97 (s, 3H). MS (M/z, M+1), 474.53.

### EXAMPLE 327

### Methyl 2-(2-chlorophenyl)-4-(3-isopropoxyphenylamino)quinazoline-6-carboxylate

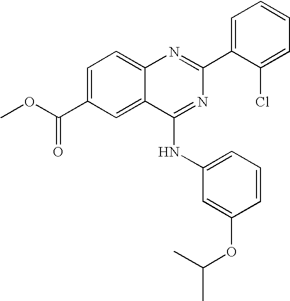

This compound was prepared according to the procedure for Example 326. MS (M/z, M+1), 448.59.

### EXAMPLE 328

### Methyl 2-(2-chlorophenyl)-4-(3,5-dimethoxyphenylamino)quinazoline-6-carboxylate

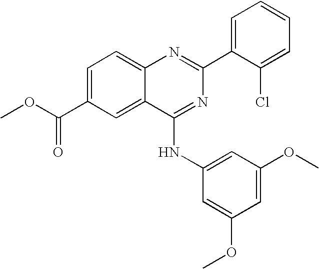

This compound was prepared according to the procedure for Example 326. MS (M/z, M+1), 450.57.

### EXAMPLE 329

### Methyl 2-(2-chlorophenyl)-4-(o-tolylamino)quinazoline-6-carboxylate

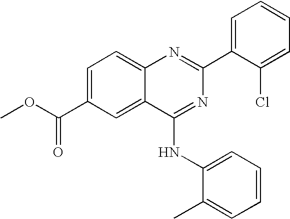

This compound was prepared according to the procedure for Example 326. ¹H NMR (400 MHz, DMSO-d₆) δ 10.90 (s, 1H), 9.35 (d, 1H), 8.40 (m, 1H), 7.89 (d, 1H), 7.62 (m, 1H), 7.35-7.52 (m, 4H), 7.20-7.32 (m, 3H), 3.95 (s, 3H), 2.24 (s, 3H). MS (M/z, M+1), 474.53.

### EXAMPLE 330

### Methyl 2-(2-chlorophenyl)-4-(5-methoxyindolin-1-yl)quinazoline-6-carboxylate

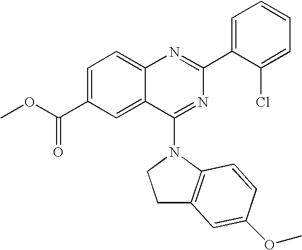

This compound was prepared according to the procedure for Example 326. MS (M/z, M+1), 446.58. ¹H NMR (400 MHz, DMSO-d₆) δ 8.86 (s, 1H), 8.30 (m, 1H), 7.86 (d, 2H), 7.75 (d, 1H), 7.60 (m, 1H), 7.42-7.50 (m, 2H), 6.93 (s, 1H), 6.73 (d, 1H), 4.61 (t, 2H), 3.90 (s, 3H), 3.72 (s, 3H), 3.18 (t, 2H).

### EXAMPLE 331

### 2-(2-Chlorophenyl)-N-o-tolylquinazolin-4-amine

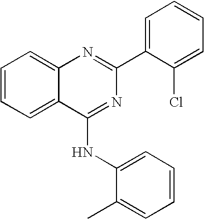

This compound was prepared according to the procedure for Example 326. MS (M/z, M+1), 346.53.

### EXAMPLE 332

### 2-(2-Chlorophenyl)-N-(4-ethoxyphenyl)quinazolin-4-amine

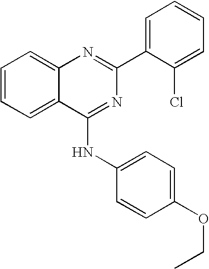

This compound was prepared according to the procedure for Example 326. MS (M/z, M+1), 376.55.

### EXAMPLE 333

### 2-(2-Chlorophenyl)-4-(4-methyl-1,4-diazepan-1-yl)quinazoline

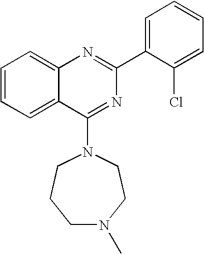

This compound was prepared according to the procedure for Example 326. ¹H NMR (400 MHz, DMSO-d₆) δ 8.11 (d, 1H), 7.74-7.80 (m, 3H), 7.47-7.55 (m, 2H), 7.41-7.45 (m, 2H), 3.90-4.10 (m, 4H), 3.75 (m, 2H), 3.52 (m, 2H), 1.97-2.03 (m, 5H). MS (M/z, M+1), 353.57.

### EXAMPLE 334

### 1-(4-(2-(2-Chlorophenyl)quinazolin-4-yl)-1,4-diazepan-1-yl)ethanone

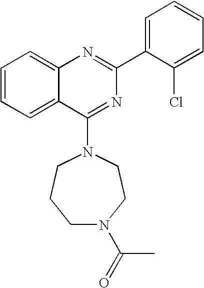

This compound was prepared according to the procedure for Example 326. MS (M/z, M+1), 381.55.

### EXAMPLE 335

### 2-(3,4-Dimethoxybenzyl)-4-(piperidin-1-yl)quinazoline

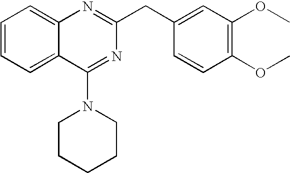

### Step 1

A mixture of anthranilamide (1.36 g), 3,4-dimethoxyphenylacetic acid (2.0 g), N-(3-dimethylamino)-N′-ethylcarbodiimide hydrochloride (2.2 g), and 1-hydroxybenzotriazole (1.4 g) in dimethylformamide (15 mL) was stirred at room temperature for 40 h. The solution was partitioned between water and ethyl acetate. The crude material was recrystallized from ethyl acetate to give 2-(3,4-dimethoxybenzyl)quinazoline-4-ol. MS (M/z, M+1), 298.52.

### Step 2

A mixture of 2-(3,4-dimethoxybenzyl)quinazolin-4-ol (1.6 g), phosphorous pentachloride (1.12 g), and phosphorus oxychloride (3 mL) in toluene (10 mL) was heated at 75° C. for 1 h. The solution was partitioned between brine (200 mL) and ethyl acetate (300 mL). The organic layer was dried over Na₂SO₄ and quickly removed by nitrogen. The crude material was dissolved in DMSO (1 mL). Piperidine (100 μL) was added and the solution was heated at 60° C. for 10 min. The solution was partitioned between brine and ethyl acetate. The crude material was purified by reverse-phase HPLC to afford the title compound. ¹H NMR (400 MHz, CDCl₃) δ 8.01 (d, 1H), 7.83 (m, 2H), 7.54 (t, 1H), 7.06 (s, 1H), 7.00 (m, 1H), 6.80 (d, 1H), 4.18 (s, 2H) 4.11 (m, 4H), 3.88 (s, 3H), 3.84 (s, 3H), 1.85 (m, 6H). MS (M/z, M+1), 364.60.

### EXAMPLE 336

### 2-(4-Methoxybenzyl)-4-(piperidin-1-yl)quinazoline

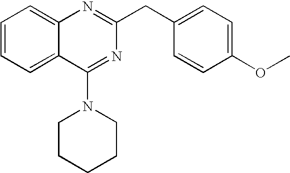

This compound was prepared according to the procedure for Example 335. MS (M/z, M+1), 334.59. ¹H NMR (400 MHz, CDCl₃) δ 8.10 (d, 1H), 7.81 (m, 2H), 7.50 (t, 1H), 7.39 (d, 2H), 6.83 (d, 2H), 4.21 (s, 2H) 4.09 (m, 4H), 3.76 (s, 3H), 1.83 (m, 6H).

### EXAMPLE 337

### 2-(2-Methylbenzyl)-4-(4-methylpiperazin-1-yl)quinazoline

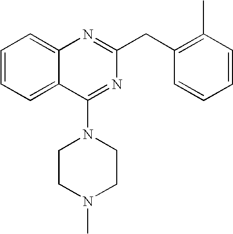

This compound was prepared according to the procedure for Example 335. MS (M/z, M+1), 333.65.

### EXAMPLE 338

### Ethyl 4-(2-(1-o-tolylethyl)quinazolin-4-yl)piperazine-1-carboxylate

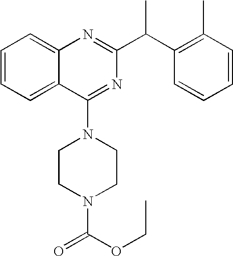

This compound was prepared according to the procedure for Example 335. ¹H NMR (400 MHz, CDCl₃) δ 8.30 (d, 1H), 7.90 (m, 2H), 7.59 (t, 1H), 7.43 (m, 1H), 7.14-7.18 (m, 3H), 4.96 (q, 1H) 4.15-4.25 (m, 6H), 3.70 (m, 4H), 2.44 (s, 3H), 1.75 (d, 3H), 1.31 (t, 3H). MS (M/z, M+1), 405.63.

### EXAMPLE 339

### 2-Benzyl-4-(piperidin-1-yl)quinazoline

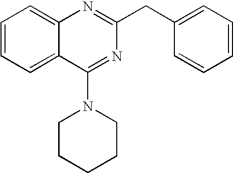

This compound was prepared according to the procedure for Example 335. ¹H NMR (400 MHz, CDCl₃) δ 8.19 (d, 1H), 7.83 (m, 2H), 7.48-7.54 (m, 2H), 7.24-7.34 (m, 4H), 4.32 (s, 2H), 4.09 (m, 4H), 1.81 (m, 6H). MS (M/z, M+1), 304.58.

### EXAMPLE 340

### Ethyl 4-(2-(2-methylbenzyl)quinazolin-4-yl)piperazine-1-carboxylate

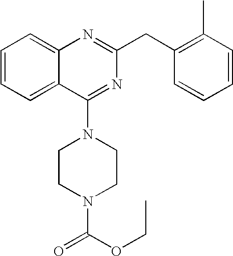

This compound was prepared according to the procedure for Example 335. ¹H NMR (400 MHz, DMSO-d₆) δ 8.17 (d, 1H), 8.00 (t, 1H), 7.90 (d, 1H), 7.66 (t, 1H), 7.30 (m, 1H), 7.11-7.22 (m, 3H), 4.26 (d, 2H), 4.07 (m, 6H), 3.57 (m, 4H), 2.38 (s, 3H), 1.18 (t, 3H). MS (M/z, M+1), 391.62.

### EXAMPLE 341

### 2-(2-Methylbenzyl)-4-(pyrrolidin-1-yl)quinazoline

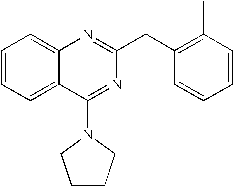

This compound was prepared according to the procedure for Example 335. MS (M/z, M+1), 304.28. ¹H NMR (400 MHz, DMSO-d₆) δ 8.19 (d, 1H), 7.65 (m, 1H), 7.61 (m, 1H), 7.35 (m, 1H), 7.22 (m, 1H), 7.11 (m, 1H), 7.05 (m, 2H), 4.00 (s, 2H), 3.77 (m, 4H), 2.47 (s, 3H), 2.39 (s, 3H), 1.91 (m, 4H).

### EXAMPLE 342

### 4-(Pyrrolidin-1-yl)-2-(1-o-tolylethyl)quinazoline

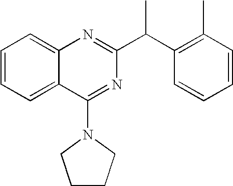

This compound was prepared according to the procedure for Example 335. MS (M/z, M+1), 318.57.

### EXAMPLE 343

### Ethyl 4-(2-(2-chlorophenyl)-6-nitroquinazolin-4-yl)piperazine-1-carboxylate

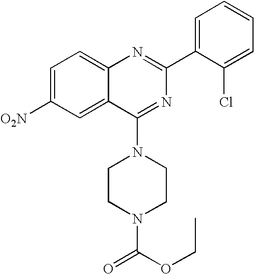

A solution of methyl 4-chloro-2-(2-chlorophenyl)-6-nitroquinazoline (50 mg), ethyl piperazine-1-carboxylate (26 mg) and diisopropylethylamine (0.2 μL) in DMSO (1 mL) was stirred at room temperature for 1 h. The solution was partitioned between water and ethyl acetate. The crude material was purified by reverse-phase HPLC to give the title compound. ¹H NMR (400 MHz, DMSO-d₆) δ 8.88 (d, 1H), 8.55 (m, 1H), 7.97 (d, 1H), 7.85 (m, 1H), 7.48-7.62 (m, 3H), 4.08 (m, 6H), 3.62 (m, 4H), 1.19 (t, 3H). MS (M/z, M+1), 442.63.

### EXAMPLE 344

### 1-(4-(2-(1-o-Tolylethyl)quinazolin-4-yl)piperazin-1-yl)ethanone

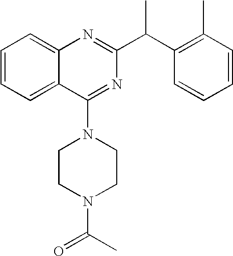

This compound was prepared according to the procedure for Example 335. ¹H NMR (400 MHz, CDCl₃) δ 8.32 (d, 1H), 7.89 (m, 2H), 7.60 (t, 1H), 7.42 (m, 1H), 7.15-7.18 (m, 3H), 4.98 (q, 1H), 4.16-4.23 (m, 4H), 3.70 (m, 4H), 2.43 (s, 3H), 2.17 (s, 3H), 1.77 (d, 3H). MS (M/z, M+1), 375.56.

### EXAMPLE 345

### Ethyl 4-(2-(naphthalen-1-ylmethyl)quinazolin-4-yl)piperazine-1-carboxylate

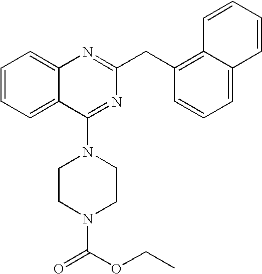

This compound was prepared according to the procedure for Example 335. MS (M/z, M+1), 427.63. ¹H NMR (400 Mhz, DMSO-d₆) δ 7.94 (d, 1H), 7.74-7.84 (m, 6H), 7.55 (m, 1H), 7.40-7.48 (m, 3H), 4.26 (s, 2H), 4.04 (q, 2H), 3.67 (m, 4H), 3.55 (m, 4H), 1.18 (t, 3H).

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf Comput. Sci. 1988, 28, 31-36. All SMILES strings used herein, as well as many IUPAC names, were generated using CambridgeSoft's ChemDraw 10.0.
O═S(NCC1=NC2=CC(C)═CC═C2C(N3CCCC3)=N1)(C4=C(C═CC═C5)C5=CC═C4)=O
O═S(NCC1=NC2=CC═C(C)C═C2C(N3CCCC3)=N1)(C4=C(C═CC═C5)C5=CC═C4)=O
O═S(NCC1=NC2=CC═CC(C)═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=CC═C4C)═O
O═S(NCC1=NC2=CC═CC═C2C(N3CCCC3)=N1)(C4=C(C═CC═C5)C5=CC(C)=C4)=O
O═S(NCC1=NC2=CC═CC═C2C(N3CCCC3)=N1)(C4=C(C═CC═C5)C5=C(C)C═C4)=O
O═S(NCC1=NC2=CC═CC═C2C(N3CCCC3)=N1)(C4=C(C═CC═C5C)C5=CC═C4)=O
O═S(NCC1=NC2=CC═CC═C2C(N3CCCC3)=N1)(C4=C(C(C)═CC═C5)C5=CC═C4)=O
O═S(NCC1=NC2=CC═CC═C2C(N3CCCC3)=N1)(C4=C(C═CC(C)═C5)C5=CC═C4)=O
O═S(NCC1=NC2=CC═CC═C2C(N3CCCC3)=N1)(C4=C(C═C(C)C═C5)C5=CC═C4)=O
O═S(NCC1=NC2=C(CC)C═CC═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=C(CC)C═C4)=O
O═S(NCC1=NC2=CC═C(C(C)(C)C)C═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=C(C(C)C)C═C4)=O
O═S(NCC1=NC2=C(C)C═CC═C2C(N3CCCC3)=N1)(C4=C(C═CC═C5)C5=C(CC)C═C4)=O
O═S(NCC1=NC2=CC═CC(C)═C2C(N3CCCC3)=N1)(C4=C(C═CC═C5C(C)(C)C)C5=CC═C4)=O
O═S(NCC1=NC2=CC═C(CC)C(C)═C2C(N3CCCC3)=N1)(C4=C(C═C(C(C)C)C═C5) C5=C(C(C)(C)C)C═C4)=O
O═S(NCC1=NC2=C(OC)C═CC═C2C(N3CCCC3)=N1)(C4=C(C═CC═C5)C5=CC═C4
O═S(NCC1=NC2=CC(OC)═CC═C2C(N3CCCC3)=N1)(C4=C(C═CC═C5)C5=CC═C4
O═S(NCC1=NC2=CC═C(OC)C═C2C(N3CCCC3)=N1)(C4=C(C═CC═C5)C5=CC═C4
O═S(NCC1=NC2=CC═CC(OC)═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=CC═C4OC)═O
O═S(NCC1=NC2=CC═CC═C2C(N3CCCC3)=N1)(C4=C(C═CC═C5)C5=CC(OC)=C4)=O
O═S(NCC1=NC2=CC═CC═C2C(N3CCCC3)=N1)(C4=C(C═CC═C5)C5=C(OC)C═C4)=O
O═S(NCC1=NC2=CC═CC═C2C(N3CCCC3)=N1)(C4=C(C═CC═C5OC)C5=CC═C4)=O
O═S(NCC1=NC2=CC═CC═C2C(N3CCCC3)=N1)(C4=C(C(OC)═CC═C5)C5=CC═C4)=O
O═S(NCC1=NC2=CC═CC═C2C(N3CCCC3)=N1)(C4=C(C═CC(OC)═C5)C5=CC═C4)=O
O═S(NCC1=NC2=CC═CC═C2C(N3CCCC3)=N1)(C4=C(C═C(OC)C═C5)C5=CC═C4)=O
O═S(NCC1=NC2=C(OCC)C═CC═C2C(N3 CCCC3)=N1)(C4=C(C═CC═C5)C5=CC═C4)=O
O═S(NCC1=NC2=CC═CC═C2C(N3CCCC3)=N1)(C4=C(C═CC═C5)C5=C(OCC)C═C4)=O
O═S(NCC1=NC2=CC═C(OC(C)(C)C)C═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=C(OC(C)C)C═C4)=O
O═S(NCC1=NC2=C(OCCCC)C═CC═C2C(N3 CCCC3)=N1)(C4=C(C═CC═C5)C5=C(OCCC)C═C4)=O
O═S(NCC1=NC2=CC═CC(OC)═C2C(N3CCCC3)=N1)(C4=C(C═CC═C5OC(C)(C)C) C5=CC═C4)=O
O═S(NCC1=NC2=CC═C(OCC)C(OC)═C2C(N3 CCCC3)=N1)(C4=C(C═C(OC(C)C)C═C5)C5=C(OC(C)(C)C)C═C4)=O
O═S(NCC1=NC2=C(F)C═CC═C2C(N3CCCC3)=N1)(C4=C(C═CC═C5)C5=CC═C4)=O
O═S(NCC1=NC2=CC(Cl)═CC═C2C(N3CCCC3)=N1)(C4=C(C═CC═C5)C5=CC═C4)=O
O═S(NCC1=NC2=CC═C(I)C═C2C(N3CCCC3)=N1)(C4=C(C═CC═C5)C5=CC═C4)=O
O═S(NCC1=NC2=CC═CC(Br)═C2C(N3CCCC3)=N1)(C4=C(C═CC═C5)C5=CC═C4)=O
O═S(NCC1=NC2=CC═CC═C2C(N3CCCC3)=N1)(C4=C(C═CC═C5)C5=CC═C4F)═O
O═S(NCC1=NC2=CC═CC═C2C(N3CCCC3)=N1)(C4=C(C═CC═C5)C5=CC(Cl)=C4)=O
O═S(NCC1=NC2=CC═CC═C2C(N3CCCC3)=N1)(C4=C(C═CC═C5)C5=C(Br)C═C4)=O
O═S(NCC1=NC2=CC═CC═C2C(N3CCCC3)=N1)(C4=C(C═CC═C51)C5=CC═C4)=O
O═S(NCC1=NC2=CC═CC═C2C(N3CCCC3)=N1)(C4=C(C(F)═CC═C5)C5=CC═C4)=O
O═S(NCC1=NC2=CC═CC═C2C(N3CCCC3)=N1)(C4=C(C═CC(Cl)═C5)C5=CC═C4)=O
O═S(NCC1=NC2=CC═CC═C2C(N3CCCC3)=N1)(C4=C(C═C(Br)C═C5)C5=CC═C4)=O
O═S(NCC1=NC2=C(I)C═CC═C2C(N3CCCC3)=N1)(C4=C(C═CC═C5)C5=CC(F)=C4)=O
O═S(NCC1=NC2=C(F)C═C(F)C═C2C(N3CCCC3)=N1)(C4=C(C═CC═C5)C5=C(Br)C═C4)=O
O═S(NCC1=NC2=CC═C(Cl)C(Cl)═C2C(N3CCCC3)=N1)(C4=C(C═CC═C5)C5=C(B r)C(F)=C4)=O
O═S(NCC1=NC2=CC(F)═C(F)C═C2C(N3CCCC3)=N1)(C4=C(C═C(F)C═C5)C5=C(F)C═C4)=O
O═S(NCC1=NC2=C(Cl)C═CC═C2C(N3CCCC3)=N1)(C4=C(C═CC═C5)C5=C(Br)C═C4)=O
O═S(NCC1=NC2=CC(I)═CC(I)═C2C(N3CCCC3)=N1)(C4=C(C═CC(F)═C5)C5=CC═C4)=O
O═S(NCC1=NC2=CC═C(Br)C(Cl)═C2C(N3CCCC3)=N1)(C4=C(C(F)═C(F)C═C5)C5=CC═C4)=O
O═S(NCC1=NC2=C([N+]([O−])═O)C═CC═C2C(N3CCCC3)=N1)(C4=C(C═CC═C5) C5=CC═C4)=O
O═S(NCC1=NC2=CC(SC)═CC═C2C(N3CCCC3)=N1)(C4=C(C═CC═C5)C5=CC═C4
O═S(NCC1=NC2=CC═C([N+]([O−])═O)C═C2C(N3CCCC3)=N1)(C4=C(C═CC═C5)C5=CC═C4)=O
O═S(NCC1=NC2=CC═CC(SC)═C2C(N3CCCC3)=N1)(C4=C(C═CC═C5)C5=CC═C4
O═S(NCC1=NC2=CC═CC═C2C(N3CCCC3)=N1)(C4=C(C═CC═C5)C5=CC═C4[N+]
O═S(NCC1=NC2=CC═CC═C2C(N3CCCC3)=N1)(C4=C(C═CC═C5)C5=CC(SC)═C4
O═S(NCC1=NC2=CC═CC═C2C(N3CCCC3)=N1)(C4=C(C═CC═C5)C5=C(SCC)C═C4)=O
O═S(NCC1=NC2=CC═CC═C2C(N3CCCC3)=N1)(C4=C(C═CC═C5[N+]([O−])═O)C5=CC═C4)=O
O═S(NCC1=NC2=CC═CC═C2C(N3CCCC3)=N1)(C4=C(C(SC)═CC═C5)C5=CC═C4
O═S(NCC1=NC2=CC═CC═C2C(N3CCCC3)=N1)(C4=C(C═CC(C#N)═C5)C5=CC═C4)=O
O═S(NCC1=NC2=CC═CC═C2C(N3CCCC3)=N1)(C4=C(C═C(SC)C═C5)C5=CC═C4
O═S(NCC1=NC2=C(C#N)C═CC═C2C(N3CCCC3)=N1)(C4=C(C═CC═C5)C5=CC(S C)=C4)=O
O═S(NCC1=NC2=C(F)C═C(F)C═C2C(N3CCCC3)=N1)(C4=C(C═CC═C5)C5=C([N+]([O−])═O)C═C4)=O
O═S(NCC1=NC2=CC═C(Cl)C(C#N)═C2C(N3CCCC3)=N1)(C4=C(C═CC═C5)C5=C(Br)C([N+]([O−])═O)=C4)=O
O═S(NCC1=NC2=CC(F)═C(F)C═C2C(N3CCCC3)=N1)(C4=C(C═C(SC)C═C5)C5=C(C#N)C═C4)=O
O═S(NCC1=NC2=C(C#N)C═CC═C2C(N3CCCC3)=N1)(C4=C(C═CC═C5)C5=C(OC)C═C4)=O
O═S(NCC1=NC2=CC([N+]([O−])═O)═CC(OC)═C2C(N3CCCC3)=N1)(C4=C(C═CC(F)═C5)C5=CC═C4)=O
O═S(NCC1=NC2=CC═C(Br)C([N+]([O−])═O)═C2C(N3CCCC3)=N1)(C4=C(C(F)═C(SC)C═C5)C5=CC═C4)=O
CN(C)C1=NC(CNS(C2=C(C═CC═C3)C3=CC═C2)(═O)═O)═NC4=CC═CC═C41
CN(CC)C1=NC(CNS(C2=C(C═CC═C3)C3=CC═C2)(═O)═O)═NC4=CC═CC═C41
CN(C(C)C)C1=NC(CNS(C2=C(C═CC═C3)C3=CC═C2)(═O)═O)═NC4=CC═CC═C41
CNC1=NC(CNS(C2=C(C═CC═C3)C3=CC═C2)(═O)═O)═NC4=CC═CC═C41
CCNC1=NC(CNS(C2=C(C═CC═C3)C3=CC═C2)(═O)═O)═NC4=CC═CC═C41
CCCNC1=NC(CNS(C2=C(C═CC═C3)C3=CC═C2)(═O)═O)═NC4=CC═CC═C41
O═S(NCC1=NC2=CC═CC═C2C(N(C)C(C)(C)C)═N1)(C3=C(C═CC═C4)C4=CC═C3)=O
O═S(NCC1=NC2=CC═CC═C2C(NC3=CC═CC═C3)=N1)(C4=C(C═CC═C5)C5=CC═C4)=O
CN(C1=CC═CC═C1)C2=NC(CNS(C3=C(C═CC═C4)C4=CC═C3)(═O)═O)═NC5=CC═CC═C52
O═S(NCC1=NC2=CC═CC═C2C(NC3=CC═CC(F)=C3)=N1)(C4=C(C═CC═C5)C5=CC═C4)=O
CN(C1=CC═C(F)C(Cl)═C1)C2=NC(CNS(C3=C(C═CC═C4)C4=CC═C3)(═O)═O)═N C5=CC═CC═C52
O═S(NCC1=NC2=CC═CC═C2C(NC3=CC═CC(C(F)(F)F)=C3)=N1)(C4=C(C═CC═C5)C5=CC═C4)=O
O═S(NCC1=NC2=CC═CC═C2C(NC3=CC═C(OC)C═C3)=N1)(C4=C(C═CC═C5)C5=CC═C4)=O
CN(C1=CC═C(C#N)C═C1)C2=NC(CNS(C3=C(C═CC═C4)C4=CC═C3)(═O)═O)═NC5=CC═CC═C52
O═S(NCC1=NC2=CC═CC═C2C(NC3=CC([N+]([O−])═O)═CC═C3)=N1)(C4=C(C═CC═C5)C5=CC═C4)=O
O═S(NCC1=NC2=CC═CC═C2C(N3CCCCC3)=N1)(C4=C(C═CC═C5)C5=CC═C4)=O
O═S(NCC1=NC2=CC═CC═C2C(N3CCCCCC3)=N1)(C4=C(C═CC═C5)C5=CC═C4)=O
CN(C1CCCCC1)C2=NC(CNS(C3=C(C═CC═C4)C4=CC═C3)(═O)═O)═NC5=CC═CC═C52
CN(CC)C1=NC(CNS(C2=C(C═CC═C3)C3=CC═C2)(═O)═O)═NC4=C(F)C═CC═C41
O═S(NCC1=NC2=CC═CC═C2C(N3CCNCC3)=N1)(C4=C(C═CC═C5)C5=CC═C4)=O
O═S(NCC1=NC2=CC═CC═C2C(NC3CC3)=N1)(C4=C(C═CC═C5)C5=CC═C4)=O
O═S(NCC1=NC2=CC═CC═C2C(NC3CCCC3)=N1)(C4=C(C═CC═C5)C5=CC═C4)=O
O═S(NCC1=NC2=CC═CC═C2C(N3CCNCCC3)=N1)(C4=C(C═CC═C5)C5=CC═C4)=O
O═S(NCC1=NC2=CC═CC═C2C(NCC3=CC═CC(F)=C3)=N1)(C4=C(C═CC═C5)C5=CC═C4)=O
CN(CC1=CC═C(F)C(Cl)═C1)C2=NC(CNS(C3=C(C═CC═C4)C4=CC═C3)(═O)═O)═NC5=CC═CC═C52
O═S(NCC1=NC2=CC═CC═C2C(NCC3=CC═CC(C(F)(F)F)=C3)=N1)(C4=C(C═CC═C5)C5=CC═C4)=O
O═S(NCC1=NC2=CC═CC═C2C(NCC3=CC═C(OC)C═C3)=N1)(C4=C(C═CC═C5)C5=CC═C4)=O
CN(CC1=CC═C(C#N)C═C1)C2=NC(CNS(C3=C(C═CC═C4)C4=CC═C3)(═O)═O)═N C5=CC═CC═C52
O═S(NCC1=NC2=CC═CC═C2C(NCC3=CC([N+]([O−])═O)═CC═C3)=N1)(C4=C(C═CC═C5)C5=CC═C4)=O
O═S(NCC1=NC2=CC═CC═C2C(N3C(C)CN(C)CC3C)═N1)(C4=C(C═CC═C5)C5=CC═C4)=O
O═S(NCC1=NC2=CC═CC═C2C(N3CCC(C)CC3)=N1)(C4=C(C═CC═C5)C5=CC═C4
O═S(NCC1=NC2=CC═CC═C2C(N3CCCC(C(C)C)CC3)=N1)(C4=C(C═CC═C5)C5=CC═C4)=O
O═S(NCC1=NC2=CC═CC═C2C(NCC3=CC═C(F)C═C3)=N1)(C4=C(C═CC═C5)C5=C(C)C═C4)=O
O═S(NCC1=NC2=CC═C(C)C═C2C(NCC3=CC═CC(C(F)(F)F)=C3)=N1)(C4=C(C═C C═C5)C5=CC═C4)=O
CN(CC1=CC═C(C#N)C═C1Cl)C2=NC(CNS(C3=C(C═CC(Br)═C4)C4=CC═C3)(═O)═O)═NC5=CC═CC(C)═C52
CN(C1CCCCC1)C2=NC(CNS(C3=CC═CC═C3)(═O)═O)═NC4=CC═CC═C42
CN(CC)C1=NC(CNS(C2=CC═CC═C2)(═O)═O)═NC3=C(F)C═CC═C31
O═S(NCC1=NC2=CC═CC═C2C(N3CCNCC3)=N1)(C4=CC═CC═C4)=O
O═S(NCC1=NC2=CC═CC═C2C(NC3CC3)=N1)(C4=CC═CC═C4)=O
O═S(NCC1=NC2=CC═CC═C2C(NC3CCCC3)=N1)(C4=CC═CC═C4)=O
O═S(NCC1=NC2=CC═CC═C2C(N3CCNCCC3)=N1)(C4=CC═CC═C4)=O
O═S(NCC1=NC2=CC═CC═C2C(NCC3=CC═C(F)C═C3)=N1)(C4=CC═CC═C4)=O
O═S(NCC1=NC2=CC═C(C)C═C2C(NCC3=CC═CC(C(F)(F)F)=C3)=N1)(C4=CC═CC═C4)=O
CN(CC1=CC═C(C#N)C═C1Cl)C2=NC(CNS(C3=CC═CC═C3)(═O)═O)═NC4=CC═C C(C)═C42
O═S(NCC1=NC2=CC═CC═C2C(NCC3=CC═CC(F)=C3)=N1)(C4=CC═C(OC)C(F)=C4)=O
CN(CC1=CC═C(F)C(Cl)═C1)C2=NC(CNS(C3=CC═C(C)C═C3)(═O)═O)═NC4=CC═CC═C42
O═S(NCC1=NC2=CC═CC═C2C(NCC3=CC═CC(C(F)(F)F)=C3)=N1)(C4=CC═CC(C C)=C4)=O
O═S(NCC1=NC2=CC═C(C)C═C2C(NCC3=CC═C(OC)C═C3)=N1)(C4=CC═CC═C4)=O
CN(CC1=CC═C(C#N)C═C1)C2=NC(CNS(C3=CC═CC═C3)(═O)═O)═NC4=CC═CC═C42
O═S(NCC1=NC2=CC═CC═C2C(NCC3=CC([N+]([O−])═O)═CC═C3)=N1)(C4=CC═CC═C4)=O
O═S(NCC1=NC2=CC═CC═C2C(N3 C(C)CN(C)CC3 C)═N1)(C4=CC═CC═C4)=O
O═S(NCC1=NC2=CC═CC═C2C(N3CCC(C)CC3)=N1)(C4=CC═CC═C4)=O
O═S(NCC1=NC2=CC═CC═C2C(N3CCCC(C(C)C)CC3)=N1)(C4=CC═CC═C4)=O
CN(C)C1=NC(CNS(C2=CC═CC═C2)(═O)═O)═NC3=CC═CC═C31
CN(CC)C1=NC(CNS(C2=CC═CC═C2)(═O)═O)═NC3=CC═CC═C31
CN(C(C)C)C1=NC(CNS(C2=CC═CC═C2)(═O)═O)═NC3=CC═CC═C31
CNC1=NC(CNS(C2=CC═CC═C2)(═O)═O)═NC3=CC═CC═C31
CCNC1=NC(CNS(C2=CC═CC═C2)(═O)═O)═NC3=CC═CC═C31
CCCNC1=NC(CNS(C2=CC═CC═C2)(═O)═O)═NC3=CC═CC═C31
O═S(NCC1=NC2=CC═CC═C2C(N(C)C(C)(C)C)═N1)(C3=CC═CC═C3)=O
O═S(NCC1=NC2=CC═CC═C2C(NC3=CC═CC═C3)=N1)(C4=CC═CC═C4)=O
CN(C1=CC═CC═C1)C2=NC(CNS(C3=CC═CC═C3)(═O)═O)═NC4=CC═CC═C42
O═S(NCC1=NC2=CC═CC═C2C(NC3=CC═CC(F)=C3)=N1)(C4=CC═CC═C4)=O
CN(C1=CC═C(F)C(Cl)═C1)C2=NC(CNS(C3=CC═C(F)C═C3)(═O)═O)═NC4=C(C)C═CC═C42
O═S(NCC1=NC2=CC═CC═C2C(NC3=CC═CC(C(F)(F)F)=C3)=N1)(C4=CC═CC═C4
O═S(NCC1=NC2=CC═CC═C2C(NC3=CC═C(OC)C═C3)=N1)(C4=CC═C(C)C═C4)=O
CN(C1=CC═C(C#N)C═C1)C2=NC(CNS(C3=CC═CC═C3)(═O)═O)═NC4=CC═CC═C42
O═S(NCC1=NC2=CC═C(Cl)C═C2C(NC3=CC([N+]([O−])═O)═CC═C3)=N1)(C4=CC═CC═C4)=O
O═S(NCC1=NC2=CC═CC═C2C(N3 CCN(C)CC3)=N1)(C4=CC═CC(C(F)(F)F)=C4)=O
O═S(NCC1=NC2=CC═CC═C2C(N3CCCCC3)=N1)(C4=CC═C(C)C═C4)=O
O═S(NCC1=NC2=CC═CC═C2C(N3CCCCCC3)=N1)(C4=C(F)C═C(F)C═C4)=O
O═S(NCC1=NC2=C([N+]([O−])═O)C═CC═C2C(N3CCCC3)=N1)(C4=CC═CC═C4)=O
O═S(NCC1=NC2=CC(SC)═CC═C2C(N3CCCC3)=N1)(C4=C(C═CC═C5)C5=CC═C4
O═S(NCC1=NC2=CC═C([N+]([O−])═O)C═C2C(N3CCCC3)=N1)(C4=C(C═CC═C5)C5=CC═C4)=O
O═S(NCC1=NC2=CC═CC(SC)═C2C(N3CCCC3)=N1)(C4=CC═CC═C4)=O
O═S(NCC1=NC2=CC═CC═C2C(N3CCCC3)=N1)(C4=CC═CC═C4[N+]([O−])═O)═O
O═S(NCC1=NC2=CC═CC═C2C(N3CCCC3)=N1)(C4=CC═CC(SC)=C4)=O
O═S(NCC1=NC2=CC═CC═C2C(N3CCCC3)=N1)(C4=CC═C(SCC)C═C4)=O
O═S(NCC1=NC2=CC═CC═C2C(N3CCCC3)=N1)(C4=C([N+]([O−])═O)C═CC═C4) ═O
O═S(NCC1=NC2=CC═CC═C2C(N3CCCC3)=N1)(C4=CC(F)═CC═C4)=O
O═S(NCC1=NC2=CC═CC═C2C(N3CCCC3)=N1)(C4=CC═C(C#N)C═C4)=O
O═S(NCC1=NC2=CC═CC═C2C(N3CCCC3)=N1)(C4=C(C(F)(F)F)C═CC═C4)=O
O═S(NCC1=NC2=C(C#N)C═CC═C2C(N3CCCC3)=N1)(C4=CC═CC(C)=C4)=O
O═S(NCC1=NC2=C(F)C═C(F)C═C2C(N3CCCC3)=N1)(C4=CC═C(C)C═C4)=O
O═S(NCC1=NC2=CC═C(Cl)C(C#N)═C2C(N3CCCC3)=N1)(C4=CC═C(Br)C([N+]([O−])═O)=C4)=O
O═S(NCC1=NC2=CC(F)═C(F)C═C2C(N3CCCC3)=N1)(C4=CC═C(C#N)C═C4)=O
O═S(NCC1=NC2=C(C#N)C═CC═C2C(N3 CCCC3)=N1)(C4=CC═C(OC)C═C4)=O
O═S(NCC1=NC2=CC([N+]([O−])═O)═CC(OC)═C2C(N3CCCC3)=N1)(C4=CC═CC═C4)=O
O═S(NCC1=NC2=CC═C(Br)C([N+]([O−])═O)═C2C(N3CCCC3)=N1)(C4=CC═CC═C4)=O
O═S(NCC1=NC2=C(OC)C═CC═C2C(N3CCCC3)=N1)(C4=CC═CC═C4)=O
O═S(NCC1=NC2=CC(OC)═CC═C2C(N3CCCC3)=N1)(C4=CC═CC═C4)=O
O═S(NCC1=NC2=CC═C(OC)C═C2C(N3CCCC3)=N1)(C4=CC═CC═C4)=O
O═S(NCC1=NC2=CC═CC(OC)═C2C(N3CCCC3)=N1)(C4=CC═CC═C4)=O
O═S(NCC1=NC2=CC═CC═C2C(N3CCCC3)=N1)(C4=CC═CC═C4OC)═O
O═S(NCC1=NC2=CC═CC═C2C(N3CCCC3)=N1)(C4=CC═CC(OC)=C4)=O
O═S(NCC1=NC2=CC═CC═C2C(N3CCCC3)=N1)(C4=CC═C(OC)C═C4)=O
O═S(NCC1=NC2=CC═CC═C2C(N3CCCC3)=N1)(C4=CC(C═CN5)=C5C═C4)=O
O═S(NCC1=NC2=CC═CC═C2C(N3CCCC3)=N1)(C4=CC(OCCO5)=C5C═C4)=O
O═S(NCC1=NC2=CC═CC═C2C(N3CCCC3)=N1)(C4=CC(OCO5)=C5C═C4)=O
O═S(NCC1=NC2=CC═CC═C2C(N3CCCC3)=N1)(C4=CC(N═CO5)=C5C═C4)=O
O═S(NCC1=NC2=C(OCC)C═CC═C2C(N3CCCC3)=N1)(C4=CC(CCC5)=C5C═C4)=O
O═S(NCC1=NC2=CC3=C(OCO3)C═C2C(N4CCCC4)=N1)(C5=CC═C(OCC)C═C5)=O
O═S(NCC1=NC2=CC═C(OC(C)(C)C)C═C2C(N3CCCC3)=N1)(C4=CC═CC═C4)=O
O═S(NCC1=NC2=CC═CC═C2C(N3CCCC3)=N1)(C4=CC═C(OC(C)C)C═C4)=O
O═S(NCC1=NC2=C(OC)C═CC═C2C(N3CCCC3)=N1)(C4=CC═C(OCCC)C═C4)=O
O═S(NCC1=NC2=CC═CC(OC)═C2C(N3CCCC3)=N1)(C4=CC═CC═C4)=O
O═S(NCC1=NC2=CC═C(OCC)C(OC)═C2C(N3 CCCC3)=N1)(C4=C(C)C═C(OC(C)(C)C)C═C4)=O
O═S(NCC1=NC2=C(C)C═CC═C2C(N3CCCC3)=N1)(C4=CC═CC═C4)=O
O═S(NCC1=NC2=CC(C)═CC═C2C(N3CCCC3)=N1)(C4=CC═CC═C4)=O
O═S(NCC1=NC2=CC═C(C)C═C2C(N3CCCC3)=N1)(C4=CC═CC═C4)=O
O═S(NCC1=NC2=CC═CC(C)═C2C(N3CCCC3)=N1)(C4=CC═CC═C4)=O
O═S(NCC1=NC2=CC═CC═C2C(N3CCCC3)=N1)(C4=CC═CC═C4C)═O
O═S(NCC1=NC2=CC═CC═C2C(N3CCCC3)=N1)(C4=CC═CC(C)=C4)=O
O═S(NCC1=NC2=CC═CC═C2C(N3CCCC3)=N1)(C4=CC═C(C)C═C4)=O
O═S(NCC1=NC2=CC═CC═C2C(N3CCCC3)=N1)(C4=CC(C)═CC═C4)=O
O═S(NCC1=NC2=CC═CC═C2C(N3CCCC3)=N1)(C4=C(C)C═CC═C4)=O
O═S(NCC1=NC2=CC═CC═C2C(N3CCCC3)=N1)(C4=CC═C(NCCN(C)C5)C5=C4)=O
O═S(NCC1=NC2=CC═CC═C2C(N3CCCCC3)=N1)(C4=CC═C(OCO5)C5=C4)=O
O═S(NCC1=NC2=CC═CC═C2C(N3CCCCCC3)=N1)(C4=CC═C(C═CN5)C5=C4)=O
O═S(NCC1=NC2=CC═CC═C2C(N3CCCC3)=N1)(C4=C(C═C(C═CC═C5)C5=C6)C6=CC═C4)=O
O═S(NCC1=NC2=CC═C(C(C)(C)C)C═C2C(N3CCCC3)=N1)(C4=CC═CC═C4)=O
O═S(NCC1=NC2=CC═CC═C2C(N3CCCC3)=N1)(C4=CC═C(C(C)C)C═C4)=O
O═S(NCC1=NC2=C(C)C═CC═C2C(N3CCCC3)=N1)(C4=C(C)C═C(C)C═C4)=O
O═S(NCC1=NC2=CC═CC═C2C(N3CCCC3)=N1)(C4=CC═C(N═CN5)C5=C4)=O
O═S(NCC1=NC2=CC═C(C)C(C)═C2C(N3CCCC3)=N1)(C4=CC═C(C═C55)C5=C4)=O
C12=CC═CC═C1C(N3CCCC3)=NC(CC4=CC═CC═C4)=N2
C12=CC═CC═C1C(N3CCCC3)=NC(C4CCCC4)=N2
CC1=CC═CC═C1C2=NC3=CC═CC═C3C(N4CCCC4)=N2
CC1=CC(CC2=NC3=CC═CC═C3C(N4CCCC4)=N2)=CC═C1
C12=CC═CC═C1C(N3CCCC3)=NC(C4CCOC4)=N2
FC1=CC(C2=NC3=CC═CC═C3C(N4CCCC4)=N2)=CC═C1
BrC(C═C1)=CC═C1CC2NC3CCCCC3C(N4CCCC4)N2
C12=CC═CC═C1C(N3 CCCC3)=NC(C4=CN═CN4)=N2
CC1=CC(C2=NC3=CC═CC═C3C(N4CCCC4)=N2)=CC═C1OC
CC(C═C1)=C(OC)C═C1CC2=NC3=CC═CC═C3C(N4CCCC4)=N2
C12=CC═CC═C1C(N3CCCC3)=NC(C4=CC═C54)=N2
CC(C)(C)C(C═C1)=CC═C1C2=NC3=CC═CC═C3C(N4CCCC4)=N2
CC(C)(C)C(C═C1)═CC═C1CC2=NC3=CC═CC═C3C(N4CCCC4)=N2
C12=CC═CC═C1C(N3 CCCC3)=NC(C4=CC═C04)=N2
O═[N+](C(C═C1)=CC═C1C2=NC3=CC═CC═C3C(N4CCCC4)=N2)[O−]
C12=CC═CC═C1C(N3CCCC3)=NC(CC4=CC═CC═C4)=N2
CSC1=CC═CC═C1C2=NC3=CC═CC═C3C(N4CCCC4)=N2
CSC1=CC(CC2=NC3=CC═CC═C3 C(N4CCCC4)=N2)=CC═C1
C12=CC═CC═C1C(N3 CCCC3)=NC(C4=CC═CN═C4)=N2
FC1=CC(C2=NC3=CC═C(F)C═C3C(N4CCCC4)=N2)=CC═C1
FC1=CC═CC═C1CC2=NC3=C(F)C═CC═C3C(N4CCCC4)=N2
C12=CC═CC═C1C(N3 CCCC3)=NC(C4=CC═NC═C4)=N2
BrC(C═C1)=CC═C1C2NC3CCCCC3C(N4CCCCC4)N2 BrC(C═C1)=CC═C1CC2=NC3=C(C)C═CC═C3C(N4CCCCC4)=N2
C12=CC═CC═C1C(N3CCCC3)=NC(C4=CC(C═CC═C5)=C5N4)=N2
CC1=CC(C2=NC3=CC═CC═C3C(N4CCN(C)CC4)=N2)=CC═C1OC
CC(C═C1)=C(OC)C═C1CC2=NC3=CC═CC═C3C(N4CCNCC4)=N2
C12=CC═CC═C1C(N3CCCC3)=NC(C4=CC(C═CC═C5)=C554)=N2
CC(C)(C)C(C═C1)=CC═C1C2=NC3=CC═CC═C3C(N4CCCCCC4)=N2
CC(C)(C)C(C═C1)=CC═C1CC2=NC3=CC═CC═C3C(N4CCCCCC4)=N2
C12=CC═CC═C1C(N3CCCC3)=NC(C4=CC(C═CC═C5)=C504)=N2
C12=CC═CC═C1C(N3CCCC3)=NC(C4=CC═CC5=C4NC═C5)=N2
C12=CC═CC═C1C(N3CCCC3)=NC(C4=CC═CC5=C4SC═C5)=N2
C12=CC═CC═C1C(N3CCCC3)=NC(C4=CC═C(C═CC═C5)C5=N4)=N2
C12=CC═CC═C1C(N3CCCC3)=NC(C4=CC═CC5=C4NC═N5)=N2
C12=CC═CC═C1C(N3CCCC3)=NC(C4=CC═CC5=C4OC═N5)=N2
CN(CC)C1=NC(C2=CC(C═CC═C3)=C3N═C2)=NC4=CC═CC═C41
FC1=CC═C(N═C(C2=CC═C(C═C53)C3=C2)N═C4N5CCCC5)C4=C1
FC1=C(N═C(C2=CC═C(C═CO3)C3=C2)N═C4N5CCCC5)C4=CC═C1
C12=CC═CC═C1C(N3 CCCC3)=NC(C4=CC═NC5=C4C═CC═C5)=N2
O═C(C1=C(C═CC═C2)C2=CC═C1)NCC3=NC4=CC═CC═C4C(N5CCCC5)=N3
O═C(NC1=CC═CC2=C1C═CC═C2)NCC3=NC4=CC═CC═C4C(N5CCCC5)=N3
O═S(NCC1=NC2=CC═CC═C2C(N3CCCC3)=N1)(NC4=CC═CC5=C4C═CC═C5)=O
O═C(C1=CC═CS1)NCC2=NC3=CC═CC═C3C(N4CCCC4)=N2
O═C(NC1=CC═CS1)NCC2=NC3=CC═CC═C3C(N4CCCC4)=N2
O═S(NCC1=NC2=CC═CC═C2C(N3CCCC3)=N1)(NC)═O
O═C(C1=CC═CN═C1)NCC2=NC3=CC═CC═C3C(N4CCCC4)=N2
O═C(NC1=CC═CC═N1)NCC2=NC3=CC═CC═C3C(N4CCCC4)=N2
O═S(NCC1=NC2=CC═CC═C2C(N3CCCC3)=N1)(NC4=CC═NC═C4)=O
O═C(C1=CC═CC═C1)NCC2=NC3=CC═CC═C3C(N4CCCC4)=N2
O═C(NC1=CC═CC═Cl)NCC2=NC3=CC═CC═C3C(N4CCCC4)=N2
O═S(NCC1=NC2=CC═CC═C2C(N3CCCC3)=N1)(NC4=CC═CC═C4)=O
C1C1=C(C(N2CCN(C(CC3)=O)C3 C2)=NC(C4=C(Cl)C═CC═C4)=N5)C5=CC═C1
FC6=C(C(N7CCN(C(OC8)=O)C8C7)=NC(C9=C(Cl)C═CC═C9)=N%10)C%10=CC═C6
ClC%11=C(C(N%12CCN(C(NC%13)=O)C%13C%12)=NC(C%14)=N%15)C%15=CC═C%11
FC%16=C(C(N%17CCN(C(N(C)C%18)=O)C%18C%17)=NC(C%19=C(Cl)C═CC═C%19)=N%20)C%20=CC═C%16
ClC%21=C(C(N%22CCN(C(CCC%23)=O)C%23C%22)=NC(C%24=C(Cl)C═CC═C %24)=N%25)C%25=CC═C%21
ClC%26=C(C(N%27CCN(C(OCC%28)=O)C%28C%27)=NC(C%29=C(F)C═CC═C%29)=N%30)C%30=CC═C%26
ClC%31=C(C(N%32CCN(C(NCC%33)=O)C%33C%32)=NC(C%34=C(F)C═CC═C%34)=N%35)C%35=CC═C%31
ClC%36=C(C(N%37CCN(C(N(C)CC%38)=O)C%3 8C%37)=NC(C%39=C(Cl)C═CC═C%39)=N%40)C%40=CC═C%36
ClC%41=C(C(N%42CCN(C(CNC%43=O)═O)C%43C%42)=NC(C%44=C(Cl)C═CC═C%44)=N%45)C%45=CC═C%41
ClC%46=C(C(N%47CCN(C(CN(C)C%48=O)═O)C%48C%47)=NC(C%49=C(Cl)C═
CC═C%49)=N%50)C%50=CC═C%46
ClC%5 l═C(C(N%52CCN(CCNC%53=O)C%53C%52)=NC(C%54=C(Cl)C═CC═C%54)=N%55)C%55=CC═C%51
ClC%56=C(C(N%57CCN(CCN(C)C%58=O)C%58C%57)=NC(C%59=C(F)C═CC═C%59)=N%60)C%60=CC═C%56
FC%61=C(C(N%62CCN(C(OC%63C)═O)C%63C%62)=NC(C%64=C(Cl)C═CC═C%64)=N%65)C%65=CC═C%61
ClC%66=C(C(N%67CCN(C(OC%68(C)C)═O)C%68C%67)=NC(C%69=C(F)C═CC═
C%69)=N%70)C%70=CC═C%66
ClC(C═CC═C1)═C1C2=NC3=CC═CC(C#C)═C3 C(N4CCN(C(OCC)═O)CC4)=N2
ClC(C═CC═C5)=C5C6=NC7=CC═CC(C═C)═C7C(N8CCN(C(OCC)═O)CC8)=N6
FC(C═CC═C9)=C9C%10═NC%11 ═CC═CC(CC)═C%11C(N%12CCN(C(C)═O)CC%12)=N%10
ClC(C═CC═C%13)=C%13C%14=NC%15=CC═CC(CCC)═C%15C(N%16CCN(C(OCC)═O)CC%16)=N%14
ClC(C═CC═C%17)=C%17C%18=NC%19=CC═CC(C(C)C)═C%19C(N%2OCCN(C(OCC)═O)CC%20)=N%18
ClC(C═CC═C%21)=C%21C%22=NC%23=CC═CC(C%24CC%24)=C%23C(N%25C CN(C(CC)═O)CC%25)=N%22
FC(C═CC═C%26)=C%26C%27=NC%28=CC═CC(CC%29CC%29)=C%28C(N%30C CN(C(OCC)═O)CC%30)=N%27
FC(C═CC═C%31)=C%31C%32=NC%33=CC═CC(C%34=NC═CS%34)=C%33C(N%35CCN(C(C)═O)CC%35)=N%32
ClC(C═CC═C%36)=C%36C%37=NC%38=CC═CC(C%39=NC═CN%39)=C%38C(N%4OCCN(C(OCC)═O)CC%40)=N%37
ClC(C═CC═C%41)=C%41C%42=NC%43=CC═CC(C%44=NC═CN%44C)═C%43C(N%45CCN(C(CCC)═O)CC%45)=N%42
ClC(C═CC═C%46)=C%46C%47=NC%48=CC═CC(C%49=CC═NC═C%49)=C%48C(N%5 OCCN(C(OC)═O)CC%50)=N%47
ClC(C═CC═C%51)=C%51 C%52=NC%53=CC═CC(C%54=CN═CC═C%54)=C%53C(N%55CCN(C(OCC)═O)CC%55)=N%52
FC(C═CC═C%56)=C%56C%57=NC%58=CC═CC(C%59=NC═CC═C%59)=C%58C(N%6OCCN(C(OCC)═O)CC%60)=N%57
ClC(C═CC═C%61)=C%61C%62=NC%63=CC═CC(C%64=NC═CC═N%64)=C%63C(N%65CCN(C(OC)═O)CC%65)=N%62
ClC1=C(C(N2CCN(C(OCC)═O)CC2)=NC(C3=C(Cl)C═C(Cl)C═C3)=N4)C4=CC═C1
ClC5=C(C(N6CCN(C(C)═O)CC6)=NC(C7=C(Cl)C═C(F)C═C7)=N8)C8=CC═C5
ClC9=C(C(N%1OCCN(C(OCC)═O)CC%10)=NC(C%11=CC(Cl)═C(Cl)C═C%11)=N%12)C%12=CC═C9
ClC%13=C(C(N%14CCN(C(C)═O)CC%14)=NC(C%15=CC(Cl)═C(Cl)C═C%15)=N%16)C%16=CC═C%13
ClC%17=C(C(N%18CCN(C(OCC)═O)CC%18)=NC(C%19=C(F)C═C(F)C═C% 19)=N%20)C%20=CC═C%7
ClC%21=C(C(N%22CCN(C(C)═O)CC%22)=NC(C%23=C(F)C═C(Cl)C═C%23)=N%24)C%24=CC═C%21
ClC%25C(C(N%26CCN(C(OCC)═O)CC%26)NC(C%27CC(F)C(F)CC%27) N%28)C%28=CC═C%25
ClC%29=C(C(N%3OCCN(C(C)═O)CC%30)=NC(C%31=CC(F)═C(F)C═C%31)=N%32)C%32=CC═C%29
FC%33C(C(N%34CCN(C(OCC)═O)CC%34)NC(C%35C(F)CC(Cl)CC%35) N%36)C%36=CC═C%33
FC%37=C(C(N%38CCN(C(C)═O)CC%38)=NC(C%39=C(Cl)C═C(F)C═C%39)=N%40)C%40=CC═C%37
FC%41=C(C(N%42CCN(C(OCC)═O)CC%42)=NC(C%43=CC(F)═C(F)C═C%43)=N %44)C%44=CC═C%41
FC%45=C(C(N%46CCN(C(C)═O)CC%46)=NC(C%47=CC(F)═C(F)C═C%47)=N%48)C%48=CC═C%45
FC%49=C(C(N%5OCCN(C(OCC)═O)CC%50)=NC(C%51=C(Cl)C═C(Cl)C═C%51)=N%52)C%52=CC═C%49
FC%53=C(C(N%54CCN(C(C)═O)CC%54)=NC(C%55=C(Cl)C═C(Cl)C═C%55)=N%56)C%56=CC═C%53
FC%57C(C(N%58CCN(C(OCC)═O)CC%58)NC(C%59CC(Cl)C(Cl)CC%59) N%60)C%60=CC═C%57
FC%61=C(C(N%62CCN(C(C)═O)CC%62)=NC(C%63=CC(Cl)=C(Cl)C═C%63)=N%64)C%64=CC═C%61

The activity of the compounds in Examples 1-357 as CB2 modulators is illustrated in the following assays. The other compounds listed above, which have not yet been made and/or tested, are predicted to have activity in these assays as well.

### Biological Activity Assays

### 1. Human CB2 Radioligand Binding Assay

This receptor binding filtration assay measures the receptor-ligand interactions of compounds by measuring ability to compete with a radiolabeled control ligand. Reactions were performed in 96 deep-well plate (Costar 3961) with a final volume of 600 ul. Compounds were added to each well either in single point (10 uM final) or dose-response. Compounds were delivered in DMSO or further diluted in incubation buffer to a final assay DMSO concentration of 1%. Incubation buffer (50 mM Tris-HCl pH 7.4, 2.5 mM EGTA, 5 mM MgCl₂, 5.0 mg/ml fatty acid free BSA) was added to each well. Following buffer addition, [³H] CP-55,940 was added. Tritiated CP-55,940 (Perkin Elmer) was used at the Kd determined from prior saturation binding experiments conducted on each lot of membranes. Membrane preparations from stably transfected CHO CB2 cells were added and the reaction was incubated for 90 minutes at 30° C. Unifilter GF/C filtration plates (Perkin Elmer) were pre-wet with 0.05% polyethylenimine. Samples were transferred to Unifilter plates and separation of unbound radioligand was achieved using a vacuum manifold (Millipore). Following thorough washing with 5 ml/well ice-cold wash buffer (50 mM Tris-HCl pH 7.4, 2.5 mM EGTA, 5 mM MgCl₂, 0.5 mg/ml fatty acid free BSA), filterplates were dried completely. Betaplate Scint Scintillation cocktail (25 ul, Perkin Elmer) was applied and plates were read using Microbeta Trilux (Wallac). Data analysis was performed in Spotfire. Activity of the positive control (100 nM CP-55,940) was set as 100% efficacy.

### 2. Human CB1 Radioligand Binding Assay

The CB1 binding assay was performed as described above except that HEK 293 EBNA cells expressing human cannabinoid receptor type 1 (Perkin Elmer cat# RBHCB1M) were used according the manufacturer's instructions. The incubation buffer was composed of 50 mM Tris-HCl pH 7.4, 2.5 mM EDTA, 5 mM MgCl₂, 5.0 mg/ml fatty acid free BSA and the wash buffer was composed of 50 mM Tris-HCl pH 7.4, 2.5 mM EDTA, 5 mM MgCl₂, 0.5 mg/ml fatty acid free BSA. Activity of the positive control (100 nM CP-55,940) was set as 100% efficacy.

### Membrane Preparation

Stable recombinant Human CB2-CHO cell membranes were prepared as follows: Chinese Hamster Ovarian (CHO) cells stably expressing human cannabinoid receptor type 2 were grown to 90% confluence in 15×100 mm culture dishes under puromycin selection (5 ug/ml) Cells were harvested from culture flasks using a cell scraper, were washed once with cold phosphate-buffered saline (calcium and magnesium free) and pelleted by centrifugation at 400 g for 5 min at 4° C. Cell pellet was washed once with cold phosphate-buffered saline and centrifuged again at 400×g for 5 min at 4° C. The pellet was suspended in ice-cold lysis buffer (10 mM Tris-HCl, 0.1 mM EDTA, containing 0.32 mM sucrose, pH 7.5) and homogenized in a chilled 7 ml glass dounce homogenizer using 50 strokes. The homogenate was centrifuged at 400 g for 15 min at 4° C. The cloudy supernatant was collected and centrifuged at 41000 g for 30 min at 4° C. The resulting pellet was washed with ice-cold sucrose-free lysis buffer and centrifuged again at 41000 g for 30 min at 4° C. The membrane pellet was suspended in sucrose-free storage buffer (10 mm Tris-HCl, 0.1 mM EDTA, pH 7.5) to a concentration of 2-3 mg protein/ml. Aliquots were flash frozen in liquid nitrogen and stored at −80° C. Concentration was determined using Dc protein assay kit (Bio-Rad).

TABLE 1
In Vitro Biological Activity Assays

| | CB2 Ligand Binding | |
|---|---|---|
| | Asssay | Selectivity, CB2 vs. CB1 |
| | + indicates EC₅₀ < 1 μM | + indicates > 10−fold |
| Example # | − indicates EC₅₀ ≧ 1 μM | − indicates ≦ 10−fold |
| 1 | + | + |
| 2 | + | + |
| 3 | + | + |
| 4 | + | − |
| 5 | + | + |
| 6 | − | + |
| 7 | + | + |
| 8 | − | + |
| 9 | − | − |
| 10 | − | + |
| 11 | − | − |
| 12 | − | − |
| 13 | + | − |
| 14 | − | − |
| 15 | + | − |
| 16 | − | − |
| 17 | − | − |
| 18 | + | + |
| 19 | + | + |
| 20 | + | + |
| 21 | − | − |
| 22 | + | − |
| 23 | − | − |
| 24 | + | − |
| 25 | − | − |
| 26 | − | − |
| 27 | + | − |
| 28 | − | + |
| 29 | + | + |
| 30 | − | + |
| 31 | − | − |
| 32 | − | − |
| 33 | + | − |
| 34 | + | − |
| 35 | + | − |
| 36 | + | + |
| 37 | − | − |
| 38 | − | − |
| 39 | + | − |
| 40 | − | − |
| 41 | − | − |
| 42 | + | + |
| 43 | + | + |
| 44 | + | − |
| 45 | + | + |
| 46 | + | + |
| 47 | NT | ND |
| 48 | − | − |
| 49 | − | − |
| 50 | − | − |
| 51 | − | − |
| 52 | − | − |
| 53 | − | + |
| 54 | + | + |
| 55 | + | − |
| 56 | − | − |
| 57 | + | + |
| 58 | + | − |
| 59 | + | − |
| 60 | + | − |
| 61 | + | − |
| 62 | + | + |
| 63 | + | − |
| 64 | + | − |
| 65 | + | − |
| 66 | + | − |
| 67 | + | − |
| 68 | + | − |
| 69 | − | − |
| 70 | − | − |
| 71 | − | − |
| 72 | − | − |
| 73 | − | − |
| 74 | − | − |
| 75 | − | − |
| 76 | − | − |
| 77 | − | − |
| 78 | + | − |
| 79 | + | − |
| 80 | + | − |
| 81 | − | − |
| 82 | − | − |
| 83 | − | − |
| 84 | − | − |
| 85 | − | − |
| 86 | + | − |
| 87 | − | − |
| 88 | − | − |
| 89 | − | − |
| 90 | − | − |
| 91 | − | − |
| 92 | − | − |
| 93 | − | + |
| 94 | − | − |
| 95 | + | + |
| 96 | + | + |
| 97 | + | + |
| 98 | + | + |
| 99 | + | + |
| 100 | − | − |
| 101 | NT | ND |
| 102 | − | − |
| 103 | + | − |
| 104 | + | − |
| 105 | + | + |
| 106 | + | + |
| 107 | − | − |
| 108 | + | + |
| 109 | + | + |
| 110 | + | + |
| 111 | + | − |
| 112 | + | − |
| 113 | − | − |
| 114 | + | + |
| 115 | − | − |
| 116 | − | − |
| 117 | + | − |
| 118 | NT | ND |
| 119 | − | − |
| 120 | − | − |
| 121 | − | − |
| 122 | + | + |
| 123 | − | − |
| 124 | − | − |
| 125 | + | − |
| 126 | + | + |
| 127 | − | − |
| 128 | + | + |
| 129 | + | − |
| 130 | + | + |
| 131 | + | − |
| 132 | + | − |
| 133 | − | − |
| 134 | + | − |
| 135 | − | − |
| 136 | − | − |
| 137 | − | − |
| 138 | + | + |
| 139 | + | − |
| 140 | + | − |
| 141 | − | − |
| 142 | + | − |
| 143 | − | − |
| 144 | − | − |
| 145 | − | − |
| 146 | − | − |
| 147 | − | − |
| 148 | − | − |
| 149 | − | − |
| 150 | + | − |
| 151 | + | − |
| 152 | + | + |
| 153 | + | + |
| 154 | + | + |
| 155 | − | − |
| 156 | − | − |
| 157 | + | + |
| 158 | − | − |
| 159 | − | + |
| 160 | + | − |
| 161 | + | − |
| 162 | + | + |
| 163 | + | − |
| 164 | + | + |
| 165 | − | + |
| 166 | + | + |
| 167 | + | + |
| 168 | + | + |
| 169 | + | + |
| 170 | + | + |
| 171 | + | + |
| 172 | + | + |
| 173 | + | + |
| 174 | + | + |
| 175 | + | − |
| 176 | + | + |
| 177 | + | − |
| 178 | + | − |
| 179 | + | + |
| 180 | + | − |
| 181 | − | − |
| 182 | − | − |
| 183 | + | − |
| 184 | + | − |
| 185 | + | − |
| 186 | + | − |
| 187 | − | − |
| 188 | − | − |
| 189 | − | − |
| 190 | − | − |
| 191 | − | − |
| 192 | − | − |
| 193 | − | − |
| 194 | + | − |
| 195 | − | − |
| 196 | − | − |
| 197 | + | + |
| 198 | − | − |
| 199 | − | − |
| 200 | − | − |
| 201 | + | + |
| 202 | + | − |
| 203 | − | − |
| 204 | + | − |
| 205 | + | + |
| 206 | − | − |
| 207 | + | − |
| 208 | + | + |
| 209 | − | − |
| 210 | − | − |
| 211 | − | − |
| 212 | − | − |
| 213 | NT | ND |
| 214 | NT | ND |
| 215 | NT | ND |
| 216 | NT | ND |
| 217 | + | + |
| 218 | − | − |
| 219 | + | + |
| 220 | − | − |
| 221 | + | + |
| 222 | + | − |
| 223 | − | − |
| 224 | − | − |
| 225 | + | + |
| 226 | + | + |
| 227 | + | − |
| 228 | − | − |
| 229 | − | − |
| 230 | − | − |
| 231 | − | − |
| 232 | + | + |
| 233 | + | − |
| 234 | − | − |
| 235 | − | − |
| 236 | − | − |
| 237 | + | + |
| 238 | + | + |
| 239 | + | + |
| 240 | + | + |
| 241 | + | + |
| 242 | + | + |
| 243 | + | + |
| 244 | + | + |
| 245 | + | + |
| 246 | + | + |
| 247 | + | + |
| 248 | + | + |
| 249 | + | + |
| 250 | + | + |
| 251 | + | + |
| 252 | − | − |
| 253 | − | − |
| 254 | + | − |
| 255 | + | + |
| 256 | + | + |
| 257 | − | − |
| 258 | − | − |
| 259 | − | − |
| 260 | + | + |
| 261 | + | + |
| 262 | − | − |
| 263 | − | − |
| 264 | − | − |
| 265 | + | + |
| 266 | + | + |
| 267 | + | + |
| 268 | + | − |
| 269 | + | + |
| 270 | − | − |
| 271 | + | − |
| 272 | − | − |
| 273 | + | − |
| 274 | + | − |
| 275 | − | + |
| 276 | − | − |
| 277 | − | − |
| 278 | + | − |
| 279 | + | + |
| 280 | + | + |
| 281 | + | + |
| 282 | + | + |
| 283 | − | − |
| 284 | − | − |
| 285 | − | − |
| 286 | + | + |
| 287 | + | + |
| 288 | + | + |
| 289 | + | − |
| 290 | + | + |
| 291 | NT | ND |
| 292 | + | + |
| 293 | − | − |
| 294 | − | − |
| 295 | − | − |
| 296 | − | − |
| 297 | − | − |
| 298 | − | − |
| 299 | − | + |
| 300 | + | + |
| 301 | − | − |
| 302 | − | − |
| 303 | − | − |
| 304 | + | − |
| 305 | + | − |
| 306 | NT | ND |
| 307 | + | − |
| 308 | + | − |
| 309 | + | + |
| 310 | + | − |
| 311 | + | + |
| 312 | + | − |
| 313 | + | − |
| 314 | − | + |
| 315 | − | − |
| 316 | + | + |
| 317 | − | − |
| 318 | − | − |
| 319 | − | − |
| 320 | − | − |
| 321 | − | − |
| 322 | − | − |
| 323 | − | − |
| 324 | − | − |
| 325 | − | − |
| 326 | − | − |
| 327 | − | − |
| 328 | − | − |
| 329 | − | + |
| 330 | − | − |
| 331 | − | − |
| 332 | − | − |
| 333 | + | − |
| 334 | + | − |
| 335 | − | − |
| 336 | − | + |
| 337 | + | − |
| 338 | − | + |
| 339 | − | − |
| 340 | − | + |
| 341 | + | − |
| 342 | − | − |
| 343 | − | − |
| 344 | + | + |
| 345 | + | + |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound having structural Formula I: 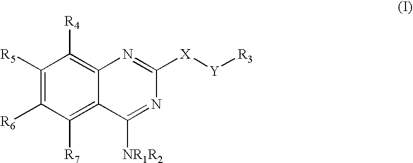
or a salt, ester, or prodrug thereof, wherein:
X is (CR₈R₉)ₙ;
Y is selected from the group consisting of —NR₁₀, —OR₁₀, and a bond;
n is an integer from 0 to 3;
R₁ and R₂ are each independently selected from the group consisting of hydrogen, acyl, alkyl, arylalkyl, C-amido, aryl, cycloalkyl, cycloalkylalkyl, heteroalkyl, heteroaryl, heteroarylalkyl, heterocycloalkyl, heterocycloalkylalkyl, perhaloalkyl, sulfonyl, and S-sulfonamide, any of which may be optionally substituted; or R¹ and R² together may form heterocycloalkyl or heteroaryl, which may be optionally substituted;
R₃ is selected from the group consisting of hydrogen, null, —C(O)R₁₁, —C(O)NHR₁₁, —S(O)₂R₁₁, —S(O₂)NHR₁₁, aryl, heteroaryl, cycloalkyl, alkyl, alkoxy, amino, aryloxy, carbamate, carboxy, heteroalkyl, haloalkyl, haloalkoxy, heterocycloalkyl, perhaloalkoxy, and perhaloalkyl, any of which may be optionally substituted;
R₄, R₅, R₆, and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₈ and R₉ are each independently selected from the group consisting of hydrogen, lower alkoxy, lower alkyl, lower perhaloalkyl, and halogen, any of which may be optionally substituted, or R₈ and R₉, taken together, are oxy or optionally substituted cycloalkyl;
R₁₀ is selected from the group consisting of hydrogen, —C(O)R₁₁, —C(O)NHR₁₁, —S(O)₂R₁₁, —S(O₂)NHR₁₁, lower alkyl, aryl, cycloalkyl, lower heteroalkyl, heteroaryl, heterocycloalkyl, and perhaloalkyl, any of which may be optionally substituted; or R³ and R¹⁰ together may form heterocycloalkyl which may be optionally substituted; and
R₁₁ is selected from the group consisting of aryl, cycloalkyl, heterocycloalkyl, and heteroaryl, any of which may be optionally substituted.

2. The compound as recited in claim 1, wherein:
n is 1;
R₈ and R₉ are hydrogen; and
Y is NR₁₀.

3. The compound as recited in claim 2, wherein:
R₁₀ is hydrogen; and
R₃ is —SO₂R₁₁.

4. The compound as recited in claim 3, having structural Formula II: 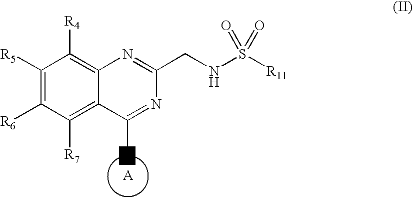
or a salt, ester, or prodrug thereof, wherein:
A is a five- to seven-membered monocyclic heterocycloalkyl which may be optionally substituted;
R₄, R₅, R₆, and R₇ are each independently selected from the group consisting of lower alkyl, lower alkoxy, cyano, cycloalkyl, halogen, hydrogen, hydroxy, lower perhaloalkoxy, lower perhaloalkyl; and
R₁₁ is selected from the group consisting of aryl and heteroaryl, any of which may be optionally substituted.

5. The compound as recited in claim 4, wherein R₁₁ is optionally substituted naphthyl.

6. The compound as recited in claim 1, wherein:
n is an integer from 0 to 1;
Y is a bond;
R₃ is selected from the group consisting of aryl, heteroaryl, cycloalkyl, and alkyl, any of which may be optionally substituted; and
R₈ and R₉ are each independently selected from the group consisting of hydrogen and lower alkyl.

7. The compound as recited in claim 6, wherein n is 0.

8. The compound as recited in claim 7, wherein R₃ is phenyl, which may be optionally substituted by one or more substituents selected from the group consisting of hydrogen, halogen, hydroxy, cyano, nitro, alkyl, haloalkyl, perhaloalkyl, alkoxy, haloalkoxy, perhaloalkoxy, acyloxy, acyl, carboxyl, amino, alkylamino, arylamino, C-amido, N-amido, carbamate, urea, N-sulfonamido, S-sulfonamido, alkylsulfonyl, thiol, and alkylthio.

9. The compound as recited in claim 8, wherein R₁ and R₂ are each independently selected from the group consisting of hydrogen, aryl, arylalkyl, cycloalkyl, cycloalkylalkyl, heteroalkyl, heteroarylalkyl, heterocycloalkylalkyl, and lower alkyl; or R¹ and R² together may form heterocycloalkyl or heteroaryl, which may be optionally substituted.

10. The compound as recited in claim 9, wherein R¹ and R² together form a heterocycloalkyl or heteroaryl, which may be optionally substituted by one or more substituents selected from the group consisting of carbamate, cycloalkyl, C-amido, lower perhaloalkyl, acyl, aryl, alkylsulfonyl, haloalkylalkyl, heteroaryl, and heterocycloalkyl.

11. The compound as recited in claim 10, wherein R¹ and R² together form a 5- or 6-membered monocyclic heterocycloalkyl, which may be optionally substituted by one or more substituents selected from the group consisting of carbamate, cycloalkyl, C-amido, lower perhaloalkyl, acyl, aryl, alkylsulfonyl, haloalkylalkyl, heteroaryl, and heterocycloalkyl.

12. A compound as recited in claim 11, having structural Formula III: 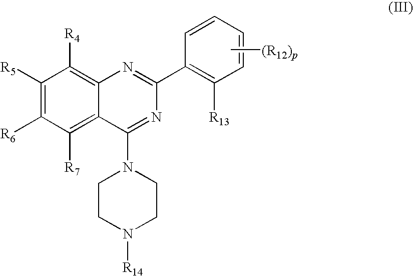
or a salt, ester, or prodrug thereof, wherein:
p is an integer from 0 to 4;
R₄, R₅, R₆, R₁₂, and R₁₃ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₇ is selected from the group consisting of halogen, hydroxy, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and
R₁₄ is selected from the group consisting of carbamate, cycloalkyl, C-amido, perhaloalkyl, acyl, aryl, alkylsulfonyl, haloalkylalkyl heteroaryl, any of which may be optionally substituted.

13. The compound as recited in claim 12, wherein:
R₄ is hydrogen;
R₅ and R₆ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxy, amino, alkylamino, thiol, and alkylthio; and
R₇ is selected from the group consisting of halogen, hydroxy, cyano, nitro, C₂-C₆ alkyl, lower alkenyl, lower alkynyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

14. The compound as recited in claim 13, wherein:
R₅ and R₆ are each independently selected from the group consisting of hydrogen, lower alkoxy, hydroxy, halogen, and lower perhaloalkyl;
R₇ is selected from the group consisting of selected from the group consisting of lower alkoxy, lower alkenyl, lower alkynyl, hydroxy, halogen, and lower perhaloalkyl; and
R₁₂ and R₁₃ are each independently selected from the group consisting of hydrogen, halogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

15. The compound as recited in claim 14, wherein:
R₁₂ is selected from the group consisting of hydrogen, halogen, and lower perhaloalkyl; and
R₁₃ is halogen.

16. The compound as recited in claim 15, wherein R₁₄ is selected from the group consisting of carbamate and acyl.

17. A compound as recited in claim 11, having structural Formula III: 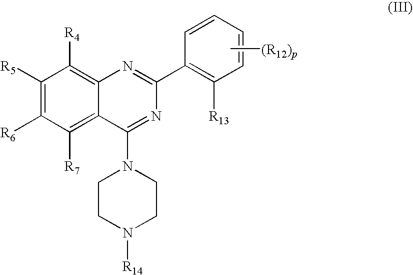
or a salt, ester, or prodrug thereof, wherein:
p is an integer from 0 to 4;
R₄ and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₅ is selected from the group consisting of hydrogen, hydroxy, cyano, nitro, C₂-C₆ alkyl, haloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₆ is selected from the group consisting of hydrogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₁₂ is selected from the group consisting of halogen, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, C₁-C₅ alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₁₃ is selected from the group consisting of hydrogen, halogen, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and
R₁₄ is selected from the group consisting of straight-chain carbamate, lower perhaloalkyl, straight-chain alkylacyl, haloalkylalkyl, heteroaryl, and heterocycloalkyl, any of which may be optionally substituted.

18. The compound as recited in claim 17, wherein:
R₄ is hydrogen;
R₅ is selected from the group consisting of hydrogen, hydroxy, cyano, nitro, C₂-C₆ alkyl, haloalkyl, lower alkoxy, lower haloalkoxy, lower perhaloalkoxy, acyloxy, acyl, amino, alkylamino, thiol, and alkylthio;
R₆ is selected from the group consisting of hydrogen, hydroxy, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and
R₇ is selected from the group consisting of hydrogen, halogen, hydroxy, cyano, nitro, lower alkyl, lower alkenyl, lower alkynyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

19. The compound as recited in claim 18, wherein:
R₅ is selected from the group consisting of hydrogen, lower alkoxy, and hydroxy;
R₆ is selected from the group consisting of hydrogen, lower alkoxy, hydroxy, and lower perhaloalkyl;
R₇ is selected from the group consisting of hydrogen, lower alkoxy, lower alkenyl, lower alkynyl, hydroxy, halogen, and lower perhaloalkyl;
R₁₂ is selected from the group consisting of halogen, cyano, nitro, C₂-C₆ alkyl, lower haloalkyl, lower perhaloalkyl, C₁-C₅ alkoxy, lower haloalkoxy, lower perhaloalkoxy, acyloxy, acyl, carboxyl, amino, alkylamino, thiol, and alkylthio; and
R₁₃ is selected from the group consisting of hydrogen, halogen, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio.

20. The compound as recited in claim 19, wherein:
R₁₂ is selected from the group consisting of halogen and lower perhaloalkyl; and
R₁₃ is halogen.

21. The compound as recited in claim 20, wherein R₁₄ is selected from the group consisting of straight-chain carbamate and acyl.

22. A compound as recited in claim 11, having structural Formula III: 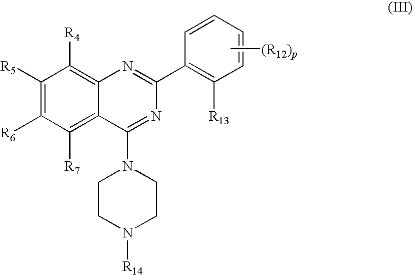
or a salt, ester, or prodrug thereof, wherein:
p is an integer from 0 to 4;
R₄, R₆, and R₇ are each independently selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₅ is selected from the group consisting of hydrogen, halogen, hydroxy, cyano, nitro, C₂-C₆ alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₁₂ is selected from the group consisting of hydrogen, halogen, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxy, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted;
R₁₃ is selected from the group consisting of halogen, cyano, nitro, alkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, C₂-C₆ alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl, any of which may be optionally substituted; and
R₁₄ is selected from the group consisting of straight-chain carbamate, cycloalkyl, C-amido, C₂-C₆ alkyl, lower perhaloalkyl, acyl, aryl, arylsulfonyl, haloalkylalkyl, heteroaryl, and heterocycloalkyl, any of which may be optionally substituted.

23. The compound as recited in claim 22, wherein:
R₄ is hydrogen;
R₅ is selected from the group consisting of hydrogen, halogen, hydroxy, cyano, nitro, C₂-C₆ alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and
R₆ and R₇ are each independently selected from the group consisting of hydrogen, halogen, hydroxy, cyano, nitro, lower alkyl, lower alkenyl, lower alkynyl, lower haloalkyl, lower perhaloalkyl, lower alkoxy, lower haloalkoxy, lower perhaloalkoxy, acyloxy, acyl, carboxy, amino, alkylamino, thiol, and alkylthio.

24. The compound as recited in claim 23, wherein:
R₅, R₆, and R₇ are each independently selected from the group consisting of hydrogen, lower alkoxy, lower alkenyl, lower alkynyl, hydroxy, halogen, and lower perhaloalkyl;
R₁₂ is selected from the group consisting of hydrogen, halogen, cyano, nitro, lower alkyl, lower haloalkyl, lower perhaloalkyl, lower alkoxy, lower haloalkoxy, lower perhaloalkoxy, acyloxy, acyl, carboxyl, amino, alkylamino, thiol, and alkylthio; and
R₁₃ is selected from the group consisting of halogen, cyano, nitro, lower alkyl, lower perhaloalkyl, C₂-C₆ alkoxy, haloalkoxy, lower perhaloalkoxy, acyloxy, acyl, carboxy, amino, alkylamino, thiol, and alkylthio.

25. The compound as recited in claim 24, wherein:
R₁₂ is selected from the group consisting of halogen and lower perhaloalkyl; and
R₁₃ is halogen.

26. The compound as recited in claim 25, wherein R₁₄ is selected from the group consisting of straight-chain carbamate and acyl.

27. A compound, as recited in claim 11, having structural Formula III: 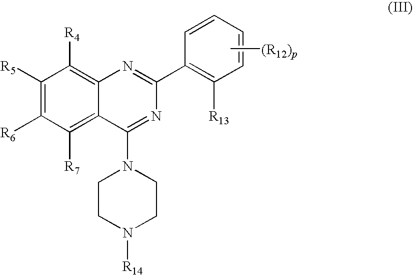
or a salt, ester, or prodrug thereof, wherein:
R₇ and R₁₃ are each independently selected from the group consisting of halogen, hydroxy, lower alkoxy, lower alkenyl, and lower alkynyl; and
R₁₄ is selected from the group consisting of carbamate, cycloalkyl, C-amido, lower perhaloalkyl, acyl, aryl, alkylsulfonyl, haloalkylalkyl, heteroaryl, and heterocycloalkyl, any of which may be optionally substituted.

28. The compound as recited in claim 27, wherein R₁₄ is selected from the group consisting of carbamate and acyl.

29. The compound as recited in claim 28, wherein R₇ and R₁₃ are each independently halogen.

30. A compound selected from the group consisting of Examples 1 to 345.

31. A pharmaceutical composition comprising a compound as recited in claim 1 together with a pharmaceutically acceptable carrier.

32. A method of modulation of CB2 comprising contacting CB2 with a compound as recited in claim 1.

33. A method of treatment of a CB2-mediated disease comprising the administration of a therapeutically effective amount of a compound as recited in claim 1 to a patient in need thereof.

34. The method as recited in claim 33 wherein said disease is selected from the group consisting of acute nociceptive pain, chronic nociceptive pain, neuropathic pain, inflammatory pain, abdominal pain, acute herpes zoster, postherpetic neuralgia, fibromyalgia, ocular pain, muscle spasm, neuromuscular disorder, atherosclerosis progression, tactile allodynia, hyperalgesia, post-surgical pain, bone fracture pain, dental pain, bunionectomy, muscular pain, mastalgia, pain from dermal injuries, lower back pain, headaches, migraine, osteoarthritis, musculoskeletal conditions, cancer pain, reflex sympathetic dystrophy/causalgia, peripheral neuropathy, diabetic neuropathy, complex regional pain syndrome, entrapment neuropathy, multiple sclerosis, rheumatoid arthritis, systemic lupus erythematosus, myasthenia gravis, autoimmune disease, malabsorption syndrome, pulmonary disease, osteoporosis, atherosclerosis, diabetes mellitus type I, inflammatory bowel disease, irritable bowel syndrome, psoriasis, tissue rejection in organ transplants, celiac disease, asthma, glaucoma, Sjogren's syndrome, chronic liver disease, acute liver disease, liver fibrosis, ischemia-reperfusion injury, hepatic encephalopathy and non-alcoholic fatty liver disease (NAFLD).

35. A method of treatment of a CB2-mediated disease comprising the administration of:
a. a therapeutically effective amount of a compound as recited in claim 1; and
b. another therapeutic agent.

36. A method for achieving an effect in a patient comprising the administration of a therapeutically effective amount of a compound as recited in claim 1 to a patient, wherein the effect is selected from the group consisting of anti-emesis, enhancement of appetite, vascular hypotension, immunomodulation, analgesia, treatment of muscle spasm, treatment of neuromuscular disorders, treatment of osteoporosis, and treatment of atherosclerosis.
